(19) United States
(12) Patent Application Publication
(10) Pub. No.: US 2025/0367209 A1
(43) Pub. Date: Dec. 4, 2025

(54) IDENTIFICATION OF NOVEL BENZOTHIAZONES AS TAU-SH3 INTERACTION INHIBITORS FOR THE TREATMENT OF ALZHEIMER’S DISEASE

(71) Applicants: SOUTHERN RESEARCH INSTITUTE, Birmingham, AL (US); THE UAB RESEARCH FOUNDATION, Birmingham, AL (US)

(72) Inventors: Corinne E. AUGELLI-SZAFRAN, Homewood, AL (US); Vibha PATHAK, Hoover, AL (US); Mark J. SUTO, Homewood, AL (US); Erik D. ROBERSON, Vestavia Hills, AL (US)

(21) Appl. No.: 18/876,144

(22) PCT Filed: Jun. 21, 2023
(86) PCT No.: PCT/US2023/025846

§ 371 Date: Dec. 17, 2024

Related U.S. Application Data
(60) Provisional application No. 63/354,425, filed on Jun. 22, 2022.

(51) Int. Cl. A61K 31/5377; C07D 401/14; C07D 413/14; C07D 417/12; C07D 417/14

(52) U.S. Cl. CPC A61K 31/5377; C07D 401/14; C07D 413/14; C07D 417/12; C07D 417/14

(57) ABSTRACT

The present disclosure is concerned with benzothiazone compounds that are capable of inhibiting Tau-SH3 signaling. The present disclosure is also concerned with methods of using these compounds for the treatment of neurological disorders such as, for example, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD). This abstract is intended as a scanning tool for purposes of searching in the particular art and is not intended to be limiting of the present invention.

### CROSS-REFERENCE TO RELATED APPLICATIONS

This Application claims the benefit of U.S. Application No. 63/354,425, filed on Jun. 22, 2022, the contents of which are incorporated herein by reference in their entirety.

### STATEMENT REGARDING FEDERALLY SPONSORED RESEARCH

This invention was made with government support under grant numbers IS10RR026478, R01NS075487. RFIAG059405, ULITR001417, P20AG068024, T32NS061455, T32NS095775, and F31AG064868 awarded by the National Institutes of Health, grant number OAC-1541310 awarded by the National Science Foundation, and grant number A2015693S awarded by the BrightFocus Foundation. The government has certain rights in the invention.

### BACKGROUND

Alzheimer's disease (AD) is the leading cause of dementia worldwide and age is the greatest risk factor, so its impact is increasing as the global population ages. Early evidence providing a mechanistic link between amyloid-β (Aβ) and Tau, the two proteins most implicated by AD pathology, was that primary neurons from Tau knockout mice are protected from Aβ-induced neurite degeneration (Rapoport, et al. (2002) Proc. Natl. Acad. Sci. USA 99, 6364-6369). This protective effective of Tau reduction translated to mouse models of AD (Roberson, et al. (2007) Science 316, 750-754; Ittner, et al. (2010) Cell 142, 387-397; Roberson, et al. (2011) J Neurosci 31, 700-11; Leroy, et al. (2012) Am. J. Pathol. 181, 1928-1940; Meilandt, et al. (2008) The Journal of Neuroscience 28, 5007; Nussbaum, et al. (2012) Nature 485, 651-655; Palop, et al. (2007) Neuron 55, 697-711), preventing both cognitive deficits and network hyperexcitability without affecting Aβ plaque load, indicating that Tau is a key downstream regulator of Aβ toxicity. These findings demonstrate that targeting Tau could be highly effective for preventing Aβ-induced neuronal dysfunction, and ongoing studies of Tau reduction suggest that its beneficial effects may even extend beyond AD. Tau reduction confers resistance to hyperexcitability and seizures induced by Aβ (Ittner, et al. (2010) Cell 142, 387-397; Roberson, et al. (2011) J Neurosci 31, 700-11) and also induces resistance to hyperexcitability outside the context of Aβ, with protective effects against hyperexcitability in models of epilepsy, Parkinson's disease, and autism (DeVos, et al. (2013) J. Neurosci. 33, 12887-12897; DeVos, et al. (2017) Sci Transl Med 9; Li, et al. (2014) Neurobiol. Aging 35, 2617-2624; Gheyara, et al. (2014) Ann. Neurol. 76, 443-456; Holth, et al. (2013) J Neurosci. 33, 1651-1659; Singh, B. et al. Tau is required for progressive synaptic and memory deficits in a transgenic mouse model of alpha-synucleinopathy. Acta Neuropathol (2019); Tai, C. et al. Tau Reduction Prevents Key Features of Autism in Mouse Models. Neuron (2020)). Thus, therapies harnessing the mechanisms underlying Tau reduction may be beneficial in multiple neurological conditions.

Diverse evidence suggests that Tau's interactions with SH3-domain containing proteins are important for Aβ-induced pathophysiology. One extensively studied Tau-SH3 interaction is Tau's interaction with Fyn kinase. Fyn's SH3 domain directly binds Tau's proline-rich domain, and the affinity of their interaction is increased when Tau is hyperphosphorylated (Lee, et al. (1998) J Cell Sci. 111, 3167-3177; Ittner, et al. (2010) Cell 142, 387-397; Reynolds, et al. (2008) J Biol. Chem. 283, 18177-86). Fyn levels are increased in AD brain (Shirazi and Wood (1993) Neuroreport 4, 435-437; Ho, et al. (2005) Neurobiol. Aging 26, 625-635 (2005)) and Aβ increases Fyn levels at the synapse and activates its kinase activity (Ittner, et al. (2010) Cell 142, 387-397). Tau reduction (Rapoport, et al. (2002) Proc. Natl. Acad. Sci. USA 99, 6364-6369; Roberson, et al. (2007) Science 316, 750-754; Rush, et al. (2020) Neurobiol Dis 134, 104668) and Fyn reduction (Lambert, et al. (1998) Proc. Natl. Acad. Sci. USA 95, 6448-6453; Chin, et al. (2004) J. Neurosci. 24, 4692-4697) produce convergent phenotypes, each protecting against AD both in vivo and in cultured neurons. Tau reduction also reduces Fyn levels at the synapse, which could counteract the synaptic increases in AD models (Ittner, et al. (2010) Cell 142, 387-397). Additionally, Fyn overexpression exacerbates cognitive deficits in an AD mouse model, and these Fyn-mediated deficits are ameliorated by Tau reduction (Roberson, et al. (2011) J Neurosci 31, 700-11). Finally, a truncated form of Tau, which binds Fyn but prevents its dendritic localization, prevents cognitive deficits and network hyperexcitability in an AD mouse model (Ittner, et al. (2010) Cell 142, 387-397). Beyond Fyn, there are multiple SH3 domain-containing proteins implicated in AD that bind Tau, such as the genetic risk factor BIN1, which also controls network hyperexcitability (Voskobiynyk, et al. (2020) eLife 9, e57354). Tau may act postsynaptically as a scaffold for its interactors, permitting Aβ to transduce toxic signaling into neurons. Thus, it is theorized that inhibiting Tau-SH3 interactions will reduce Aβ-induced neuronal dysfunction.

Despite these implications, the development of small molecule therapeutics capable of selectively targeting Tau-SH3 have remained elusive. These needs and others are met by the present invention.

### SUMMARY

In accordance with the purpose(s) of the invention, as embodied and broadly described herein, the invention, in one aspect, relates to compounds and compositions for use in the prevention and treatment of neurological disorders such as, for example, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD).

Disclosed are compounds having a structure represented by a formula:

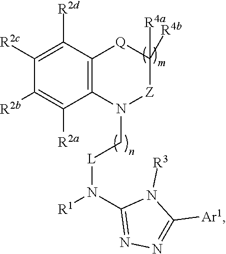

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

Also disclosed are compounds having a structure represented by a formula:

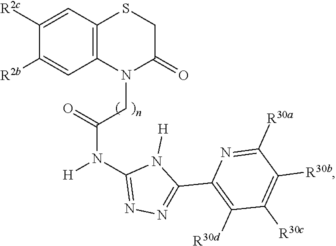

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

Also disclosed are compounds selected from the group consisting of.

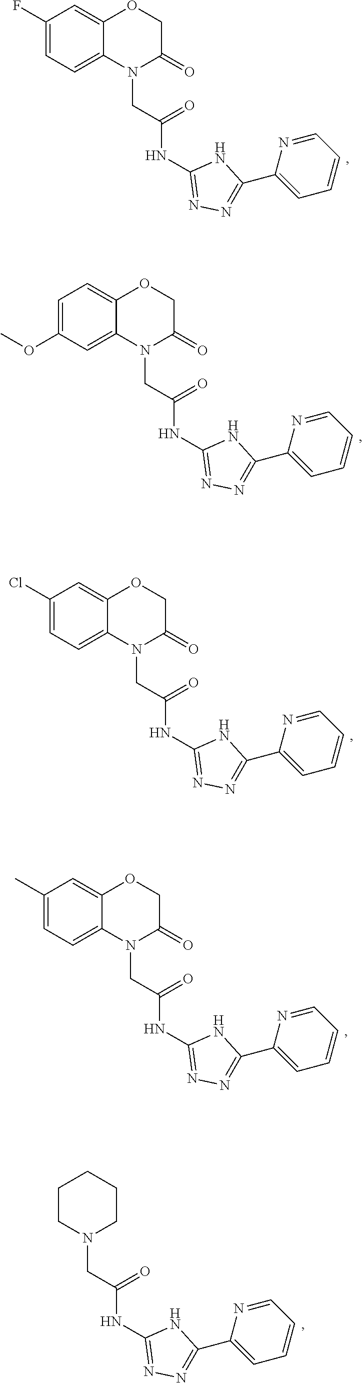 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1)Nc1nnc(-c2ccccn2)[nH]1]

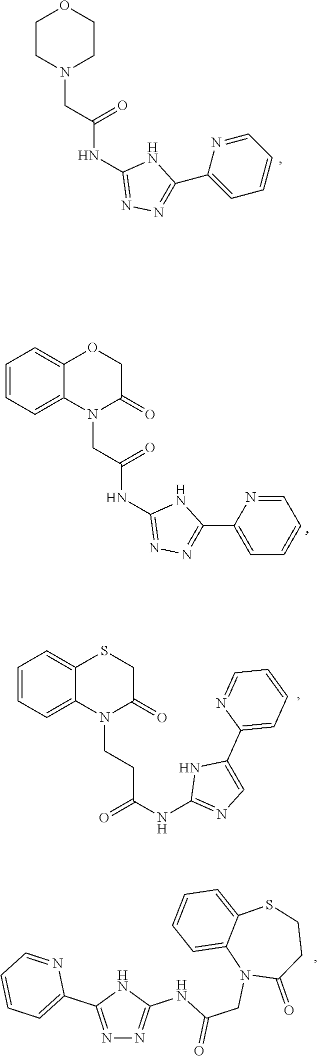 [SMILES: O=C(CN1CCOCC1)Nc1nnc(-c2ccccn2)[nH]1 ; O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1]

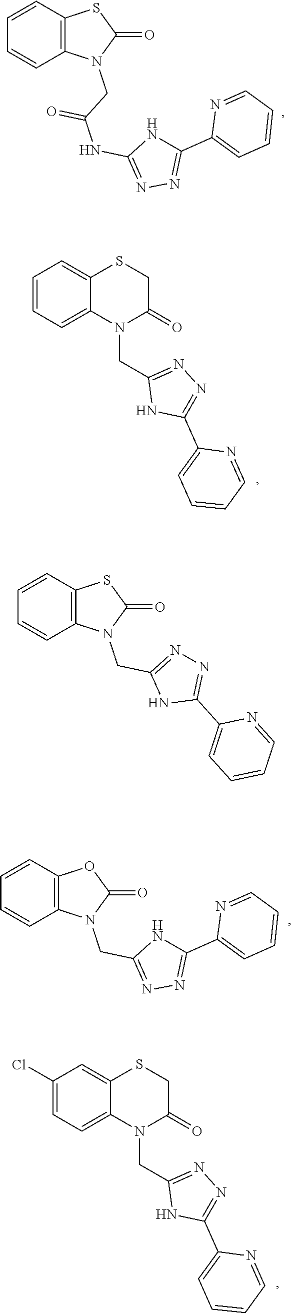 [SMILES: O=C(Cn1c(=O)sc2ccccc21)Nc1nnc(-c2ccccn2)[nH]1 ; O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1 ; O=C1CSc2cc(Cl)ccc2N1Cc1nnc(-c2ccccn2)[nH]1]

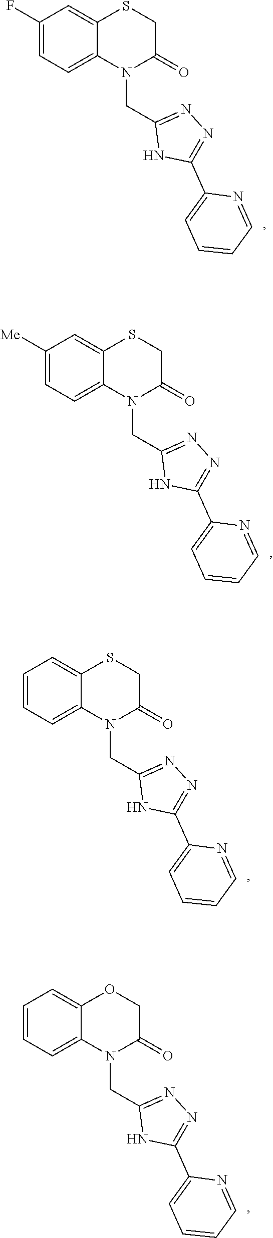 [SMILES: O=C1CSc2cc(F)ccc2N1Cc1nnc(-c2ccccn2)[nH]1 ; Cc1ccc2c(c1)SCC(=O)N2Cc1nnc(-c2ccccn2)[nH]1 ; O=C1CSc2ccccc2N1Cc1nnc(-c2ccccn2)[nH]1 ; O=C1COc2ccccc2N1Cc1nnc(-c2ccccn2)[nH]1]

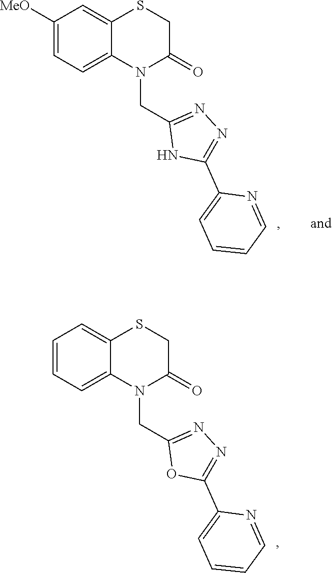 [SMILES: COc1ccc2c(c1)SCC(=O)N2Cc1nnc(-c2ccccn2)[nH]1 ; O=C1CSc2ccccc2N1Cc1nnc(-c2ccccn2)o1]

or a pharmaceutically acceptable salt thereof.

Also disclosed are pharmaceutical compositions comprising a therapeutically effective amount of at least one disclosed compound and a pharmaceutically acceptable carrier.

Also disclosed are pharmaceutical compositions comprising an effective amount of a compound having a structure:

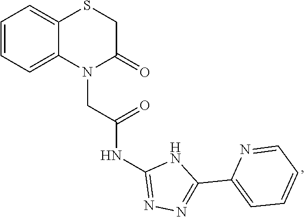 [SMILES: O=C(CN1C(=O)CSc2ccccc21)Nc1nnc(-c2ccccn2)[nH]1]

or a pharmaceutically acceptable salt thereof.

Also disclosed are methods for the treatment of a neurological disorder in a subject, the method comprising the step of administering to the subject an effective amount of at least one disclosed compound.

Also disclosed are methods for the treatment of a neurological disorder in a subject, the method comprising the step of administering to the subject an effective amount of a compound having a structure represented by a formula:

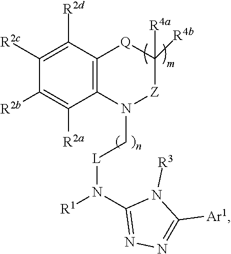

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²¹, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, or a pharmaceutically acceptable salt thereof.

Also disclosed are methods for modifying Tau-SH3 signaling in a subject, the method comprising the step of administering to the subject an effective amount of at least one disclosed compound.

Also disclosed are methods for modifying Tau-SH3 signaling in at least one cell, the method comprising the step of contacting at least one cell with an effective amount of at least one disclosed compound.

Also disclosed are kits comprising at least one disclosed compound and one or more of: (a) at least one agent associated with the treatment of a neurological disorder; (b) instructions for administering the compound in connection with treating a neurological disorder; and (c) instructions for treating a neurological disorder.

Also disclosed are kits comprising a compound having a structure represented by a formula:

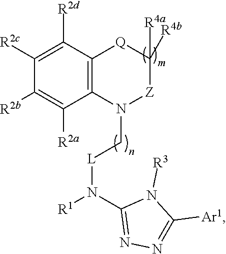

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, or a pharmaceutically acceptable salt thereof, and one or more of. (a) at least one agent associated with the treatment of a neurological disorder; (b) instructions for administering the compound in connection with treating a neurological disorder; and (c) instructions for treating a neurological disorder.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1A-E show representative data illustrating that compound no. 55 has attractive drug-like properties and prevents Aβ toxicity.

FIG. 2 shows a representative structure illustrating a proposed structure-activity relationship (SAR) approach towards compound no. 55.

FIG. 3A-F show representative data illustrating that the exemplary compounds inhibit Tau-Fyn interaction in cells, as well as ameliorate Aβ toxicity.

FIG. 4 shows representative data illustrating the MTT assay results of exemplary compounds.

FIG. 5A-E show representative data illustrating that compound no. 69 binds Tau and not FynSH3.

FIG. 6A-I show representative data illustrating that compound no. 69 is a selective Tau-SH3 interaction inhibitor.

FIG. 7A-D show representative data pertaining to the controls for SH3 plasma.

FIG. 8A-G show representative data illustrating that compound no. 69 ameliorates Aβ-induced neurite degeneration and network hyperexcitability.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used in the specification and in the claims, the term “comprising” can include the aspects “consisting of” and “consisting essentially of”.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, “IC₅₀,” is intended to refer to the concentration of a substance (e.g., a compound or a drug) that is required for 50% inhibition of a biological process, or component of a process, including a protein, subunit, organelle, ribonucleoprotein, etc. In one aspect, an IC₅₀ can refer to the concentration of a substance that is required for 50% inhibition in vivo, as further defined elsewhere herein. In a further aspect, IC₅₀ refers to the half maximal (50%) inhibitory concentration (IC) of a substance.

As used herein, “EC₅₀,” is intended to refer to the concentration of a substance (e.g., a compound or a drug) that is required for 50% agonism of a biological process, or component of a process, including a protein, subunit, organelle, ribonucleoprotein, etc. In one aspect, an EC₅₀ can refer to the concentration of a substance that is required for 50% agonism in vivo, as further defined elsewhere herein. In a further aspect, EC₅₀ refers to the concentration of agonist that provokes a response halfway between the baseline and maximum response.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-ALS agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkvl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as -OA¹-OA²- or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylanmino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -S¹A¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)_{0_4}OR^{o}—O(CH₂)₀₋₄R^{o}, —O—(CH₂)_{0_4}C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)_{0_4}O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)_{0_4}OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)_{0_4}OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o})₂; SiR^{o}₃; —(C_{1 4} straight or branched alkylene)O—N(R^{o})₂; or —(C₁₋₄ straight or branched alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently halogen, -(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂₋₃O—, wherein each independent occurrence of R*is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxvlic acids, and various protecting groups known in the art (see, for example, “Protective Groups in Organic Synthesis,” T. W. Greene, P. G. M. Wuts, Wiley-lnterscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

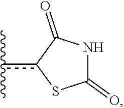

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral carbons, it can have more than two optical isomers and can exist in diastereoisomeric forms. For example, when there are two chiral carbons, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an a-hydrogen can exist in an equilibrium of the keto form and the enol form.

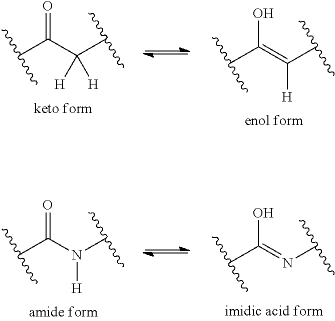 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomenc forms, N¹-unsubstituted, 3-A³ and N¹-unsubstituted, 5-A³ as shown below.

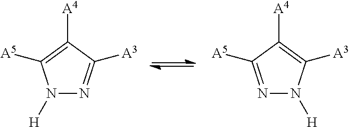

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

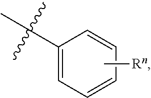

which is understood to be equivalent to a formula:

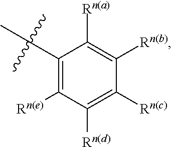

wherein n is typically an integer. That is, R is understood to represent five independent substituents, R^{n(a)}, R^{n(b)}, R^{n(e)}, R^{n(d)}, R^{n(e)}. By “independent substituents,” it is meant that each R substituent can be independently defined. For example, if in one instance Rn(a) is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating disorders associated with a neurological disorder such as, for example, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD).

In one aspect, the disclosed compounds exhibit modification of Tau-SH3 signaling. In a further aspect, the disclosed compounds exhibit inhibition of Tau-SH3 signaling.

In one aspect, the disclosed compounds exhibit modification of Tau-Fyn signaling. In a further aspect, the disclosed compounds exhibit inhibition of Tau-Fyn signaling.

In one aspect, the compounds of the invention are useful in modifying Tau-SH3 signaling in a mammal. In a further aspect, the compounds of the invention are useful in modifying Tau-SH3 signaling in at least one cell.

In one aspect, the compounds of the invention are useful in modifying Tau-Fyn signaling in a mammal. In a further aspect, the compounds of the invention are useful in modifying Tau-Fyn signaling in at least one cell.

In one aspect, the compounds of the invention are useful in the treatment of neurological disorders, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

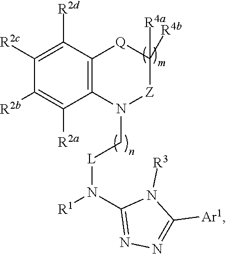

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

In one aspect, disclosed are compounds having a structure represented by a formula:

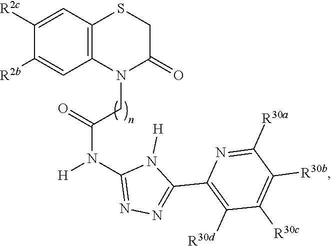

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

In one aspect, disclosed are compounds selected from the group consisting of:

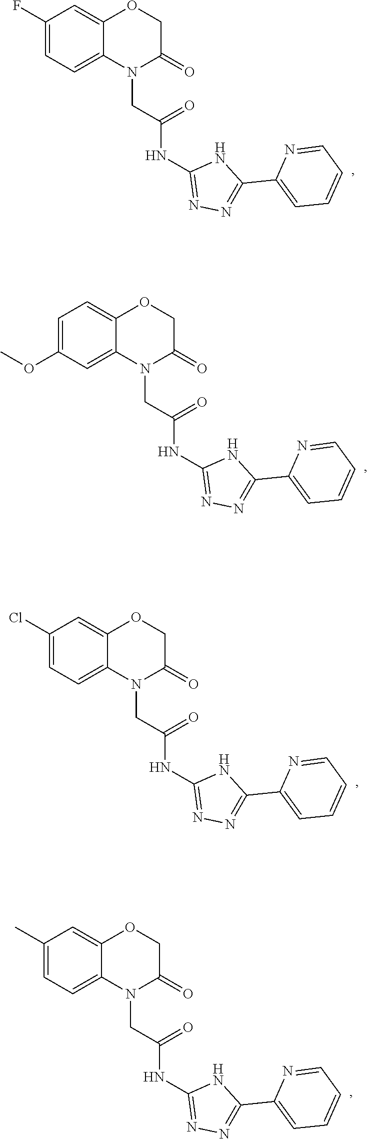 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1]

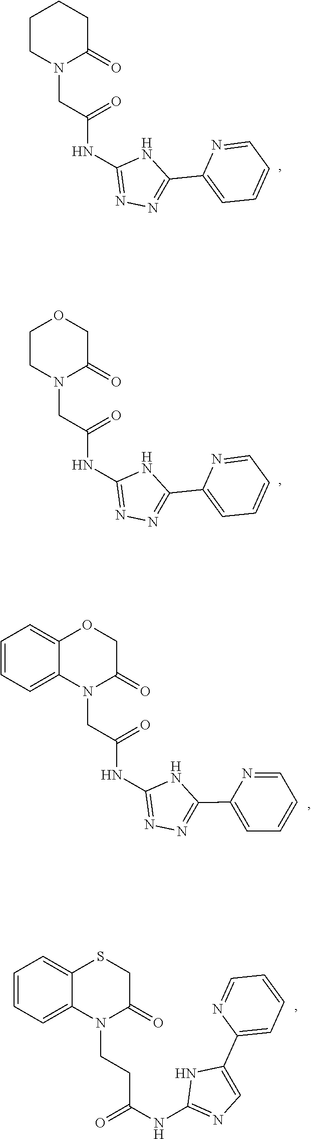 [SMILES: O=C(CN1CCCCC1=O)Nc1nnc(-c2ccccn2)[nH]1 ; O=C(CN1CCOCC1=O)Nc1nnc(-c2ccccn2)[nH]1 ; O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1]

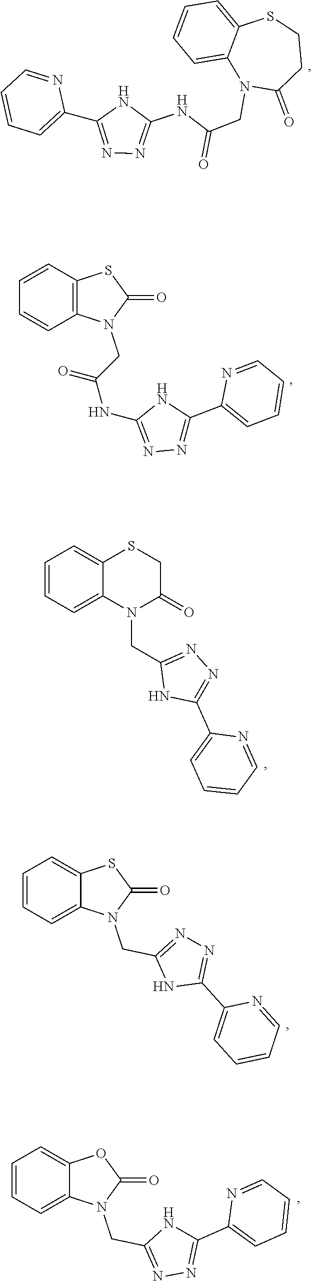 [SMILES: O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1 ; O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1]

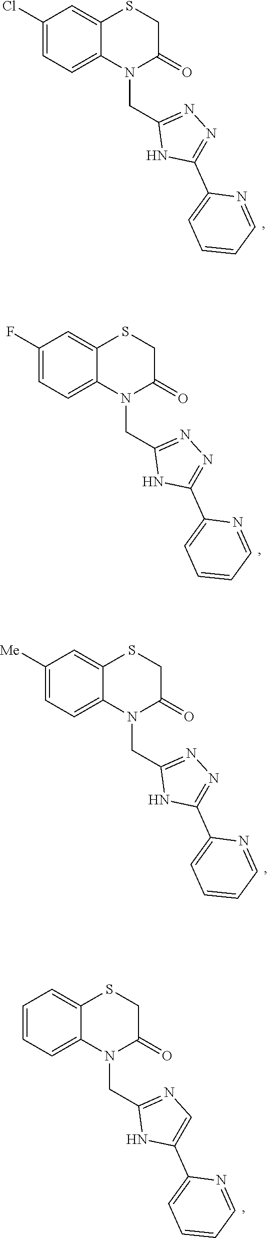 [SMILES: O=C1CSc2cc(Cl)ccc2N1Cc1nnc(-c2ccccn2)[nH]1 ; O=C1CSc2cc(F)ccc2N1Cc1nnc(-c2ccccn2)[nH]1 ; Cc1ccc2c(c1)SCC(=O)N2Cc1nnc(-c2ccccn2)[nH]1 ; O=C1CSc2ccccc2N1Cc1ncc(-c2ccccn2)[nH]1]

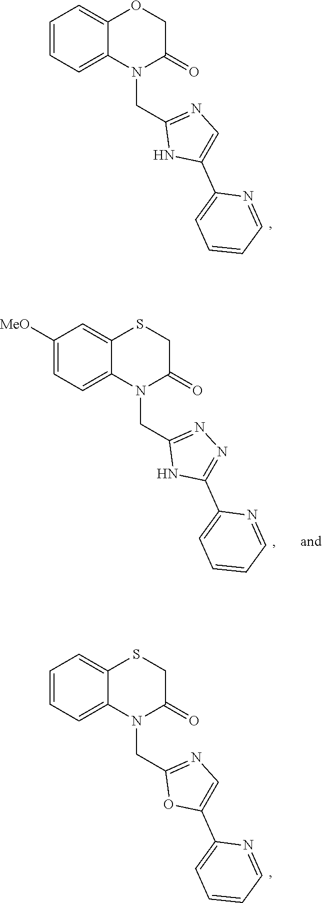 [SMILES: O=C1COc2ccccc2N1Cc1ncc(-c2ccccn2)[nH]1 ; COc1ccc2c(c1)SCC(=O)N2Cc1nnc(-c2ccccn2)[nH]1 ; O=C1CSc2ccccc2N1Cc1ncc(-c2ccccn2)o1]

or a pharmaceutically acceptable salt thereof.

In a further aspect, the compound has a structure represented by a formula:

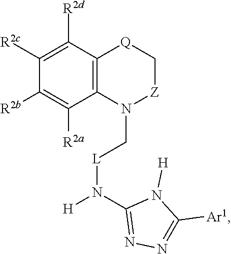

or a pharmaceutically acceptable salt thereof.

In a further aspect, the compound has a structure represented by a formula:

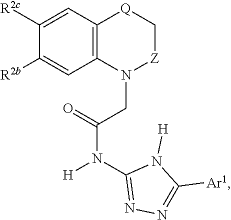

or a pharmaceutically acceptable salt thereof.

In a further aspect, the compound has a structure represented by a formula:

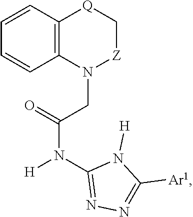

or a pharmaceutically acceptable salt thereof.

In a further aspect, the compound has a structure represented by a formula:

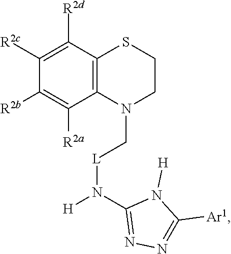

or a pharmaceutically acceptable salt thereof.

In a further aspect, the compound has a structure selected from the group consisting of:

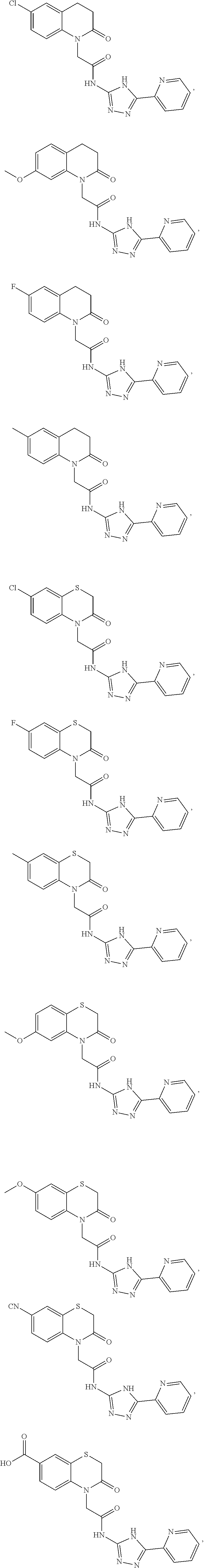

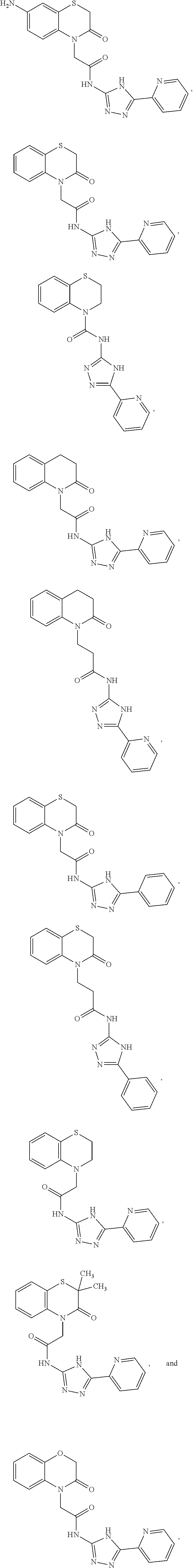

or a pharmaceutically acceptable salt thereof.

In a further aspect, the compound is:

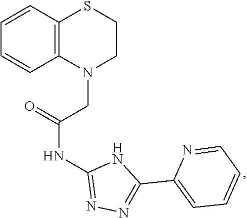 [SMILES: O=C(CN1CCSc2ccccc21)Nc1nnc(-c2ccccn2)[nH]1]

or a pharmaceutically acceptable salt thereof.

In a further aspect, the compound has a structure represented by a formula:

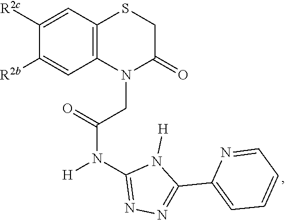

or a pharmaceutically acceptable salt thereof.

In a further aspect, the compound has a structure represented by a formula:

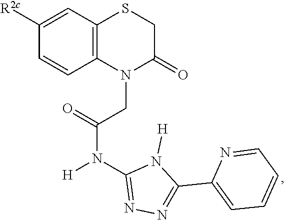

or a pharmaceutically acceptable salt thereof.

In a further aspect, the compound is selected from the group consisting of:

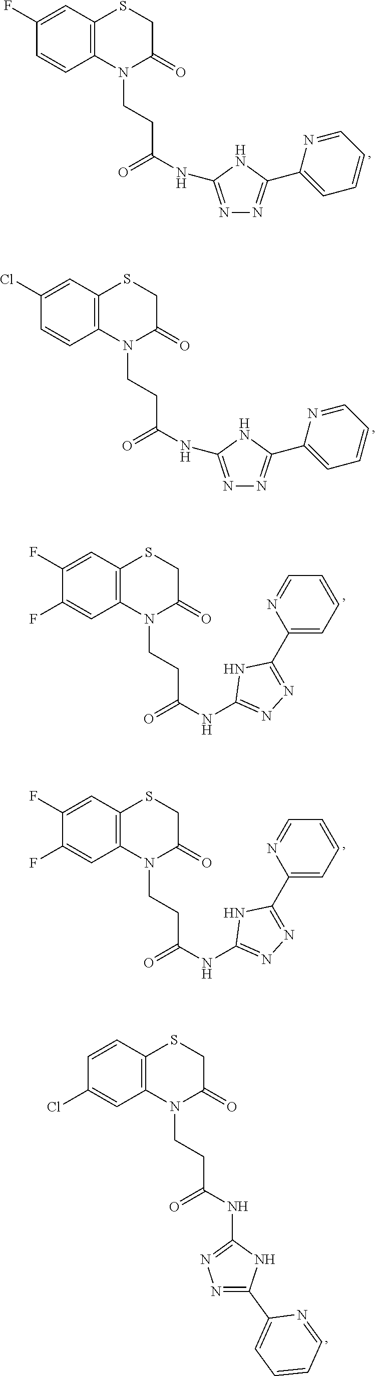 [SMILES: O=C(CCN1C(=O)CSc2cc(F)ccc21)Nc1nnc(-c2ccccn2)[nH]1 ; O=C(CCN1C(=O)CSc2cc(Cl)ccc21)Nc1nnc(-c2ccccn2)[nH]1 ; O=C(CCN1C(=O)CSc2cc(F)c(F)cc21)Nc1nnc(-c2ccccn2)[nH]1 ; O=C(CCN1C(=O)CSc2cc(F)c(F)cc21)Nc1nnc(-c2ccccn2)[nH]1 ; O=C(CCN1C(=O)CSc2ccc(Cl)cc21)Nc1nnc(-c2ccccn2)[nH]1]

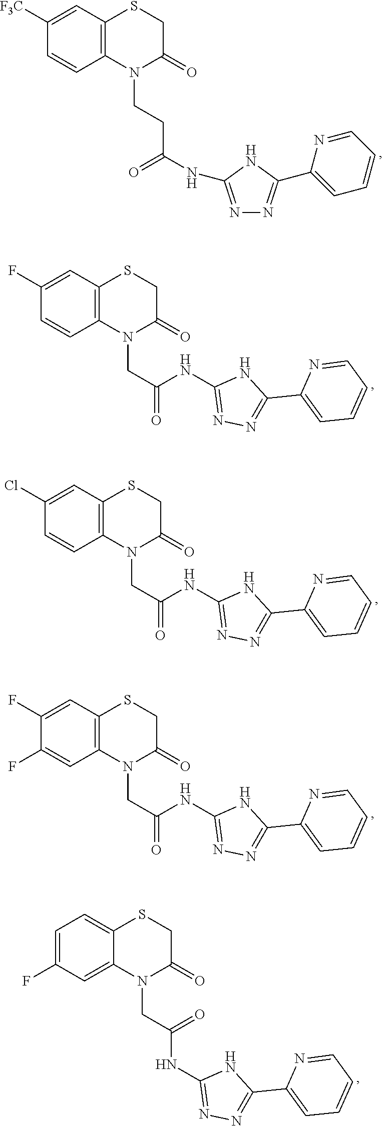 [SMILES: O=C(CCN1C(=O)CSc2cc(C(F)(F)F)ccc21)Nc1nnc(-c2ccccn2)[nH]1 ; O=C(CN1C(=O)CSc2cc(F)ccc21)Nc1nnc(-c2ccccn2)[nH]1 ; O=C(CN1C(=O)CSc2cc(Cl)ccc21)Nc1nnc(-c2ccccn2)[nH]1 ; O=C(CN1C(=O)CSc2cc(F)c(F)cc21)Nc1nnc(-c2ccccn2)[nH]1 ; O=C(CN1C(=O)CSc2ccc(F)cc21)Nc1nnc(-c2ccccn2)[nH]1]

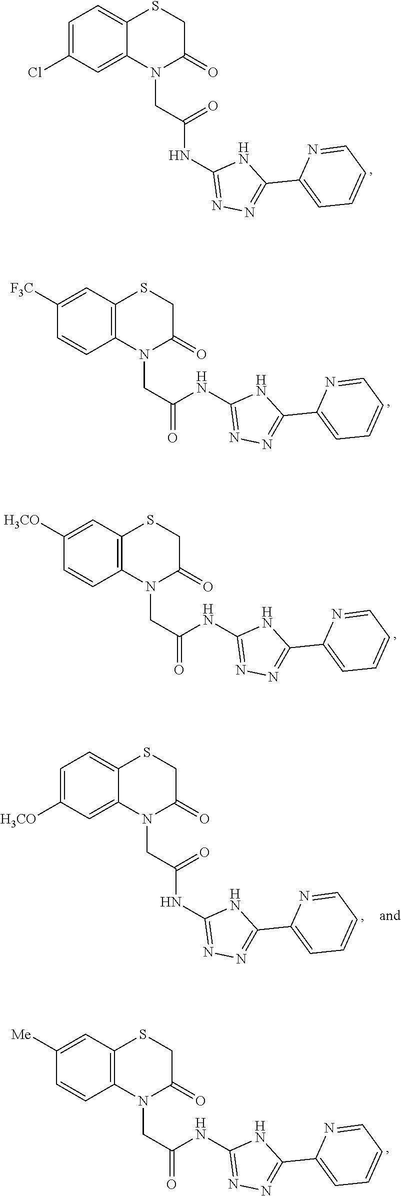 [SMILES: O=C(CN1C(=O)CSc2ccc(Cl)cc21)Nc1nnc(-c2ccccn2)[nH]1 ; O=C(CN1C(=O)CSc2cc(C(F)(F)F)ccc21)Nc1nnc(-c2ccccn2)[nH]1 ; COc1ccc2c(c1)SCC(=O)N2CC(=O)Nc1nnc(-c2ccccn2)[nH]1 ; COc1ccc2c(c1)N(CC(=O)Nc1nnc(-c3ccccn3)[nH]1)C(=O)CS2 ; Cc1ccc2c(c1)SCC(=O)N2CC(=O)Nc1nnc(-c2ccccn2)[nH]1]

or a pharmaceutically acceptable salt thereof.

In one aspect, each of n and m is independently 0, 1, or 2. In a further aspect, each of n and m is independently 0 or 1. In a still further aspect, each of n and m is independently 0 or 2. In yet a further aspect, each of n and m is independently 1 or 2. In an even further aspect, each of n and m is 2. In a still further aspect, each of n and m is 1. In yet a further aspect, each of n and m is 0.

In a further aspect, n is 0 and m is 0, 1, or 2. In a still further aspect, n is 0 and m is 0 or 1. In an even further aspect, n is 0 and m is 1. In a still further aspect, n is 0 and m is 2. In a further aspect, n is 1 and m is 0, 1, or 2. In a still further aspect, n is 1 and m is 0 or 1. In yet a further aspect, n is 1 and m is 0. In a still further aspect, n is 1 and m is 2. In a further aspect, n is 2 and m is 0, 1, or 2. In a still further aspect, n is 2 and m is 0 or 1. In yet a further aspect, n is 2 and m is 0. In an even further aspect, n is 2 and m is 1.

In a further aspect, m is 0 and n is 0, 1, or 2. In a still further aspect, m is 0 and n is 0 or 1. In a further aspect, m is 1 and n is 0, 1, or 2. In a further aspect, m is 2 and n is 0, 1, or 2.

In one aspect, n is 0, 1, or 2. In a further aspect, n is 0 or 1. In a still further aspect, n is 0 or 2. In yet a further aspect, n is 1 or 2. In an even further aspect, n is 0. In a still further aspect, n is 1. In yet a further aspect, n is 2.

### A. Q Groups

In one aspect, Q is selected from the group consisting of —O—, —S—, and —CH₂—. In a further aspect, Q is selected from the group consisting of —O— and —S—. In a still further aspect, Q is selected from the group consisting of —S— and —CH₂—. In yet a further aspect, Q is selected from the group consisting of —O— and —CH₂—. In an even further aspect, Q is —O—. In a still further aspect, Q is —S—. In yet a further aspect, Q is —CH₂—.

b. Z Groups

In one aspect, Z is selected from the group consisting of —C(O)—, —CH(OH)—, and —CH₂—. In a further aspect, Z is selected from the group consisting of —C(O)— and —CH(OH)—. In a still further aspect, Z is selected from the group consisting of —CH(OH)— and —CH₂—. In yet a further aspect, Z is selected from the group consisting of —C(O)— and —CH₂—. In a still further aspect, Z is —C(O)—. In yet a further aspect, —CH(OH)—. In an even further aspect, Z is —CH₂—.

c. L Groups

In one aspect, L is selected from the group consisting of —C(O)— and —CH₂—. In a further aspect, L is —C(O)—. In a still further aspect, L is —CH₂—.

d. R¹ and R³ Groups

In one aspect, each of R¹ and R³ is independently selected from the group consisting of hydrogen and C1-C4 alkyl. In a further aspect, each of R¹ and R³ is independently selected from the group consisting of hydrogen, methyl, ethyl, n-propyl, and isopropyl. In a still further aspect, each of R¹ and R³ is independently selected from the group consisting of hydrogen, methyl, and ethyl. In yet a further aspect, each of R¹ and R³ is independently selected from the group consisting of hydrogen and ethyl. In an even further aspect, each of R¹ and R³ is independently selected from the group consisting of hydrogen and methyl.

In a further aspect, each of R¹ and R³ is hydrogen.

In various aspects, each of R¹ and R³ is C1-C4 alkyl. In a further aspect, each of R¹ and R³ is independently selected from the group consisting of methyl, ethyl, n-propyl, and isopropyl. In a still further aspect, each of R¹ and R³ is independently selected from the group consisting of methyl and ethyl. In yet a further aspect, each of R¹ and R³ is ethyl. In an even further aspect, each of R¹ and R³ is methyl.

In a further aspect, R¹ is hydrogen and R³ is selected from the group consisting of hydrogen and C1-C4 alkyl. In a still further aspect, R¹ is hydrogen and R³ is selected from the group consisting of hydrogen, methyl, ethyl, n-propyl, and isopropyl. In yet a further aspect, R¹ is hydrogen and R³ is selected from the group consisting of hydrogen, methyl, and ethyl. In an even further aspect, R¹ is hydrogen and R³ is selected from the group consisting of hydrogen and methyl.

In a further aspect, R³ is hydrogen and R¹ is selected from the group consisting of hydrogen and Ct-C4 alkyl. In a still further aspect, R³ is hydrogen and R¹ is selected from the group consisting of hydrogen, methyl, ethyl, n-propyl, and isopropyl. In yet a further aspect, R³ is hydrogen and R¹ is selected from the group consisting of hydrogen, methyl, and ethyl. In an even further aspect, R³ is hydrogen and R¹ is selected from the group consisting of hydrogen and methyl.

e. R^{2A}, R^{2B}, R^{2C}, and R^{2D} Groups

In one aspect, each of R^{2a}, R^{2b}, R^{2c}, and R^{2a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, Ct-C4 alkylamino, (C1-C4)(C1-C4) dialkylamino, C1-C4 aminoalkyl, —C(O)R²⁰, —CO₀R²¹, —C(O)NR²², and —SO₂R²³. In a further aspect, each of R^{2a}, R^{2b}, R^{2c}, and R^{2d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2a}, R^{2b}, R^{2c}, and R^{2d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³.

In yet a further aspect, each of R^{2a}, R^{2b}, R^{2c}, and R^{2a} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In one aspect, each of R^{2b} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2b} and R^{2c} is not hydrogen. In a further aspect, each of R^{2b} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2b} and R^{2c} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2b} and R^{2c} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₀R²¹, —C(O)NR²², and —SO₂R²³.

In various aspects, each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 hydroxyalkyl, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2a}, R^{2b}, R^{2c}, and R^{2d} is independently selected from the group consisting of hydrogen, halogen, —CN, —NH₂, —OH, —NO₂, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2a}, R^{2b}, R^{2c}, and R^{2d} is independently selected from the group consisting of hydrogen, halogen, —CN, —NH₂, —OH, —NO₂, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2a}, R^{2b}, R^{2c}, and R^{2d} is independently selected from the group consisting of hydrogen, halogen, —CN, —NH₂, —OH, —NO₂, methyl, —CH₂OH, —OCH₃, —NHCH₃, —N(CH₃)₂, CH₂NH₂, —C(O)R²⁰, —CO₂R²¹, —C(O)NR²², and —SO₂R²³.

In various aspects, each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2a}, R^{2b}, R^{2c}, and R^{2d} is independently selected from the group consisting of hydrogen, halogen. —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²¹, —CO₂R²¹, —C(O)NR²², and —SO₂R²³. In a still further aspect, each of R^{2a}, R^{2b}, R^{2c}, and R^{2d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2a}, R^{2b}, R^{2c}, and R^{2d} is independently selected from the group consisting of hydrogen, halogen, —CN, —NH₂, —OH, —NO₂, methyl, —CH₂F, —CH₂Cl, —CH₂CN, —NHCH₃, —N(CH₃)₂, —CH₂NH₂, —C(O)R²⁰, —CO₂R²¹, —C(O)NR²², and —SO₂R²³.

In various aspects, each of R^{2a}, R^{2b}, R^{2c}, and R^{2a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —C(O)R²⁰, —CO₂R²¹, —C(O)NR²², and —SO₂R²³. In a further aspect, each of R^{2a}, R^{2b}, R^{2c}, and R^{2d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —C(O)R²⁰, —CO₂R²¹, —C(O)NR²², and —SO₂R²³.

In a still further aspect, each of R^{2a}, R^{2b}, R^{2c}, and R^{2d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —C(O)R²⁰, —CO₂R²¹, —C(O)NR²², and —SO₂R²³. In yet a further aspect, each of R^{2a}, R^{2b}, R^{2c}, and R^{2d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —C(O)R²⁰, —CO₂R²¹, —C(O)NR²², and —SO₂R²³.

In various aspects, each of R^{2a}, R^{2b}, R^{2c}, and R^{2d} is independently selected from the group consisting of hydrogen, halogen, C1-C4 alkyl, C2-C4 alkenyl, C1-C4 hydroxyalkyl, C1-C4 alkoxy, C1-C4 alkylamino, (C1-C4)(C1-C4) dialkylamino, and C1-C4 aminoalkyl. In a further aspect, each of R^{2a}, R^{2b}, R^{2c}, and R^{2d} is independently selected from the group consisting of hydrogen, halogen, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, and R^{2d} is independently selected from the group consisting of hydrogen, halogen, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, and R^{2d} is independently selected from the group consisting of hydrogen, halogen, methyl, —CH₂OH, —OCH₃, —NHCH₃, —N(CH₃)₂, and —CH₂NH₂.

In various aspects, each of R^{2a}, R^{2b}, R^{2c}, and R^{2d} is independently selected from the group consisting of hydrogen, halogen, C1-C4 alkyl, C2-C4 alkenyl, C1-C4 haloalkyl, C1-C4 cyanoalkyl, C1-C4 alkylamino, (C1-C4)(C1-C4) dialkylamino, and C1-C4 aminoalkyl. In a further aspect, each of R^{2a}, R^{2b}, R^{2c}, and R^{2d} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{2a}, R^{2b}, R^{2c}, and R^{2a} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —NHCH₃, —NHCH₂CH₃, —N(CH₃)₂, —N(CH₃)CH₂CH₃, —CH₂NH₂, and —CH₂CH₂NH₂. In yet a further aspect, each of R^{2a}, R^{2b}, R^{2c}, and R^{2d} is independently selected from the group consisting of hydrogen, halogen, methyl, —CH₂F, —CH₂Cl, —CH₂CN, —NHCH₃, —N(CH₃)₂, and —CH₂NH₂.

In various aspects, each of R^{2a}, R^{2b}, R^{2c}, and R^{2d} is independently selected from the group consisting of hydrogen, halogen, C1-C4 alkyl, C2-C4 alkenyl, C1-C4 haloalkyl, C1-C4 cyanoalkyl, C1-C4 hydroxyalkyl, C1-C4 haloalkoxy, and C1-C4 alkoxy. In a further aspect, each of R^{2a}, R^{2b}, R^{2c}, and R^{2d} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, and —OCH(CH₃)₂. In a still further aspect, each of R^{2a}, R^{2b}, R^{2c}, and R^{2d} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, and —OCH₂CH₃. In yet a further aspect, each of R^{2a}, R^{2b}, R^{2c}, and R^{2d} is independently selected from the group consisting of hydrogen, halogen, methyl, —CH₂F, —CH₂Cl, —CH₂CN, —CH₂OH, —OCF₃, —OCHF₃, —OCH₂F, and —OCH₃.

In various aspects, each of R^{2b} and R^{2c} is independently selected from the group consisting of hydrogen, halogen, —CN, —NH₂, —OH, —NO₂, C1-C4 alkyl, C2-C4 alkenyl, C1-C4 hydroxyalkyl, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2b} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₀R²¹, —C(O)NR²², and —SO₂R²³. In a still further aspect, each of R^{2b} and R^{2c} is independently selected from the group consisting of hydrogen, halogen, —CN, —NH₂, —OH, —NO₂, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2b} and R^{2C} is independently selected from the group consisting of hydrogen, halogen, —CN, —NH₂, —OH, —NO₂, methyl, —CH₂OH, —OCH₃, —NHCH₃, —N(CH₃)₂, —CH₂NH₂, —C(O)R²⁰, —CO₂R²¹, —C(O)NR²², and —SO₂R²³.

In various aspects, each of R^{2b} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2b} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2b} and R^{2c} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2b} and R^{2c} is independently selected from the group consisting of hydrogen, halogen, —CN, —NH₂, —OH, —NO₂, methyl, —CH₂F, —CH₂Cl, —CH₂CN, —NHCH₃, —N(CH₃)₂, —CH₂NH₂, —C(O)R²⁰, —CO₂R²¹, —C(O)NR²², and —SO₂R²³.

In various aspects, each of R^{2b} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —C(O)R²⁰, —CO₂R²¹, —C(O)NR²², and —SO₂R²³. In a further aspect, each of R^{2b} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —C(O)R²⁰, —CO₂R²¹, —C(O)NR²², and —SO₂R²³. In a still further aspect, each of R^{2b} and R^{2e} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —C(O)R²⁰, —CO₂R²¹, —C(O)NR²², and —SO₂R²³. In yet a further aspect, each of R^{2b} and R^{2e} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —C(O)R²⁰, —CO₂R²¹, —C(O)NR²², and —SO₂R²³.

In various aspects, each of R^{2b} and R^{2c} is independently selected from the group consisting of hydrogen, halogen, C1-C4 alkyl, C2-C4 alkenyl, C1-C4 hydroxyalkyl, C1-C4 alkoxy, C1-C4 alkylamino, (C1-C4)(C1-C4) dialkylamino, and C1-C4 aminoalkyl. In a further aspect, each of R^{2b} and R^{2c} is independently selected from the group consisting of hydrogen, halogen, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of each of R^{2b} and R^{2c} is independently selected from the group consisting of hydrogen, halogen, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{2b} and R^{2c} is independently selected from the group consisting of hydrogen, halogen, methyl, —CH₂OH, —OCH₃, —NHCH₃, —N(CH₃)₂, and —CH₂NH₂.

In various aspects, each of R^{2b} and R^{2c} is independently selected from the group consisting of hydrogen, halogen, C1-C4 alkyl, C2-C4 alkenyl, C1-C4 haloalkyl, C1-C4 cyanoalkyl, C1-C4 alkylamino, (C1-C4)(C1-C4) dialkylamino, and C1-C4 aminoalkyl. In a further aspect, each of R^{2b} and R^{2c} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, CH₂CH₂CH₂NH₂, and —CH(CH₃)CH₂NH₂. In a still further aspect, each of R^{2b} and R^{2c} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —NHCH₃, —NHCH₂CH₃, —N(CH₃)₂, —N(CH₃)CH₂CH₃, —CH₂NH₂, and —CH₂CH₂NH₂. In yet a further aspect, each of R^{2b} and R^{2c} is independently selected from the group consisting of hydrogen, halogen, methyl, —CH₂F, —CH₂Cl, —CH₂CN, —NHCH₃, —N(CH₃)₂, and —CH₂NH₂.

In various aspects, each of R^{2b} and R^{2c} is independently selected from the group consisting of hydrogen, halogen, C1-C4 alkyl, C2-C4 alkenyl, C1-C4 haloalkyl, C1-C4 cyanoalkyl, C1-C4 hydroxyalkyl, C1-C4 haloalkoxy, and C1-C4 alkoxy. In a further aspect, each of R^{2b} and R^{2c} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, and —OCH(CH₃)₂. In a still further aspect, each of R^{2b} and R^{2c} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, and —OCH₂CH₃. In yet a further aspect, each of R^{2b} and R^{2e} is independently selected from the group consisting of hydrogen, halogen, methyl, —CH₂F, —CH₂Cl, —CH₂CN, —CH₂OH, —OCF₃, —OCHF₃, —OCH₂F, and —OCH₃.

In a further aspect, each of R^{2a}, R^{2b}, R^{2c}, and R^{2a} is hydrogen.

In a further aspect, each of R^{2b} and R^{2c} is hydrogen. In a still further aspect, each of R^{2b} and R^{2d} is hydrogen. In yet a further aspect, each of R^{2a} and R^{2b} is hydrogen. In an even further aspect, each of R^{2a} and R^{2c} is hydrogen. In a still further aspect, each of R^{2a} and R^{2d} is hydrogen. In yet a further aspect, each of R^{2c} and R^{2d} is hydrogen.

In a further aspect, each of R^{2a}, R^{2b}, R^{2c}, and R^{2d} is selected from the group consisting of hydrogen and halogen. In a still further aspect, each of R^{2a}, R^{2b}, R^{2c}, and R^{2d} is selected from the group consisting of hydrogen, —F, —Cl, and —Br. In yet a further aspect, each of R^{2a}, R^{2b}, R^{2c}, and R^{2d} is selected from the group consisting of hydrogen, —F, and —Cl. In an even further aspect, each of R^{2a}, R^{2b}, R^{2c}, and R^{2d} is selected from the group consisting of hydrogen and —Cl. In a still further aspect, each of R^{2a}, R^{2b}, R^{2c}, and R^{2d} is selected from the group consisting of hydrogen and —F.

In a further aspect, each of R^{2b} and R^{2c} is selected from the group consisting of hydrogen and halogen. In a still further aspect, each of R^{2b} and R^{2c} is selected from the group consisting of hydrogen, —F, —Cl, and —Br. In yet a further aspect, each of R^{2b} and R^{2c} is selected from the group consisting of hydrogen, —F, and —Cl. In an even further aspect, each of R^{2b} and R^{2c} is selected from the group consisting of hydrogen and —Cl. In a still further aspect, each of R^{2b} and R^{2c} is selected from the group consisting of hydrogen and —F.

In various aspects, each of R^{2a} and R^{2b} is hydrogen and each of R^{2c} and R^{2a} is independently selected from the group consisting of hydrogen, halogen, —CN, —NH₂, —OH, NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2a} and R^{2b} is hydrogen and each of R^{2c} and R^{2d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2a} and R^{2b} is hydrogen and each of R^{2e} and R^{2a} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²² and —SO₂R²³. In yet a further aspect, each of R^{2a} and R^{2b} is hydrogen and each of R^{2e} and R^{2a} is independently selected from the group consisting of hydrogen, halogen, —CN, —NH₂, —OH, NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In various aspects, each of R^{2a} and R^{2c} is hydrogen and each of R^{2b} and R^{2a} is independently selected from the group consisting of hydrogen, halogen, —CN, —NH₂, —OH. —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₀R²¹, —C(O)NR²², and —SO₂R²³. In a further aspect, each of R^{2a} and R^{2c} is hydrogen and each of R^{2b} and R^{2a} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2a} and R^{2c} is hydrogen and each of R^{2b} and R^{2a} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₂, —OCH₂F, —OCH₂CH₂F, —OCH₃, —OCH₂CH, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²² and —SO₂R²³. In yet a further aspect, each of R^{2a} and R^{2c} is hydrogen and each of R^{2b} and R^{2a} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In various aspects, each of R^{2a} and R^{2d} is hydrogen and each of R^{2b} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2a} and R^{2d} is hydrogen and each of R^{2b} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²¹, —CO₀R²¹, —C(O)NR²², and —SO₂R²³. In a still further aspect, each of R^{2a} and R^{2d} is hydrogen and each of R^{2b} and R^{2c} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²² and —SO₂R²³. In yet a further aspect, each of R^{2a} and R^{2d} is hydrogen and each of R^{2b} and R^{2c} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²¹, —CO₂R²¹, —C(O)NR²², and —SO₂R²³.

In various aspects, each of R^{2b} and R^{2c} is hydrogen and each of R^{2a} and R^{2a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2b} and R^{2c} is hydrogen and each of R^{2a} and R^{2d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2b} and R^{2c} is hydrogen and each of R^{2a} and R^{2d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²²¹ and —SO₂R²³. In yet a further aspect, each of R^{2b} and R^{2e} is hydrogen and each of R^{2a} and R^{2d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In various aspects, each of R^{2b} and R^{2d} is hydrogen and each of R^{2a} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³. In a further aspect, each of R^{2b} and R^{2d} is hydrogen and each of R^{2a} and R^{2c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2b} and R^{2d} is hydrogen and each of R^{2a} and R^{2c} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R²⁰, —CO₂R²¹, —C(O)NR²², and —SO₂R²³. In yet a further aspect, each of R^{2b} and R^{2a} is hydrogen and each of R^{2a} and R^{2c} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In various aspects, each of R^{2c} and R^{2d} is hydrogen and each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R², —C(O)NR²², and —SO₂R²³. In a further aspect, each of R^{2c} and R^{2d} is hydrogen and each of R^{2a} and R^{2b} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃. —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R²⁰, —CO₂R²¹, —C(O)NR²², and —SO₂R²³. In a still further aspect, each of R^{2c} and R^{2d} is hydrogen and each of R^{2a} and R^{2b} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, N(CH₃)₂, —N(CH₃)CH₂CH₃, —CH₂NH₂, —CH₂CH₂NH₂, —C(O)R²¹, —CO₂R²¹, —C(O)NR²² and —SO₂R²³. In yet a further aspect, each of R^{2c} and R^{2d} is hydrogen and each of R^{2a} and R^{2b}is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R²⁰, —CO₂R²¹, —C(O)NR²², and —SO₂R²³.

In a further aspect, each of R^{2b} and R^{2c} is independently selected from the group consisting of hydrogen, halogen, C1-C4 alkyl, C1-C4 haloalkyl, and C1-C4 alkoxy. In a still further aspect, each of R^{2b} and R^{2c} is independently selected from the group consisting of hydrogen, —F, —Cl, methyl, ethyl, n-propyl, isopropyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —OCH₃, —OCH₂CH₃, —OCH₂CH₂CH₃, and —OCH(CH₃)₂. In yet a further aspect, each of R^{2b} and R^{2c} is independently selected from the group consisting of hydrogen, —F, —Cl, methyl, ethyl, —CH₂F, —CH₂CH₂F, —CH₂Cl, —CH₂CH₂Cl, —OCH₃, and —OCH₂CH₃. In an even further aspect, each of R^{2b} and R^{2e} is independently selected from the group consisting of hydrogen, —F, —Cl, methyl, —CH₂F, —CH₂Cl, and —OCH₃.

In a further aspect, each of R^{2b} and R^{2c} is independently selected from hydrogen, halogen, and C1-C4 haloalkyl. In a still further aspect, each of R^{2b} and R^{2c} is independently selected from hydrogen, —F, —Cl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In a still further aspect, each of R^{2b} and R^{2c} is independently selected from hydrogen, —F, —Cl, —CH₂F, CH₂CH₂F, —CH₂Cl, and —CH₂CH₂C1. In yet a further aspect, each of R^{2b} and R^{2c} is independently selected from hydrogen, —F, —Cl, —CH₂F, and —CH₂C1.

In a further aspect, R^{2b} is hydrogen and R^{2C} is selected from the group consisting of halogen, C1-C4 alkyl, C1-C4 haloalkyl, and C1-C4 alkoxy. In a still further aspect, R^{2b} is hydrogen and R^{2c} is selected from the group consisting of —F, —Cl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In yet a further aspect, R^{2b} is hydrogen and R^{2c} is selected from the group consisting of —F, —Cl, —CH₂F, —CH₂CH₂F, —CH₂Cl, and —CH₂CH₂C1. In an even further aspect, R^{2b} is hydrogen and R^{2c} is selected from the group consisting of —F, —Cl, —CH₂F, and —CH₂C1.

In a further aspect, R^{2b} is hydrogen and R^{2C} is halogen. In a still further aspect, R^{2b} is hydrogen and R^{2c} is selected from the group consisting of —F, —Cl, and —Br. In yet a further aspect, R^{2b} is hydrogen and R^{2c} is selected from the group consisting of —F and —Cl.

In an even further aspect, R^{2b} is hydrogen and R^{2c} is —C1. In a still further aspect, R^{2b} is hydrogen and R^{2c} is —F.

f. R^{4A} and R^{4B} Groups

In one aspect, each occurrence of R^{4a} and R^{4b}, when present, is independently selected from hydrogen, C1-C4 alkyl, and C1-C4 haloalkyl. In a further aspect, each occurrence of R^{4a} and R^{4b}, when present, is independently selected from hydrogen, methyl, ethyl, n-propyl, isopropyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In a still further aspect, each occurrence of R^{4a} and R^{4b}, when present, is independently selected from hydrogen, methyl, ethyl, —CH₂F, —CH₂CH₂F, —CH₂Cl, and —CH₂CH₂C1. In yet a further aspect, each occurrence of R^{4a} and R^{4b}, when present, is independently selected from hydrogen, methyl, —CH₂F, and —CH₂C1.

In various aspects, R^{4a}, when present, is hydrogen and R^{4b}, when present, is selected from hydrogen, C1-C4 alkyl, and C1-C4 haloalkyl. In a further aspect, R^{4a}, when present, is hydrogen and R^{4b}, when present, is selected from hydrogen, methyl, ethyl, n-propyl, isopropyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In a still further aspect, R^{4a}, when present, is hydrogen and R^{4b}, when present, is selected from hydrogen, methyl, ethyl, —CH₂F, —CH₂CH₂F, —CH₂C, and —CH₂CH₂C1. In yet a further aspect, R^{4a}, when present, is hydrogen and R^{4b}, when present, is selected from hydrogen, methyl, —CH₂F, and —CH₂C1.

In various aspects, R^{4b}, when present, is hydrogen and R^{4a}, when present, is selected from hydrogen, C1-C4 alkyl, and C1-C4 haloalkyl. In a further aspect, R^{4b}, when present, is hydrogen and R^{4a}, when present, is selected from hydrogen, methyl, ethyl, n-propyl, isopropyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In a still further aspect, R^{4b}, when present, is hydrogen and R^{4a}, when present, is selected from hydrogen, methyl, ethyl, —CH₂F, —CH₂CH₂F, —CH₂Cl, and —CH₂CH₂C1. In yet a further aspect, R^{4b}, when present, is hydrogen and R^{4a}, when present, is selected from hydrogen, methyl, —CH₂F, and —CH₂C1.

In a further aspect, each occurrence of R^{4a} and R^{4b}, when present, is hydrogen.

g. R²⁰, R²¹, R²², and R²³ Groups

In one aspect, each occurrence of R²⁰, R²¹, R²², and R²³, when present, is independently selected from the group consisting of hydrogen and C1-C4 alkyl. In a further aspect, each occurrence of R²⁰, R²¹, R²², and R²³, when present, is independently selected from the group consisting of hydrogen, methyl, ethyl, n-propyl, and isopropyl. In a still further aspect, each occurrence of R²⁰, R²¹, R²², and R²³, when present, is independently selected from the group consisting of hydrogen, methyl, and ethyl. In yet a further aspect, each occurrence of R²⁰, R²¹, R²², and R²³, when present, is independently selected from the group consisting of hydrogen and ethyl. In an even further aspect, each occurrence of R²⁰, R²¹, R²², and R²³, when present, is independently selected from the group consisting of hydrogen and methyl.

In a further aspect, each occurrence of R²⁰, R²¹, R²², and R²³, when present, is hydrogen.

In various aspects, each occurrence of R²⁰, R²¹, R²², and R²³, when present, is C1-C4 alkyl. In a further aspect, each occurrence of R²⁰, R²¹, R²², and R²³, when present, is independently selected from the group consisting of methyl, ethyl, n-propyl, and isopropyl. In a still further aspect, each occurrence of R²⁰, R²¹, R²², and R²³, when present, is independently selected from the group consisting of methyl and ethyl. In yet a further aspect, each occurrence of R²⁰, R²¹, R²², and R²³, when present, is ethyl. In an even further aspect, each occurrence of R²⁰, R²¹, R²², and R²³, when present, is methyl.

In a further aspect, R²⁰, when present, is hydrogen and each occurrence of R²¹, R²², and R²³, when present, is independently selected from the group consisting of hydrogen and C1-C4 alkyl. In a still further aspect, R²⁰, when present, is hydrogen and each occurrence of R²¹, R²², and R²³, when present, is independently selected from the group consisting of hydrogen, methyl, ethyl, n-propyl, and isopropyl. In yet a further aspect, R²⁰, when present, is hydrogen and each occurrence of R²¹, R²², and R²³, when present, is independently selected from the group consisting of hydrogen, methyl, and ethyl. In an even further aspect, R²⁰, when present, is hydrogen and each occurrence of R²¹, R²², and R²³, when present, is independently selected from the group consisting of hydrogen and methyl.

In a further aspect, R²¹, when present, is hydrogen and each occurrence of R²⁰, R²², and R²³, when present, is independently selected from the group consisting of hydrogen and C1-C4 alkyl. In a still further aspect, R²¹, when present, is hydrogen and each occurrence of R²⁰, R²², and R²³, when present, is independently selected from the group consisting of hydrogen, methyl, ethyl, n-propyl, and isopropyl. In yet a further aspect, R²¹, when present, is hydrogen and each occurrence of R²⁰, R²², and R²³, when present, is independently selected from the group consisting of hydrogen, methyl, and ethyl. In an even further aspect, R²¹, when present, is hydrogen and each occurrence of R²⁰, R²², and R²³, when present, is independently selected from the group consisting of hydrogen and methyl.

In a further aspect, R²², when present, is hydrogen and each occurrence of R²⁰, R²¹, and R²³, when present, is independently selected from the group consisting of hydrogen and C1-C4 alkyl. In a still further aspect, R²², when present, is hydrogen and each occurrence of R²⁰, R²¹, and R²³, when present, is independently selected from the group consisting of hydrogen, methyl, ethyl, n-propyl, and isopropyl. In yet a further aspect, R²², when present, is hydrogen and each occurrence of R²⁰, R²¹, and R²³, when present, is independently selected from the group consisting of hydrogen, methyl, and ethyl. In an even further aspect, R²², when present, is hydrogen and each occurrence of R²⁰, R²¹, and R²³, when present, is independently selected from the group consisting of hydrogen and methyl.

In a further aspect, R²³, when present, is hydrogen and each occurrence of R²⁰, R²¹, and R²², when present, is independently selected from the group consisting of hydrogen and C1-C4 alkyl. In a still further aspect, R²³, when present, is hydrogen and each occurrence of R²⁰, R²¹, and R²², when present, is independently selected from the group consisting of hydrogen, methyl, ethyl, n-propyl, and isopropyl. In yet a further aspect, R²³, when present, is hydrogen and each occurrence of R²⁰, R²¹, and R²², when present, is independently selected from the group consisting of hydrogen, methyl, and ethyl. In an even further aspect, R²³ when present, is hydrogen and each occurrence of R²⁰, R²¹, and R²², when present, is independently selected from the group consisting of hydrogen and methyl.

h. R²⁴, R²⁵, R²⁶, and R²⁷ Groups

In one aspect, each occurrence of R²⁴, R²⁵, R²⁶, and R²⁷, when present, is independently selected from the group consisting of hydrogen and C1-C4 alkyl. In a further aspect, each occurrence of R²⁴, R²⁵, R²⁶, and R²⁷, when present, is independently selected from the group consisting of hydrogen, methyl, ethyl, n-propyl, and isopropyl. In a still further aspect, each occurrence of R²⁴, R²⁵, R²⁶, and R²⁷, when present, is independently selected from the group consisting of hydrogen, methyl, and ethyl. In yet a further aspect, each occurrence of R²⁴, R²⁵, R²⁶, and R²⁷, when present, is independently selected from the group consisting of hydrogen and ethyl. In an even further aspect, each occurrence of R²⁴, R²⁵, R²⁶, and R²⁷, when present, is independently selected from the group consisting of hydrogen and methyl.

In a further aspect, each occurrence of R²⁴, R²⁵, R²⁶, and R²⁷, when present, is hydrogen.

In various aspects, each occurrence of R²⁴, R²⁵, R²⁶, and R²⁷, when present, is C1-C4 alkyl. In a further aspect, each occurrence of R²⁴, R²⁵, R²⁶, and R²⁷, when present, is independently selected from the group consisting of methyl, ethyl, n-propyl, and isopropyl. In a still further aspect, each occurrence of R²⁴, R²⁵, R²⁶, and R²⁷, when present, is independently selected from the group consisting of methyl and ethyl. In yet a further aspect, each occurrence of R²⁴, R²⁵, R²⁶, and R²⁷, when present, is ethyl. In an even further aspect, each occurrence of R²⁴, R²⁵, R²⁶, and R²⁷, when present, is methyl.

In a further aspect, R²⁴, when present, is hydrogen and each occurrence of R²⁵, R²⁶, and R²⁷, when present, is independently selected from the group consisting of hydrogen and C1-C4 alkyl. In a still further aspect, R²⁴, when present, is hydrogen and each occurrence of R²⁵, R²⁶, and R²⁷, when present, is independently selected from the group consisting of hydrogen, methyl, ethyl, n-propyl, and isopropyl. In yet a further aspect, R²⁴, when present, is hydrogen and each occurrence of R²⁵, R²⁶, and R²⁷, when present, is independently selected from the group consisting of hydrogen, methyl, and ethyl. In an even further aspect, R²⁴, when present, is hydrogen and each occurrence of R²⁵, R²⁶, and R²⁷, when present, is independently selected from the group consisting of hydrogen and methyl.

In a further aspect, R²⁵, when present, is hydrogen and each occurrence of R²⁴, R²⁶, and R²⁷, when present, is independently selected from the group consisting of hydrogen and C1-C4 alkyl. In a still further aspect, R²⁵, when present, is hydrogen and each occurrence of R²⁴, R²⁶, and R²⁷, when present, is independently selected from the group consisting of hydrogen, methyl, ethyl, n-propyl, and isopropyl. In yet a further aspect, R²⁵, when present, is hydrogen and each occurrence of R²⁴, R²⁶, and R²⁷, when present, is independently selected from the group consisting of hydrogen, methyl, and ethyl. In an even further aspect, R²⁵ when present, is hydrogen and each occurrence of R²⁴, R²⁶, and R²⁷, when present, is independently selected from the group consisting of hydrogen and methyl.

In a further aspect, R²⁶, when present, is hydrogen and each occurrence of R²⁴, R²⁵ and R²⁷, when present, is independently selected from the group consisting of hydrogen and C1-C4 alkyl. In a still further aspect, R²⁶, when present, is hydrogen and each occurrence of R²⁴, R²⁵, and R²⁷, when present, is independently selected from the group consisting of hydrogen, methyl, ethyl, n-propyl, and isopropyl. In yet a further aspect, R²⁶, when present, is hydrogen and each occurrence of R²⁴, R²⁵, and R²⁷, when present, is independently selected from the group consisting of hydrogen, methyl, and ethyl. In an even further aspect, R²⁶, when present, is hydrogen and each occurrence of R²⁴, R²⁵, and R²⁷, when present, is independently selected from the group consisting of hydrogen and methyl.

In a further aspect, R²⁷, when present, is hydrogen and each occurrence of R²⁴, R²⁵, and R²⁶, when present, is independently selected from the group consisting of hydrogen and C1-C4 alkyl. In a still further aspect, R²⁷, when present, is hydrogen and each occurrence of R²⁴, R²⁵, and R²⁶, when present, is independently selected from the group consisting of hydrogen, methyl, ethyl, n-propyl, and isopropyl. In yet a further aspect, R²⁷, when present, is hydrogen and each occurrence of R²⁴, R²⁵, and R²⁶, when present, is independently selected from the group consisting of hydrogen, methyl, and ethyl. In an even further aspect, R²⁷, when present, is hydrogen and each occurrence of R²⁴, R²⁵, and R²⁶, when present, is independently selected from the group consisting of hydrogen and methyl.

i. R^{30A}, R^{30B}, R^{30C}, and R^{30D} Groups

In one aspect, each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a}, R^{30b}, R^{30c}, and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —S₂R⁴³. In a still further aspect, each of R^{30a}, R^{30b}, R^{30c}, and R^{30d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴², and —SO₂R⁴³. In yet a further aspect, each of R^{30a}, R^{30b}, R^{30c}, and R^{30d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30a}, R^{30b}, R^{30c} and R^{30d} is independently selected from the group consisting of hydrogen, halogen, —CN, —NH₂, —OH, —NO₂, C1-C4 alkyl, C2-C4 alkenyl, C1-C4 hydroxyalkyl, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a}, R^{30b}, R^{30c}, and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —SO₂R⁴³. In a still further aspect, each of R^{30a}, R^{30b}, R^{30c}, and R^{30d} is independently selected from the group consisting of hydrogen, halogen, —CN, —NH₂, —OH, —NO₂, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴², and —SO₂R⁴³. In yet a further aspect, each of R^{30a}, R^{30b}, R^{30c}, and R^{30d} is independently selected from the group consisting of hydrogen, halogen, —CN, —NH₂, —OH, —NO₂, methyl, —CH₂OH, —OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a}, R^{30b}, R^{30c}, and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —SO₂R⁴³. In a still further aspect, each of R^{30a}, R^{30b}, R^{30c}, and R^{30d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₀R⁴¹, —C(O)NR⁴², and —SO₂R⁴³. In yet a further aspect, each of R³⁰, R^{30b}, R^{30c}, and R^{30d} is independently selected from the group consisting of hydrogen, halogen, —CN, —NH₂, —OH, —NO₂, methyl, —CH₂F, —CH₂Cl, —CH₂CN, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, —C(O)R⁴⁰, —CO₂R⁴¹, —C(O)NR⁴², and —SO₂R⁴³. In a further aspect, each of R^{30a}, R^{30b}, R^{30c}, and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —C(O)R⁴⁰, —CO₂R⁴¹, —C(O)NR⁴², and —SO₂R⁴³. In a still further aspect, each of R^{30a}, R^{30b}, R^{30c}, and R^{30d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, OCF₃, —OCHF₃, —OCH₂F, —OCH₂CH₂F, —OCH₃, —OCH₂CH₃, —C(O)R⁴⁰, —CO₂R⁴¹, —C(O)NR⁴², and —SO₂R⁴³. In yet a further aspect each of R^{30a}, R^{30b}, R^{30c}, and R^{30d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —C(O)R⁴⁰, —CO₂R⁴¹, —C(O)NR⁴², and —SO₂R⁴³.

In various aspects, each of R^{30a}, R^{30b}, R^{30c}, and R^{30d} is independently selected from the group consisting of hydrogen, halogen, C1-C4 alkyl, C2-C4 alkenyl, C1-C4 hydroxyalkyl, C1-C4 alkoxy, C1-C4 alkylamino, (C1-C4)(C1-C4) dialkylamino, and C1-C4 aminoalkyl. In a further aspect, each of R^{30a}, R^{30b}, R^{30c}, and R^{30d} is independently selected from the group consisting of hydrogen, halogen, methyl, ethyl, n-propyl, isopropyl, ethenyl, propenyl, isopropenyl, —CH₂OH, —CH₂CH₂OH, —CH₂CH₂CH₂OH, —CH(CH₃)CH₂OH, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{30a}, R^{30b}, R^{30c}, and R^{30d} is independently selected from the group consisting of hydrogen, halogen, methyl, ethyl, ethenyl, —CH₂OH, —CH₂CH₂OH, —OCH₃, —OCH₂CH₃, —NHCH₃, —NHCH₂CH₃, —N(CH₃)₂, —N(CH₃)CH₂CH₃, —CH₂NH₂, and —CH₂CH₂NH₂. In yet a further aspect, each of R^{30a}, R^{30b}, R^{30c}, and R^{30d} is independently selected from the group consisting of hydrogen, halogen, methyl, —CH₂OH, —OCH₃, —NHCH₃, —N(CH₃)₂, and —CH₂NH₂.

In various aspects, each of R^{30a}, R^{30b}, R^{30c}, and R^{30d} is independently selected from the group consisting of hydrogen, halogen, C1-C4 alkyl, C2-C4 alkenyl, C1-C4 haloalkyl, C1-C4 cyanoalkyl, C1-C4 alkylamino, (C1-C4)(C1-C4) dialkylamino, and C1-C4 aminoalkyl. In a further aspect, each of R^{30a}, R^{30b}, R^{30c}, and R^{30d} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, and —CH(CH₃)CH₂NH₂. In a still further aspect, each of R^{30a}, R^{30b}, R^{30c}, and R^{30d} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —NHCH₃, NHCH₂CH₃, —N(CH₃)₂, —N(CH₃)CH₂CH₃, —CH₂NH₂, and —CH₂CH₂NH₂. In yet a further aspect, each of R^{30a}, R^{30b}, R^{30c}, and R^{30d} is independently selected from the group consisting of hydrogen, halogen, methyl, —CH₂F, —CH₂Cl, —CH₂CN, —NHCH₃, —N(CH₃)₂, and —CH₂NH₂.

In various aspects, each of R^{30a}, R^{30b}, R^{30c}, and R^{30d} is independently selected from the group consisting of hydrogen, halogen, C1-C4 alkyl, C2-C4 alkenyl, C1-C4 haloalkyl, C1-C4 cyanoalkyl, C1-C4 hydroxyalkyl, C1-C4 haloalkoxy, and C1-C4 alkoxy. In a further aspect, each of R^{30a}, R^{30b}, R^{30c}, and R^{30d} is independently selected from the group consisting of hydrogen, halogen, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, OCH₂CH₃, —OCH₂CH₂CH₃, and —OCH(CH₃)₂. In a still further aspect, each of R^{30a}, R^{30b}, R^{30c}, and R^{30d} is independently selected from the group consisting of hydrogen, halogen, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, and —OCH₂CH₃. In yet a further aspect, each of R^{30a}, R^{30b}, R^{30c}, and R^{30d} is independently selected from the group consisting of hydrogen, halogen, methyl, —CH₂F, —CH₂Cl, —CH₂CN, —CH₂OH, —OCF₃, —OCHF₃, —OCH₂F, and —OCH₃.

In a further aspect, each of R^{30a}, R^{30b}, R^{30c}, and R^{30d} is hydrogen.

In a further aspect, each of R^{30b} and R^{30c} is hydrogen. In a still further aspect, each of R^{30b} and R^{30d} is hydrogen. In yet a further aspect, each of R^{30a} and R^{30b} is hydrogen. In an even further aspect, each of R^{30a} and R^{30c} is hydrogen. In a still further aspect, each of R^{2a} and R^{2a} is hydrogen. In yet a further aspect, each of R^{30c} and R^{30d} is hydrogen.

In a further aspect, each of R^{30a}, R^{30b}, R^{30c}, and R^{30d} is selected from the group consisting of hydrogen and halogen. In a still further aspect, each of R^{30a}, R^{30b}, R^{30c}, and R^{30d} is selected from the group consisting of hydrogen, —F, —Cl, and —Br. In yet a further aspect, each of R^{30a}, R^{30b}, R^{30c}, and R^{30d} is selected from the group consisting of hydrogen, —F, and —Cl. In an even further aspect, each of R^{30a}, R^{30b}, R^{30c}, and R^{30d} is selected from the group consisting of hydrogen and —Cl. In a still further aspect, each of R^{30a}, R^{30b}, R^{30c}, and R^{30d} is selected from the group consisting of hydrogen and —F.

In various aspects, each of R^{30a} and R^{30b} is hydrogen and each of R^{30c} and R^{30a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a} and R^{30b} is hydrogen and each of R^{30c} and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, CH(CH₃)CH₂NH₂, —C(O)R⁴⁰, —CO₂R⁴¹, —C(O)NR⁴², and —SO₂R⁴³. In a still further aspect, each of R^{30a} and R^{30b} is hydrogen and each of R^{30c} and R^{30d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴² and —SO₂R⁴³. In yet a further aspect, each of R^{30a} and R^{30b} is hydrogen and each of R^{30c} and R^{30d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30a} and R^{30c} is hydrogen and each of R^{30b} and R^{30a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a} and R^{30c} is hydrogen and each of R^{30b} and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —SO₂R⁴³. In a still further aspect, each of R^{30a} and R^{30c} is hydrogen and each of R^{30b} and R^{30d} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴² and —SO₂R⁴³. In yet a further aspect, each of R^{30a} and R^{30c} is hydrogen and each of R^{30b} and R^{30d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30a} and R^{30d} is hydrogen and each of R^{30b} and R^{30c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30a} and R^{30d} is hydrogen and each of R^{30b} and R^{30c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —SO₂R⁴³. In a still further aspect, each of R^{30a} and R^{30d} is hydrogen and each of R^{30b} and R^{30c} is independently selected from the group consisting of hydrogen, halogen, —CN, —NH₂, —OH, —NO₂, methyl, ethyl, ethenyl, —CH₂F, —CH₂CH₂F, —CH₂C, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, N(CH₃)₂, —N(CH₃)CH₂CH₃, —CH₂NH₂, —CH₂CH₂NH₂, —C(O)R⁴⁰, —CO₂R⁴¹, —C(O)NR⁴² and —SO₂R⁴³. In yet a further aspect, each of R^{30a} and R^{30d} is hydrogen and each of R^{30b} and R^{30c} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30b} and R^{30c} is hydrogen and each of R^{30a} and R^{30a} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30b} and R^{30c} is hydrogen and each of R^{30a} and R^{30d} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₀R⁴¹, —C(O)NR⁴², and —SO₂R⁴³. In a still further aspect, each of R^{30b} and R^{30c} is hydrogen and each of R^{30a} and R^{30d} is independently selected from the group consisting of hydrogen, halogen, —CN, —NH₂, —OH, —NO₂, methyl, ethyl, ethenyl, —CH₂F, —CH₂CH₂F, —CH₂CL, —CH₂CH₂CL, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴², and —SO₂R⁴³. In yet a further aspect, each of R^{30b} and R^{30c} is hydrogen and each of R^{30a} and R^{30d} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R⁴⁰, —CO₂R⁴¹, —C(O)NR⁴², and —SO₂R⁴³.

In various aspects, each of R^{30b} and R^{30d} is hydrogen and each of R^{30a} and R^{30c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30b} and R^{30d} is hydrogen and each of R^{30a} and R^{30c} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, —NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₂R⁴¹, —C(O)NR⁴², and —SO₂R⁴³. In a still further aspect, each of R^{30b} and R^{30d} is hydrogen and each of R^{30a} and R^{30c} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂CL, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₀R⁴¹, —C(O)NR⁴², and —SO₂R⁴³. In yet a further aspect, each of R^{30b} and R^{30d} is hydrogen and each of R^{30a} and R^{30c} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R40, —CO₂R⁴¹, —C(O)NR⁴², and —SO₂R⁴³.

In various aspects, each of R^{30c} and R^{30d} is hydrogen and each of R^{30a} and R^{30b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³. In a further aspect, each of R^{30c} and R^{30d} is hydrogen and each of R^{30a} and R^{30b} is independently selected from the group consisting of hydrogen, halogen, —CN, —NH₂, —OH, —NO₂, methyl, ethyl, n-propyl, isopropyl, ethenyl, propenyl, isopropenyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCHF₃, —OCH₂F, —OCH₂CH₂F, —OCH₂CH₂CH₂F, —OCH(CH₃)CH₂F, —OCH₃, —OCH₂CH₃, —OCH₂CH₂CH₃, —OCH(CH₃)₂, NHCH₃, —NHCH₂CH₃, —NHCH₂CH₂CH₃, —NHCH(CH₃)₂, —N(CH₃)₂, —N(CH₃)CH₂CH₃, —N(CH₃)CH₂CH₂CH₃, —N(CH₃)CH(CH₃)₂, —CH₂NH₂, —CH₂CH₂NH₂, —CH₂CH₂CH₂NH₂, —CH(CH₃)CH₂NH₂, —C(O)R⁴⁰, —CO₀R⁴¹, —C(O)NR⁴², and —SO₂R⁴³. In a still further aspect, each of R^{30c} and R^{30d} is hydrogen and each of R^{30a} and R^{30b} is independently selected from the group consisting of hydrogen, halogen, —CN, —NH₂, —OH, —NO₂, methyl, ethyl, ethenyl, —CH₂F, —CH₂CH₂F, —CH₂Cl, —CH₂CH₂Cl, —CH₂CN, —CH₂CH₂CN, —CH₂OH, —CH₂CH₂OH, —OCF₃, —OCHF₃, —OCH₂F, —OCH₂CH₂F, —OCH₃, —OCH₂CH₃, —NHCH₃, —NHCH₂CH₃, —N(CH₃)₂, —N(CH₃)CH₂CH₃, —CH₂NH₂, —CH₂CH₂NH₂, —C(O)R⁴⁰, —CO₂R⁴¹, —C(O)NR⁴² and —SO₂R⁴³. In yet a further aspect, each of R^{30c} and R^{30d} is hydrogen and each of R^{30a} and R^{30b} is independently selected from the group consisting of hydrogen, halogen, —CN, —NH₂, —OH, —NO₂, methyl, —CH₂F, —CH₂Cl, —CH₂CN, —CH₂OH, —OCF₃, —OCHF₃, —OCH₂F, —OCH₃, —NHCH₃, —N(CH₃)₂, —CH₂NH₂, —C(O)R⁴⁰, —CO₂R⁴¹, —C(O)NR⁴², and —SO₂R⁴³.

In a further aspect, each of R^{30a}, R^{30b}, R^{30c}, and R^{30d} is independently selected from the group consisting of hydrogen, halogen, C1-C4 alkyl, C1-C4 haloalkyl, and C1-C4 alkoxy. In a still further aspect, each of R^{30a}, R^{30b}, R^{30c}, and R^{30d} is independently selected from the group consisting of hydrogen, —F, —Cl, methyl, ethyl, n-propyl, isopropyl, —CH₂F, —CH₂CH₂F, —CH₂CH₂CH₂F, —CH(CH₃)CH₂F, —CH₂Cl, —CH₂CH₂Cl, —CH₂CH₂CH₂Cl, —CH(CH₃)CH₂Cl, —OCH₃, —OCH₂CH₃, —OCH₂CH₂CH₃, and —OCH(CH₃)₂. In yet a further aspect, each of R^{30a}, R^{30b}, R^{30c}, and R^{30d} is independently selected from the group consisting of hydrogen, —F, —Cl, methyl, ethyl, —CH₂F, —CH₂CH₂F, —CH₂Cl, —CH₂CH₂Cl, —OCH₃, and —OCH₂CH₃. In an even further aspect, each of R^{30a}, R^{30b}, R^{30c}, and R^{30d} is independently selected from the group consisting of hydrogen, —F, —Cl, methyl, —CH₂F, —CH₂Cl, and —OCH₃.

In a further aspect, each of R^{30a}, R^{30b}, R^{30c}, and R^{30d} is independently selected from hydrogen, halogen, and C1-C4 haloalkyl. In a still further aspect, each of R^{30a}, R^{30b}, R^{30c}, and R^{30d} is independently selected from hydrogen, —F, —Cl, —CH₂F, —CH₂CH₂F, —CH₂CH₂CH₂F, —CH(CH₃)CH₂F, —CH₂Cl, —CH₂CH₂Cl, —CH₂CH₂CH₂Cl, and —CH(CH₃)CH₂C1. In a still further aspect, each of R^{30a}, R^{30b}, R^{30c}, and R^{30d} is independently selected from hydrogen, —F, —Cl, —CH₂F, —CH₂CH₂F, —CH₂Cl, and —CH₂CH₂C1. In yet a further aspect, each of R^{30a}, R^{30b}, R^{30c}, and R^{30d} is independently selected from hydrogen, —F, —Cl, —CH₂F, and —CH₂Cl.

j. R⁴⁰, R⁴¹, R⁴², and R⁴³Groups

In one aspect, each occurrence of R⁴⁰, R⁴¹, R⁴², and R⁴³, when present, is independently selected from the group consisting of hydrogen and C1-C4 alkyl. In a further aspect, each occurrence of R⁴⁰, R⁴¹, R⁴², and R⁴³, when present, is independently selected from the group consisting of hydrogen, methyl, ethyl, n-propyl, and isopropyl. In a still further aspect, each occurrence of R⁴⁰, R⁴¹, R⁴², and R⁴³, when present, is independently selected from the group consisting of hydrogen, methyl, and ethyl. In yet a further aspect, each occurrence of R⁴⁰, R⁴¹, R⁴, and R⁴³, when present, is independently selected from the group consisting of hydrogen and ethyl. In an even further aspect, each occurrence of R⁴⁰, R⁴¹, R⁴², and R⁴³, when present, is independently selected from the group consisting of hydrogen and methyl.

In a further aspect, each occurrence of R⁴⁰, R⁴¹, R⁴², and R⁴³, when present, is hydrogen.

In various aspects, each occurrence of R⁴⁰, R⁴¹, R⁴², and R⁴³, when present, is C1-C4 alkyl. In a further aspect, each occurrence of R⁴⁰, R⁴¹, R⁴², and R⁴³, when present, is independently selected from the group consisting of methyl, ethyl, n-propyl, and isopropyl.

In a still further aspect, each occurrence of R⁴⁰, R⁴¹, R⁴², and R⁴³, when present, is independently selected from the group consisting of methyl and ethyl. In yet a further aspect, each occurrence of R⁴⁰, R⁴¹, R⁴², and R⁴³, when present, is ethyl. In an even further aspect, each occurrence of R⁴⁰, R⁴¹, R⁴², and R⁴³, when present, is methyl.

In a further aspect, R⁴⁰, when present, is hydrogen and each occurrence of R⁴¹, R⁴², and R⁴³, when present, is independently selected from the group consisting of hydrogen and C1-C4 alkyl. In a still further aspect, R⁴⁰, when present, is hydrogen and each occurrence of R⁴¹, R⁴², and R⁴³, when present, is independently selected from the group consisting of hydrogen, methyl, ethyl, n-propyl, and isopropyl. In yet a further aspect, R⁴⁰, when present, is hydrogen and each occurrence of R⁴¹, R⁴², and R⁴³, when present, is independently selected from the group consisting of hydrogen, methyl, and ethyl. In an even further aspect, R⁴⁰, when present, is hydrogen and each occurrence of R⁴¹, R⁴², and R⁴³, when present, is independently selected from the group consisting of hydrogen and methyl.

In a further aspect, R⁴¹, when present, is hydrogen and each occurrence of R⁴⁰, R⁴², and R⁴³, when present, is independently selected from the group consisting of hydrogen and C1-C4 alkyl. In a still further aspect, R⁴¹, when present, is hydrogen and each occurrence of R⁴⁰, R⁴², and R⁴³, when present, is independently selected from the group consisting of hydrogen, methyl, ethyl, n-propyl, and isopropyl. In yet a further aspect, R⁴¹, when present, is hydrogen and each occurrence of R⁴⁰, R⁴², and R⁴³, when present, is independently selected from the group consisting of hydrogen, methyl, and ethyl. In an even further aspect, R⁴¹, when present, is hydrogen and each occurrence of R⁴⁰, R⁴², and R⁴³, when present, is independently selected from the group consisting of hydrogen and methyl.

In a further aspect, R⁴², when present, is hydrogen and each occurrence of R⁴⁰, R⁴¹, and R⁴³, when present, is independently selected from the group consisting of hydrogen and C1-C4 alkyl. In a still further aspect, R⁴², when present, is hydrogen and each occurrence of R⁴⁰, R⁴¹, and R⁴³, when present, is independently selected from the group consisting of hydrogen, methyl, ethyl, n-propyl, and isopropyl. In yet a further aspect, R⁴², when present, is hydrogen and each occurrence of R⁴⁰, R⁴¹, and R⁴³, when present, is independently selected from the group consisting of hydrogen, methyl, and ethyl. In an even further aspect, R⁴², when present, is hydrogen and each occurrence of R⁴⁰, R⁴¹, and R⁴³, when present, is independently selected from the group consisting of hydrogen and methyl.

In a further aspect, R⁴³, when present, is hydrogen and each occurrence of R⁴⁰, R⁴¹, and R⁴², when present, is independently selected from the group consisting of hydrogen and C1-C4 alkyl. In a still further aspect, R⁴³, when present, is hydrogen and each occurrence of R⁴⁰, R⁴¹, and R⁴², when present, is independently selected from the group consisting of hydrogen, methyl, ethyl, n-propyl, and isopropyl. In yet a further aspect, R⁴³, when present, is hydrogen and each occurrence of R⁴⁰, R⁴¹, and R⁴², when present, is independently selected from the group consisting of hydrogen, methyl, and ethyl. In an even further aspect, R⁴³, when present, is hydrogen and each occurrence of R⁴⁰, R⁴¹, and R⁴², when present, is independently selected from the group consisting of hydrogen and methyl.

k. Ar¹ Groups

In one aspect, Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a further aspect, Ar¹ is selected from the group consisting of aryl and heteroaryl, and is substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a still further aspect, Ar¹ is selected from the group consisting of aryl and heteroaryl, and is substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In yet a further aspect, Ar¹ is selected from the group consisting of aryl and heteroaryl, and is monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In an even further aspect, Ar¹ is selected from the group consisting of aryl and heteroaryl, and is unsubstituted.

In various aspects, Ar¹ is aryl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. Examples of aryls include, but are not limited to, phenyl, naphthyl, and phenanthrenyl. In a further aspect, Ar¹ is aryl substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₀R²⁵, —C(O)NR²⁶, and —SO₂R²⁷. In a still further aspect, Ar¹ is aryl substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R^{2s}, —C(O)NR²⁶, and —SO₂R²⁷. In yet a further aspect, Ar¹ is aryl monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²¹, —C(O)NR²⁶, and —SO₂R²⁷. In an even further aspect, Ar¹ is unsubstituted aryl.

In various aspects, Ar¹ is monocyclic aryl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₀R²⁵, —C(O)NR²⁶, and —SO₂R²⁷. In a further aspect, Ar¹ is monocyclic aryl substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a still further aspect, Ar¹ is monocyclic aryl substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In yet a further aspect, Ar¹ is monocyclic aryl monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In an even further aspect, Ar¹ is unsubstituted monocyclic aryl.

In various aspects, Ar¹ is heteroaryl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. Examples of heteroaryls include, but are not limited to, pyrrolyl, furanyl, thiophenyl, indolyl, benzofuranyl, benzothiophenyl, triazolyl, imidazolyl, oxazolyl, thiazolyl, pyrazolyl, isoxazolyl, isothiazolyl, pyridinyl, quinolinyl, and isoquinolinyl. In a further aspect, Ar¹ is heteroaryl substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a still further aspect, Ar¹ is heteroaryl substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In yet a further aspect, Ar¹ is heteroaryl monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In an even further aspect, Ar¹ is unsubstituted heteroaryl.

In various aspects, Ar¹ is pyridinyl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a further aspect, Ar¹ is pyridinyl substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a still further aspect, Ar¹ is pyridinyl substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In yet a further aspect, Ar¹ is pyridinyl monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In an even further aspect, Ar¹ is unsubstituted pyridinyl.

In various aspects, Ar¹ is 2-pyridinyl substituted with 0, 1, 2, or 3 groups independently selected from the group consisting of hydrogen, halogen, —CN, —NH₂, —OH, NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In a further aspect, Ar¹ is 2-pyridinyl substituted with 0, 1, or 2 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₀R²⁵, —C(O)NR²⁶, and —SO₂R²⁷. In a still further aspect, Ar¹ is 2-pyridinyl substituted with 0 or 1 groups selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In yet a further aspect, Ar¹ is 2-pyridinyl monosubstituted with a group selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷. In an even further aspect, Ar¹ is unsubstituted 2-pyridinyl.

### 2. Example Compounds

In one aspect, a compound can be present as one or more of the following structures:

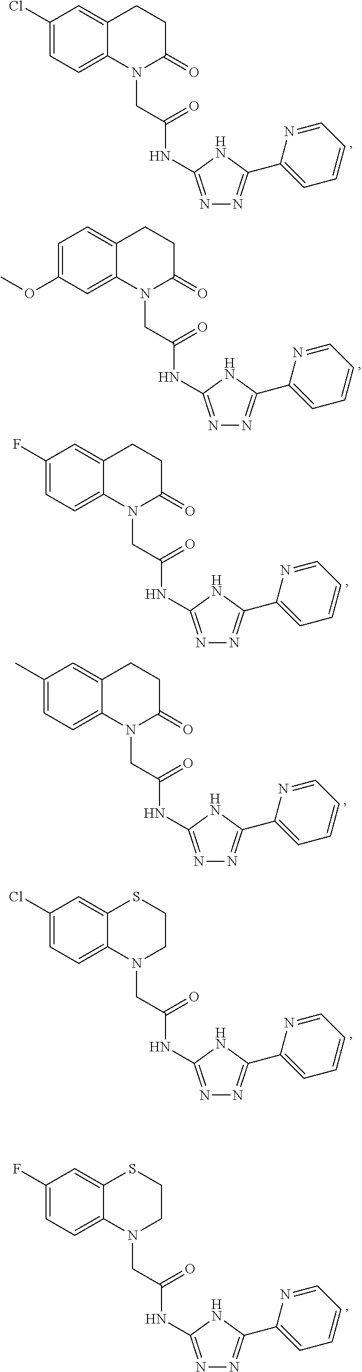 [SMILES: O=C(CN1C(=O)CCc2cc(Cl)ccc21)Nc1nnc(-c2ccccn2)[nH]1 ; COc1ccc2c(c1)N(CC(=O)Nc1nnc(-c3ccccn3)[nH]1)C(=O)CC2 ; O=C(CN1C(=O)CCc2cc(F)ccc21)Nc1nnc(-c2ccccn2)[nH]1 ; Cc1ccc2c(c1)CCC(=O)N2CC(=O)Nc1nnc(-c2ccccn2)[nH]1 ; O=C(CN1CCSc2cc(Cl)ccc21)Nc1nnc(-c2ccccn2)[nH]1 ; O=C(CN1CCSc2cc(F)ccc21)Nc1nnc(-c2ccccn2)[nH]1]

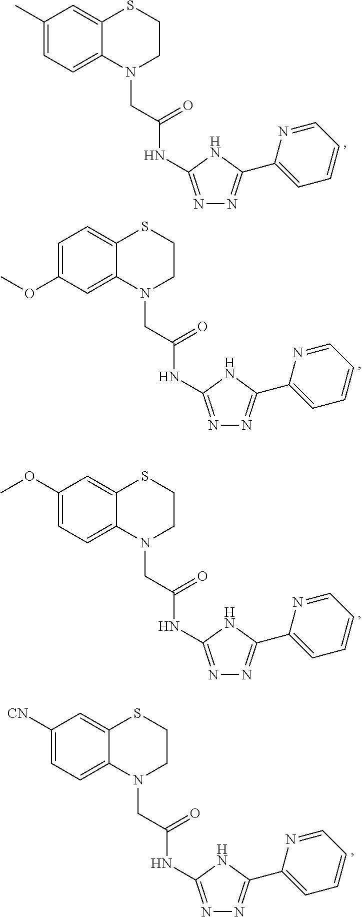

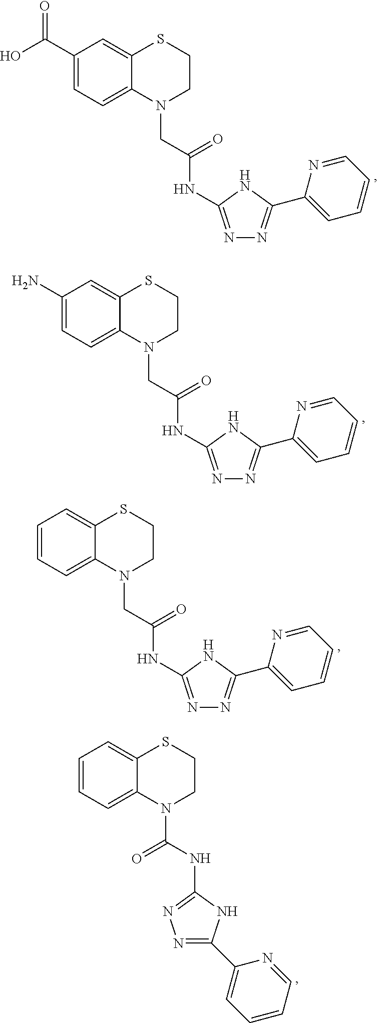

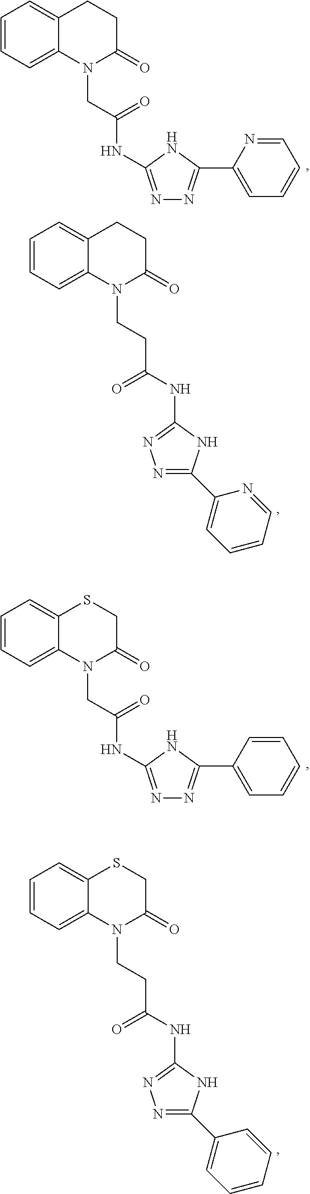

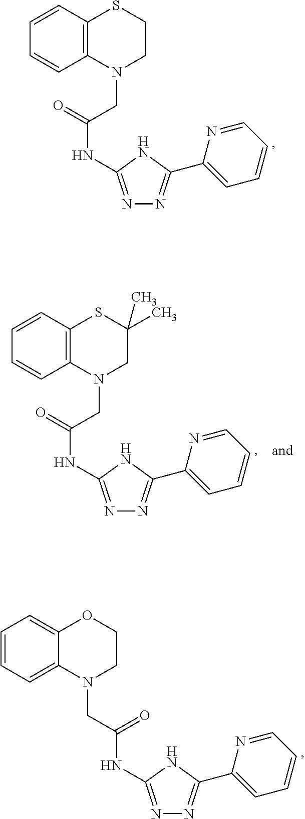 [SMILES: O=C(CN1CCSc2ccccc21)Nc1nnc(-c2ccccn2)[nH]1 ; CC1(C)CN(CC(=O)Nc2nnc(-c3ccccn3)[nH]2)c2ccccc2S1 ; O=C(CN1CCOc2ccccc21)Nc1nnc(-c2ccccn2)[nH]1]

or a pharmaceutically acceptable salt thereof.

In one aspect, a compound can be present as:

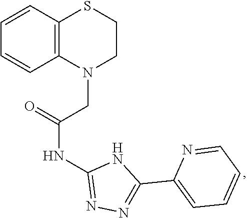 [SMILES: O=C(CN1CCSc2ccccc21)Nc1nnc(-c2ccccn2)[nH]1]

or a pharmaceutically acceptable salt thereof.

In one aspect, a compound can be present as one or more of the following structures:

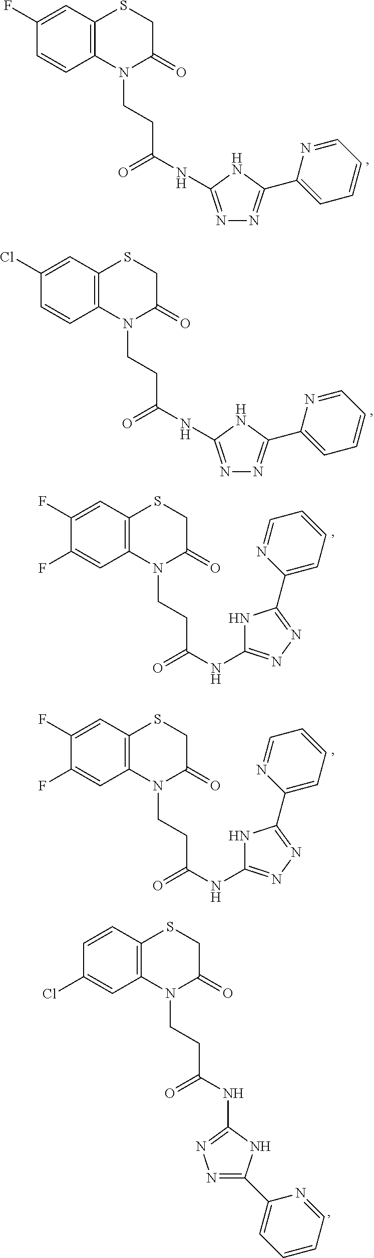

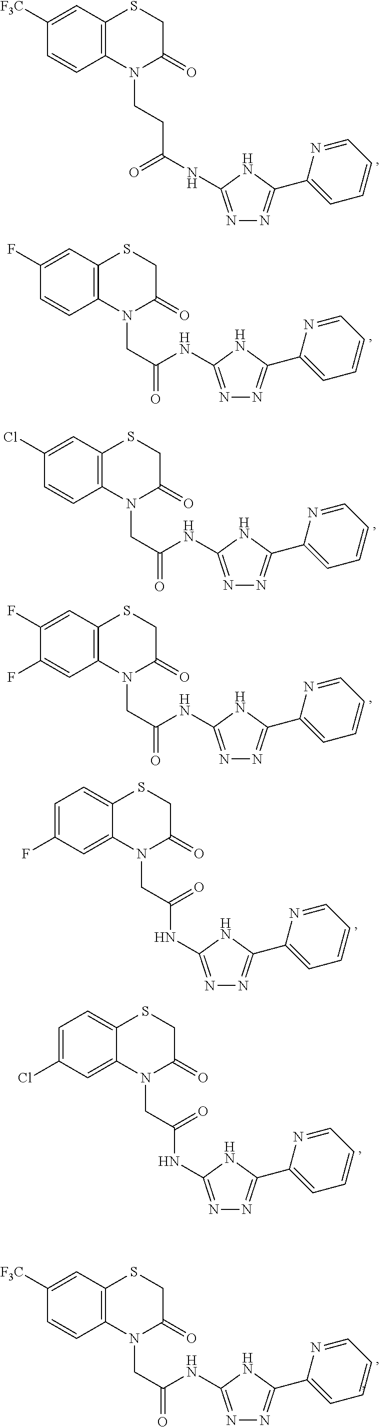

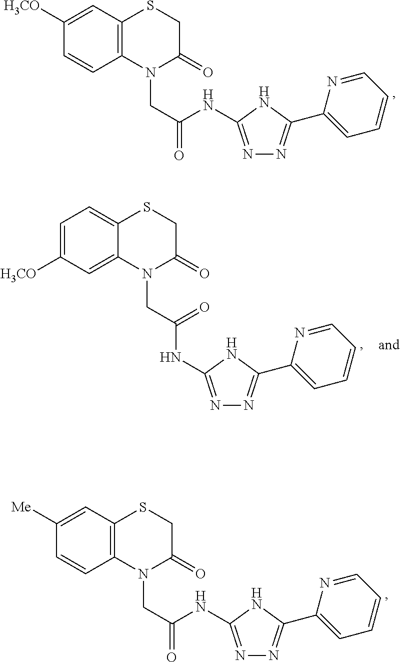 [SMILES: COc1ccc2c(c1)SCC(=O)N2CC(=O)Nc1nnc(-c2ccccn2)[nH]1 ; COc1ccc2c(c1)N(CC(=O)Nc1nnc(-c3ccccn3)[nH]1)C(=O)CS2 ; Cc1ccc2c(c1)SCC(=O)N2CC(=O)Nc1nnc(-c2ccccn2)[nH]1]

or a pharmaceutically acceptable salt thereof.

In one aspect, a compound can be present as one or more of the following structures:

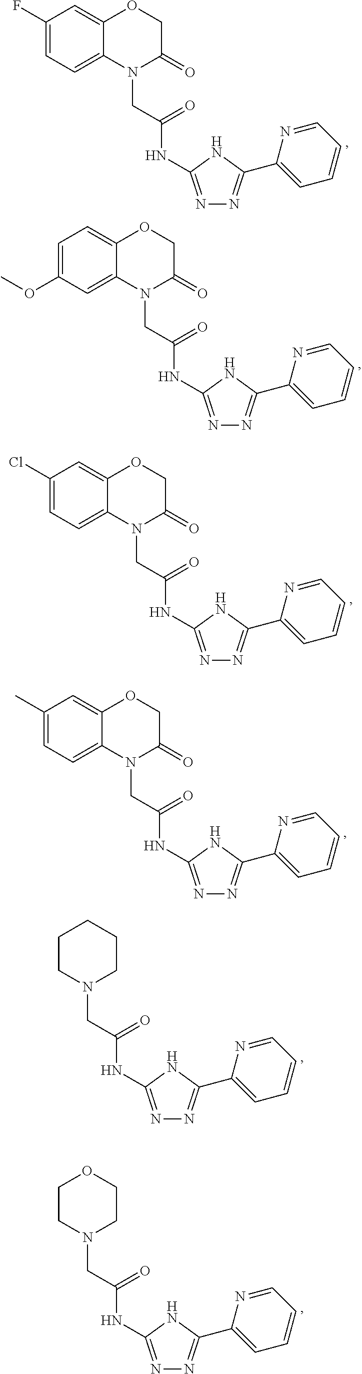 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1)Nc1nnc(-c2ccccn2)[nH]1 ; O=C(CN1CCOCC1)Nc1nnc(-c2ccccn2)[nH]1]

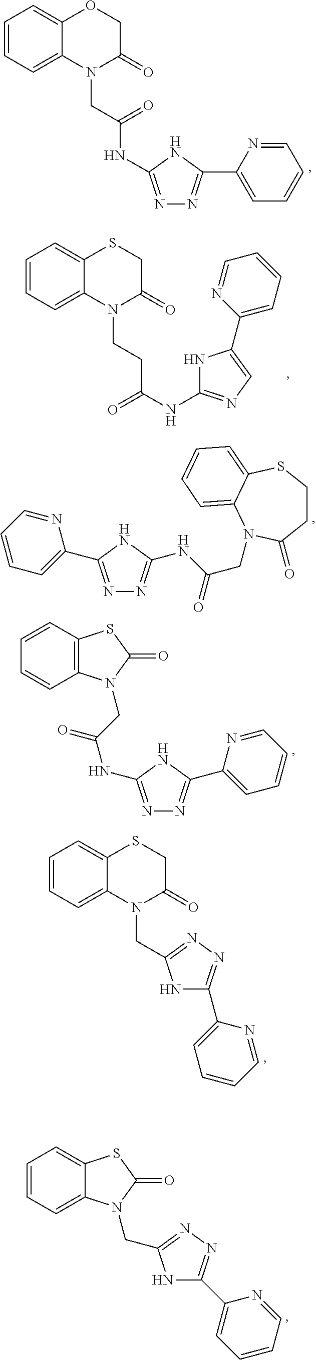

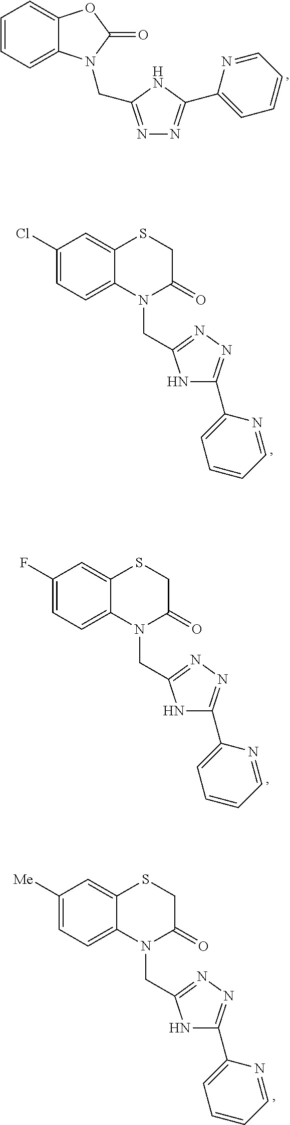 [SMILES: O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1 ; Cc1ccc2c(c1)SCC(=O)N2Cc1nnc(-c2ccccn2)[nH]1]

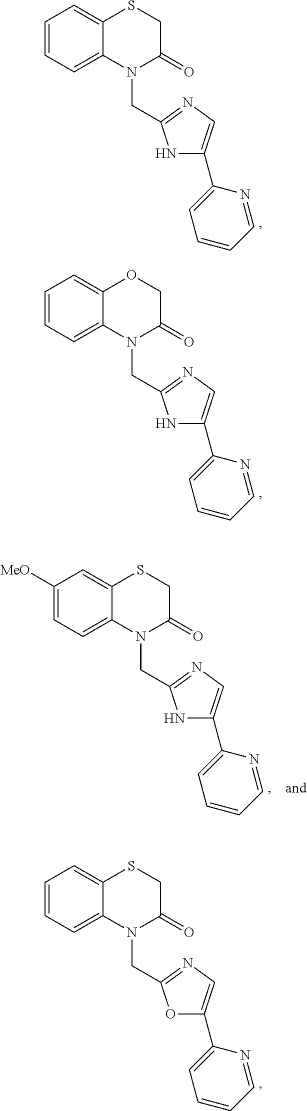 [SMILES: O=C1CSc2ccccc2N1Cc1ncc(-c2ccccn2)[nH]1 ; O=C1COc2ccccc2N1Cc1ncc(-c2ccccn2)[nH]1 ; COc1ccc2c(c1)SCC(=O)N2Cc1ncc(-c2ccccn2)[nH]1 ; O=C1CSc2ccccc2N1Cc1ncc(-c2ccccn2)o1]

or a pharmaceutically acceptable salt thereof.

In one aspect, a compound can be present as:

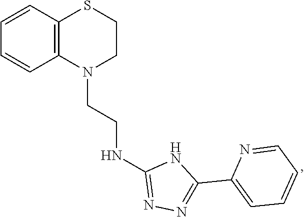 [SMILES: c1ccc(-c2nnc(NCCN3CCSc4ccccc43)[nH]2)nc1]

or a pharmaceutically acceptable salt thereof.

### 3. Prophetic Compound Examples

The following compound examples are prophetic, and can be prepared using the synthesis methods described herein above and other general methods as needed as would be known to one skilled in the art. It is anticipated that the prophetic compounds would be active as Tau-SH3 interaction inhibitors, and such activity can be determined using the assay methods described herein below.

In one aspect, a compound can be selected from:

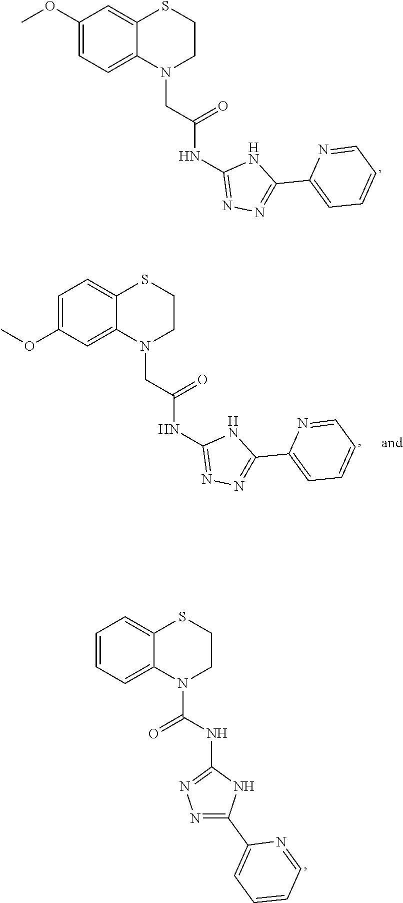 [SMILES: COc1ccc2c(c1)SCCN2CC(=O)Nc1nnc(-c2ccccn2)[nH]1 ; COc1ccc2c(c1)N(CC(=O)Nc1nnc(-c3ccccn3)[nH]1)CCS2 ; O=C(Nc1nnc(-c2ccccn2)[nH]1)N1CCSc2ccccc21]

or a pharmaceutically acceptable salt thereof.

It is contemplated that one or more compounds can optionally be omitted from the disclosed invention.

It is understood that the disclosed compounds can be used in connection with the disclosed methods, compositions, kits, and uses.

It is understood that pharmaceutical acceptable derivatives of the disclosed compounds can be used also in connection with the disclosed methods, compositions, kits, and uses. The pharmaceutical acceptable derivatives of the compounds can include any suitable derivative, such as pharmaceutically acceptable salts as discussed below, isomers, radiolabeled analogs, tautomers, and the like.

### C. Pharmaceutical Compositions

In one aspect, disclosed are pharmaceutical compositions comprising a disclosed compound, or a pharmaceutically acceptable salt thereof and a pharmaceutically acceptable carrier.

In one aspect, disclosed are pharmaceutical compositions comprising an effective amount of at least one compound having a structure represented by a formula:

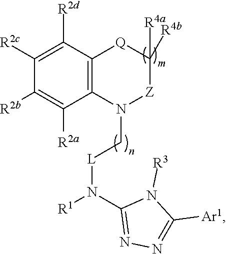

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

In one aspect, disclosed are pharmaceutical compositions comprising an effective amount of at least one compound having a structure represented by a formula:

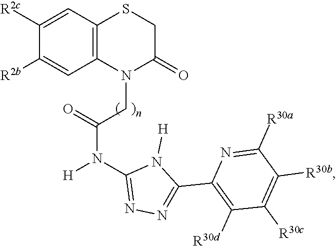

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

In one aspect, disclosed are pharmaceutical compositions comprising a therapeutically effective amount of at least one compound selected from the group consisting of:

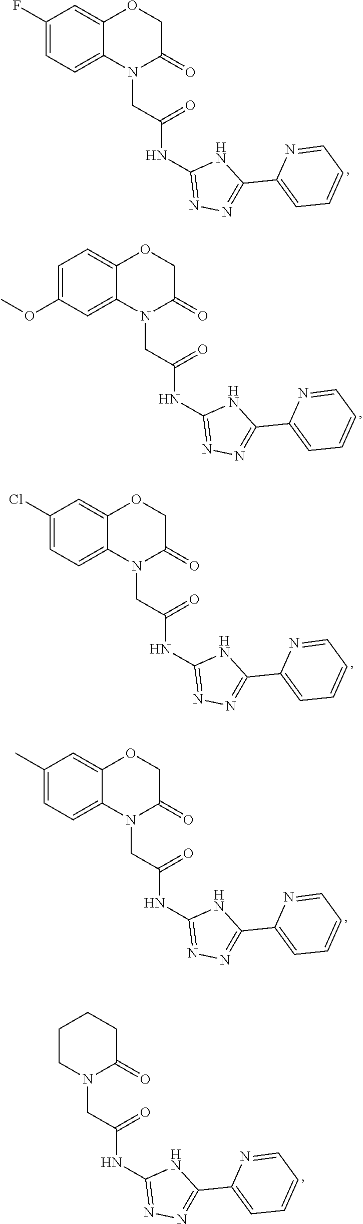 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1=O)Nc1nnc(-c2ccccn2)[nH]1]

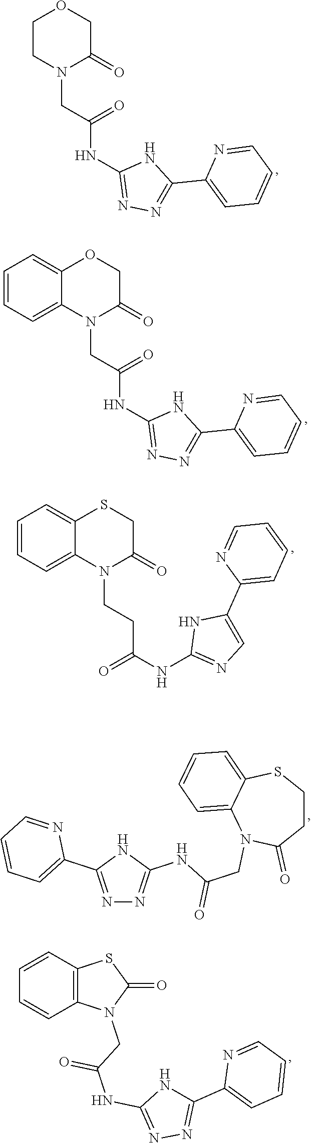 [SMILES: O=C(CN1CCOCC1=O)Nc1nnc(-c2ccccn2)[nH]1 ; O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1]

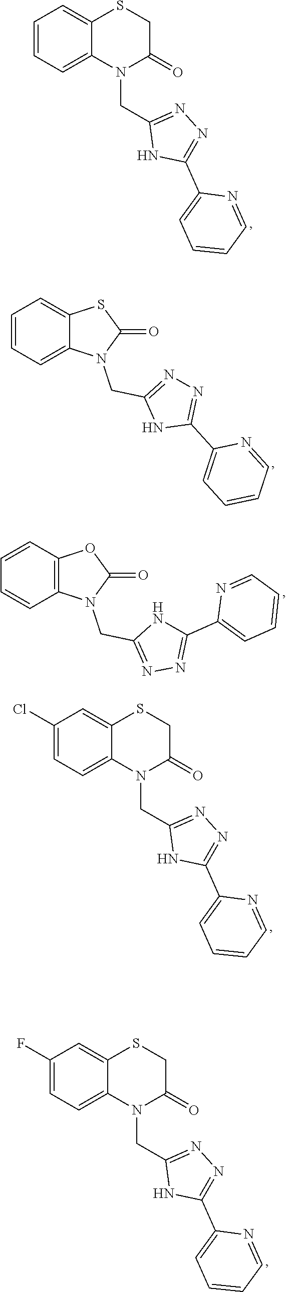

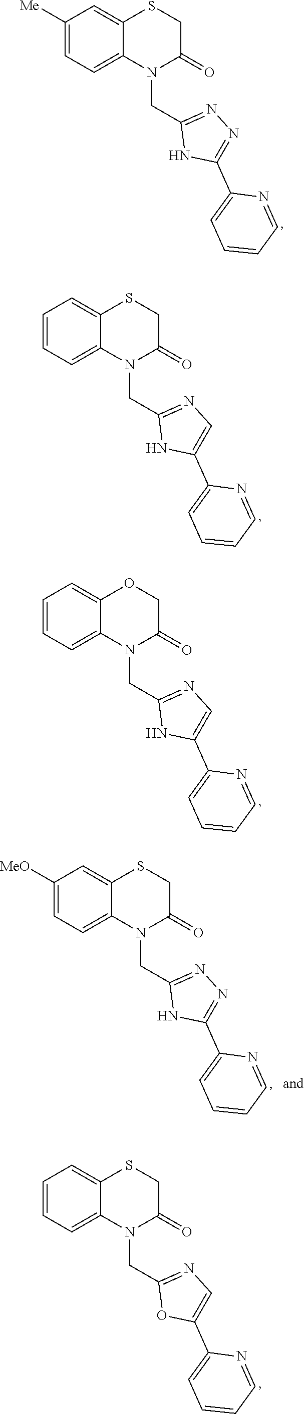 [SMILES: Cc1ccc2c(c1)SCC(=O)N2Cc1nnc(-c2ccccn2)[nH]1 ; O=C1CSc2ccccc2N1Cc1ncc(-c2ccccn2)[nH]1 ; O=C1COc2ccccc2N1Cc1ncc(-c2ccccn2)[nH]1 ; COc1ccc2c(c1)SCC(=O)N2Cc1nnc(-c2ccccn2)[nH]1 ; O=C1CSc2ccccc2N1Cc1ncc(-c2ccccn2)o1]

or a pharmaceutically acceptable salt thereof.

In one aspect, disclosed are pharmaceutical compositions comprising an effective amount of a compound having a structure:

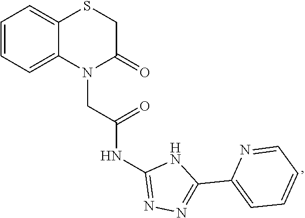 [SMILES: O=C(CN1C(=O)CSc2ccccc21)Nc1nnc(-c2ccccn2)[nH]1]

or a pharmaceutically acceptable salt thereof.

In various aspects, the compounds and compositions of the invention can be administered in pharmaceutical compositions, which are formulated according to the intended method of administration. The compounds and compositions described herein can be formulated in a conventional manner using one or more physiologically acceptable carriers or excipients. For example, a pharmaceutical composition can be formulated for local or systemic administration, e.g., administration by drops or injection into the ear, insufflation (such as into the ear), intravenous, topical, or oral administration.

The nature of the pharmaceutical compositions for administration is dependent on the mode of administration and can readily be determined by one of ordinary skill in the art. In various aspects, the pharmaceutical composition is sterile or sterilizable. The therapeutic compositions featured in the invention can contain carriers or excipients, many of which are known to skilled artisans. Excipients that can be used include buffers (for example, citrate buffer, phosphate buffer, acetate buffer, and bicarbonate buffer), amino acids, urea, alcohols, ascorbic acid, phospholipids, polypeptides (for example, serum albumin), EDTA, sodium chloride, liposomes, mannitol, sorbitol, water, and glycerol. The nucleic acids, polypeptides, small molecules, and other modulatory compounds featured in the invention can be administered by any standard route of administration. For example, administration can be parenteral, intravenous, subcutaneous, or oral. A modulatory compound can be formulated in various ways, according to the corresponding route of administration. For example, liquid solutions can be made for administration by drops into the ear, for injection, or for ingestion; gels or powders can be made for ingestion or topical application. Methods for making such formulations are well known and can be found in, for example, Remington's Pharmaceutical Sciences, 18th Ed., Gennaro, ed., Mack Publishing Co., Easton, PA 1990.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

In a further aspect, an effective amount is a therapeutically effective amount. In a still further aspect, an effective amount is a prophylactically effective amount.

In a further aspect, the pharmaceutical composition is administered to a mammal. In a still further aspect, the mammal is a human. In an even further aspect, the human is a patient.

In a further aspect, the pharmaceutical composition is used to treat a neurological disorder such as, for example, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD). In a still further aspect, the pharmaceutical composition is used to treat ALS.

In a further aspect, the pharmaceutical composition is used to treat a neurological disorder is associated with dysregulation of Tau-SH3 signaling. In a still further aspect, the pharmaceutical composition is used to treat a neurological disorder is associated with dysregulation of Tau-Fyn signaling. In yet a further aspect, the pharmaceutical composition is used to treat a neurological disorder associated with activation of Tau-SH3 signaling. In an even further aspect, the pharmaceutical composition is used to treat a neurological disorder associated with activation of Tau-Fyn signaling. In a still further aspect, the pharmaceutical composition is used to treat a neurological disorder is associated with dysfunction of brain-derived neurotropic factor.

It is understood that the disclosed compositions can be prepared from the disclosed compounds. It is also understood that the disclosed compositions can be employed in the disclosed methods of using.

### D. Methods Of Making A Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Routes I-III, as described and exemplified below. The following examples are provided so that the invention might be more fully understood, are illustrative only, and should not be construed as limiting.

### 1. Route I

In one aspect, substituted benzothiazone analogs can be prepared as shown below.

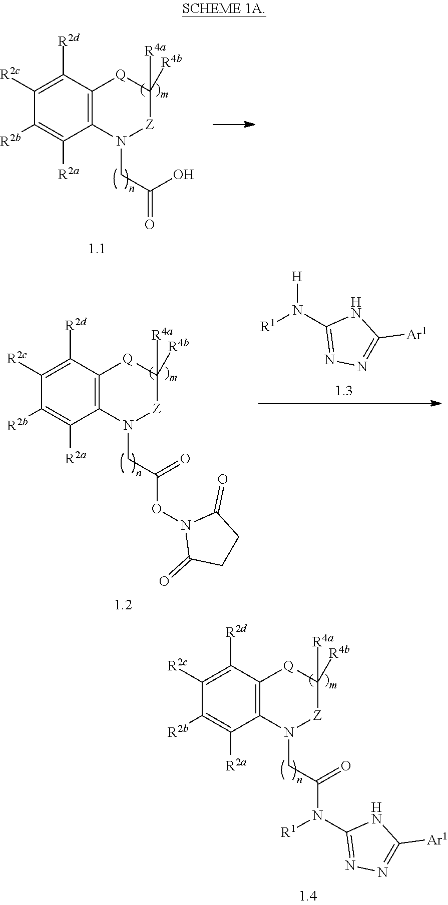

Compounds are represented in generic form, wherein PG is an amine protecting group and with other substituents as noted in compound descriptions elsewhere herein. A more specific example is set forth below.

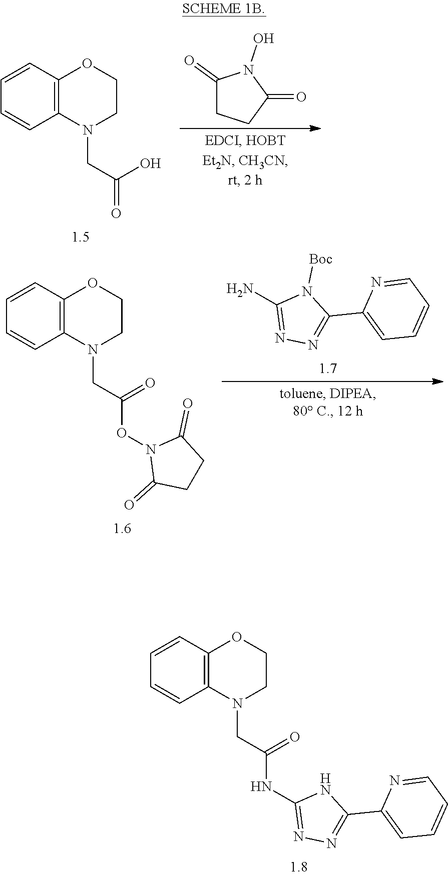 [SMILES: O=C(O)CN1CCOc2ccccc21 ; O=C1CCC(=O)N1O ; O=C(CN1CCOc2ccccc21)ON1C(=O)CCC1=O ; CC(C)(C)OC(=O)n1c(N)nnc1-c1ccccn1 ; O=C(CN1CCOc2ccccc21)Nc1nnc(-c2ccccn2)[nH]1]

In one aspect, compounds of type 1.8, and similar compounds, can be prepared according to reaction Scheme 1B above. Thus, compounds of type 1.6 can be prepared by a coupling reaction between an appropriate carboxylic acid, e.g., 1.5 as shown above, and an appropriate hydroxylamine, e.g., 1-hydroxypyrrolidine-2,5-dione as shown above.

Appropriate carboxylic acids and appropriate hydroxylamines are commercially available or prepared by methods known to one skilled in the art. The coupling reaction is carried out in the presence of an appropriate coupling agent, e.g., 1-ethyl-3-(3-dimethylaminopropyl)carbodiimide (EDCI), an appropriate activating agent, e.g., hydroxybenzotriazole (HOBt), and an appropriate base, e.g., triethylamine, in an appropriate solvent, e.g., acetonitrile. Compounds of type 1.8 can be prepared by a substitution reaction of an appropriate activated carboxylic acid, e.g. 1.6 as shown above, and an appropriate amine, e.g., 1.7 as shown above. Appropriate amines are commercially available or prepared by methods known to one skilled in the art. The substitution reaction is carried out in the presence of an appropriate base, e.g., diisopropylethylamine (DIPEA), in an appropriate solvent, e.g., toluene, at an appropriate temperature, e.g., 80° C. As can be appreciated by one skilled in the art, the above reaction provides an example of a generalized approach wherein compounds similar in structure to the specific reactants above (compounds similar to compounds of type 1.1, 1.2, and 1.3), can be substituted in the reaction to provide substituted benzothiazone analogs similar to Formula 1.4.

### 2. Route II

In one aspect, substituted benzothiazone analogs can be prepared as shown below.

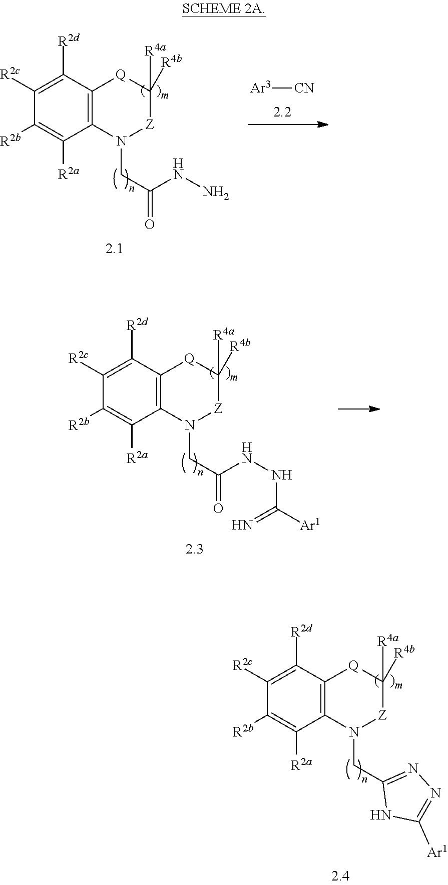

Compounds are represented in generic form, with substituents as noted in compound descriptions elsewhere herein. A more specific example is set forth below.

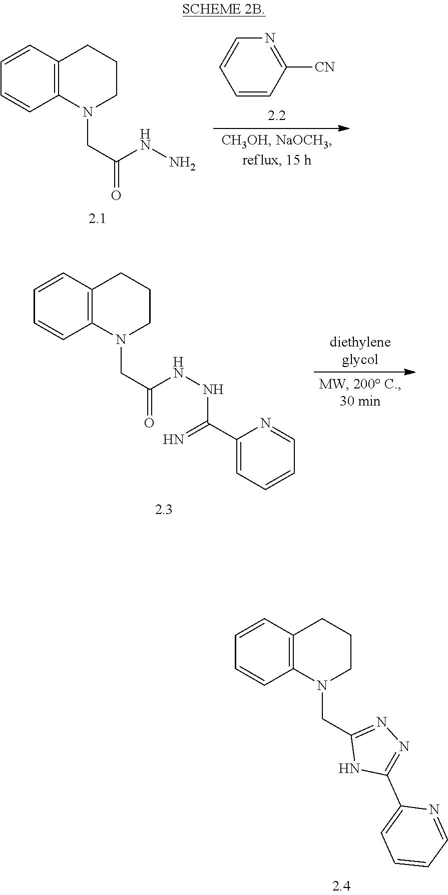 [SMILES: NNC(=O)CN1CCCc2ccccc21 ; N#Cc1ccccn1 ; N=C(NNC(=O)CN1CCCc2ccccc21)c1ccccn1 ; c1ccc(-c2nnc(CN3CCCc4ccccc43)[nH]2)nc1]

In one aspect, compounds of type 2.4, and similar compounds, can be prepared according to reaction Scheme 2B above. Thus, compounds of type 2.3 can be prepared by a substitution reaction between an appropriate hydrazine, e.g., 2.1 as shown above, and an appropriate cyanide, e.g., 2.2 as shown above. Appropriate hydrazines and appropriate cyanides are commercially available or prepared by methods known to one skilled in the art. The substitution reaction is carried out in the presence of an appropriate base, e.g., sodium methoxide, in an appropriate solvent, e.g., methanol, for an appropriate period of time, e.g., 15 hours, at an appropriate temperature, e.g., reflux. Compounds of type 2.4 can be prepared by a cyclization reaction of an appropriate acetohydrazide, e.g., 2.3 as shown above. The cyclization reaction is carried out in the presence of an appropriate solvent, e.g., diethylene glycol, at an appropriate temperature, e.g., 200° C., for an appropriate period of time, e.g., 30 minutes. As can be appreciated by one skilled in the art, the above reaction provides an example of a generalized approach wherein compounds similar in structure to the specific reactants above (compounds similar to compounds of type 2.1, 2.2, and 2.3), can be substituted in the reaction to provide substituted benzothiazone analogs similar to Formula 2.4.

### 3. Route III

In one aspect, substituted benzothiazone analogs can be prepared as shown below.

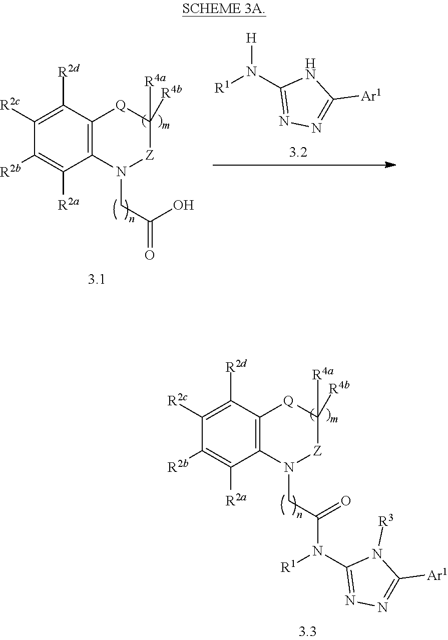

Compounds are represented in generic form, with substituents as noted in compound descriptions elsewhere herein. A more specific example is set forth below.

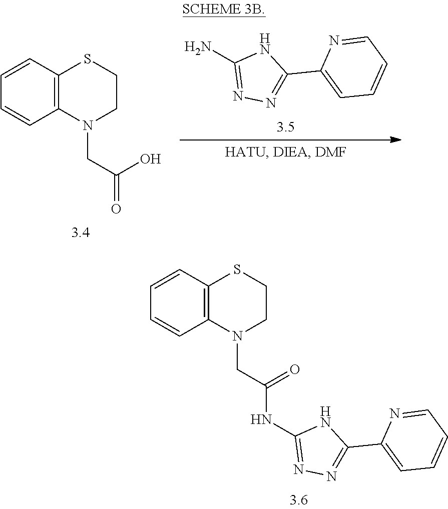 [SMILES: O=C(O)CN1CCSc2ccccc21 ; Nc1nnc(-c2ccccn2)[nH]1 ; O=C(CN1CCSc2ccccc21)Nc1nnc(-c2ccccn2)[nH]1]

In one aspect, compounds of type 3.3, and similar compounds, can be prepared according to reaction Scheme 3B above. Thus, compounds of type 3.6 can be prepared by a coupling reaction between an appropriate carboxylic acid, e.g., 3.4 as shown above, and an appropriate amine, e.g., 3.5 as shown above. Appropriate carboxylic acids and appropriate amines are commercially available or prepared by methods known to one skilled in the art. The coupling reaction is carried out in the presence of an appropriate coupling agent, e.g., (1-[bis(dimethylamino)methylene]-1H-1,2,3-triazolo[4,5-b]pyridinium 3-oxid hexafluorophosphate (HATU) and an appropriate base, e.g., diisopropylethylamine (DIEA) in an appropriate solvent, e.g., dimethylformamide (DMF). As can be appreciated by one skilled in the art, the above reaction provides an example of a generalized approach wherein compounds similar in structure to the specific reactants above (compounds similar to compounds of type 3.1 and 3.2), can be substituted in the reaction to provide substituted benzothiazone analogs similar to Formula 3.4.

### E. Methods Of Using The Compounds

The compounds and pharmaceutical compositions of the invention are useful in treating or controlling disorders associated with neurological disorders and in particular, Alzheimer's disease.

Examples of neurological disorders for which the compounds and compositions can be useful in treating, include, but are not limited to, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD).

To treat or control the disorder, the compounds and pharmaceutical compositions comprising the compounds are administered to a subject in need thereof, such as a vertebrate, e.g., a mammal, a fish, a bird, a reptile, or an amphibian. The subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a neurological disorder, such as Alzheimer's disease.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of a neurological disorder, such as Alzheimer's disease.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 Kg or more, a daily dosage of about 10 mg to about 10,000 mg, preferably from about 200 mg to about 1,000 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

### 1. Treatment Methods

The compounds disclosed herein are useful for treating or controlling disorders associated with a neurological disorder, in particular, Alzheimer's disease. Thus, provided is a method comprising administering a therapeutically effective amount of a composition comprising a disclosed compound to a subject. In a further aspect, the method can be a method for treating a neurological disorder. In a still further aspect, the method can be a method for treating a neuromuscular disorder.

a. Treating A Neurological Disorder

In one aspect, disclosed are methods of treating a neurological disorder in a subject having the neurological disorder, the method comprising the step of administering to the subject a therapeutically effective amount of at least one disclosed compound, or a pharmaceutically acceptable salt thereof.

In one aspect, disclosed are methods for the treatment of a neurological disorder in a subject, the method comprising the step of administering to the subject an effective amount of a compound having a structure represented by a formula:

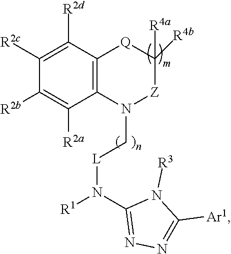

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, or a pharmaceutically acceptable salt thereof.

In one aspect, disclosed are methods for the treatment of a neurological disorder in a subject, the method comprising the step of administering to the subject an effective amount of at least one compound having a structure represented by a formula:

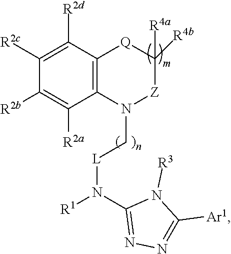

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

In one aspect, disclosed are methods for the treatment of a neurological disorder in a subject, the method comprising the step of administering to the subject an effective amount of at least one compound having a structure represented by a formula:

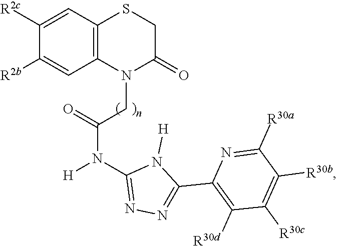

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

In one aspect, disclosed are methods for the treatment of a neurological disorder in a subject, the method comprising the step of administering to the subject an effective amount of at least one compound selected from the group consisting of:

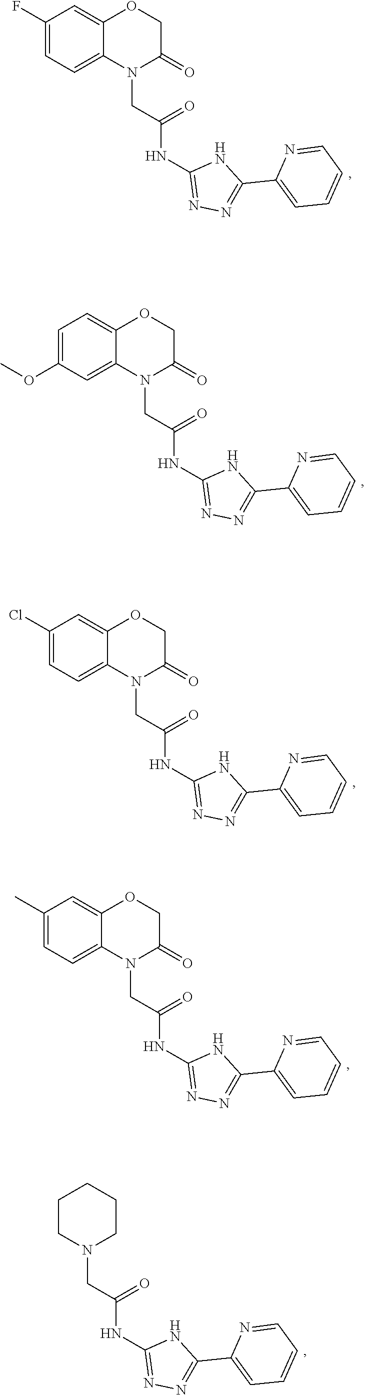 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1)Nc1nnc(-c2ccccn2)[nH]1]

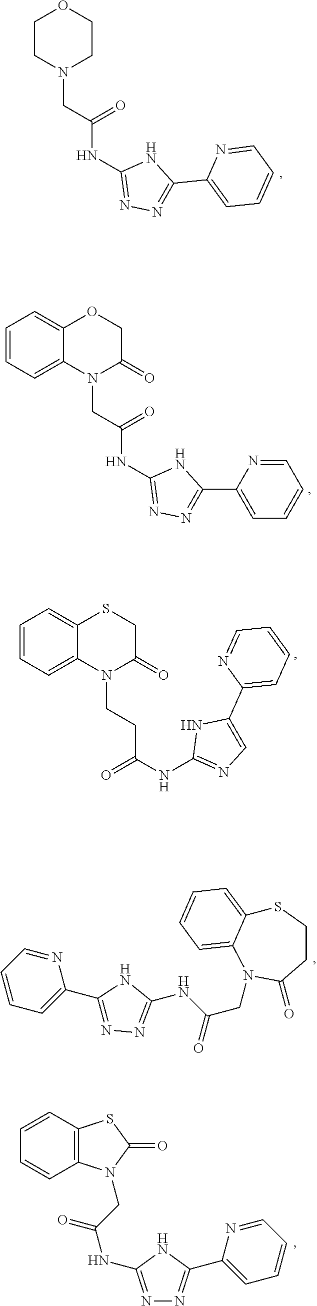 [SMILES: O=C(CN1CCOCC1)Nc1nnc(-c2ccccn2)[nH]1 ; O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1]

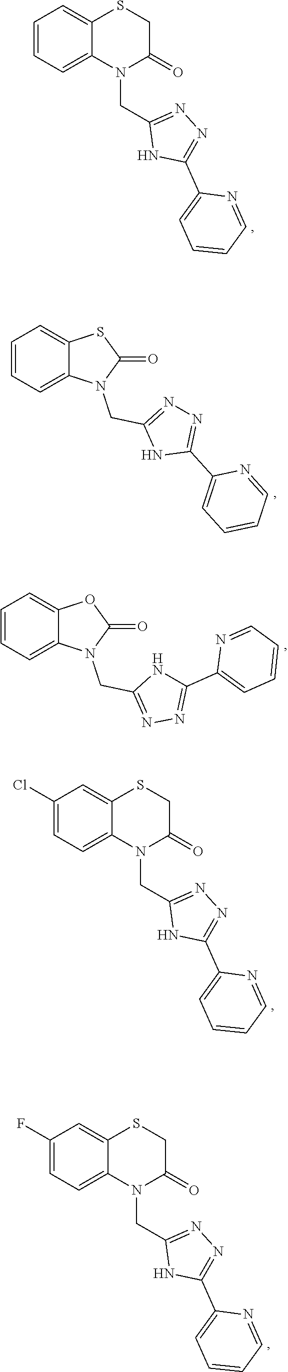 [SMILES: O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1]

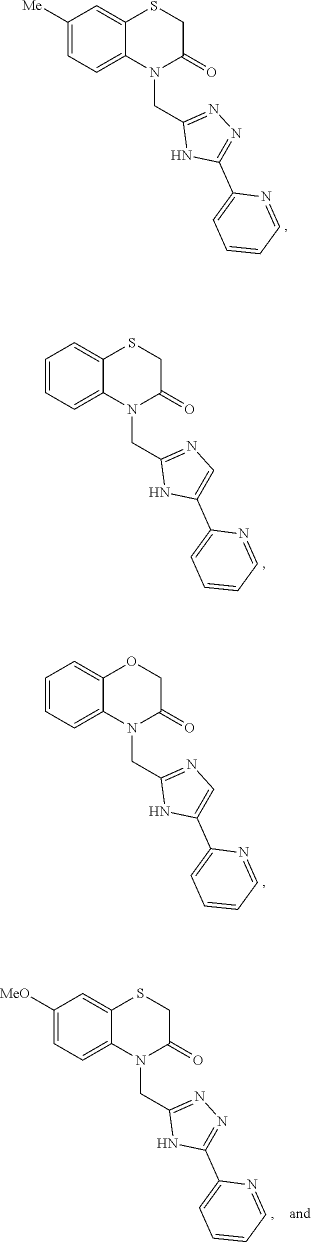 [SMILES: Cc1ccc2c(c1)SCC(=O)N2Cc1nnc(-c2ccccn2)[nH]1 ; O=C1CSc2ccccc2N1Cc1ncc(-c2ccccn2)[nH]1 ; O=C1COc2ccccc2N1Cc1ncc(-c2ccccn2)[nH]1 ; COc1ccc2c(c1)SCC(=O)N2Cc1nnc(-c2ccccn2)[nH]1]

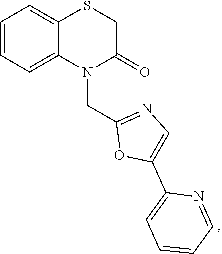 [SMILES: O=C1CSc2ccccc2N1Cc1ncc(-c2ccccn2)o1]

or a pharmaceutically acceptable salt thereof.

Examples of neurological disorders include, but are not limited to, amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD).

In a further aspect, the compound is selected from:

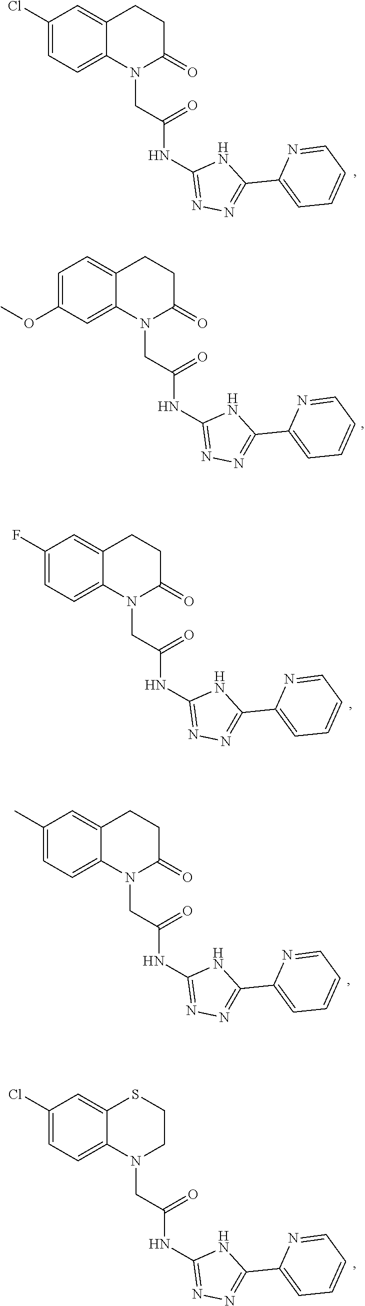 [SMILES: O=C(CN1C(=O)CCc2cc(Cl)ccc21)Nc1nnc(-c2ccccn2)[nH]1 ; COc1ccc2c(c1)N(CC(=O)Nc1nnc(-c3ccccn3)[nH]1)C(=O)CC2 ; O=C(CN1C(=O)CCc2cc(F)ccc21)Nc1nnc(-c2ccccn2)[nH]1 ; Cc1ccc2c(c1)CCC(=O)N2CC(=O)Nc1nnc(-c2ccccn2)[nH]1 ; O=C(CN1CCSc2cc(Cl)ccc21)Nc1nnc(-c2ccccn2)[nH]1]

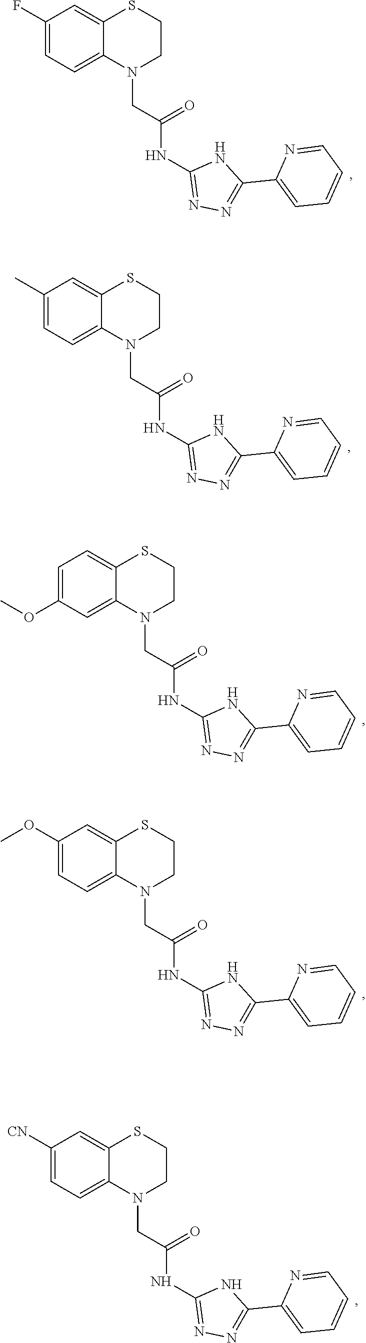

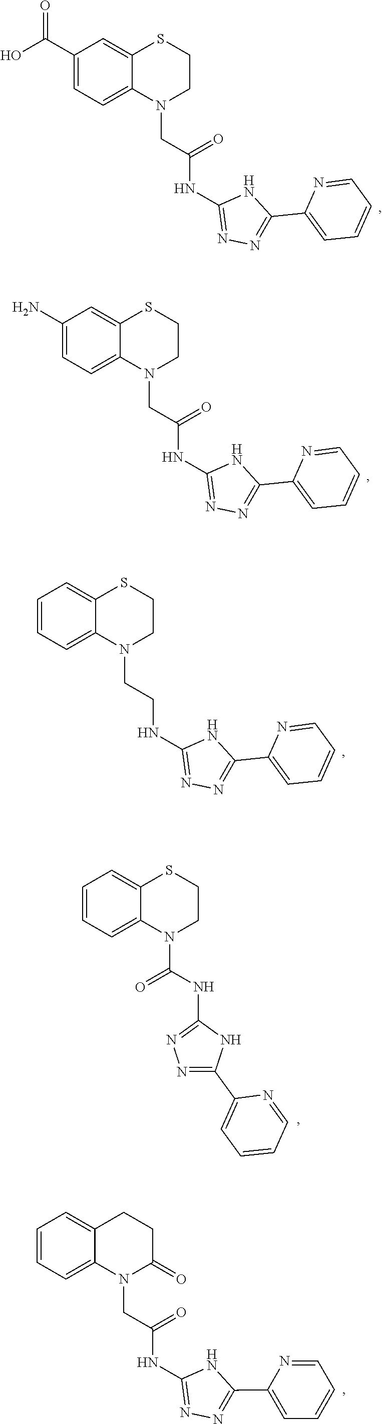 [SMILES: O=C(CN1CCSc2cc(C(=O)O)ccc21)Nc1nnc(-c2ccccn2)[nH]1 ; Nc1ccc2c(c1)SCCN2CC(=O)Nc1nnc(-c2ccccn2)[nH]1 ; c1ccc(-c2nnc(NCCN3CCSc4ccccc43)[nH]2)nc1 ; O=C(Nc1nnc(-c2ccccn2)[nH]1)N1CCSc2ccccc21 ; O=C(CN1C(=O)CCc2ccccc21)Nc1nnc(-c2ccccn2)[nH]1]

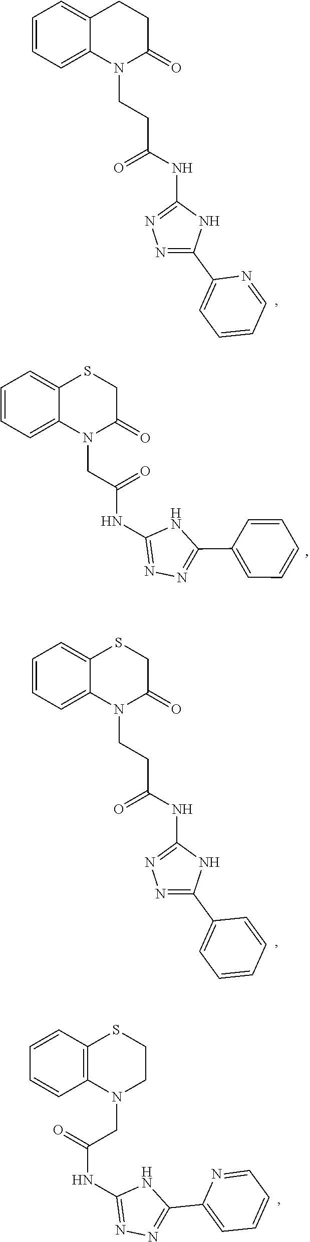 [SMILES: O=C(CCN1C(=O)CCc2ccccc21)Nc1nnc(-c2ccccn2)[nH]1 ; O=C(CN1C(=O)CSc2ccccc21)Nc1nnc(-c2ccccc2)[nH]1 ; O=C(CCN1C(=O)CSc2ccccc21)Nc1nnc(-c2ccccc2)[nH]1 ; O=C(CN1CCSc2ccccc21)Nc1nnc(-c2ccccn2)[nH]1]

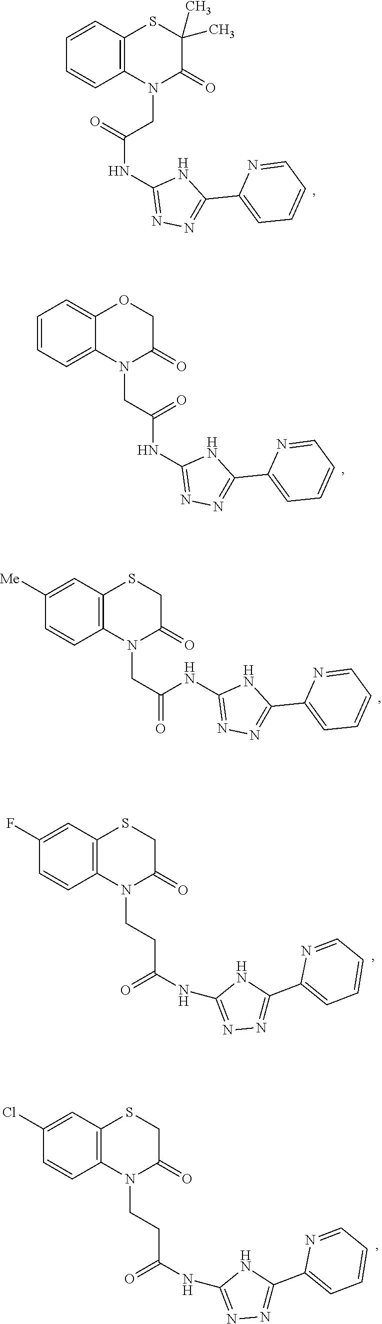 [SMILES: CC1(C)Sc2ccccc2N(CC(=O)Nc2nnc(-c3ccccn3)[nH]2)C1=O ; O=C(CN1C(=O)COc2ccccc21)Nc1nnc(-c2ccccn2)[nH]1 ; Cc1ccc2c(c1)SCC(=O)N2CC(=O)Nc1nnc(-c2ccccn2)[nH]1 ; O=C(CCN1C(=O)CSc2cc(F)ccc21)Nc1nnc(-c2ccccn2)[nH]1 ; O=C(CCN1C(=O)CSc2cc(Cl)ccc21)Nc1nnc(-c2ccccn2)[nH]1]

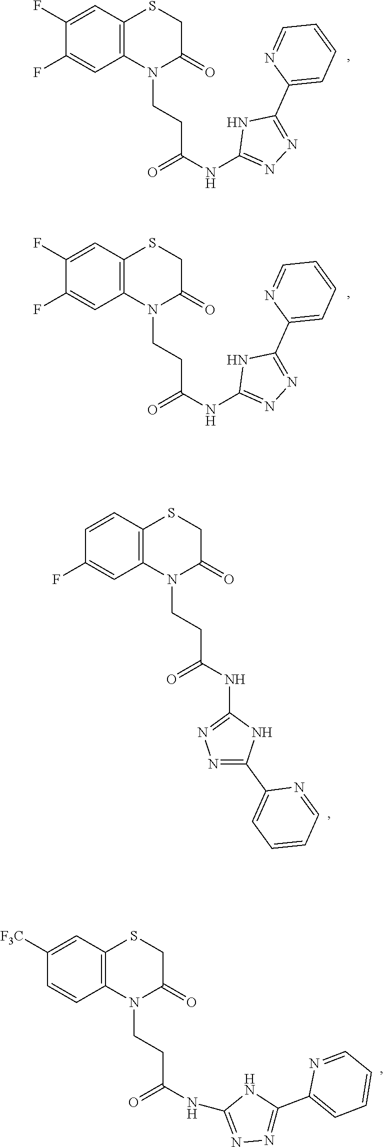 [SMILES: O=C(CCN1C(=O)CSc2cc(F)c(F)cc21)Nc1nnc(-c2ccccn2)[nH]1 ; O=C(CCN1C(=O)CSc2cc(F)c(F)cc21)Nc1nnc(-c2ccccn2)[nH]1 ; O=C(CCN1C(=O)CSc2ccc(F)cc21)Nc1nnc(-c2ccccn2)[nH]1 ; O=C(CCN1C(=O)CSc2cc(C(F)(F)F)ccc21)Nc1nnc(-c2ccccn2)[nH]1]

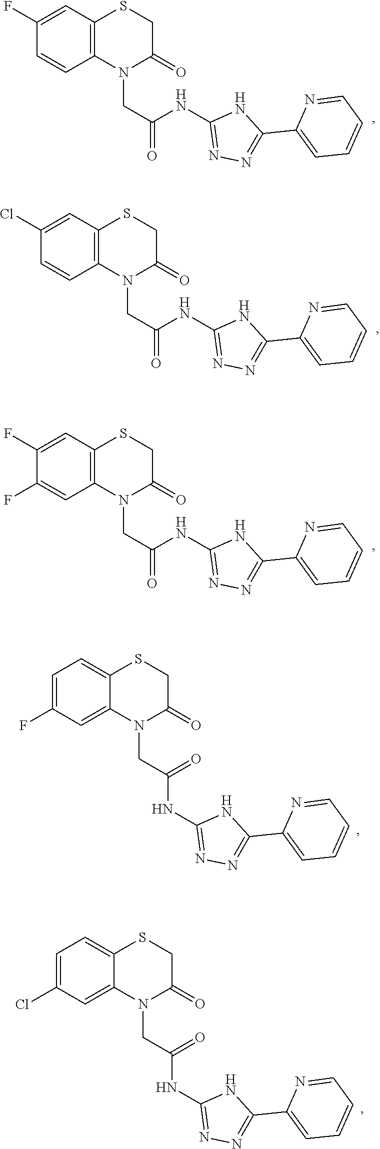 [SMILES: O=C(CN1C(=O)CSc2cc(F)ccc21)Nc1nnc(-c2ccccn2)[nH]1 ; O=C(CN1C(=O)CSc2cc(Cl)ccc21)Nc1nnc(-c2ccccn2)[nH]1 ; O=C(CN1C(=O)CSc2cc(F)c(F)cc21)Nc1nnc(-c2ccccn2)[nH]1 ; O=C(CN1C(=O)CSc2ccc(F)cc21)Nc1nnc(-c2ccccn2)[nH]1 ; O=C(CN1C(=O)CSc2ccc(Cl)cc21)Nc1nnc(-c2ccccn2)[nH]1]

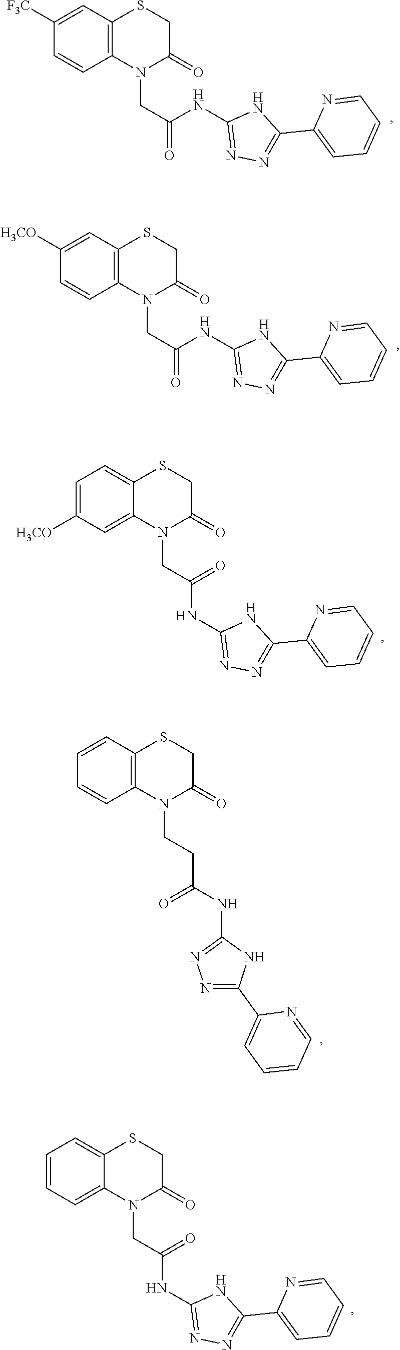 [SMILES: O=C(CN1C(=O)CSc2cc(C(F)(F)F)ccc21)Nc1nnc(-c2ccccn2)[nH]1 ; COc1ccc2c(c1)SCC(=O)N2CC(=O)Nc1nnc(-c2ccccn2)[nH]1 ; COc1ccc2c(c1)N(CC(=O)Nc1nnc(-c3ccccn3)[nH]1)C(=O)CS2 ; O=C(CCN1C(=O)CSc2ccccc21)Nc1nnc(-c2ccccn2)[nH]1 ; O=C(CN1C(=O)CSc2ccccc21)Nc1nnc(-c2ccccn2)[nH]1]

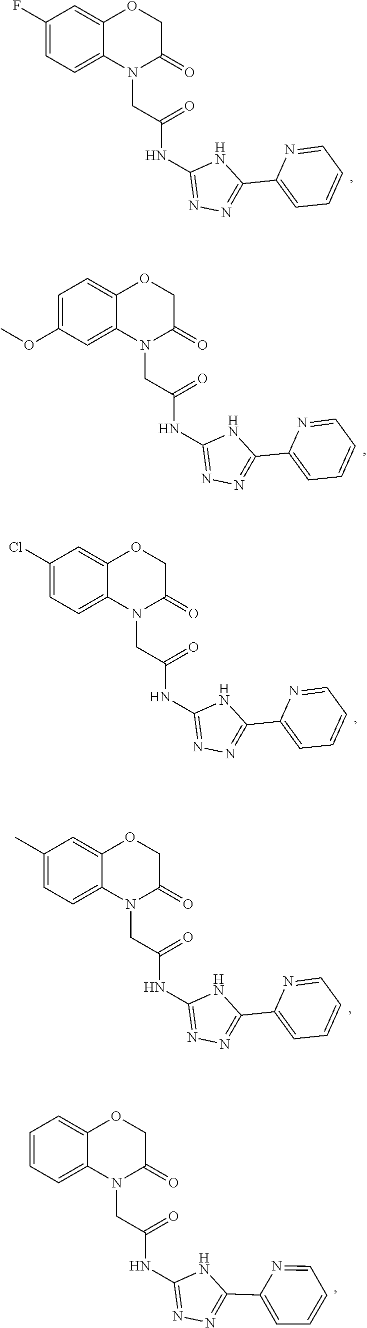 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1C(=O)COc2ccccc21)Nc1nnc(-c2ccccn2)[nH]1]

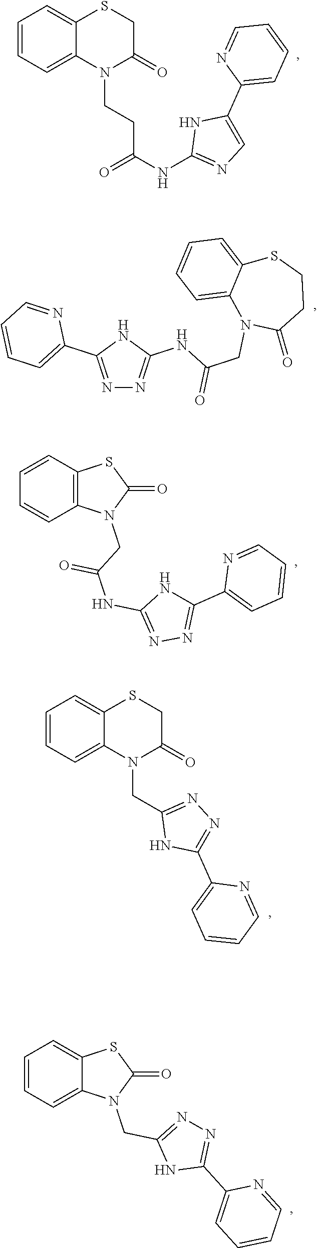 [SMILES: O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1 ; O=C1CSc2ccccc2N1Cc1nnc(-c2ccccn2)[nH]1 ; O=c1sc2ccccc2n1Cc1nnc(-c2ccccn2)[nH]1]

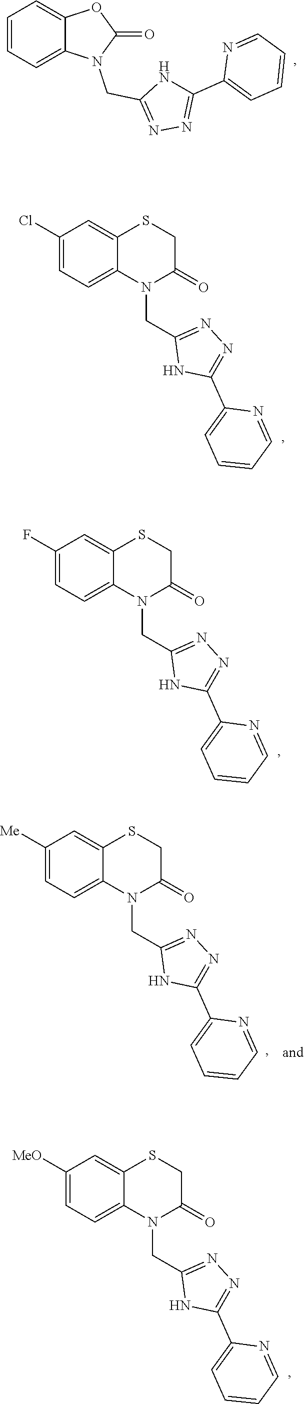 [SMILES: O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1 ; Cc1ccc2c(c1)SCC(=O)N2Cc1nnc(-c2ccccn2)[nH]1 ; COc1ccc2c(c1)SCC(=O)N2Cc1nnc(-c2ccccn2)[nH]1]

or a pharmaceutically acceptable salt thereof.

In a further aspect, the subject has been diagnosed with a need for treatment of the neurological disorder prior to the administering step.

In a further aspect, the subject is a mammal. In a still further aspect, the mammal is a human.

In a further aspect, the method further comprises the step of identifying a subject in need of treatment of the neurological disorder.

In a further aspect, the disorder is associated with dysregulation of Tau-SH3 signaling. In a still further aspect, the neurological disorder is associated with activation and/or over-activation of Tau-SH3 signaling.

In a further aspect, the disorder is associated with dysregulation of Tau-Fyn signaling. In a still further aspect, the neurological disorder is associated with activation and/or over-activation of Tau-Fyn signaling.

In a further aspect, the neurological disorder is selected from amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD). In yet a further aspect, the neurological disorder is Alzheimer's disease.

In a further aspect, the effective amount is a therapeutically effective amount.

In a still further aspect, the effective amount is a prophylactically effective amount.

In a further aspect, the method further comprises the step of administering a therapeutically effective amount of at least one agent associated with the treatment of a neurological disorder. In a still further aspect, the at least one agent is selected from a cholinesterase inhibitor, an antiepileptic agent, an antidepressant, memantine, rilutek, radicava, levodopa, carbidopa, a dopamine agonist, a MAO-B inhibitor, a catechol-O-methyltransferase inhibitor, an anticholinergic, spinraza, tetrabenazine, an antipsychotic agent, levetiracetam, clonazepam, an antipsychotic agent, a mood-stabilizing agent, and amantadine.

In a further aspect, the at least one compound and the at least one agent are administered sequentially. In a still further aspect, the at least one compound and the at least one agent are administered simultaneously.

In a further aspect, the at least one compound and the at least one agent are co-formulated. In a still further aspect, the at least one compound and the at least one agent are co-packaged.

### 2. Methods Of Modifying Tau-SH3 Signaling In A Subject

In one aspect, disclosed are methods of modifying Tau-SH3 signaling in a subject, the method comprising the step of administering to the subject an effective amount of at least one disclosed compound, or a pharmaceutically acceptable salt thereof. In a further aspect, the method modifies Tau-Fyn signaling in a subject.

In one aspect, disclosed are methods of modifying Tau-ySH3 signaling in a subject, the method comprising the step of administering to the subject an effective amount of at least one compound having a structure represented by a formula:

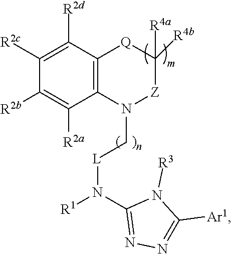

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

In one aspect, disclosed are methods of modifying Tau-SH3 signaling in a subject, the method comprising the step of administering to the subject an effective amount of at least one compound having a structure represented by a formula:

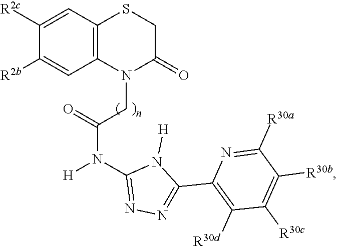

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

In one aspect, disclosed are methods for modifying Tau-SH3 signaling in a subject, the method comprising the step of administering to the subject an effective amount of at least one compound selected from the group consisting of:

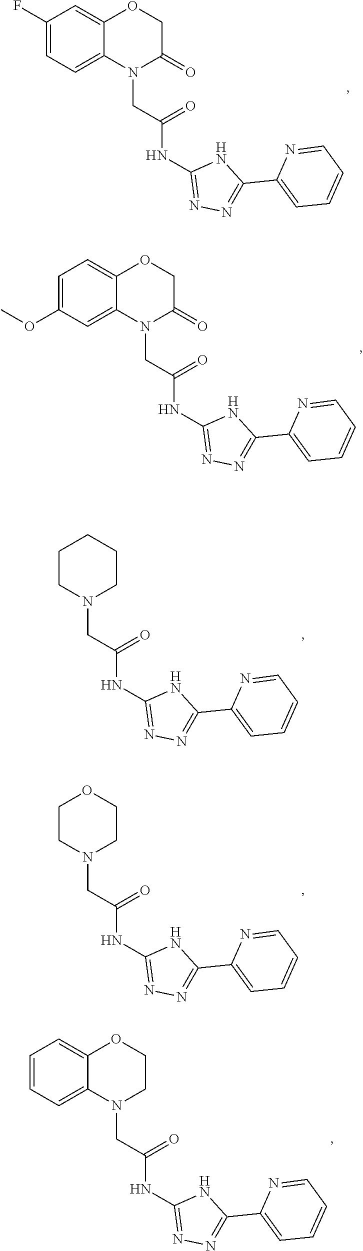 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1CCCCC1)Nc1nnc(-c2ccccn2)[nH]1 ; O=C(CN1CCOCC1)Nc1nnc(-c2ccccn2)[nH]1 ; O=C(CN1CCOc2ccccc21)Nc1nnc(-c2ccccn2)[nH]1]

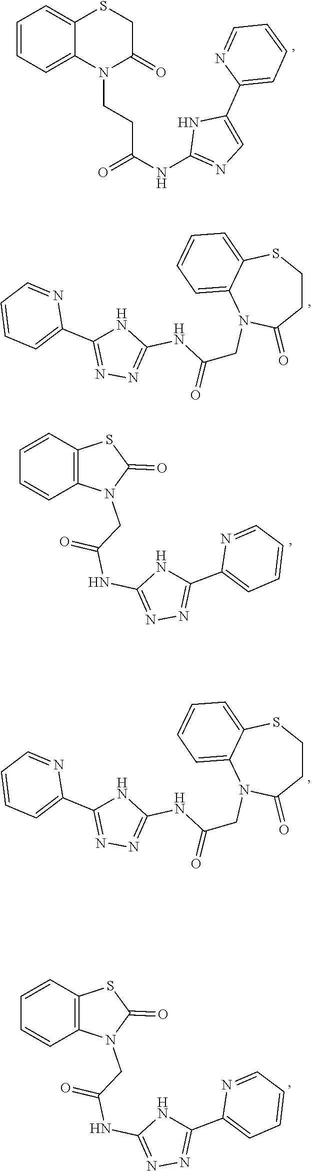 [SMILES: O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1]

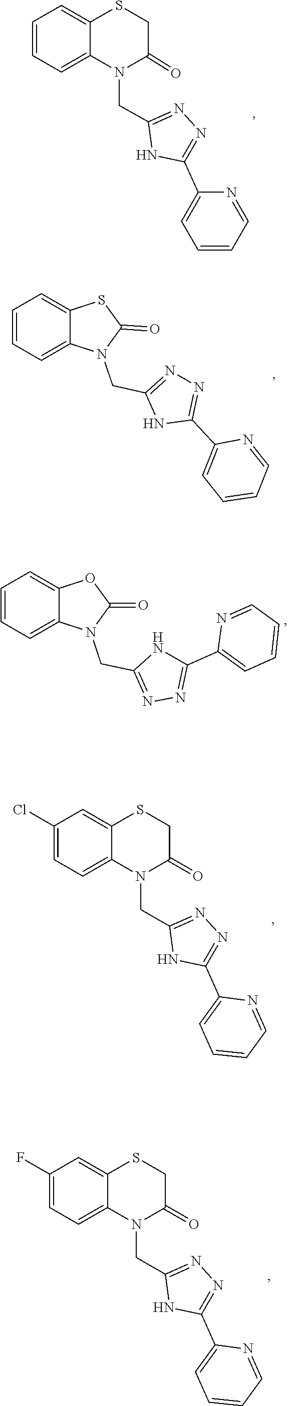 [SMILES: O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1]

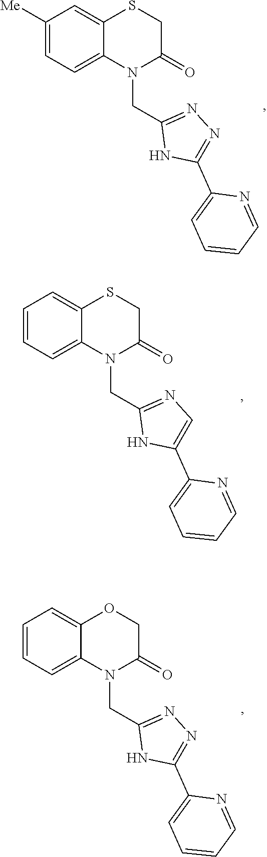 [SMILES: Cc1ccc2c(c1)SCC(=O)N2Cc1nnc(-c2ccccn2)[nH]1 ; O=C1CSc2ccccc2N1Cc1ncc(-c2ccccn2)[nH]1 ; O=C1COc2ccccc2N1Cc1nnc(-c2ccccn2)[nH]1]

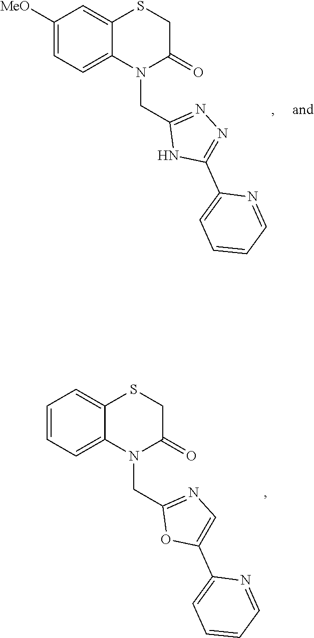 [SMILES: COc1ccc2c(c1)SCC(=O)N2Cc1nnc(-c2ccccn2)[nH]1 ; O=C1CSc2ccccc2N1Cc1ncc(-c2ccccn2)o1]

or a pharmaceutically acceptable salt thereof.

In a further aspect, modifying is inhibiting.

In a further aspect, the compound exhibits inhibition of Tau-SH3 signaling. In a still further aspect, the compound exhibits a decrease in Tau-SH3 signaling. In yet a further aspect, the Tau-SH3 signaling is Tau-Fyn signaling.

In a further aspect, the compound exhibits inhibition of Tau-SH3 signaling with an IC₅₀ of less than about 30 μM. In a still further aspect, the compound exhibits inhibition of Tau-SH3 signaling with an IC₅₀ of less than about 25 μM. In yet a further aspect, the compound exhibits inhibition of Tau-SH3 signaling with an IC₅₀ of less than about 20 μM. In an even further aspect, the compound exhibits inhibition of Tau-SH3 signaling with an IC₅₀ of less than about 15 μM. In a still further aspect, the compound exhibits inhibition of Tau-SH3 signaling with an IC₅₀ of less than about 10 μM. In yet a further aspect, the compound exhibits inhibition of Tau-SH3 signaling with an IC₅₀ of less than about 5 μM. In an even further aspect, the compound exhibits inhibition of Tau-SH3 signaling with an IC₅₀ of less than about 1 μM. In a still further aspect, the compound exhibits inhibition of Tau-SH3 signaling with an IC₅₀ of less than about 0.5 μM.

In a further aspect, the compound exhibits inhibition of Tau-Fyn signaling with an IC₅₀ of less than about 30 μM. In a still further aspect, the compound exhibits inhibition of Tau-Fyn signaling with an IC₅₀ of less than about 25 μM. In yet a further aspect, the compound exhibits inhibition of Tau-Fyn signaling with an IC₅₀ of less than about 20 μM. In an even further aspect, the compound exhibits inhibition of Tau-Fyn signaling with an IC₅₀ of less than about 15 μM. In a still further aspect, the compound exhibits inhibition of Tau-Fyn signaling with an IC₅₀ of less than about 10 μM. In yet a further aspect, the compound exhibits inhibition of Tau-Fyn signaling with an IC₅₀ of less than about 5 μM. In an even further aspect, the compound exhibits inhibition of Tau-Fyn signaling with an IC₅₀ of less than about 1 μM. In a still further aspect, the compound exhibits inhibition of Tau-Fyn signaling with an IC₅₀ of less than about 0.5 μM.

In a further aspect, the subject is a mammal. In a still further aspect, the subject is a human.

In a further aspect, the subject has been diagnosed with a need for treatment of a neurological disorder prior to the administering step. In a still further aspect, the method further comprises the step of identifying a subject in need of treatment of a neurological disorder.

In a further aspect, the subject has been diagnosed with a need for treatment of a disorder associated with Tau-SH3 signaling dysfunction prior to the administering step. In a still further aspect, the method further comprises the step of identifying a subject in need of treatment of a disorder associated with Tau-SH3 signaling dysfunction. In yet a further aspect, the disorder associated with Tau-SH3 signaling dysfunction is a neurological disorder.

In a further aspect, the subject has been diagnosed with a need for modifying Tau-SH3 signaling prior to the administering step. In a still further aspect, the subject has been diagnosed with a need for inhibiting Tau-SH3 signaling prior to the administering step.

### 3. Methods Of Modifying Tau-SH3 Signaling In At Least One Cell

In one aspect, disclosed are methods for modifying Tau-SH3 signaling in at least one cell, the method comprising the step of contacting the at least one cell with an effective amount of at least one disclosed compound, or a pharmaceutically acceptable salt thereof. In a further aspect, the method modifies Tau-Fyn signaling in at least one cell.

In one aspect, disclosed are methods for modifying Tau-SH3 signaling in at least one cell, the method comprising the step of contacting at least one cell with an effective amount of at least one compound having a structure represented by a formula:

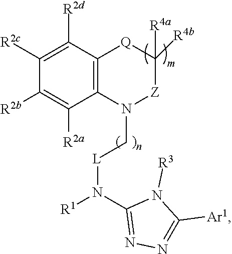

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof.

In one aspect, disclosed are methods for modifying Tau-SH3 signaling in at least one cell, the method comprising the step of contacting at least one cell with an effective amount of at least one compound having a structure represented by a formula:

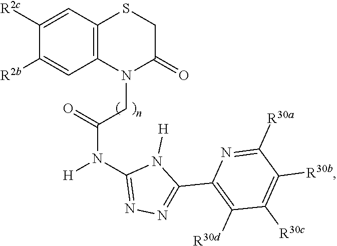

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof.

In one aspect, disclosed are methods for modifying Tau-SH3 signaling in at least one cell, the method comprising the step of contacting at least one cell with an effective amount of at least one compound selected from the group consisting of

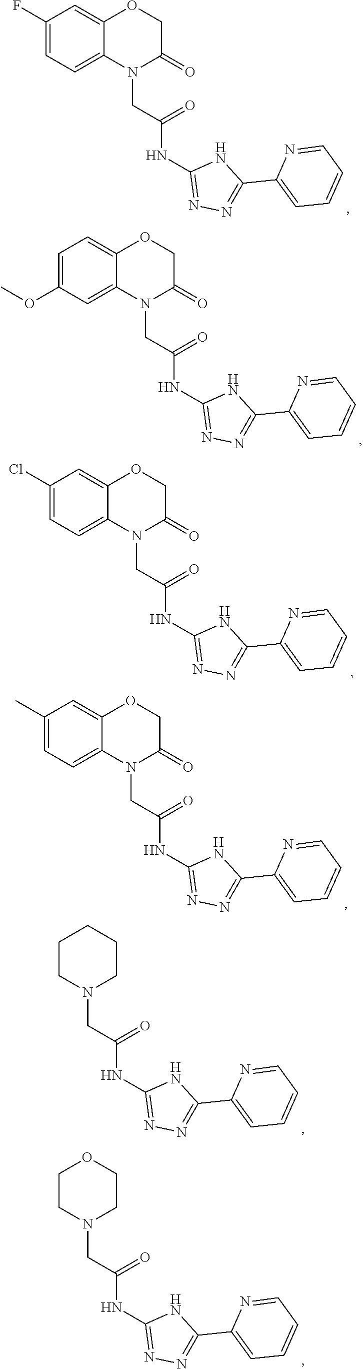 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1)Nc1nnc(-c2ccccn2)[nH]1 ; O=C(CN1CCOCC1)Nc1nnc(-c2ccccn2)[nH]1]

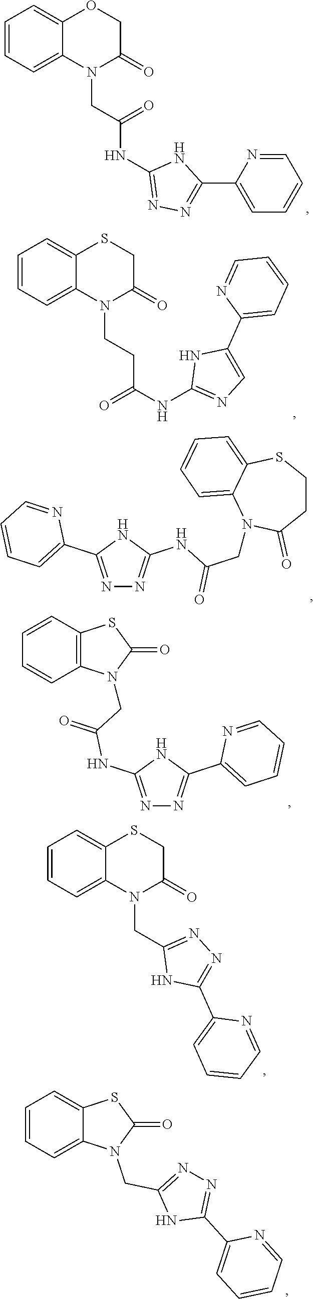

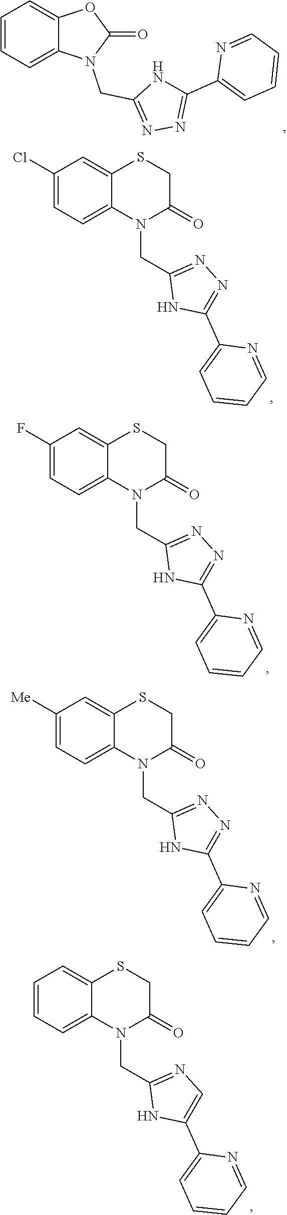

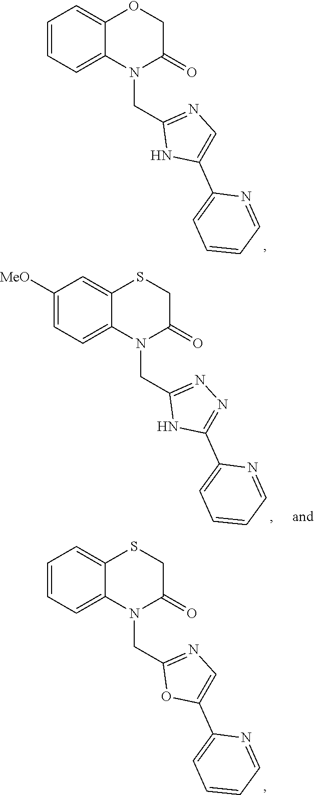 [SMILES: O=C1COc2ccccc2N1Cc1ncc(-c2ccccn2)[nH]1 ; COc1ccc2c(c1)SCC(=O)N2Cc1nnc(-c2ccccn2)[nH]1 ; O=C1CSc2ccccc2N1Cc1ncc(-c2ccccn2)o1]

or a pharmaceutically acceptable salt thereof.

In a further aspect, modifying is inhibiting.

In a further aspect, the cell is mammalian. In a still further aspect, the cell is human. In yet a further aspect, the cell has been isolated from a mammal prior to the contacting step.

In a further aspect, contacting is via administration to a subject.

In a further aspect, the subject has been diagnosed with a need for modification of Tau-SH3 signaling prior to the administering step. In a still further aspect, the subject has been diagnosed with a need for treatment of a disorder associated with Tau-SH3 signaling dysfunction. In yet a further aspect, the Tau-SH3 signaling is Tau-Fyn signaling.

### 4. Use Of Compounds

In one aspect, the invention relates to the use of a disclosed compound or a product of a disclosed method. In a further aspect, a use relates to the manufacture of a medicament for the treatment of a neurological disorder in a subject.

Also provided are the uses of the disclosed compounds and products. In one aspect, the invention relates to use of at least one disclosed compound; or a pharmaceutically acceptable salt, hydrate, solvate, or polymorph thereof. In a further aspect, the compound used is a product of a disclosed method of making.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, for use as a medicament.

In a further aspect, the use relates to a process for preparing a pharmaceutical composition comprising a therapeutically effective amount of a disclosed compound or a product of a disclosed method of making, or a pharmaceutically acceptable salt, solvate, or polymorph thereof, wherein a pharmaceutically acceptable carrier is intimately mixed with a therapeutically effective amount of the compound or the product of a disclosed method of making.

In various aspects, the use relates to a treatment of a neurological disorder in a subject. Also disclosed is the use of a compound for antagonism of a neurological disorder. In one aspect, the use is characterized in that the subject is a human. In one aspect, the use is characterized in that the disorder is a neurological disorder.

In a further aspect, the use relates to the manufacture of a medicament for the treatment of a neurological disorder in a subject.

In a further aspect, the use relates to antagonism of a neurological disorder in a subject.

It is understood that the disclosed uses can be employed in connection with the disclosed compounds, products of disclosed methods of making, methods, compositions, and kits. In a further aspect, the invention relates to the use of a disclosed compound or a disclosed product in the manufacture of a medicament for the treatment of a neurological disorder in a mammal. In a further aspect, the neurological disorder is Alzheimer's disease.

### 5. Manufacture Of A Medicament

In one aspect, the invention relates to a method for the manufacture of a medicament for treating a neurological disorder in a subject having the neurological disorder, the method comprising combining a therapeutically effective amount of a disclosed compound or product of a disclosed method with a pharmaceutically acceptable carrier or diluent.

As regards these applications, the present method includes the administration to an animal, particularly a mammal, and more particularly a human, of a therapeutically effective amount of the compound effective in the treatment of a neurological disorder. The dose administered to an animal, particularly a human, in the context of the present invention should be sufficient to affect a therapeutic response in the animal over a reasonable period. One skilled in the art will recognize that dosage will depend upon a variety of factors including the condition of the animal and the body weight of the animal.

The total amount of the compound of the present disclosure administered in a typical treatment is preferably between about 10 mg/kg and about 1000 mg/kg of body weight for mice, and between about 100 mg/kg and about 500 mg/kg of body weight, and more preferably between 200 mg/kg and about 400 mg/kg of body weight for humans per daily dose. This total amount is typically, but not necessarily, administered as a series of smaller doses over a period of about one time per day to about three times per day for about 24 months, and preferably over a period of twice per day for about 12 months.

The size of the dose can also be determined by the route, timing and frequency of administration as well as the existence, nature and extent of any adverse side effects that might accompany the administration of the compound and the desired physiological effect. It will be appreciated by one of skill in the art that various conditions or disease states, in particular chronic conditions or disease states, may require prolonged treatment involving multiple administrations.

Thus, in one aspect, the invention relates to the manufacture of a medicament comprising combining a disclosed compound or a product of a disclosed method of making, or a pharmaceutically acceptable salt, solvate, or polymorph thereof, with a pharmaceutically acceptable catier or diluent.

### 6. Kits

In one aspect, the invention relates to a kit comprising at least one disclosed compound and one or more of (a) at least one agent associated with the treatment of a neurological disorder; (b) instructions for administering the compound in connection with treating a neurological disorder; and (c) instructions for treating a neurological disorder.

In one aspect, disclosed are kits comprising a compound having a structure represented by a formula:

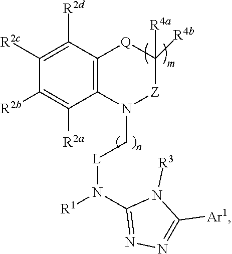

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —COR²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, or a pharmaceutically acceptable salt thereof, and one or more of: (a) at least one agent associated with the treatment of a neurological disorder; (b) instructions for administering the compound in connection with treating a neurological disorder; and (c) instructions for treating a neurological disorder.

In one aspect, disclosed are kits comprising at least one compound having a structure represented by a formula:

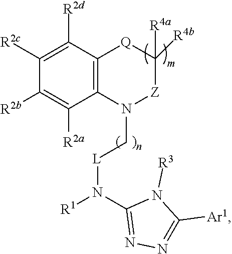

wherein each of n and m is independently 0, 1, or 2; wherein Q is selected from the group consisting of —O—, —S—, and —CH₂—; wherein Z is selected from the group consisting of —C(O)—, —CH(OH)—, and —CH₂—; wherein L is selected from the group consisting of —C(O)— and —CH₂—; wherein each of R¹ and R³ is independently selected from the group consisting of hydrogen and C1-C4 alkyl; wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³; wherein each occurrence of R²⁰, R²¹, R²², and R²³, when present, is independently selected from the group consisting of hydrogen and C1-C4 alkyl; and wherein each occurrence of R^{4a} and R^{4b}, when present, is independently selected from hydrogen, C1-C4 alkyl, and C1-C4 haloalkyl; wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and wherein each occurrence of R²⁴, R²⁵, R²⁶, and R²⁷, when present, is independently selected from the group consisting of hydrogen and C1-C4 alkyl, provided that when Q is —O— then Z is —CH(OH)— or —CH₂—, and provided that when Q is —O— Z is —CH₂—, then each of R^{2a}, R^{2b}, R^{2c}, and R^{2d} is hydrogen, and Ar¹ is 2-pyridinyl, and provided that when Q is —CH₂ then Z is —C(O)—, and provided that when n is 1 or 2, m is 1, L is —C(O)—, Q is —S—, and Ar¹ is 2-pyridinyl, then Z is —CH₂—, or a pharmaceutically acceptable salt thereof, and one or more of: (a) at least one agent associated with the treatment of a neurological disorder; (b) instructions for administering the compound in connection with treating a neurological disorder; and (c) instructions for treating a neurological disorder.

In one aspect, disclosed are kits comprising at least one compound having a structure represented by a formula:

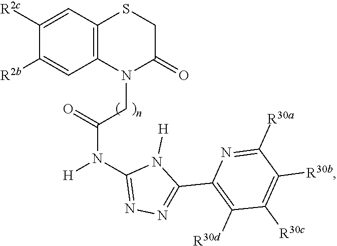

wherein n is 0, 1, or 2; wherein each of R^{2a} and R^{2b} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³, provided that at least one of R^{2a} and R^{2b} is not hydrogen; wherein each occurrence of R²⁰, R²¹, R²², and R²³, when present, is independently selected from the group consisting of hydrogen and C1-C4 alkyl; and wherein each of R^{30a}, R^{30b}, R^{30c}, and R^{30d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R⁴⁰, —CO₂R⁴¹, —C(O)NR⁴², and —SO₂R⁴³, wherein each occurrence of R⁴⁰, R⁴¹, R⁴², and R⁴³, when present, is independently selected from the group consisting of hydrogen and C1-C4 alkyl, or a pharmaceutically acceptable salt thereof, and one or more of: (a) at least one agent associated with the treatment of a neurological disorder; (b) instructions for administering the compound in connection with treating a neurological disorder; and (c) instructions for treating a neurological disorder.

In one aspect, disclosed are kits comprising at least one compound selected from:

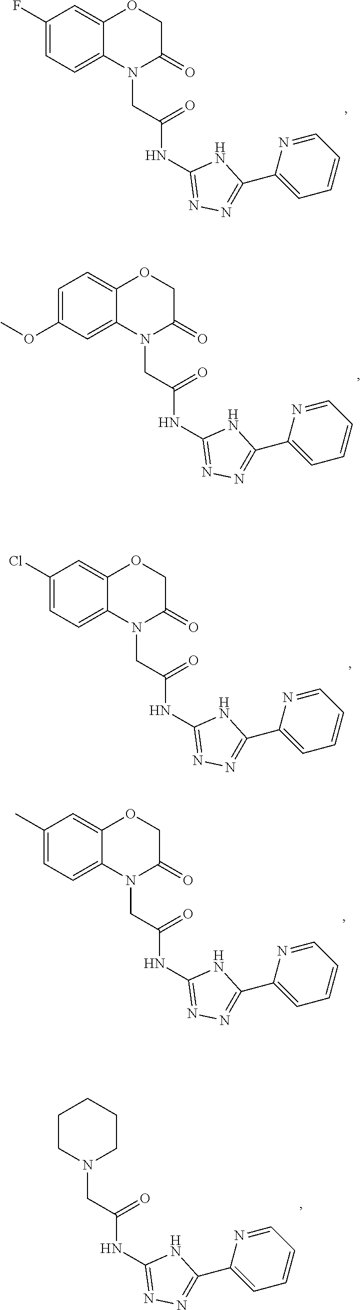 [SMILES: O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1 ; O=C(CN1CCCCC1)Nc1nnc(-c2ccccn2)[nH]1]

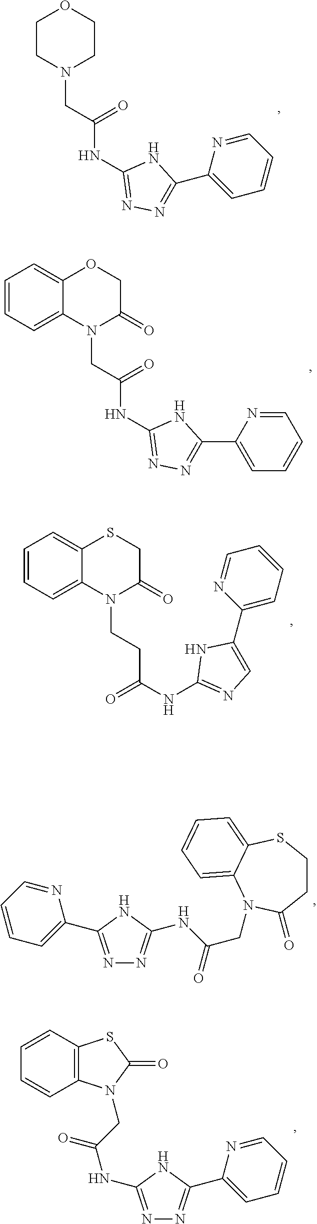 [SMILES: O=C(CN1CCOCC1)Nc1nnc(-c2ccccn2)[nH]1 ; O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1]

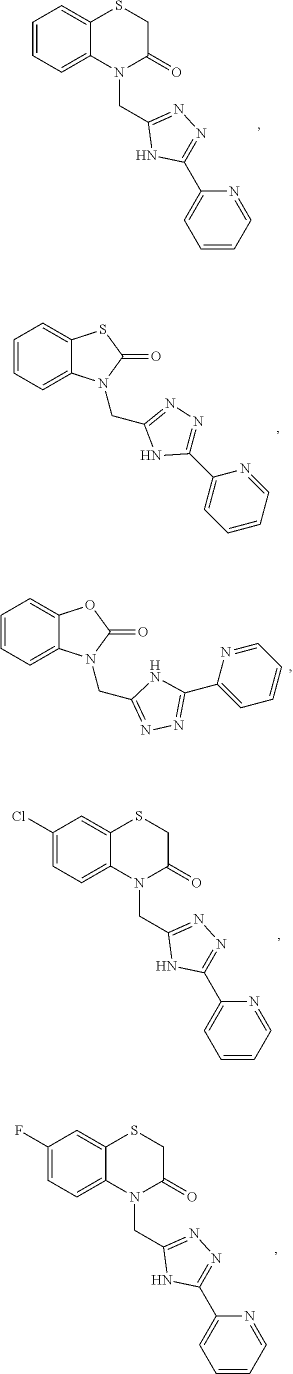 [SMILES: O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1]

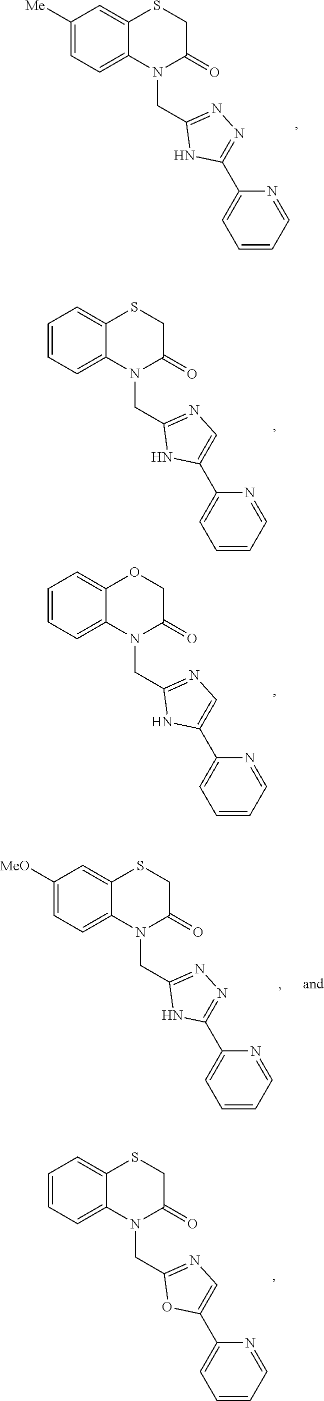 [SMILES: Cc1ccc2c(c1)SCC(=O)N2Cc1nnc(-c2ccccn2)[nH]1 ; O=C1CSc2ccccc2N1Cc1ncc(-c2ccccn2)[nH]1 ; O=C1COc2ccccc2N1Cc1ncc(-c2ccccn2)[nH]1 ; COc1ccc2c(c1)SCC(=O)N2Cc1nnc(-c2ccccn2)[nH]1 ; O=C1CSc2ccccc2N1Cc1ncc(-c2ccccn2)o1]

or a pharmaceutically acceptable derivative thereof, and one or more of (a) at least one agent associated with the treatment of a neurological disorder; (b) instructions for administering the compound in connection with treating a neurological disorder; and (c) instructions for treating a neurological disorder.

In a further aspect, the neurological disorder is selected from amyotrophic lateral sclerosis (ALS), Alzheimer's disease, epilepsy, autism spectrum disorders, Parkinson's disease, spinal muscular atrophy, traumatic brain injury, vascular dementia, Huntington's disease, mental retardation, and attention deficit and hyperactivity disorder (ADHD). In a further aspect, the neurological disorder is ALS. In a still further aspect, the neurological disorder is Alzheimer's disease.

In a further aspect, the agent associated with the treatment of a neurological disorder is selected from a cholinesterase inhibitor, an antiepileptic agent, an antidepressant, memantine, rilutek, radicava, levodopa, carbidopa, a dopamine agonist, a MAO-B inhibitor, a catechol-O-methyltransferase inhibitor, an anticholinergic, spinraza, tetrabenazine, an antipsychotic agent, levetiracetam, clonazepam, an antipsychotic agent, a mood-stabilizing agent, and amantadine.

In a further aspect, the at least one compound and the at least one agent associated with the treatment of a neurological disorder are co-formulated. In a further aspect, the at least one compound and the at least one agent associated with the treatment of a neurological disorder are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### F. Examples

A peptide inhibitor of Tau-SH3 interactions was recently developed (Rush, et al. (2020) Neurobiol Dis 134, 104668). Two high-content measures of Aβ toxicity were used in primary neurons, and a proximity ligation assay was used to measure Tau-Fyn binding. It was found that the peptide inhibits endogenous Tau-Fyn interaction in neurons and ameliorates Aβ toxicity (Rush, et al. (2020) Neurobiol Dis 134, 104668), validating this approach to prevent AR-induced dysfunction. An AlphaScreen assay for Tau-FynSH3 interaction inhibitors was also developed, and used to perform a high-throughput screen (HTS) of ˜50,000 compounds (Cochran, et al. (2014) J. Biomol. Screen. 19, 1338-1349). Of these, several with promising drug-like properties were found (Cochran, et al. (2014) J. Biomol. Screen. 19, 1338-1349).

Here, the development of small molecules that are cell-permeable, potently inhibit Tau-SH3 interactions, and ameliorate Aβ toxicity and network hyperexcitability is described. Upon identifying a top hit from a high-throughput screen (HTS), a medicinal chemistry approach was used to produce a lead compound. Next, the lead compounds effect on Aβ-induced dysfunction, its binding partner, and whether it inhibited multiple Tau-SH3 interactions was evaluated. Finally, studies were performed to determine whether inhibition of Tau-SH3 interactions is sufficient to prevent Aβ-induced network hyperexcitability. Without being bound by theory, these findings support the hypothesis that inhibiting Tau-SH3 interactions is an attractive therapeutic approach to prevent Aβ-induced neuronal dysfunction, and demonstrate that cell-permeable small molecules can be developed that inhibit Tau-SH3 interactions.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Chemistry Experimentals

A summary of the exemplary compounds prepared as described herein is shown below in Table 1.

TABLE 1

| SN | Structure |
|---|---|
| 1 | 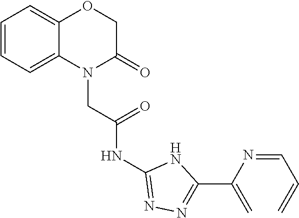 [SMILES: C=C/C=N\C(=C)c1nnc(NC(=O)CN2C(=O)COc3ccccc32)[nH]1] |
| 2 | 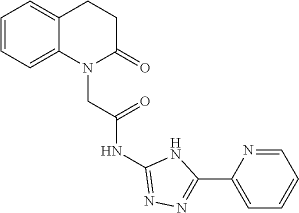 [SMILES: O=C(CN1C(=O)CCc2ccccc21)Nc1nnc(-c2ccccn2)[nH]1] |
| 3 | 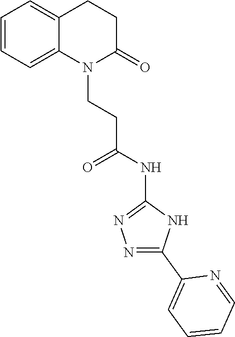 [SMILES: O=C(CCN1C(=O)CCc2ccccc21)Nc1nnc(-c2ccccn2)[nH]1] |
| 4 | 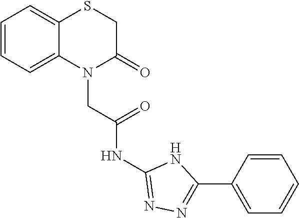 [SMILES: O=C(CN1C(=O)CSc2ccccc21)Nc1nnc(-c2ccccc2)[nH]1] |
| 5 | 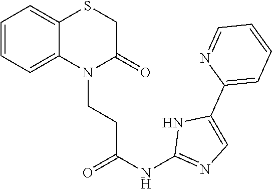 [SMILES: O=C(CCN1C(=O)CSc2ccccc21)Nc1ncc(-c2ccccn2)[nH]1] |
| 6 | 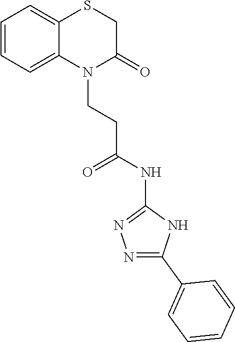 [SMILES: O=C(CCN1C(=O)CSc2ccccc21)Nc1nnc(-c2ccccc2)[nH]1] |
| 7 | 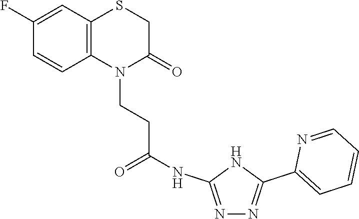 [SMILES: O=C(CCN1C(=O)CSc2cc(F)ccc21)Nc1nnc(-c2ccccn2)[nH]1] |
| 8 | 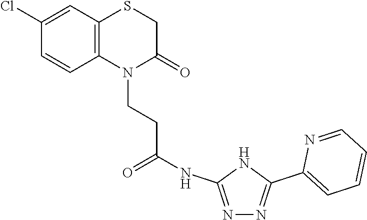 [SMILES: O=C(CCN1C(=O)CSc2cc(Cl)ccc21)Nc1nnc(-c2ccccn2)[nH]1] |
| 9 | 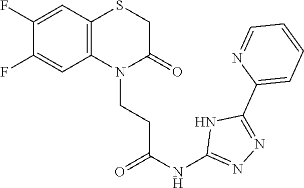 [SMILES: O=C(CCN1C(=O)CSc2cc(F)c(F)cc21)Nc1nnc(-c2ccccn2)[nH]1] |
| 10 | 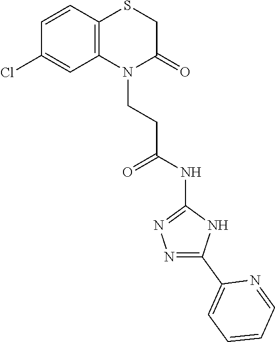 [SMILES: O=C(CCN1C(=O)CSc2ccc(Cl)cc21)Nc1nnc(-c2ccccn2)[nH]1] |
| 11 | 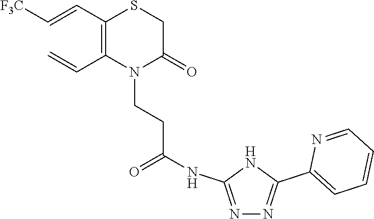 [SMILES: C=CC1=C(/C=C/C(F)(F)F)SCC(=O)N1CCC(=O)Nc1nnc(-c2ccccn2)[nH]1] |
| 12 | 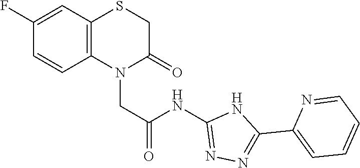 [SMILES: O=C(CN1C(=O)CSc2cc(F)ccc21)Nc1nnc(-c2ccccn2)[nH]1] |
| 13 | 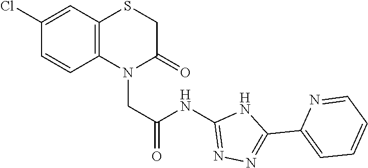 [SMILES: O=C(CN1C(=O)CSc2cc(Cl)ccc21)Nc1nnc(-c2ccccn2)[nH]1] |
| 14 | 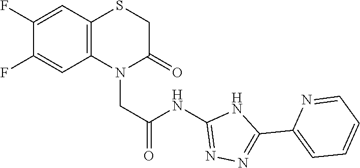 [SMILES: O=C(CN1C(=O)CSc2cc(F)c(F)cc21)Nc1nnc(-c2ccccn2)[nH]1] |
| 15 | 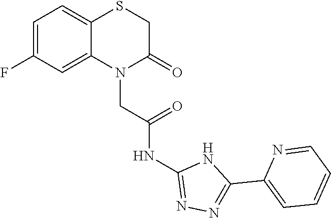 [SMILES: O=C(CN1C(=O)CSc2ccc(F)cc21)Nc1nnc(-c2ccccn2)[nH]1] |
| 16 | 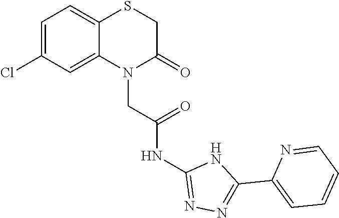 [SMILES: O=C(CN1C(=O)CSc2ccc(Cl)cc21)Nc1nnc(-c2ccccn2)[nH]1] |
| 17 | 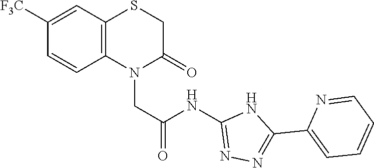 [SMILES: O=C(CN1C(=O)CSc2cc(C(F)(F)F)ccc21)Nc1nnc(-c2ccccn2)[nH]1] |
| 18 | 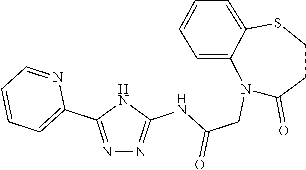 |
| 19 | 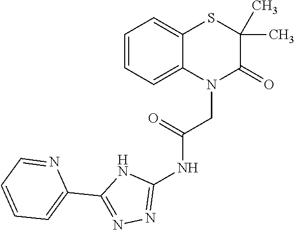 [SMILES: CC1(C)Sc2ccccc2N(CC(=O)Nc2nnc(-c3ccccn3)[nH]2)C1=O] |
| 20 | 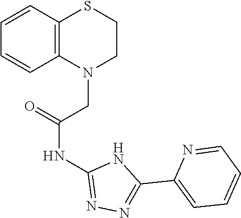 [SMILES: O=C(CN1CCSc2ccccc21)Nc1nnc(-c2ccccn2)[nH]1] |
| 21 | 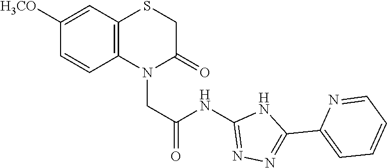 [SMILES: COc1ccc2c(c1)SCC(=O)N2CC(=O)Nc1nnc(-c2ccccn2)[nH]1] |
| 22 | 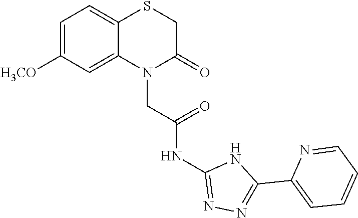 [SMILES: COc1ccc2c(c1)N(CC(=O)Nc1nnc(-c3ccccn3)[nH]1)C(=O)CS2] |
| 23 | 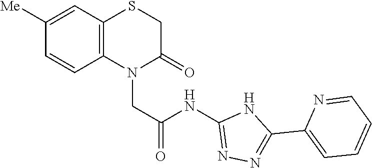 [SMILES: Cc1ccc2c(c1)SCC(=O)N2CC(=O)Nc1nnc(-c2ccccn2)[nH]1] |
| 24 | 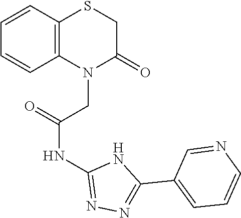 [SMILES: O=C(CN1C(=O)CSc2ccccc21)Nc1nnc(-c2cccnc2)[nH]1] |
| 25 | 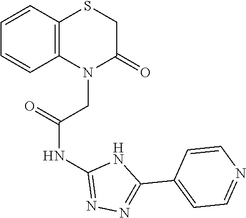 [SMILES: O=C(CN1C(=O)CSc2ccccc21)Nc1nnc(-c2ccncc2)[nH]1] |
| 26 | 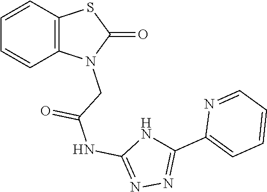 [SMILES: O=C(Cn1c(=O)sc2ccccc21)Nc1nnc(-c2ccccn2)[nH]1] |
| 27 | 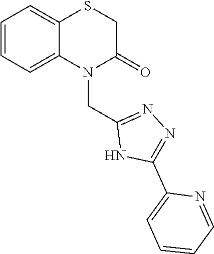 [SMILES: O=C1CSc2ccccc2N1Cc1nnc(-c2ccccn2)[nH]1] |
| 28 | 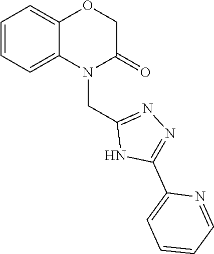 [SMILES: O=C1COc2ccccc2N1Cc1nnc(-c2ccccn2)[nH]1] |
| 29 | 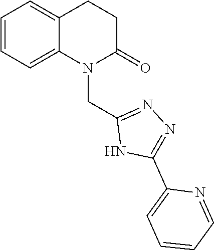 [SMILES: O=C1CCc2ccccc2N1Cc1nnc(-c2ccccn2)[nH]1] |
| 30 | 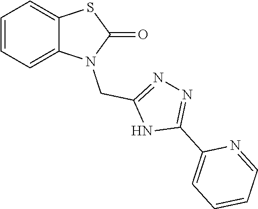 [SMILES: O=c1sc2ccccc2n1Cc1nnc(-c2ccccn2)[nH]1] |
| 31 | 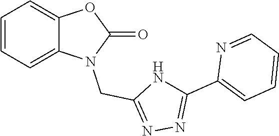 [SMILES: O=c1oc2ccccc2n1Cc1nnc(-c2ccccn2)[nH]1] |
| 32 | 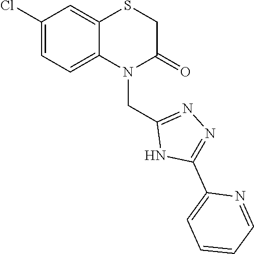 [SMILES: O=C1CSc2cc(Cl)ccc2N1Cc1nnc(-c2ccccn2)[nH]1] |
| 33 | 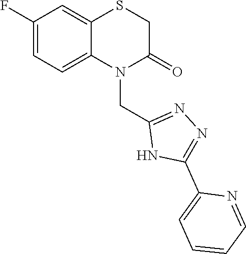 [SMILES: O=C1CSc2cc(F)ccc2N1Cc1nnc(-c2ccccn2)[nH]1] |
| 34 | 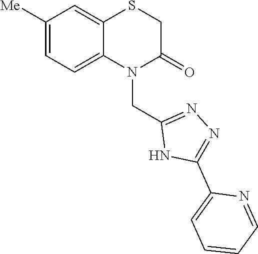 [SMILES: Cc1ccc2c(c1)SCC(=O)N2Cc1nnc(-c2ccccn2)[nH]1] |
| 35 | 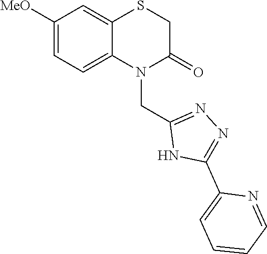 [SMILES: COc1ccc2c(c1)SCC(=O)N2Cc1nnc(-c2ccccn2)[nH]1] |
| 36 | 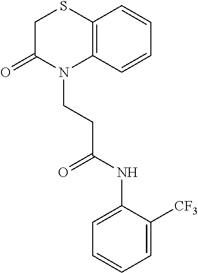 [SMILES: O=C(CCN1C(=O)CSc2ccccc21)Nc1ccccc1C(F)(F)F] |
| 37 | 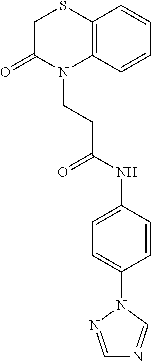 [SMILES: O=C(CCN1C(=O)CSc2ccccc21)Nc1ccc(-n2cncn2)cc1] |
| 38 | 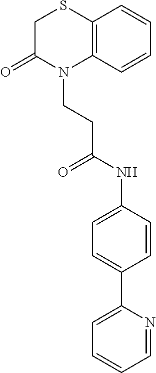 [SMILES: O=C(CCN1C(=O)CSc2ccccc21)Nc1ccc(-c2ccccn2)cc1] |
| 39 | 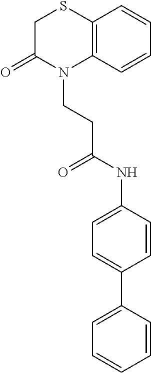 [SMILES: O=C(CCN1C(=O)CSc2ccccc21)Nc1ccc(-c2ccccc2)cc1] |
| 40 | 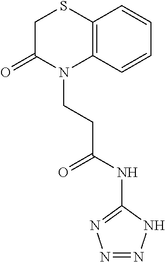 [SMILES: O=C(CCN1C(=O)CSc2ccccc21)Nc1nnn[nH]1] |
| 41 | 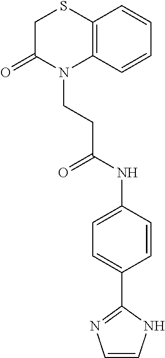 [SMILES: O=C(CCN1C(=O)CSc2ccccc21)Nc1ccc(-c2ncc[nH]2)cc1] |
| 42 | 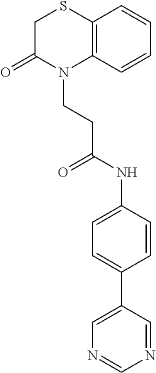 [SMILES: O=C(CCN1C(=O)CSc2ccccc21)Nc1ccc(-c2cncnc2)cc1] |
| 43 | 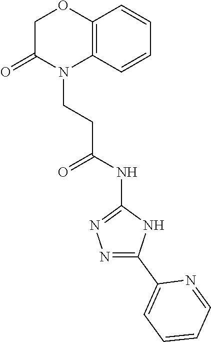 [SMILES: O=C(CCN1C(=O)COc2ccccc21)Nc1nnc(-c2ccccn2)[nH]1] |
| 44 | 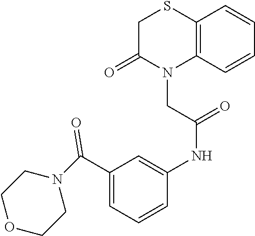 [SMILES: O=C(CN1C(=O)CSc2ccccc21)Nc1cccc(C(=O)N2CCOCC2)c1] |
| 45 | 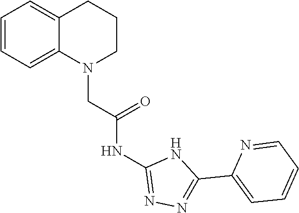 [SMILES: O=C(CN1CCCc2ccccc21)Nc1nnc(-c2ccccn2)[nH]1] |
| 46 | 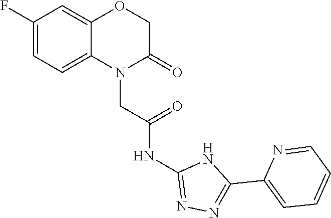 [SMILES: O=C(CN1C(=O)COc2cc(F)ccc21)Nc1nnc(-c2ccccn2)[nH]1] |
| 47 | 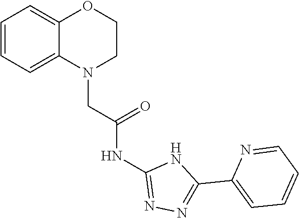 [SMILES: O=C(CN1CCOc2ccccc21)Nc1nnc(-c2ccccn2)[nH]1] |
| 48 | 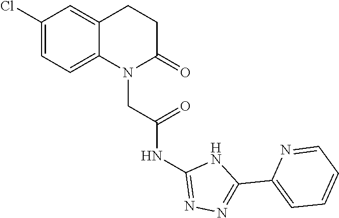 [SMILES: O=C(CN1C(=O)CCc2cc(Cl)ccc21)Nc1nnc(-c2ccccn2)[nH]1] |
| 49 | 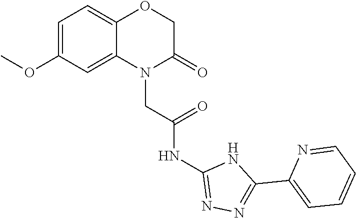 [SMILES: COc1ccc2c(c1)N(CC(=O)Nc1nnc(-c3ccccn3)[nH]1)C(=O)CO2] |
| 50 | 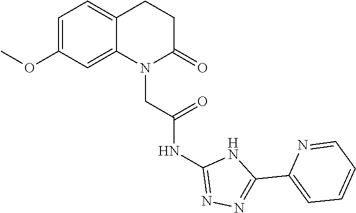 [SMILES: COc1ccc2c(c1)N(CC(=O)Nc1nnc(-c3ccccn3)[nH]1)C(=O)CC2] |
| 51 | 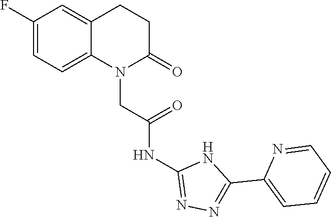 [SMILES: O=C(CN1C(=O)CCc2cc(F)ccc21)Nc1nnc(-c2ccccn2)[nH]1] |
| 52 | 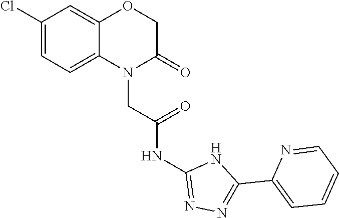 [SMILES: O=C(CN1C(=O)COc2cc(Cl)ccc21)Nc1nnc(-c2ccccn2)[nH]1] |
| 53 | 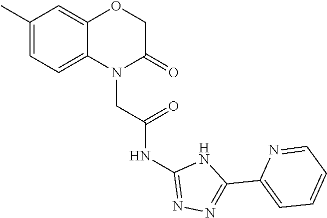 [SMILES: Cc1ccc2c(c1)OCC(=O)N2CC(=O)Nc1nnc(-c2ccccn2)[nH]1] |
| 54 | 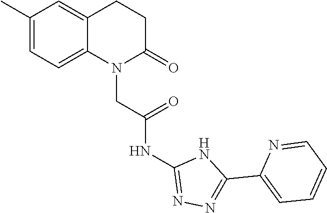 [SMILES: Cc1ccc2c(c1)CCC(=O)N2CC(=O)Nc1nnc(-c2ccccn2)[nH]1] |
| 55 | 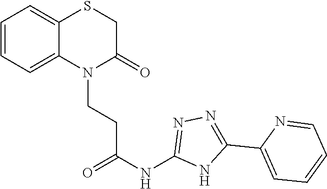 [SMILES: O=C(CCN1C(=O)CSc2ccccc21)Nc1nnc(-c2ccccn2)[nH]1] |
| 56 | 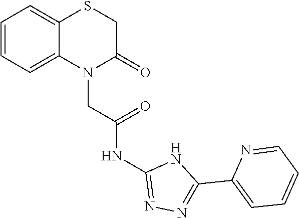 [SMILES: O=C(CN1C(=O)CSc2ccccc21)Nc1nnc(-c2ccccn2)[nH]1] |
| 57 | 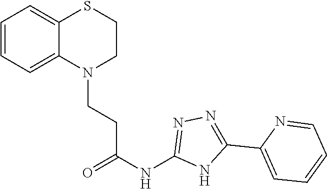 [SMILES: O=C(CCN1CCSc2ccccc21)Nc1nnc(-c2ccccn2)[nH]1] |
| 58 | 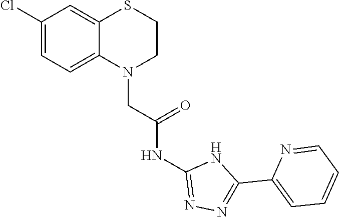 [SMILES: O=C(CN1CCSc2cc(Cl)ccc21)Nc1nnc(-c2ccccn2)[nH]1] |
| 59 | 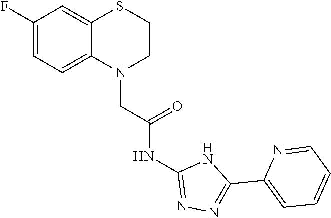 [SMILES: O=C(CN1CCSc2cc(F)ccc21)Nc1nnc(-c2ccccn2)[nH]1] |
| 60 | 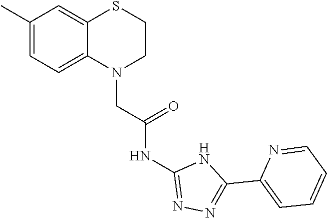 [SMILES: Cc1ccc2c(c1)SCCN2CC(=O)Nc1nnc(-c2ccccn2)[nH]1] |
| 61 | 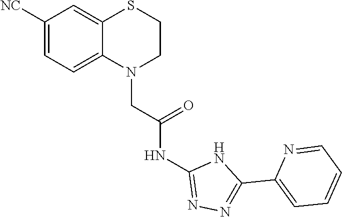 [SMILES: N#Cc1ccc2c(c1)SCCN2CC(=O)Nc1nnc(-c2ccccn2)[nH]1] |
| 62 | 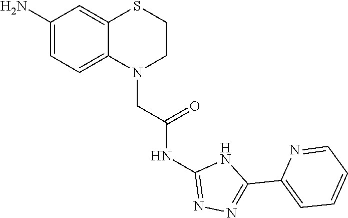 [SMILES: Nc1ccc2c(c1)SCCN2CC(=O)Nc1nnc(-c2ccccn2)[nH]1] |
| 63 | 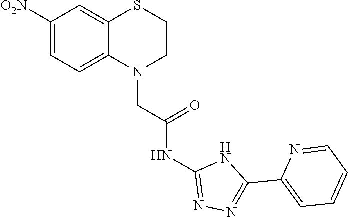 [SMILES: O=C(CN1CCSc2cc([N+](=O)[O-])ccc21)Nc1nnc(-c2ccccn2)[nH]1] |
| 64 | 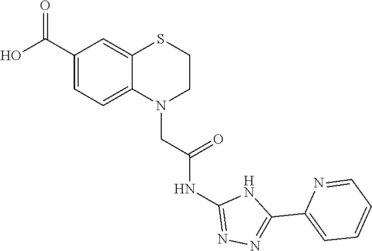 [SMILES: O=C(CN1CCSc2cc(C(=O)O)ccc21)Nc1nnc(-c2ccccn2)[nH]1] |
| 65 | 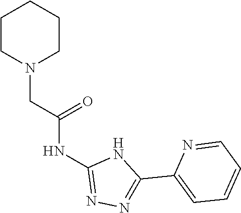 [SMILES: O=C(CN1CCCCC1)Nc1nnc(-c2ccccn2)[nH]1] |
| 66 | 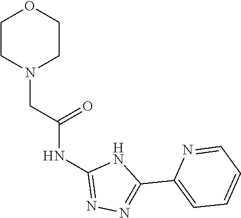 [SMILES: O=C(CN1CCOCC1)Nc1nnc(-c2ccccn2)[nH]1] |
| 67 | 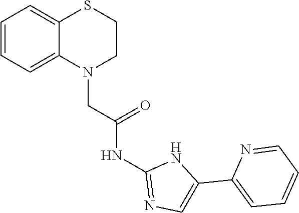 [SMILES: O=C(CN1CCSc2ccccc21)Nc1ncc(-c2ccccn2)[nH]1] |
| 68 | 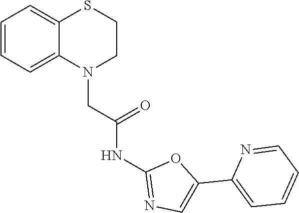 [SMILES: O=C(CN1CCSc2ccccc21)Nc1ncc(-c2ccccn2)o1] |
| 69 | 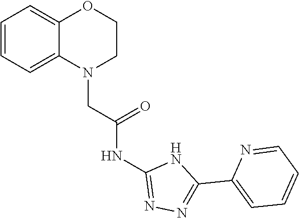 [SMILES: O=C(CN1CCOc2ccccc21)Nc1nnc(-c2ccccn2)[nH]1] |

a. General Experimental

All reactions were carried out in an oven- or flame-dried glassware under argon atmosphere using standard gas-tight syringe, cannula, and septa. The reaction temperatures were measured externally. Stirring was achieved with oven dried magnetic bars. All the reactions were done in anhydrous solvents (DMF, THF, CH₂Cl₂, 1,4-Dioxane, 1-Butanol, CHCl₃, DME) purchased from Sigma-Aldrich. Microwave reactions were performed in CEM discover Labmate System with Intelligent Technology for Focused Microwave Synthesizer (Explorer 48). All commercially purchased reagents were used without purification. The reactions were monitored by thin-layer chromatography (TLC) on a pre-coated silica gel (60 F254) glass plates from EMD Millipore and visualized using UV light (254 nm). Purification of the compounds was performed on Teledyne-ISCO Combiflash Rf 200 purification system. Used Redisep Rf® normal phase silica gel columns 230-400 mesh. Proton NMR spectra were recorded on a Varian Unity 400 NMR spectrometer operating at 400 MHz calibrated to the solvent peak and TMS peak. The chemical formula and Exact Mass for target compounds were determined from the (M+H)⁺ by high resolution mass spectroscopy using an Agilent 6210 Electrospray Time of Flight. The purity of the final compounds were checked by HPLC using Method A: Agilent 1100 LC equipped with a diode array UV detector and monitored at multiple wavelengths, Bondclone 10μ C18 column using Solvent A: H₂O, Solvent B: CH₃CN, 1.0 ml/min; 30 min linear gradient from 10-90% B. Method B: Eclipse plus-C18 column (3.5 μm, 4.6×100 mm) using Solvent A: H₂O, Solvent B: CH₃CN (both containing 0.05% TFA) from 10-90% gradient in 15 min or Method C: Shim-Pack XR-ODS C18-column (2.2 μm, 3.0×50 mm) using Solvent A: H₂O, Solvent B: CH₃CN(both containing 0.05% TFA) from 10-90% gradient in 5 min. ESI-MS spectra were recorded on a BioTof-2 time-of-flight mass spectrometer.

b. General Scheme For The Synthesis Of Compounds 1-26, 43, 45-54 (ROUTE 1)

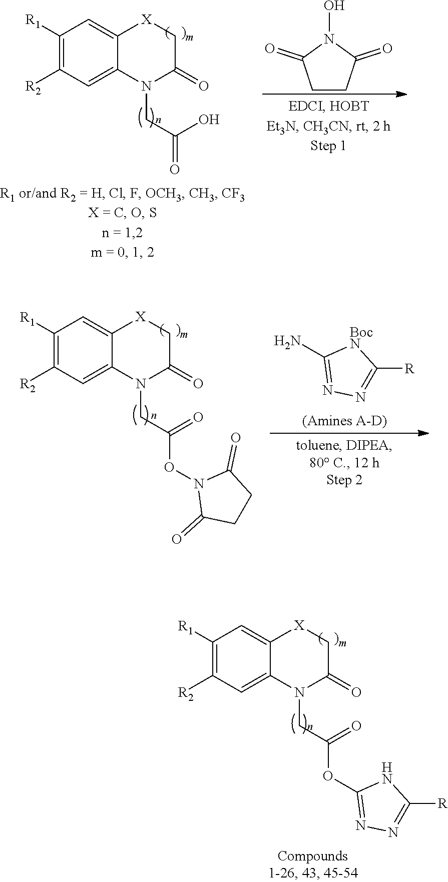

Step 1: To a stirred solution of commercially available corresponding starting acid (2.41 mmol) and 1-hydroxypyrrolidine-2,5-dione (2.41 mmol) in CH₃CN (10 mL) were added EDCI·HCl (2.89 mmol), HOBt (2.89 mmol), and Et₃N (4.82 mmol) at room temperature under nitrogen atmosphere. The resulting suspension was further stirred at room temperature for 2 h. The reaction mixture was diluted with water (50 mL) and extracted with ethyl acetate (3×50 mL). The combined organic layer was washed with brine solution (2×50 mL), dried over anhydrous Na₂SO₄, filtered and concentrated under reduced pressure to give crude intermediate compound. This crude product was used directly in the next step without purification.

Step 2: To a stirred solution of corresponding crude compound from Step 1 (1.31 mmol) and corresponding amine (A-D) (1.35 mmol) in toluene (25 mL) was added DIPEA (2.62 mmol) at room temperature under nitrogen atmosphere. The resulting suspension was stirred at 80° C. for 16 h. The reaction mixture was diluted with water (25 mL) and extracted with ethyl acetate (3×25 mL). The combined organic layers were washed with brine solution (1×50 mL), dried over anhydrous Na₂SO₄, filtered and concentrated under reduced pressure to give the crude product. The crude product was purified by column chromatography (5-10% CH₃OH—CH₂Cl₂) to afford target compounds 1-26, 43 and 48-54 in 14-25% yield, as an off-white solid.

### i. 2-(3-Oxo-2H-BENZO[B][1,4]OXAZIN-4(311)-YL)-N-(5-(PYRIDIN-2-YL)-4H-1,2,4-TRIAZOL-3-YL)ACETAMIDE (1)

This compound was prepared by the reaction of commercially available 2-(3-oxo-2,3-dihydro-4H-benzo[b][1,4]oxazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), to give pure compound 1 (14% yield). ¹H NMR (400 MHz, DMSO-d₆) δ 13.76 (brs, 1H), 11.06 (brs, 1H), 8.65 (d, J=3.9 Hz, 1H), 8.00-7.88 (m, 2H), 7.45-7.41 (m, 1H), 7.02-7.01 (m, 4H), 4.84 (s, 2H), 4.69 (s, 2H). ESI-MS (m/z)=351.1 (M+H)+. Purity by HPLC: 95.6% (Method B).

### ii. 2-(2-OXO-3,4-DIHYDROQUINOLIN-1(2H)-YL)-N-(5-(PYRIDIN-2-YL)-4H-1,2,4-TRIAZOL-3-YL)ACETAMIDE (2)

This compound was prepared by the reaction of commercially available 2-(2-oxo-3,4-dihydroquinolin-1(2H)-yl)acetic acid and tert-butyl 3-amino-5-(pyridin-2-yl)-4H-1,2,4-triazole-4-carboxylate (Amine B) using route 1. The crude product was purified by column chromatography (using silica, 5-10% CH₃OH—CH₂Cl₂), to give pure compound 2 (23% yield). ¹H NMR (400 MHz, DMSO-d₆) δ 13.53 (brs, 1H), 10.80 (brs, 1H), 8.64 (d, J=4.8 Hz, 1H), 8.00-7.87 (m, 2H), 7.42 (t, J=5.7 Hz, 1H), 7.20 (t, J=10 Hz, 2H), 7.01-6.93 (m, 2H), 4.80 (s, 2H), 2.91 (q, J=6.6 Hz, 2H), 2.61 (q, J=5.4 Hz, 2H). ESI-MS (m/z)=349.1 (M+H)⁺. Purity by HPLC: 98.9% (Method B).

### iii. 3-(2-Oxo-3,4-DIHYDROQUINOLIN-1(2H)-YL)-N-(5-(PYRIDIN-2-YL)-4H-1,2,4-TRIAZOL-3-YL)PROPANAMIDE (3)

This compound was prepared by the reaction of commercially available 3-(2-oxo-3,4-dihydroquinolin-1(2H)-yl)propanoic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), to give pure compound 3 (25% yield). ¹H NMR (400 MHz, DMSO-d₆) δ 8.63 (d, J=6.0 Hz, 1H), 7.98-7.86 (m, 3H), 7.41 (brs, 1H), 7.26-7.18 (m, 3H), 7.00-6.95 (m, 1H), 4.21 (t, J=7.2 Hz, 2H), 2.85 (t, J=7.8 Hz, 2H), 2.73 (t, J=7.2 Hz, 2H), 2.55-2.49 (m, 2H). ESI-MS (m/z)=363.1 (M+H)⁺. Purity by HPLC: 97.1% (Method B).

### iv. 2-(3-Oxo-2H-BENZO[B][1,4]THIAZIN-4(3H)-YL)-N-(5-PHENYL-4H-1,2,4-TRIAZOL-3-YL)ACETAMIDE (4)

This compound was prepared by the reaction of commercially available 2-(3-oxo-2,3-dihydro-4H-benzo[b][1,4]thiazin-4-yl)acetic acid and tert-butyl 3-amino-5-phenyl-4H-1,2,4-triazole-4-carboxylate (Amine A) using route 1. The crude product was purified by column chromatography (5-10% CH₃OH—CH₂Cl₂), to give pure compound 4 (16% yield). ¹H NMR (400 MHz, DMSO-d₆) δ 13.30 (brs, 1H), 11.74 (brs, 1H), 7.95 (d, J=8.80 Hz, 2H), 7.47-7.39 (m, 4H), 7.28-7.24 (m, 1H), 7.23-7.20 (m, 1H), 7.19-7.15 (m, 1H), 4.84 (s, 2H), 3.55 (s, 2H). ESI-MS (m/z)=366.1 (M+H)⁺. Purity by HPLC: 98.4% (Method B).

### v. 3-(3-Oxo-2H-BENZO[B][1,4]THIAZIN-4(3H)-YL)-N-(5-(PYRIDIN-2-YL)-1H-IMIDAZOL-2-YL)PROPANAMIDE (5)

This compound was prepared by the reaction of commercially available 3-(3-oxo-2,3-dihydro-4H-benzo[b][1,4]thiazin-4-yl)propanoic acid and commercially available 5-(pyridin-3-yl)-1H-imidazol-2-amine using route 1. The crude product was purified by column chromatography (5-10% CH₃OH—CH₂Cl₂), to give pure compound 5 (16% yield). ¹H NMR (400 MHz, DMSO-d₆) δ 11.78 (s, 1H), 11.40 (s, 1H), 8.47 (d, J=3.6 Hz, 1H), 7.75-7.70 (m, 2H), 7.41 (dd, J=6.0 Hz, J=12.4 Hz, 2H), 7.16 (t, J=5.6 Hz, 1H), 7.07 (t, J=7.2 Hz, 1H), 4.25 (t, J=7.2 Hz, 2H), 3.51 (s, 2H), 2.73-2.71 (m, 2H), 2.54 (s, 2H). ESI-MS (m/z)=380.1 (M+H)+. Purity by HPLC: 97.6% (Method B).

### vi. 3-(3-Oxo-2H-BENZO[B][1,4]THIAZIN-4(3H)-YL)-N-(5-PHENYL-4H-1,2,4-TRIAZOL-3-YL)PROPANAMIDE (6)

This compound was prepared by the reaction of commercially available 3-(3-oxo-2,3-dihydro-4H-benzo[b][1,4]thiazin-4-yl)propanoic acid and tert-butyl 3-amino-5-phenyl-4H-1,2,4-triazole-4-carboxylate (Amine A) using route 1. The crude product was purified by column chromatography (5-10% CH₃OH—CH₂Cl₂), to give pure compound 6 (16% yield). ¹H NMR (400 MHz, DMSO-d₆) δ 13.49 (s, 1H), 11.74 (s, 1H), 7.95 (d, J=7.2 Hz, 2H), 7.46-7.41 (m, 5H), 7.31 (t, J=7.6 Hz, 1H), 7.07 (t, J=7.6 Hz, 1H), 4.26 (t, J=7.2 Hz, 2H), 3.52 (s, 2H), 2.75 (t, J=7.2 Hz, 2H). ESI-MS (m/z)=380.0 (M+H)+. Purity by HPLC: 98.8% (Method B).

### vii. 3-(7-FLUORO-3-Oxo-2H-BENZO[B][1,4]THIAZIN-4(3H)-YL)-N-(5-(PYRIDIN-2-YL)-4H-1,2,4-TRIAZOL-3-YL)PROPANAMIDE (7)

This compound was prepared by the reaction of commercially available 3-(7-fluoro-3-oxo-2,3-dihydro-4H-benzo[b][1,4]thiazin-4-yl)propanoic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), to give pure compound 7 (34% yield). ¹H NMR (300 MHz, DMSO-d₆) δ 8.64 (brs, 1H), 7.97-7.89 (m, 2H), 7.42-7.10 (m, 4H), 4.25 (brs, 2H), 3.49 (brs, 2H), 2.76-2.74 (m, 2H). ESI-MS (m/z)=399.1 (M+H)⁺. Purity by HPLC: 93.4% (Method B).

### viii. 3-(7-CHLORO-3-OXO-2H-BENZO[B][1,4]THIAZIN-4(3H)-YL)-N-(5-(PYRIDIN-2-YL)-4H-1,2,4-TRIAZOL-3-YL)PROPANAMIDE (8)

This compound was prepared by the reaction of commercially available 3-(7-chloro-3-oxo-2,3-dihydro-4H-benzo[b][1,4]thiazin-4-yl)propanoic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), to give pure compound 8 (25% yield). ¹H NMR (300 MHz, DMSO-d₆) δ 8.64 (brs, 1H), 7.97-7.89 (m, 2H), 7.46-7.29 (m, 4H), 4.25 (brs, 2H), 3.50 (s, 2H), 274 (t, J=9.2 Hz, 2H). ESI-MS (m/z)=415.0 (M+H)+. Purity by HPLC: 92.7% (Method B).

### ix. 3-(6,7-DIFLUORO-3-OXO-2H-BENZO[B][1,4]THIAZIN-4(3H)-YL)-N-(5-(PYRIDIN-2-YL)-4H-1,2,4-TRIAZOL-3-YL)PROPANAMIDE (9)

This compound was prepared by the reaction of commercially available 3-(6,7-difluoro-3-oxo-2,3-dihydro-4H-benzo[b][1,4]thiazin-4-yl)propanoic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), to give pure compound 9 (25% yield). ¹H NMR (300 MHz, DMSO-d₆) δ 8.63 (brs, 1H), 7.94-7.87 (m, 2H), 7.58-7.39 (m, 3H), 4.24 (brs, 2H), 3.50-3.48 (in, 2H), 274 (t, J=9.2 Hz, 2H). ESI-MS (m/z)=417.1 (M+H)⁺. Purity by HPLC: 90.84% (Method B).

### x. 3-(6-CHLORO-3-OXO-2H-BENZO[B][1,4]THIAZIN-4(3H)-YL)-N-(5-(PYRIDIN-2-YL)-4H-1,2,4-TRIAZOL-3-YL)PROPANAMIDE (10)

This compound was prepared by the reaction of commercially available 3-(6-chloro-3-oxo-2,3-dihydro-4H-benzo[b][1,4]thiazin-4-yl)propanoic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂), 140 mg, 46% yield, HPLC: 81.9%, off-white solid. Further purified by Boc protection then de-protection. ESI-MS (m/z)=415.0 (M+H)⁺. To a stirred solution of crude compound were added Et₃N (1.01 mmol), and Boc anhydride (0.33 mmol) at room temperature and the resulting suspension was stirred at room temperature for 24 h. The reaction mixture was concentrated under reduced pressure to get the crude product. The crude product was purified by column chromatography (10-20% CH₃OH—CH₂Cl₂), to give pure compound 10 (50% yield). ¹H NMR (300 MHz, DMSO-d₆) δ 8.64 (brs, 1H), 7.97-7.88 (m, 2H), 7.46-7.38 (m, 3H), 7.08 (d, J=8.1 Hz, 1H), 4.26-4.24 (brs. 2H), 3.49 (m, 2H), 276 (t, J=9.2 Hz, 2H). ESI-MS (m/z)=415.0 (M+H)⁺. Purity by HPLC: 94.2% (Method B).

### xi. 3-(3-Oxo-7-(TRIFLUOROMETHYL)-2H-BENZO[B][1,4]THIAZIN-4(3H)-YL)-N-(5-(PYRIDIN-2-YL)-4H-1,2,4-TRIAZOL-3-YL)PROPANAMIDE (11)

This compound was prepared by the reaction of commercially available 3-(3-oxo-7-(trifluoromethyl)-2,3-dihydro-4H-benzo[b][1,4]thiazin-4-yl)propanoic acid and tert-butyl 3-amino-5-(pyridin-2-yl)-4H-1,2,4-triazole-4-carboxylate (Amine B) using route 1. The crude product was purified by column chromatography (10-20% CH₃OH—CH₂Cl₂) to give pure compound 11 (27% yield). ¹H NMR (300 MHz, DMSO-d₆) δ 8.63 (brs, 1H), 7.97-7.88 (m, 2H), 7.74 (brs, 1H), 7.58 (brs, 1H), 7.41 (brs, 1H), 4.31 (t, J=10.0 Hz, 2H), 3.56 (s, 2H), 2.77 (t, J=10.0 Hz, 2H). ESI-MS (m/z)=449.0 (M+H)⁺. Purity by HPLC: 93.2%(Method B).

### xii. 2-(7-FLUORO-3-oxo-2H-BENZO[B][1,4]THIAZIN-4(3H)-YL)-N-(5-(PYRIDIN-2-YL)-4H-1,2,4-TRIAZOL-3-YL)ACETAMIDE (12)

This compound was prepared by the reaction of commercially available 2-(7-fluoro-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 12 (25% yield). ¹H NMR (300 MHz, DMSO-d₆) δ 8.65 (brs, 1H), 8.00-7.91 (m, 2H), 7.43 (brs, 1H), 7.29 (d, J=11.2 Hz, 1H), 7.22-7.06 (m, 3H), 4.83 (s, 2H), 3.57 (s, 2H). ESI-MS (m/z)=385.0 (M+H)⁺. Purity by HPLC: 98.5% (Method B).

### xiii. 2-(7-CHLORO-3-OXO-2H-BENZO[B][1,4]THIAZIN-4(3H)-YL)-N-(5-(PYRIDIN-2-YL)-4H-1,2,4-TRIAZOL-3-YL)ACETAMIDE (13)

This compound was prepared by the reaction of commercially available 2-(7-chloro-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 13 (29% yield). ¹H NMR (300 MHz, DMSO-d₆) δ 8.65 (d, J=3.6 Hz, 2H), 8.00-7.88 (m, 4H), 7.49-7.43 (m, 4H), 7.31-7.27 (m, 2H), 7.14 (d, J=8.7 Hz, 2H), 4.84 (s, 2H), 3.58 (s, 2H). ESI-MS (m/z)=401.0 (M+H)⁺. Purity by HPLC: 97.7% (Method B).

### xiv. 2-(6,7-DIFLUORO-3-OXO-2H-BENZO[B][1,4]THIAZIN-4(3H)-YL)-N-(5-(PYRIDIN-2-YL)-4H-1,2,4-TRIAZOL-3-YL)ACETAMIDE (14)

This compound was prepared by the reaction of commercially available 2-(6,7-difluoro-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 14 (27% yield). ¹H NMR (300 MHz, DMSO-d₆) δ 8.66 (brs, 1H), 8.00-7.91 (m, 4H), 7.56-7.50 (m, 1H), 7.45-7.43 (m, 1H), 7.34-7.16 (m, 1H), 4.86 (s, 2H), 3.58 (s, 2H). ESI-MS (m/z)=401.0 (M+H)⁺. Purity by HPLC: 95.40% (Method B).

### xv. 2-(6-FLUORO-3-OXO-2H-BENZO[B][1,4]THIAZIN-4(3H)-YL)-N-(5-(PYRIDIN-2-YL)-4H-1,2,4-TRIAZOL-3-YL)ACETAMIDE (15)

This compound was prepared by the reaction of commercially available 2-(6-fluoro-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 15 (49.1% yield). ¹H NMR (300 MHz, DMSO-d₆) δ 8.65 (d, J=6.0 Hz, 1H), 8.00-7.88 (m, 2H), 7.46-7.41 (m, 2H), 7.07-6.87 (m, 2H), 4.87 (s, 2H), 3.55 (s, 2H). ESI-MS (m/z)=385.0 (M+H)⁺. Purity by HPLC: 97.1% (Method B).

### xvi. 2-(6-CHLORO-3-OXO-2H-BENZO[B][1,4]THIAZIN-4(3H)-YL)-N-(5-(PYRIDIN-2-YL)-4H-1,2,4-TRIAZOL-3-YL)ACETAMIDE (16)

This compound was prepared by the reaction of commercially available 2-(6-chloro-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 16 (35% yield). ¹H NMR (300 MHz, DMSO-d₆) δ 8.65 (d, J=3.6 Hz, 1H), 8.00 (d, J=7.8 Hz, 1H), 7.91 (t, J=7.2 Hz, 1H), 7.43 (d, J=8.1 Hz, 2H), 7.23 (s, 1H), 7.10 (d, J=11.2 Hz, 1H), 4.88 (s, 2H), 3.57 (s, 2H). ESI-MS (m/z)=401.0 (M+H)⁺. Purity by HPLC: 98.43% (Method B).

### xvii. 2-(3-OXO-7-(TRIFLUOROMETHYL)-2H-BENZO[B][1,4]THIAZIN-4(3H)-YL)-N-(5-(PYRIDIN-2-YL)-4H-1,2,4-TRIAZOL-3-YL)ACETAMIDE (17)

This compound was prepared by the reaction of commercially available 2-(3-oxo-7-(trifluoromethyl)-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 17 (13% yield). ¹H NMR (300 MHz, DMSO-d₆) δ 8.65 (d, J=1.6 Hz, 1H), 7.99 (d, J=10.4 Hz, 1H), 7.91 (t, J=9.6 Hz, 1H), 7.76 (s, 1H), 7.58 (d, J=11.2 Hz, 1H), 7.44 (d, J=8.4 Hz, 1H), 7.32 (d, J=11.6 Hz, 1H), 4.91 (s, 2H), 3.64 (s, 2H). ESI-MS (m/z)=435.1 (M+H)⁺. Purity by HPLC: 95.3% (Method B).

### xviii. 2-(4-OXO-3,4-DIHYDROBENZO[B][1,4]THIAZEPIN-5(21)-YL)-N-(5-(PYRIDIN-2-YL)-4H-1,2,4-TRIAZOL-3-YL)ACETAMIDE (18)

This compound was prepared by the reaction of commercially available 2-(4-oxo-3,4-dihydrobenzo[b][1,4]thiazepin-5(2H)-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 18 (19% yield). ¹H NMR (300 MHz, DMSO-d₆) δ 8.65 (d, J=3.6 Hz, 1H), 8.00-7.88 (m, 2H), 7.62-7.43 (m, 4H), 7.28-7.25 (m, 1H), 4.58 (brs, 2H), 3.34 (t, J=6.3 Hz, 2H), 2.54-2.52 (m, 2H), (2H). ESI-MS (m/z)=381.0 (M+H)⁺. Purity by HPLC: 99% (Method B).

### xix. 2-(2,2-DIMETHYL-3-OXO-2H-BENZO[B][1,4]THIAZIN-4(3H)-YL)-N-(5-(PYRIDIN-2-YL)-4H-1,2,4-TRIAZOL-3-YL)ACETAMIDE (19)

This compound was prepared by the reaction of commercially available 2-(2,2-dimethyl-3-oxo-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 19 (19% yield). ¹H NMR (300 MHz, DMSO-d₆) δ 8.66 (brs, 1H), 8.00-7.90 (m, 2H), 7.43-7.26 (m, 3H), 7.11-7.04 (m, 2H), 4.86 (s, 2H), 1.38 (s, 6H). ESI-MS (m/z)=395.0 (M+H)⁺. Purity by HPLC: 97.9% (Method B).

### xx. 2-(2H-BENZO[B][1,4]THIAZIN-4(3H)-YL)-N-(5-(PYRIDIN-2-YL)-4H-1,2,4-TRIAZOL-3-YL)ACETAMIDE (20)

This compound was prepared by the reaction of commercially available 2-(2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The reaction mixture was cooled and obtained solid product was filtered and triturated with methanol (10 mL) for 3 times to get the pure compound 20 (27% yield). ¹H NMR (300 MHz, DMSO-d₆) δ 8.64 (s, 1H), 7.97-7.89 (m, 2H), 7.42 (brs, 1H), 6.96-6.88 (m, 2H), 6.59-6.57 (m, 2H), 4.32 (s, 2H), 3.72 (d, J=1.8 Hz, 2H), 3.10 (d, J=1.8 Hz, 2H). ESI-MS (m/z)=353.1 (M+H)⁺. Purity by HPLC: 96.9% (Method B).

### xxi. 2-(7-METHOXY-3-OXO-2H-BENZO[B][1,4]THIAZIN-4(3H)-YL)-N-(5-(PYRIDIN-2-YL)-4H-1,2,4-TRIAZOL-3-YL)ACETAMIDE (21)

This compound was prepared by the reaction of commercially available 2-(7-methoxy-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 21 (12% yield). ¹H NMR (300 MHz, DMSO-d₆) δ 8.64 (brs, 1H), 8.00-7.90 (m, 2H), 7.42 (brs, 1H), 7.07-6.98 (m, 2H), 6.84 (dd, J=2.4 Hz, J=9.0 Hz, 1H), 4.79 (s, 2H), 3.74 (s, 3H), 3.53 (s, 2H). ESI-MS (m/z)=397.1 (M+H)⁺. Purity by HPLC: 98.04% (Method B).

### xxii. 2-(6-METHOXY-3-OXO-2H-BENZO[B][1,4]THIAZIN-4(3H)-YL)-N-(5-(PYRIDIN-2-YL)-4H-1,2,4-TRIAZOL-3-YL)ACETAMIDE (22)

This compound was prepared by the reaction of commercially available 2-(6-methoxy-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 22 (27.2% yield). ¹HNMR (300 MHz, DMSO-d₆) δ 8.65 (brs, 1H), 8.00-7.90 (m, 2H), 7.43 (brs, 1H), 7.30 (d, J=8.4 Hz, 1H), 6.69 (d, J=8.1 Hz, 2H), 4.84 (s, 2H), 3.75 (s, 3H), 3.50 (s, 2H). ESI-MS (m/z)=397.1 (M+H)⁺. Purity by HPLC: 98.3% (Method B).

### xxiii. 2-(7-METHYL-3-OXO-2H-BENZO[B][1,4]THIAZIN-4(3H)-YL)-N-(5-(PYRIDIN-2-YL)-4H-1,2,4-TRIAZOL-3-YL)ACETAMIDE (23)

This compound was prepared by the reaction of commercially available 2-(7-methyl-2,3-dihydro-4H-benzo[b][1,4]thiazin-4-yl)acetic acid and tert-butyl 3-amino-5-(pyridin-2-yl)-4H-1,2,4-triazole-4-carboxylate (Amine B) using route 1. The crude product was purified by column chromatography (5-10% CH₃OH—CH₂Cl₂) to give pure compound 23 (11% yield). ¹H NMR (300 MHz, DMSO-d₆) δ 8.64 (brs, 1H), 8.00-7.91 (m, 2H), 7.43 (brs, 1H), 7.21 (brs, 1H), 7.08-7.02 (m, 2H), 6.81 (s, 2H), 3.52 (s, 2H), 2.53 (s, 3H). ESI-MS (m/z)=381.0 (M+H)⁺. Purity by HPLC: 99.66% (Method B).

### xxiv. 2-(3-OXO-2,3-DIHYDRO-4H-BENZO[B][1,4]THIAZIN-4-YL)-N-(5-(PYRIDIN-3-YL)-4H-1,2,4-TRIAZOL-3-YL)ACETAMIDE (24)

This compound was prepared by the reaction of commercially available 2-(3-oxo-2,3-dihydro-4H-benzo[b][1,4]thiazin-4-yl)acetic acid and tert-butyl 3-amino-5-(pyridin-3-yl)-4H-1,2,4-triazole-4-carboxylate (Amine C) using route 1. The crude product was purified by column chromatography (5-10% CH₃OH—CH₂Cl₂) to give pure compound 24 (45% yield). ¹H NMR (300 MHz, DMSO-d₆) δ 9.12 (s, 1H), 8.59 (d, J=4.5 Hz, 1H), 8.25 (d, J=7.8 Hz, 1H), 7.48-7.40 (m, 2H), 7.26 (t, J=7.8 Hz, 1H), 7.15 (d, J=7.8 Hz, 2H), 7.05 (t, J=7.5 Hz, 1H), 4.85 (s, 2H), 3.55 (s, 2H). ESI-MS (m/z)=365.0 (M+H)⁺. Purity by HPLC: 90.91% (Method B).

### xxv. 2-(3-Oxo-2,3-DIHYDRO-4H-BENZO[B][1,4]THIAZIN-4-YL)-N-(5-(PYRIDIN-4-YL)-4H-1,2,4-TRIAZOL-3-YL)ACETAMIDE (25)

This compound was prepared by the reaction of commercially available 2-(3-oxo-2,3-dihydro-4H-benzo[b][1,4]thiazin-4-yl)acetic acid and tert-butyl 3-amino-5-(pyridin-4-yl)-4H-1,2,4-triazole-4-carboxylate (Amine D) using route 1. The crude product was purified by column chromatography (10-20% CH₃OH—CH₂Cl₂) to give pure compound 25 (50% yield). ¹H NMR (400 MHz, DMSO-d₆) δ 8.66 (dd, J=1.6 Hz, J=4.8 Hz, 2H), 7.86 (dd, J=1.6 Hz, J=4.4 Hz, 2H), 7.44 (dd, J=1.6 Hz, J=8.0 Hz, 1H), 7.30-7.28 (m, 1H), 7.21-7.19 (m, 1H), 7.09-7.08 (m, 1H), 4.85 (s, 2H), 3.59 (s, 2H). ESI-MS (m/z)=365.0 (M+H)⁺. Purity by HPLC: 99.85% (Method B)

### xxvi. 2-(2,2-DIMETHYL-3-oxo-2H-BENZO[B][1,4]THIAZIN-4(3H)-YL)-N-(5-(PYRIDIN-2-YL)-4H-1,2,4-TRIAZOL-3-YL)ACETAMIDE (26)

This compound was prepared by the reaction of commercially available 2-(2-oxobenzo[d]thiazol-3(2H)-yl)acetic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 26 (37% yield). ¹HNMR (300 MHz, DMSO-d₆) δ 8.66 (d, J=3.6 Hz, 1H), 8.01-7.89 (m, 2H), 7.63 (d, J=7.5 Hz, 1H), 7.44-7.17 (m, 4H), 4.94 (s, 2H). ESI-MS (m/z)=351.1 (M+H)⁺. Purity by HPLC: 96.7% (Method B).

### xxvii. 3-(3-Oxo-2,3-DIHYDRO-4H-BENZO[B][1,4]OXAZIN-4-YL)-N-(5-(PYRIDIN-2-YL)-4H-1,2,4-TRIAZOL-3-YL)PROPANAMIDE (43)

This compound was prepared by the reaction of commercially available 3-(3-oxo-2,3-dihydro-4H-benzo[b][1,4]oxazin-4-yl)propanoic acid and tert-butyl 3-amino-5-(pyridin-2-yl)-4H-1,2,4-triazole-4-carboxylate (Amine B) using route 1. The reaction mixture was cooled and the resulting precipitate was triturated with methanol (10 mL) for 3 times, filtered to give pure compound 43 (37% yield). ¹HNMR (300 MHz, DMSO-d₆) δ 8.64 (ddd, J=4.7, 1.8, 0.9 Hz, 1H), 7.96-7.86 (m, 2H), 7.67-7.60 (m, 2H), 7.49-7.42 (m, 2H), 7.11-6.98 (m, 3H), 4.62 (s, 2H), 4.29 (dd, J=8.2, 6.7 Hz, 2H), 3.34-3.31 (m, 2H). ESI-MS (m/z)=365 (M+H)⁺. Purity by HPLC: 99% (Method B).

### xxviii. 2-(3,4-DIHYDROQUINOLIN-1(2H)-YL)-N-(5-(PYRIDIN-2-YL)-4H-1,2,4-TRIAZOL-3-YL)ACETAMIDE (45)

This compound was prepared by the reaction 2-(3,4-dihvdroquinolin-1(2H)-yl)acetic acid² (prepared by the procedure described in reference 2) and reacted with tert-butyl 3-amino-5-(pyridin-2-yl)-4H-1,2,4-triazole-4-carboxylate (Amine B) using route 1. The crude was purified by Preparative HPLC (Column: XSelect CSH Prep C18 OBD Column, 5 μm, 19×150 mm. Mobile Phase A: Water (0.1% formic acid); Mobile Phase B: CAN. Flow rate: 25 mL/min. Gradient: 20% B to 43% B in 7 min; 254/220 nm). This resulted in pure compound 45 in 20% yield. ¹H NMR (300 MHz, DMSO-d₆) δ 14.45 (s, 1H), 11.85 (s, 1H), 8.75-8.60 (m, 1H), 8.03-7.85 (m, 2H), 7.59-7.36 (m, 1H), 7.01-6.87 (m, 2H), 6.57-6.40 (m, 2H), 4.22 (s, 2H), 3.44 (t, J=6.0 Hz, 2H), 2.72 (t, J=6.2 Hz, 2H), 1.92 (s, 2H). ESI-MS (m/z): 335 (M+H)⁺. Purity by HPLC: 99% (Method C).

### xxix. 2-(7-FLUORO-3-OXO-2,3-DIHYDRO-4H-BENZO[B][1,4]OXAZIN-4-YL)-N-(5-(PYRIDIN-2-YL)-4H-1,2,4-TRIAZOL-3-YL)ACETAMIDE (46)

This compound was prepared by the reaction 2-(7-fluoro-3-oxo-2,3-dihydro-4H-benzo[b][1,4]oxazin-4-yl)acetic acid¹ (prepared by the procedure described in reference 1) and tert-butyl 3-amino-5-(pyridin-2-yl)-4H-1,2,4-triazole-4-carboxylate (Amine B) using route 1. The crude was purified by Preparative HPLC (Column: XSelect CSH Prep C18 OBD Column, 5 μm, 19×150 mm; Mobile Phase A: Water (0.1% formic acid), Mobile Phase B: Acetonitrile; Flow rate: 25 mL/min; Gradient: 12% B to 30% B in 10 min; 254/220 nm). This resulted in pure compound 46 in 30% yield as white solid. ¹H NMR (300 MHz, DMSO-d₆) δ 8.68 (d, J=4.9 Hz, 1H), 8.06-7.91 (m, 2H), 7.54-7.45 (m, 1H), 7.17-7.07 (m, 1H), 7.01 (dd, J=9.4, 2.8 Hz, 1H), 6.91 (td, J=8.7, 2.9 Hz, 1H), 4.94-4.74 (m, 4H). ESI-MS (m/z): 369 (M+H)⁺. Purity by HPLC: 98% (Method C).

### xxx. 2-(2,3-DIHYDRO-4H-BENZO[B][1,4]OXAZIN-4-YL)-N-(5-(PYRIDIN-2-YL)-4H-1,2,4-TRIAZOL-3-YL)ACETAMIDE (47)

This compound was prepared by the reaction 2-(2,3-dihydro-4H-benzo[b][1,4]oxazin-4-yl)acetic acid² (prepared by the procedure described in reference 2) and tert-butyl 3-amino-5-(pyridin-2-yl)-4H-1,2,4-triazole-4-carboxylate (Amine B) using route 1. The crude was purified by Preparative HPLC (Column: XSelect CSH Prep C18 OBD Column, 5 μm, 19×50 mm; Mobile Phase A: Water (0.1% formic acid), Mobile Phase B: acetonitrile; Flow rate: 25 mL/min; Gradient: 13% B to 30% B in 10 min; 254/220 nm). This resulted pure compound 47 in 15% yield. ¹H NMR (300 MHz, DMSO-d₆) δ 14.03 (s, 1H), 11.56 (s, 1H), 8.67 (d, J=4.9 Hz, 1H), 8.04-7.85 (m, 2H), 7.46 (t, J=6.1 Hz, 1H), 6.79-6.65 (m, 2H), 6.63-6.49 (m, 2H), 4.29-4.20 (m, 4H), 3.52 (t, J=4.4 Hz, 2H). ESI-MS (m/z): 337 (M+H)⁺. Purity by HPLC: 96% (Method C).

### xxxi. 2-(6-CHLORO-2-OXO-3,4-DIHYDROQUINOLIN-1(2H)-YL)-N-(5-(PYRIDIN-2-YL)-4H-1,2,4-TRIAZOL-3-YL)ACETAMIDE (48)

This compound was prepared by the reaction 2-(6-chloro-2-oxo-3,4-dihydroquinolin-1(2H)-yl)acetic (prepared by the procedure described in reference 1) and tert-butyl 3-amino-5-(pyridin-2-yl)-4H-1,2,4-triazole-4-carboxylate (Amine B) using route 1.

The crude was purified by Preparative HPLC (Column: Xselect CSH F-Phenyl OBD column 5 μm, 19×250 mm; Mobile Phase A: Water (0.1% formic acid), Mobile Phase B: acetonitrile; Flow rate: 25 mL/min; Gradient: 10% B to 20% B in 15 min; 254/220 nm). This resulted in pure compound 48 in 30% yield. ¹H NMR (300 MHz, DMSO-d₆) δ 8.67 (d, J=4.8 Hz, 1H), 8.05-7.89 (m, 2H), 7.47 (t, J=6.1 Hz, 1H), 7.36 (d, J=2.5 Hz, 1H), 7.29 (dd, J=8.7, 2.5 Hz, 1H), 7.01 (d, J=8.6 Hz, 1H), 4.77 (s, 2H), 2.94 (t, J=7.3 Hz, 2H), 2.67-2.57 (m, 2H). ESI-MS (m/z): 383 (M+H)⁺. Purity by HPLC: 99% (Method C).

### xxxii. 2-(6-METHOXY-3-OXO-2,3-DIHYDRO-4H-BENZO[B][1,4]OXAZIN-4-YL)-N-(5-(PYRIDIN-2-YL)-4H-1,2,4-TRIAZOL-3-YL)ACETAMIDE (49)

This compound was prepared by the reaction 2-(6-methoxy-3-oxo-2,3-dihydro-4H-benzo[b][1,4]oxazin-4-yl)acetic (prepared by the procedure described in reference 1) and tert-butyl 3-amino-5-(pyridin-2-yl)-4H-1,2,4-triazole-4-carboxylate (Amine B) using route 1. The crude was purified by Preparative HPLC (Column: Xselect CSH F-Phenyl OBD column 19×250 mm, 5 μm; Mobile Phase A: Water (0.1% formic acid), Mobile Phase B: acetonitrile; Flow rate: 25 mL/min; Gradient: 10% B to 20% B in 15 min; 254/220 nm). This resulted in pure compound 49 in 55% yield. ¹H NMR (300 MHz, DMSO-d₆) δ 14.52(13.70) (s, 1H), 12.11 (10.86) (s, 1H), 8.68 (s, 1H), 8.10-7.80 (m, 2H), 7.48 (s, 1H), 6.98 (d, J=8.7 Hz, 1H), 6.80-6.50 (m, 2H), 4.85 (s, 2H), 4.65 (s, 2H), 3.72 (s, 3H). ESI-MS (m/z): 381 (M+H)⁺. Purity by HPLC: 100% (Method C).

### xxxiii. 2-(7-METHOXY-2-OXO-3,4-DIHYDROQUINOLIN-1(2H)-YL)-N-(5-(PYRIDIN-2-YL)-4H-1,2,4-TRIAZOL-3-YL)ACETAMIDE (50)

This compound was prepared by the reaction 2-(7-methoxy-2-oxo-3,4-dihydroquinolin-1(2H)-yl)acetic acid¹ (prepared by the procedure described in reference 1) and tert-butyl 3-amino-5-(pyridin-2-yl)-4H-1,2,4-triazole-4-carboxylate (Amine B) using route 1. The crude was purified by Preparative HPLC (Column: Xselect CSH F-Phenyl OBD column 19×250 mm, 5 μm; Mobile Phase A: Water (0.1% formic acid), Mobile Phase B: acetonitrile; Flow rate: 25 mL/min; Gradient: 10% B to 20% B in 15 min; 254/210 nm). This resulted in pure compound 50 in 51% yield as white solid. ¹H NMR (300 MHz, DMSO-d₆) δ 8.68 (d, J=4.6 Hz, 1H), 8.05-7.85 (m, 2H), 7.47 (s, 1H), 7.16 (d, J=8.2 Hz, 1H), 6.70-6.50 (m, 2H), 4.79 (s, 2H), 3.72 (s, 3H), 2.85 (t, J=7.2 Hz, 2H), 2.59 (t, J=8.7, 5.9 Hz, 2H). ESI-MS (m/z): 379 (M+H)⁺. Purity by HPLC: 100% (Method C).

### xxxiv. 2-(6-FLUORO-2-OXO-3,4-DIHYDROQUINOLIN-1(2H)-YL)-N-(5-(PYRIDIN-2-YL)-4H-1,2,4-TRIAZOL-3-YL)ACETAMIDE (51)

This compound was prepared by the reaction 2-(6-fluoro-2-oxo-3,4-dihydroquinolin-1(2H)-yl)acetic acid¹ (prepared by the procedure described in reference 1) and tert-butyl 3-amino-5-(pyridin-2-yl)-4H-1,2,4-triazole-4-carboxylate (Amine B) using route 1. The crude was purified by Preparative HPLC (Column: Xselect CSH F-Phenyl OBD column 19×250 mm, 5 μm; Mobile Phase A: Water (0.1% formic acid), Mobile Phase B: acetonitrile; Flow rate: 25 mL/min; Gradient: 10% B to 20% B in 15 min; 254/220 nm). This resulted in pure compound 51 in 20% yield as white solid. ¹H NMR (300 MHz, DMSO-d₆) δ 8.68 (d, J=4.8 Hz, 1H), 8.05-7.91 (m, 2H), 7.47 (s, 1H), 7.21-6.95 (m, 3H), 4.78 (s, 2H), 2.93 (t, J=7.4 Hz, 2H), 2.68-2.56 (m, 2H). ESI-MS (m/z): 367 (M+H)⁺. Purity by HPLC: 98% (Method C).

### xxxv. 2-(7-CHLORO-3-OXO-2,3-DIHYDRO-4H-BENZO[B][1,4]OXAZIN-4-YL)-N-(5-(PYRIDIN-2-YL)-4H-1,2,4-TRIAZOL-3-YL)ACETAMIDE (52)

This compound was prepared by the reaction 2-(7-chloro-3-oxo-2,3-dihydro-4H-benzo[b][1,4]oxazin-4-yl)acetic (prepared by the procedure described in reference 1) and tert-butyl 3-amino-5-(pyridin-2-yl)-4H-1,2,4-triazole-4-carboxylate (Amine B) using route 1. The crude was purified by Preparative HPLC (Column: XSelect CSH Prep C18 OBD Column, 5 μm, 19×150 mm; Mobile Phase A: Water (0.1% formic acid), Mobile Phase B: acetonitrile; Flow rate: 25 mL/min; Gradient: 17% B to 43% B in 7 min; 254/220 nm). This resulted in pure compound 52 in 10% yield as white solid. ¹H NMR (300 MHz, DMSO-d₆) δ 14.51 (s, 1H), 12.13 (s, 1H), 8.80-8.60 (m, 1H), 8.10-7.82 (m, 2H), 7.60-7.35 (m, 1H), 7.21-7.03 (m, 3H), 4.89-4.66 (m, 4H). ESI-MS (m/z): 385 (M+H)⁺. Purity by HPLC: 99% (Method C).

### xxxvi. 2-(7-METHYL-3-OXO-2,3-DIHYDRO-4H-BENZO[B][1,4]OXAZIN-4-YL)-N-(5-(PYRIDIN-2-YL)-4H-1,2,4-TRIAZOL-3-YL)ACETAMIDE (53)

This compound was prepared by the reaction 2-(7-methyl-3-oxo-2,3-dihydro-4H-benzo[b][1,4]oxazin-4-yl)acetic acid¹ (prepared by the synthesis described in reference 1) and tert-butyl 3-amino-5-(pyridin-2-yl)-4H-1,2,4-triazole-4-carboxylate (Amine B) using route 1. The crude was purified by Preparative HPLC (Column: Xselect CSH OBD Column 30×150 mm 5 μm; Mobile Phase A: Water (0.1% formic acid), Mobile Phase B: acetonitrile; Flow rate: 60 mL/min; Gradient: 12% B to 42% B in 7 min; 254/220 nm). This resulted in pure compound 53 in 52% yield as white solid. ¹H NMR (300 MHz, DMSO-d₆) δ 8.68 (d, J=4.8 Hz, 1H), 8.06-7.91 (m, 2H), 7.49 (s, 1H), 6.96 (d, J=8.0 Hz, 1H), 6.91-6.81 (m, 2H), 4.81 (s, 2H), 4.70 (s, 2H), 2.25 (s, 3H). EST-MS (m/z): 365 (M+H)⁺. Purity by HPLC: 99% (Method C).

### xxxvii. 2-(6-METHYL-2-OXO-3,4-DIHYDROQUINOLIN-1(2H)-YL)-N-(5-(PYRIDIN-2-YL)-4H-1,2,4-TRIAZOL-3-YL)ACETAMIDE (54)

This compound was prepared by the reaction 2-(6-methyl-2-oxo-3,4-dihydroquinolin-1(2H)-yl)acetic acid¹ (prepared by the procedure described in reference 1) and tert-butyl 3-amino-5-(pyridin-2-yl)-4H-1,2,4-triazole-4-carboxylate (Amine B) using route 1. The crude was purified by Prep-HPLC (Column: Xselect CSH OBD Column 30×150 mm; 5 μm; Mobile Phase A: Water (0.1% formic acid), Mobile Phase B: acetonitrile; Flow rate: 60 mL/min; Gradient: 14% B to 34% B in 7 min; 254/220 nm). This resulted in pure compound 54 in 55% yield as a white solid. ¹H NMR (300 MHz, DMSO-d₆) δ 14.46 (13.65) (s, 1H), 12.03 (10.72) (s, 1H), 8.68 (s, 1H), 8.10-7.80 (m, 2H), 7.60-7.35 (m, 1H), 7.10-7.00 (m, 2H), 6.92-6.75 (m, 1H), 4.77 (s, 2H), 2.88 (t, J=7.4 Hz, 2H), 2.59 (t, J=7.3 Hz, 2H), 2.25 (s, 3H). ESI-MS (m/z): 363 (M+H)⁺. Purity by HPLC: 99% (Method C).

### c. SYNTHESIS OF INTERMEDIATE AMINES (A-D)

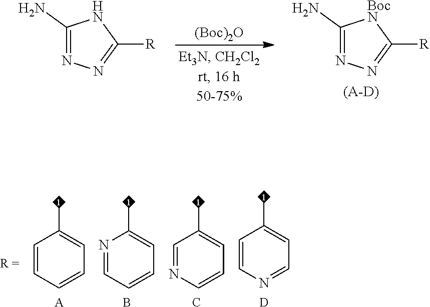

To a stirred solution of commercially available corresponding triazole amine (500 mg, 3.10 mmol) in CH₂Cl₂(10 mL), Et₃N (1.27 mL, 9.31 mmol) and Boc anhydride (0.47 g, 2.17 mmol) were added in one portion at room temperature. The resulting suspension was stirred at room temperature for 24 h. The reaction mixture was concentrated under reduced pressure to get the crude product. The crude product was purified by column chromatography (20-30% Ethylacetate-Hexanes) to afford Boc-protected amines in 50-75% yield.

### i. TERT-BUTYL 3-AMINO-5-PHENYL-4H-1,2,4-TRIAZOLE-4-CARBOXYLATE (A)

This compound was prepared starting from commercially available 5-phenyl-4H-1,2,4-triazol-3-amine. The crude product was purified by flash column chromatography (using Silica, 20-30% Ethylacetate-Hexanes) to afford tert-butyl 3-amino-5-phenyl-4H-1,2,4-triazole-4-carboxylate (1d) in 57% yield as an off-white solid. ESI-MS (m/z)=161.1 (M-Boc)⁺.

### ii. TERT-BUTYL 3-AMINO-5-(PYRIDIN-2-YL)-4H-1,2,4-TRIAZOLE-4-CARBOXYLATE (B)

This compound was prepared starting from commercially available 5-(pyridin-2-yl)-4H-1,2,4-triazol-3-amine. The crude product was purified by column chromatography (20-30% Ethylacetate-Hexanes) to afford tert-butyl 3-amino-5-(pyridin-2-yl)-4H-1,2,4-triazole-4-carboxylate (1d) in 60% yield as an off-white solid. ¹H NMR (400 MHz, DMSO-d₆) δ 8.66-8.64 (m, 1H), 7.97-7.88 (m, 2H), 7.47-7.40 (m, 3H), 1.60 (s, 9H). ESI-MS (m/z)=262.1 (M+H)⁺.

### iii. TERT-BUTYL 3-AMINO-5-(PYRIDIN-3-YL)-4H-1,2,4-TRIAZOLE-4-CARBOXYLATE (C)

This compound was prepared starting from commercially available 5-(pyridin-3-yl)-4H-1,2,4-triazol-3-amine. The crude product was purified by column chromatography (20-30% Ethyl acetate-Hexanes) to afford tert-butyl 3-amino-5-(pyridin-3-yl)-4H-1,2,4-triazole-4-carboxylate (if) in 62% yield as an off-white solid. ¹H NMR (400 MHz, DMSO-d₆) δ 9.09 (s, 1H), 8.65 (t, J=3.2 Hz, 1H), 8.24 (q, J=1.6 Hz, J=8.0 Hz, 1H), 7.52-7.48 (m, 3H), 1.61 (s, 9H). ESI-MS (m/z)=262.2 (M+H)¹.

### iv. TERT-BUTYL 3-AMINO-5-(PYRIDIN-4-YL)-4H-1,2,4-TRIAZOLE-4-CARBOXYLATE (D)

This compound was prepared starting from commercially available 5-(pyridin-4-yl)-4H-1,2,4-triazol-3-amine. The crude product was purified by column chromatography (20-30% Ethyl acetate-Hexanes) to afford tert-butyl 3-amino-5-(pyridin-4-yl)-4H-1,2,4-triazole-4-carboxylate (1g) in 75% yield as an off-white solid. ¹H NMR (400 MHz, DMSO-d₆) δ 8.68 (dd, J=1.6 Hz, J=4.4 Hz, 2H), 7.83 (dd, J=1.6 Hz, J=4.4 Hz, 2H), 7.48 (s, 1H), 1.61 (s, 9H). ESI-MS (m/z)=262.1 (M+H)⁺.

### d. GENERAL SCHEME FOR THE SYNTHESIS OF COMPOUNDS 27-35 (ROUTE 2)

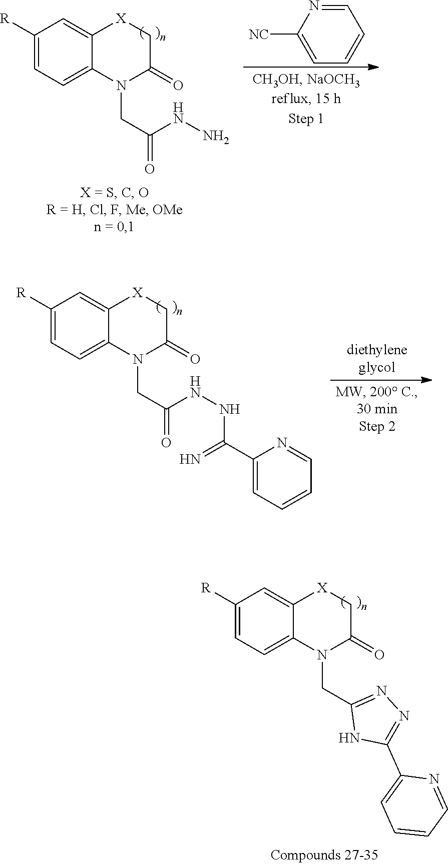

Step 1: To a stirred solution of picolinonitrile (6.22 mmol) in Methanol (10 mL), NaOMe Solution (30% in CH₃OH) (0.45 mL) was added at room temperature under nitrogen atmosphere. The reaction mixture was stirred at 85° C. for 3 h and appropriately substituted intermediate acetohydrazide³ (6.22 mmol) (prepared by the synthetic procedure reported in reference 3) was added in portions. The resulting mixture was refluxed overnight and white precipitate was collected by filtration, washed with methanol and dried to afford corresponding intermediate iminopyridine acotohydrazide in 15-50% yields.

Step 2: Corresponding iminopyridine acetohydrazide intermediate (0.58 mmol) was dissolved in diethylene glycol (3 mL) at room temperature under a nitrogen atmosphere. The reaction mixture was stirred at 200° C. for 30 min under microwave irradiation, diluted with water (25 mL) and the resulting solid was collected by filtration, washed with water and dried to afford target compounds in 27-35 in 30-80% yields.

### i. 4-((5-(PYRIDIN-2-YL)-4H-1,2,4-TRIAZOL-3-YL)METHYL)-2H-BENZO[B][1,4]THIAZIN-3(4H)-ONE (27)

Step 1: N-(imino(pyridin-2-yl)methyl)-2-(3-oxo-2H-benzo[b][1,4]thiazin-4(3H)-yl)acetohydrazide was prepared by the reaction of 2-(3-oxo-2H-benzo[b][1,4]thiazin-4(3H)-yl)acetohydrazide and picolinonitrile in 33% yield as off-white solid. ESI-MS (m/z)=342 (M+H)⁺.

Step 2: Compound 27 was prepared by the reaction of intermediate N-(imino(pyridin-2-yl)methyl)-2-(3-oxo-2H-benzo[b][1,4]thiazin-4(3H)-yl)acetohydrazide and diethylene glycol using route 2 in 76% yield as brown solid. ¹HNMR (300 MHz, DMSO-d₆) δ 14.32 (brs, 1H), 8.65 (s, 1H), 8.02-7.94 (m, 2H), 7.47-7.32 (m, 3H), 7.24-7.01 (m, 2H), 5.23 (s, 2H), 3.54 (s, 2H). ESI-MS (m/z)=324.1 (M+H)⁺. Purity by HPLC: 95.04% (Method B).

### ii. 4-((5-(PYRIDIN-2-YL)-4H-1,2,4-TRIAZOL-3-YL)METHYL)-2H-BENZO[B][1,4]OXAZIN-3(4H)-ONE (28)

Step 1: N-(imino(pyridin-2-yl)methyl)-2-(3-oxo-2H-benzo[b][1,4]oxazin-4(3H)-yl)acetohydrazide was prepared by the reaction of 2-(3-oxo-2H-benzo[b][1,4]oxazin-4(3H)-yl)acetohydrazide and piccolinonitrile in 61% yield as off-white solid. ESI-MS (m/z)=326 (M+H)⁺.

Step 2: Compound 28 was prepared by the reaction of intermediate N-(imino(pyridin-2-yl)methyl)-2-(3-oxo-2H-benzo[b][1,4]oxazin-4(3H)-yl)acetohydrazide and diethylene glycol using route 2 in 74% yield as brown solid. ¹HNMR (300 MHz, DMSO-d₆) δ 14.34 (brs, 1H), 8.64 (s, 1H), 8.01-7.92 (m, 2H), 7.45 (brs, 1H), 7.18 (brs, 1H), 6.97 (d, J=2.7 Hz, 3H), 5.23 (s, 2H), 4.70 (s, 2H). ESI-MS (m/z)=308.1 (M+H)¹. Purity by HPLC: 97.9% (Method B).

### iii. 1-((5-(PYRIDIN-2-YL)-4H-1,2,4-TRIAZOL-3-YL)METHYL)-3,4-DIHYDROQUINOLIN-2(1H)-ONE (29)

Step 1: N-(imino(pyridin-2-yl)methyl)-2-(2-oxo-3,4-dihydroquinolin-1(2H)-yl)acetohydrazide was prepared by the reaction of 2-(2-oxo-3,4-dihydroquinolin-1(2H)-yl)acetohydrazide and picolinonitrile in 48% yield as off-white solid. EST-MS (m/z)=324.1 (M+H)⁺.

Step 2: Compound 29 was prepared by the reaction of intermediate N-(imino(pyridin-2-yl)methyl)-2-(2-oxo-3,4-dihydroquinolin-1(2H)-yl)acetohydrazide and diethylene glycol using route 2 in 75.5% yield as brown solid. ¹H NMR (300 MHz, DMSO-d₆) δ 14.26 (brs, 1H), 8.63 (s, 1H), 8.02-7.91 (m, 2H), 7.44 (brs, 1H), 7.21-7.08 (m, 3H), 6.95 (t, J=6.9 Hz, 1H), 5.20 (s, 2H), 2.93 (t, J=7.2 Hz, 2H), 2.63 (t, J=6.9 Hz, 2H). ESI-MS (m/z)=306.1 (M+H)⁺. Purity by HPLC: 97.68%. (Method B).

### iv. 3-((5-(PYRIDIN-2-YL)-4H-1,2,4-TRIAZOL-3-YL)METHYL)BENZO[D]THIAZOL-2(3H)-ONE (30)

Step 1: N-(imino (pyridin-2-yl)methyl)-2-(2-oxobenzo[d]thiazol-3(2H)-yl)acetohydrazide was prepared by the reaction of 2-(2-oxobenzo[d]thiazol-3(2H)-yl)acetohydrazide and picolinonitrile in 51% yield as off-white solid. ESI-MS (m/z)=328.1 (M+H)⁺.

Step 2: Compound 30 was prepared by the reaction of intermediate N-(imino(pyridin-2-yl)methyl)-2-(2-oxobenzo[d]thiazol-3(2H)-yl)acetohydrazide and diethylene glycol using route 2 in 67% yield as brown solid. ¹H NMR (300 MHz, DMSO-d₆) δ 14.32 (brs, 1H), 8.65 (s, 1H), 8.02-7.94 (m, 2H), 7.47-7.32 (m, 3H), 7.24-7.01 (m, 2H), 5.23 (s, 2H), 3.54 (s, 2H). ESI-MS (m/z)=310.1 (M+H)⁺. Purity by HPLC: 99%. (Method B).

### v. 3-((5-(PYRIDIN-2-YL)-4H-1,2,4-TRIAZOL-3-YL)METHYL)BENZO[D]OXAZOL-2(3H)-ONE (31)

Step 1: N-(imino(pyridin-2-yl)methyl)-2-(2-oxobenzo[d]oxazol-3(2H)-yl)acetohydrazide was prepared by the reaction of 2-(2-oxobenzo[d]oxazol-3(2H)-yl)acetohydrazide and picolinonitrile 50% yield as off-white solid. ESI-MS (m/z)=312.3 (M+H)⁺.

Step 2: Compound 31 was prepared by the reaction of intermediate N-(imino(pyridin-2-yl)methyl)-2-(2-oxobenzo[d]oxazol-3(2H)-yl)acetohydrazide and diethylene glycol using route 2 in 50% yield as brown solid. ¹HNMR (300 MHz, DMSO-d₆) δ 14.44 (brs, 1H), 8.65 (s, 1H), 8.01-7.90 (m, 2H), 7.47 (brs, 1H), 7.32 (d, J=7.5 Hz, 1H), 7.20-7.12 (m, 3H), 5.15 (s, 2H). ESI-MS (m/z)=294.0 (M+H)⁺. Purity by HPLC: 95.17%.

### vi. 7-CHLORO-4-((5-(PYRIDIN-2-YL)-4H-1,2,4-TRIAZOL-3-YL)METHYL)-2H-BENZO[B][1,4]THIAZIN-3(4H)-ONE (32)

Step 1: 2-(7-chloro-3-oxo-2H-benzo[b][1,4]thiazin-4(3H)-yl)-N-(imino(pyridin-2-yl)methyl)acetohydrazide was prepared by the reaction 2-(7-chloro-3-oxo-2H-benzo[b][1,4]thiazin-4(3H)-yl)acetohydrazide and piccolinonitrile in 20% yield as off-white solid. ESI-MS (m/z)=376.0 (M+H)⁺.

Step 2: Compound 32 was prepared by the reaction of intermediate 2-(7-chloro-3-oxo-2H-benzo[b][1,4]thiazin-4(3H)-yl)-N′-(imino(pyridin-2-yl)methyl)acetohydrazide and diethylene glycol using route 2 in 25% yield as brown solid. ¹HNMR (300 MHz, DMSO-d₆) δ 14.28 (brs, 1H), 8.65 (d, J=4.2 Hz, 1H), 8.01-7.90 (m, 2H), 7.46 (s, 2H), 7.37-7.25 (m, 2H), 5.23 (s. 2H),3.59 (s, 2H). ESI-MS (m/z)=358.1 (M+H)⁺. Purity by HPLC: 94.68%. (Method B).

### vii. 7-FLUORO-4-((5-(PYRIDIN-2-YL)-4H-1,2,4-TRIAZOL-3-YL)METHYL)-2H-BENZO[B][1,4]THIAZIN-3(4H)-ONE (33)

Step 1: 2-(7-Fluoro-3-oxo-2H-benzo[b][1,4]thiazin-4(3H)-yl)acetohydrazide was prepared by the reaction 2-(7-fluoro-3-oxo-2H-benzo[b][1,4]thiazin-4(3H)-yl)acetohydrazide and piccolinonitrile in 25% yield as off-white solid. ESI-MS (m/z)=360.1 (M+H)⁺.

Step 2: Compound 33 was prepared by the reaction of intermediate 2-(7-fluoro-3-oxo-2H-benzo[b][1,4]thiazin-4(3H)-yl)acetohydrazide and diethylene glycol using route 2 in 73% yield as brown solid. ¹HNMR (300 MHz, DMSO-d₆) δ 14.35 (brs, 1H), 8.65 (s, 1H), 8.02-7.94 (m, 2H), 7.47-7.06 (m, 4H), 5.23 (s, 2H), 3.57 (s, 2H). ESI-MS (m/z)=342.0 (M+H)⁺. Purity by HPLC: 97.3%. (Method B).

### viii. 7-METHYL-4-((5-(PYRIDIN-2-YL)-4H-1,2,4-TRIAZOL-3-YL)METHYL)-2H-BENZO[B][1,4]THIAZIN-3(4H)-ONE (34)

Step 1: N-(imino(pyridin-2-yl)methyl)-2-(7-methyl-3-oxo-2H-benzo[b][1,4]thiazin-4(3H)-yl)acetohydrazide was prepared by the reaction 2-(7-methyl-3-oxo-2H-benzo[b][1,4]thiazin-4(3H)-yl)acetohydrazide and piccolinonitrile in 50% yield as off-white solid. ESI-MS (m/z)=356.1 (M+H)⁺.

Step 2: Compound 34 was prepared by the reaction of intermediate N-(imino(pyridin-2-yl)methyl)-2-(7-methyl-3-oxo-2H-benzo[b][1,4]thiazin-4(3H)-yl)acetohydrazide and diethylene glycol using route 2 in 28% yield as brown solid. ¹HNMR (300 MHz, DMSO-d₆) δ 14.31 (brs, 1H), 8.64 (s, 1H), 8.01-7.93 (m, 2H), 7.46 (brs, 1H), 7.23-7.01 (m, 3H), 5.21 (s, 2H), 3.52 (s, 2H), 2.22 (s, 3H). ESI-MS (m/z)=338.1 (M+H)⁺. Purity by HPLC: 96.6%. (Method B).

### ix. 7-METHOXY-4-((5-(PYRIDIN-2-YL)-4H-1,2,4-TRIAZOL-3-YL)METHYL)-2H-BENZO[B][1,4]THIAZIN-3(4H)-ONE (35)

Step 1: N-(imino(pyridin-2-yl)methyl)-2-(7-methoxy-3-oxo-2,3-dihydro-4H-benzo[b][1,4]thiazin-4-yl)acetohydrazide was prepared by the reaction 2-(7-methoxy-3-oxo-2H-benzo[b][1,4]thiazin-4(3H)-yl)acetohydrazide and piccolinonitrile in 40% yield as off-white solid. ESI-MS (m/z)=372.4 (M+H)⁺.

Step 2: Compound 34 was prepared by the reaction of intermediate N-(imino(pyridin-2-yl)methyl)-2-(7-methoxy-3-oxo-2,3-dihydro-4H-benzo[b][1,4]thiazin-4-yl)acetohydrazide and diethylene glycol using route 2 in 15% yield as brown solid. ¹HNMR (300 MHz, DMSO-d) δ 14.26 (brs, 1H), 8.64 (s, 1H), 8.02-7.92 (m, 2H), 7.45 (brs, 1H), 7.27 (d, J=9.0 Hz, 1H), 6.95 (s, 1H), 6.81 (d, J=6.6 Hz, 1H), 5.19 (s, 2H), 3.72 (s, 3H), 3.52 (s, 2H). ESI-MS (m/z)=354.0 (M+H)⁺. Purity by HPLC: 98.01%. (Method B).

### e. GENERAL SCHEME FOR THE SYNTHESIS OF COMPOUNDS 36-42,44 (ROUTE 3)

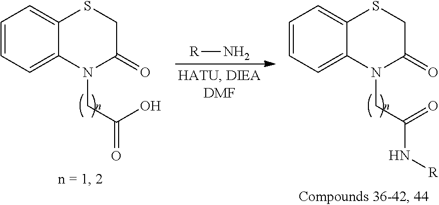

To a solution of corresponding acid (1.0 mmol) in anhydrous DMF (3 mL), HATU (1.5 mmol) and DIEA (2.0 mmol) were added and the reaction mixture was stirred for 10 min at rt. Appropriate amine was added and the reaction mixture was stirred at rt for 18 h. Reaction mixture was concentrated and crude product was purified by column chromatography using 0-10% methanol in dichloromethane.

### i. 3-(3-OXO-2,3-DIHYDRO-4H-BENZO[B][1,4]THIAZIN-4-YL)-N-(2-(TRIFLUOROMETHYL)PHENYL) PROPANAMIDE (36)

This compound was prepared by the reaction of commercially available 3-(3-oxo-2,3-dihydro-4H-benzo[b][1,4]thiazin-4-yl)propanoic acid and 2-(trifluoromethyl)aniline using route 3. The crude product was purified by column chromatography (5-10% CH₃OH—CH₂Cl₂) to give pure compounds (45% yield) as an off-white solid. ¹H NMR (400 MHz, CDCl₃) δ 8.00 (d, J=8.2 Hz, 1H), 7.71-7.58 (m, 2H), 7.54 (dddt, J=8.2, 7.6, 1.6, 0.7 Hz, 1H), 7.39-7.33 (m, 1H), 7.30-7.20 (m, 3H), 7.04 (ddd, J=7.7, 4.9, 3.7 Hz, 1H), 4.43-4.36 (m, 2H), 3.40 (s, 2H), 2.87-2.75 (m, 2H). ESI-MS (m/z)=381 (M+H)⁺. Purity by HPLC: 96% (Method A).

### ii. N-(4-(1H-1,2,4-TRIAZOL-1-YL)PHENYL)-3-(3-oxo-2,3-DIHYDRO-4H-BENZO[B][1,4]THIAZIN-4-YL)PROPANAMIDE (37)

This compound was prepared by the reaction of commercially available 3-(3-oxo-2,3-dihydro-4H-benzo[b][1,4]thiazin-4-yl)propanoic acid and 4-(1H-1,2,4-triazol-1-yl)aniline using route 3. The crude product was purified by column chromatography (5-10% CH₃OH—CH₂Cl₂) to give pure compound (50% yield) as an off-white solid. ¹H NMR (400 MHz, DMSO-d₆) δ 10.20 (s, 1H), 9.18 (s, 1H), 8.18 (s, 1H), 7.78-7.67 (m, 4H), 7.41 (td, J=8.2, 7.7, 1.4 Hz, 2H), 7.30 (ddd, J=8.3, 7.4, 1.6 Hz, 1H), 7.05 (td, J=7.5, 1.2 Hz, 1H), 4.28-4.20 (m, 2H), 3.50 (s, 2H), 2.70-2.63 (m, 2H). ESI-MS (m/z)=380 (M+H)⁺. Purity by HPLC: 98% (Method A).

### iii. 3-(3-Oxo-2,3-DIHYDRO-4H-BENZO[B][1,4]THIAZIN-4-YL)-N-(4-(PYRIDIN-2-YL)PHENYL)PROPANAMIDE (38)

This compound was prepared by the reaction of commercially available 3-(3-oxo-2,3-dihydro-4H-benzo[b][1,4]thiazin-4-yl)propanoic acid and 4-(pyridin-2-yl)aniline using route 3. The crude product was purified by column chromatography (5-10% CH₃OH—CH₂Cl₂) to give pure compound (45% yield) as an off-white solid. ¹H NMR (400 MHz, DMSO-d₆) δ 10.14 (s, 1H), 8.61 (ddd, J=4.8, 1.8, 0.9 Hz, 1H), 8.06-8.00 (m, 2H), 7.92-7.79 (m, 2H), 7.70-7.64 (m, 2H), 7.41 (ddd, J=10.9, 8.1, 1.4 Hz, 2H), 7.33-7.25 (m, 2H), 7.05 (td, J=7.5, 1.2 Hz, 1H), 4.28-4.21 (m, 2H), 3.50 (s, 2H), 2.69-2.64 (m, 2H). ESI-MS (m/z)=390 (M+H)⁺. Purity by HPLC: 98% (Method A).

### iv. N-([1,1′-BIPHENYL]-4-YL)-3-(3-Oxo-2,3-DIHYDRO-4H-BENZO[B][1,4]THIAZIN-4-YL)PROPANAMIDE (39)

This compound was prepared by the reaction of commercially available 3-(3-oxo-2,3-dihydro-4H-benzo[b][1,4]thiazin-4-yl)propanoic acid and [1,1′-biphenyl]-4-amine using route 3. The crude product was purified by column chromatography (5-10% CH₃OH—CH₂Cl₂) to give pure compound (60% yield) as an off-white solid. ¹H NMR (400 MHz, DMSO-d₆) δ 10.08 (s, 1H), 7.67-7.56 (m, 7H), 7.45-7.38 (m, 5H), 7.34-7.26 (m, 2H), 7.05 (td, J=7.5, 1.0 Hz, 1H), 4.28-4.20 (m, 2H), 3.50 (s, 2H), 2.69-2.62 (m, 2H). ESI-MS (m/z)=389 (M+H)⁺. Purity by HPLC: 99% (Method A).

### v. 3-(3-OXO-2,3-DIHYDRO-4H-BENZO[B][1,4]THIAZIN-4-YL)-N-(1H-TETRAZOL-5-YL)PROPANAMIDE (40)

This compound was prepared by the reaction of commercially available 3-(3-oxo-2,3-dihydro-4H-benzo[b][1,4]thiazin-4-yl)propanoic acid and 1H-tetrazol-5-amine using route 3. The crude product was purified by column chromatography (5-10% CH₃OH—CH₂Cl₂) to give pure compound (30% yield) as an off-white solid. ¹H NMR (400 MHz, DMSO-d₆) δ 11.98 (s, 1H), 7.39 (dt, J=8.3, 2.0 Hz, 2H), 7.28 (ddd, J=8.5, 7.3, 1.5 Hz, 1H), 7.05 (td, J=7.5, 1.2 Hz, 1H), 4.24 (t, J=7.5 Hz, 2H), 3.49 (s, 2H), 2.79-2.72 (m, 2H). ESI-MS (m/z)=305 (M+H)⁺. Purity by HPLC: 98% (Method A).

### vi. N-(4-(1H-IMIDAZOL-2-YL)PHENYL)-3-(3-Oxo-2,3-DIHYDRO-4H-BENZO[B][1,4]THIAZIN-4-YL)PROPANAMIDE (41)

This compound was prepared by the reaction of commercially available 3-(3-oxo-2,3-dihydro-4H-benzo[b][1,4]thiazin-4-yl)propanoic acid and 4-(1H-imidazol-2-yl)aniline using route 3. The crude product was purified by column chromatography (5-10% CH₃OH—CH₂Cl₂) to give pure compound (25% yield) as an off-white solid. ¹H NMR (400 MHz, DMSO-d₆) δ 12.34 (s, 1H), 10.09 (s, 1H), 7.85-7.79 (m, 2H), 7.62-7.57 (m, 2H), 7.41 (td, J=8.3, 7.9, 1.4 Hz, 2H), 7.30 (ddd, J=7.9, 7.3, 1.5 Hz, 1H), 7.18 (s, 1H), 7.08-6.93 (m, 2H), 4.27-4.19 (m, 2H), 3.50 (s, 2H), 2.68-2.61 (m, 2H). ESI-MS (m/z)=380 (M+H)⁺. Purity by HPLC: 90% (Method A).

### vii. 3-(3-OXO-2,3-DIHYDRO-4H-BENZO[B][1,4]THIAZIN-4-YL)-N-(4-(PYRIMIDIN-5-YL)PHENYL) PROPANAMIDE (42)

This compound was prepared by the reaction of commercially available 3-(3-oxo-2,3-dihydro-4H-benzo[b][1,4]thiazin-4-yl)propanoic acid and 4-(pyrimidin-5-yl)aniline using route 3. The crude product was purified by column chromatography (5-10% CH₃OH—CH₂Cl₂) to give pure compound (40% yield) as an off-white solid. ¹H NMR (400 MHz, DMSO-d₆) δ 10.17 (s, 1H), 9.12 (s, 1H), 9.10 (s, 2H), 7.79-7.67 (m, 4H), 7.41 (ddd, J=9.4, 8.0, 1.4 Hz, 2H), 7.31 (ddd, J=8.4, 7.3, 1.5 Hz, 1H), 7.05 (td, J=7.5, 1.1 Hz, 1H), 4.27-4.21 (m, 2H), 3.50 (s, 2H), 2.70-2.62 (m, 2H). ESI-MS (m/z)=391 (M+H)⁺. Purity by HPLC: 96% (Method A).

### viii. N-(3-(MORPHOLINE-4-CARBONYL)PHENYL)-2-(3-OXO-2,3-DIHYDRO-4H-BENZO[B][1,4]THIAZIN-4-YL)ACETAMIDE (44)

This compound was prepared by the reaction of commercially available 2-(3-oxo-2,3-dihydro-4H-benzo[b][1,4]thiazin-4-yl)acetic acid and 3-(morpholinomethyl)aniline using route 3. The crude product was purified by column chromatography (5-10% CH₃OH—CH₂Cl₂) to give pure compound (50% yield) as an off-white solid. ¹H NMR (400 MHz, DMSO-d₆) δ 8.75 (s, 1H), 7.66 (t, J=1.8 Hz, 1H), 7.56 (ddd, J=8.2, 2.2, 1.0 Hz, 1H), 7.40-7.17 (m, 4H), 7.09-7.01 (m, 2H), 4.71 (s, 2H), 3.73 (s, 2H), 3.62 (h, J=6.1, 5.6 Hz, 4H), 3.47 (s, 2H), 3.11 (q, J=7.4 Hz, 2H). ESI-MS (m/z)=412 (M+H)⁺. HPLC: 98% (Method A).

### 2. Biological Methods

a. Primary Neuron Amyloid-B Toxicity Assays

Primary neuron cultures: All animal procedures were approved by the Institutional Animal Care and Use Committee at the University of Alabama at Birmingham (UAB). As before (Rush, et al. (2020) Neurobiol Dis 134, 104668; Voskobiynyk, et al. (2020) eLife 9, e57354), primary rat neurons were isolated from timed-pregnant Sprague-Dawley rats. Briefly, dams were euthanized with isoflurane anesthesia asphyxiation, and hippocampal tissue was isolated from E19 embryos in Hibernate E, then digested in papain for 10 minutes at 37° C. Neurons were triturated into a single cell suspension in Neurobasal supplemented with B-27, L-Glutamine, and 10% FBS, then plated in 96-well plates coated with PDL and laminin at 30,000 neurons per well in 200 uL medium or in 24-well coated plates with or without coverslips at 50,000 per well. Cultures were maintained at 37° C. with 5% CO₂. Twenty-four hours after plating (DIV1), two 50% media changes with Neurobasal plus B-27 and L-Glutamine without FBS, and 5 μM cytosine β-D-arabinofuranoside was added to prevent glial proliferation. 50% media changes were performed at DIV7 and DIV14 with serum-free supplemented Neurobasal and experiments were started at DIV19.

Aβ oligomer preparation: As before (Rush, et al. (2020) Neurobiol Dis 134, 104668), lyophilized recombinant Aβ₄₂ was dissolved in HFIP, dried overnight, then stored at −20° C. until oligomerization. To oligomerize, Aβ was dissolved in DMSO to 1 mM, then diluted to 100 M in PBS, then sonicated and left on ice for 24 hours. Immediately prior to use, Aβ was centrifuged at 4° C. for 10 minutes at 14,000×g. Vehicle was prepared as well in identical fashion except lacking the Aβ peptide.

Aβ toxicity assays: The modified MTT and MAP2 assays were adapted from a recent study (Rush, et al. (2020) Neurobiol Dis 134, 104668). Briefly, at DIV19, media on the neurons was reduced to 100 L, then compound was added to final concentration of 0 μM, 0.3 μM, 3 μM, or 15 μM with even amounts of DMSO. After 90-minute pretreatment with compound, 2.5 μM Aβ was added.

The modified MTT assay was performed 24 hours after Aβ application. 100 μM MTT was applied to neurons and incubated at 37° C. for one hour. Media was removed, then replaced with 15 μL 1.6% Tween20 in PBS and incubated on a shaker at 100 rpm for 5 minutes. The Tween was then removed and transferred to a 384-well clear-bottom plate and replaced with 15 μL isopropanol. Plates were again agitated for 5 minutes at RT, then isopropanol was transferred to the 384-well plate. Absorbance was then read at 590 nm on a plate reader, with 660 nm used as a reference. The ratio of Tween soluble MTT to isopropanol soluble (Tween-insoluble) MTT was calculated for each sample, then normalized to the vehicle group average.

For the MAP2 ICC assay, neurons were fixed in 4% PFA and 4% sucrose at 37° C. for 30 minutes, then washed 3×5 minutes in PBS. Neurons were blocked (5% normal horse serum, 5% normal goat serum, 1% bovine serum albumin, and 0.5% saponin in 1×PBS) for 1 hour, then incubated overnight at 4° C. with MAP2 (1:5,000) antibody. Neurons were washed 3×5 minutes in rinse buffer (0.5% normal horse serum, 0.5% normal goat serum, 0.05% saponin in 1× PBS), then secondary antibody was applied for one hour at RT. Neurons were washed 2×5 minutes, then 3×5 minutes in PBS, and stored in the dark at 4° C. until imaging. Images were taken on an Operetta high-content imager. Intact neurite length was measured in an unbiased, automated manner using Harmony software, averaged from four images from each well. For analysis, total intact neurite length for each well from each condition was normalized to the vehicle group average.

b. Oral Administration Of Drugs

Compounds were sent to Pharmaron to determine if they were blood-brain barrier permeable. Compounds were formulated to 1 mg/mL in 10% NMP, 60% PEG400, 30% saline, then male CD1 mice were treated PO at 5 mg/kg. Brain and plasma concentrations were measured using HPLC-MS at 1 hour, 2 hours, and 4 hours post-treatment.

c. Proximity Ligation Assays

Endogenous Tau-Fyn PL, in primary neurons: All PLA protocols were adapted from our recent study (Rush, et al. (2020) Neurobiol Dis 134, 104668).

For the endogenous Tau-Fyn PLA, neurons were grown on coverslips as described above to DIV20, then treated with compound no. 69 or vehicle, and fixed with PFA 24 hours later. Dako Tau (1:1000) and Fyn15 (1:250) antibodies were used. Duolink In Situ Fluorescence kit was used for PLA. Briefly, coverslips with fixed cells were permeabilized for 10 minutes in 0.25% Triton X-100, then blocked in 5% NGS in PBS for one hour at room temperature. Coverslips were incubated overnight at 4° C. with primary antibody in 1% NGS, then washed 3×5 minutes in PBS. Coverslips were then incubated for 1 hour at 37° C. in PLA probes (Anti-Mouse minus, Anti-Rabbit plus). Coverslips were washed in Wash Buffer A at room temperature 2×5 minutes, then incubated for 1 hour at 37° C. in Ligation Buffer, then washed 2×2 minutes in Wash Buffer A. Coverslips were incubated in Amplification Buffer for 100 minutes at 37° C., then washed with Wash Buffer B 2×10 minutes, 0.01 Wash Buffer B for 1 minute, then PBS for 5 minutes, then mounted in Prolong Diamond with DAPI. After setting, 5-7 images of each coverslip were taken on an epifluorescent microscope. To quantify PLA puncta, PLA fluorescence was thresholded, then ImageJ particle analyzer was run, specifying size and circularity to exclude any non-punctate signal. Quantified values were then normalized to values for vehicle-treated slides from that experiment. Experimenters were blinded during image acquisition and analysis.

Tau-Fyn PLA screening in HEK-293 cells: For Tau-Fyn PLA in HEK-293 cells, we used the same plasmids as before (Rush, et al. (2020) Neurobiol Dis 134, 104668): human 4R2N Tau with an N-terminal mKate2 tag in a pcDNA3.1 vector and human Fyn with an N-terminal Myc tag in a pCMV-Sport6 vector grown in Stellar E. coli. HEK-293 cells were plated on coverslips coated with PDL and laminin in a 24-well plate in DMEM supplemented with 10% FBS and 1% PenStrep. Cells were transfected with 125 ng of Tau-mKate2 and Fyn plasmids with 1.5 μL Fugene in plain DMEM. After 24 hours, compound was applied at 15 μM. Twenty-four hours after application, cells were fixed. PLA protocol was the same as above except for different primary antibodies (Tau5 1:500, Fyn3 1:250). After image acquisition, PLA density was calculated as PLA puncta (as above) divided by cell area to account for differences in confluency. Cell area was defined with ImageJ as % Area after theresholding Tau-mKate2 fluorescence because it filled the cells evenly. Puncta/cell area was then normalized to vehicle-treated slides from that experiment.

Tau-Flag PLA for Tau-SH3 interactions: To clone the SH3 constructs, a C-terminal Flag tag was added to the Tau construct using PCR, then the Tau sequence was replaced with the desired SH3 domain using In-Fusion HD cloning kit (Takara Bio). Next, an N-terminal GST tag was added to help expression using In-Fusion, resulting in GST-SH3-Flag plasmids. As a negative control, restriction enzymes were used to remove the FynSH3 from the FynSH3 construct, resulting in GST-Flag. All sequences were confirmed with Sanger sequencing, and expression in HEK-293 cells was verified by western blot and immunocytochemistry. Plating and experimental timeline was the same as above. Cells were transfected with 125 ng of Tau-mKate2 and 500 ng SH3 plasmid with 1.5 μL Fugene. compound no. 69 was applied to final concentration 15 μM. For Tau-SH3 PLA, we used Dako Tau (1:1000) and Flag (1:1000) primary antibodies.

Tau-Tubulin PLA: Untransfected HEK-293 cells were used to measure endogenous Tau-tubulin interaction. Cells were grown for 48 hours before applying 15 M compound no. 69 for 24 hours. Cells were fixed and PLA was run as above except with Dako Tau (1:1000) and α-tubulin (1:2000) primary antibodies.

d. Western Blot

Like before (Rush, et al. (2020) Neurobiol Dis 134, 104668), at DIV13, neurons were treated with 15 μM compound no. 69 or Vehicle. 24 hours later, cells were harvested in lysis buffer (50 mM Tris, 150 mM NaCl, 5 mM EDTA, 0.1% SDS, 0.1% Triton-X-100, and 0.5% sodium deoxycholate) and centrifuged to remove cellular debris. Samples were diluted in LDS and reducing agent and heated at 70° C. for 10 minutes. Samples were run on 4-12% bis-tris gels in MOPS buffer with 5 μg protein per well, determined by a Bradford assay, then transferred to a PDVF membrane. Membrane was blocked for 1 hour in 50% Odysey blocking buffer then probed overnight with Fyn3 (1:1000) antibody at 4° C. Membrane was washed 3× in TBST, then incubated for 1 hour at RT in IRDye 700 or 800 conjugated secondary and scanned on an Odyssey Scanner. Images were quantified in ImageJ. After probing for GAPDH (1:5,000), the blot was stripped with Restore PLUS Western Blot Sripping Buffer for 30 minutes at room temperature, washed in TBST, and probed for DAKO Tau (1:10,000).

e. Kinase Panel

Compounds were dissolved in 100% DMSO to 1000× testing concentration: 3 mM for compound no. 69 and compound no. 57 and 30 μM for saracatinib/AZD0530. Compounds were tested on Eurofins DiscoverX ScanEDGE panel, which tested kinase activity of 97 kinases. Since Fyn was not included in that panel, a custom panel was completed for Fyn kinase activity. Activity was defined as compound leading to <35% of control activity to control for multiple comparisons. Interaction maps were generated with the TREEspot tool by DiscoverX.

f. Protein Purification

Human Fyn-SH3 domain with GST-tag at the N-terminus and a Strep-tag II (WSHPQFES) at the C-terminus (GST-Fyn-SH3-Strep) was cloned into pPR-IBA1 vector (IBA Lifesciences) (Cochran, et al. (2014) J. Biomol. Screen. 19, 1338-1349). Codon-optimized human 4R2N Tau for E. coli expression with a Strep-tag II at the C-terminus (His-Tau-Strep) was cloned into pET-28a(+) vector (Novagen). The constructs were transformed into E. coli BL-21(DE3) competent cells (Novagen). For GST-Fyn-SH3-Strep purification, a single colony was inoculated in Luria-Bertani (LB) broth at 37° C. overnight. 10 mL cell culture was added into 500 mL LB medium and cultured at 37° C. 0.5 mM Isopropyl β-D-1-thiogalactopyranoside (IPTG) was added into the culture when OD600 reached 0.7. Cells were further cultured at 25° C. for 4 hours and harvested. Cell pellet was resuspended in lysis buffer (20 mM Tris pH 7.9, 300 mM NaCl, 1 mM TCEP) with 1 tablet of EDTA-free protease inhibitors (ThermoFisher) and 1 mg/ml lysozyme. Cells were disrupted by sonication and debris was removed by centrifugation at 9500 rpm for 1 hour. Supernatant was loaded onto a glutathione-sepharose column (Cytiva). The column was washed in lysis buffer and protein eluted in lysis buffer containing 25 mM reduced glutathione. For His-Tau-Strep protein purification, cells were inoculated in LB broth medium and cultured at 37° C. overnight. A 10 mL overnight culture was added to 500 mL of auto-induction medium (Studier, F. W. (2005) Protein Expr Purif 41, 207-34) and cells were cultured at 37° C. for about 5 hours. The temperature was lowered to 18° C., and cells were cultured overnight. Cells were harvested and resuspended in lysis buffer with 20 mM imidazole, protease inhibitors, and lysozyme. Cells were disrupted by sonication and cell pellets were spun down at 9500 rpm for 1 hour. Supernatant was loaded onto Ni-NTA column (Cytiva) and the column was washed in lysis buffer with 20 mM imidazole. His-Tau-Strep protein was eluted in lysis buffer containing 400 mM imidazole. Both eluted GST-Fyn-SH3-Strep and His-Tau-Strep protein solutions were passed through G-25 desalting column (Cytiva) with lysis buffer and loaded onto high capacity Strep-Tactin column (IBA Lifesciences). The columns were washed in Buffer W (IBA Life sciences) and proteins were eluted in Buffer E (IBA life sciences). The eluted proteins were passed through G-25 desalting column again. Both GST-Fyn-Strep and His-Tau-Strep proteins were concentrated and diluted 1:1 in ethylene glycol as a cryoprotectant for storage at −80° C.

g. Alphascreen Preincubation Assay

AlphaScreen was conducted with 10 g/mL of glutathione donor beads (PerkinElmer, Waltham, MA) and nickel chelate acceptor beads (PerkinElmer) in 10 mM Bis-Tris (pH 7.0), 1 mM TCEP, 0.02% casein, and 0.1% Tween-20. Compounds were plated first followed by addition of one the proteins at 600 nM and incubation for 30 min. The second protein was added and incubated for 30 mn, then the beads were added simultaneously and the final mixture was incubated for 2 hours. Experiments were conducted in 384-well plates (6007290; PerkinElmer) and results were read on a PHERAstar plate reader (BMG Labtech, Cary, NC).

h. Isothermal Titration Calorimetry

Purified full-length Tau and FynSH3 were dialyzed into 20 mM Tris and 150 mM NaCl, pH 8.0 with Slide-A-Lyzer G2 Dialysis Cassettes with 10K MWCO (ThermoFisher #87729). Cassettes were hydrated in buffer for 2 minutes before purified protein was injected using an 18-gauge needle. Cassettes were placed in 500 mL buffer with a stir bar and stirred for two hours. Buffer was discarded and replaced for two more hours then replaced again and the samples were dialyzed overnight, all at 4° C. Protein concentrations were measured on a Nanodrop2000 (ThermoFisher) based on absorbance at 280 nm. Before ITC, solutions were brought to 5% DMSO to match amount needed for compound no. 69 preparation.

ITC experiments were performed on an Auto-iTC200 system (Malvern Instruments, Westborough, MA). ITC sample cell contained ˜120 μM full-length Tau, FynSH3, or Tau-PxxP_{5/6} (titrand), and the syringe contained 1 mM compound no. 69 (titrant). Each titration experiment consisted of 16 injections of 2.5 ELL of titrant into titrand at 25° C. Background mixing heat was determined from injections of titrant into the same buffer without titrand. Data analysis was performed using the built-in analysis module in Origin 7 provided by the ITC manufacturer. The normalized titration heat was fitted to a “one set of sites” model to determine the best-fit binding parameters.

i. Molecular Docking And Dynamics

Fyn structure and ligand preparation: A 2.6 Å crystal structure of human FynSH2/SH3 domains (PDBID:1G83) (Arold, et al. (2001) J Biol Chem 276, 17199-205) was energy minimized by steepest descent followed by conjugate gradient in AMBER18 using the ff14sb force field in preparation for virtual docking. The minimized structure of Fyn was loaded into AutoDockTools, where nonpolar hydrogen atoms, water molecules, and counterions were removed and Kollman charges appended. The prepared Fyn was then output in PDBQT format for molecular docking. A model of synthetic VSL12 peptide (PDBID:4EIK) was used as a positive control analogue for the known PxxP binding site on FynSH3.

OpenBabel software was used to generate 3D SDF structures of compound no. 69 for docking. The conformationally randomized 3D structures were loaded into AutoDockTools, where nonpolar hydrogen atoms, water molecules, and counterions were removed, and Gasteiger charges added to convert from SDF to PDBQT format. AutoDock Vina molecular docking software was run to predict top binding modes for compound no. 69 bound to FynSH2/SH3. Docking was performed in a grid box centered on, and fully containing, the entire SH3 domain with an exhaustiveness setting of 32. In molecular docking, compound no. 69's rotatable bonds were treated as flexible while the Fyn model was rigid. Docked conformations for compound no. 69 were ranked according to the calculated binding energy (in kcal/mol) from Vina.

Molecular dynamics simulations: The top conformation of compound no. 69 docked to Fyn was used for MD simulations in AMBER18 (Case, D. A. et al. AMBER 2015. (University of California, San Francisco, 2015), using the ff14SB force field (Maier, et al. (2015) J Chem Theory Comput 11, 3696-3713) to parameterize Fyn and VSL12 and the generalized AMBER force field (GAFF) (Wang, et al. (2004) J Comput Chem 25, 1157-74) for compound no. 69. MD simulations were performed in a periodic box with 2 nm of solvent between the protein edge and the box boundary to reduce periodicity artifacts. The periodic box was filled with TIP3P computational water and 150 mM NaCl added at random positions to approximate physiologic conditions. Additional Cl⁻ ions were added to neutralize the protein charge. First, steepest descent minimization of the solvent water was performed, with the protein, counterions, and ligand molecule restrained. Next, the solvent was equilibrated with the protein, counterions, and ligand molecule restrained at constant number-pressure-temperature at 50 K and 1 bar for 20 ps. The system was heated via 10 ps constant number-volume-temperature MD simulations at 50, 100, 150, 200, 250, and 300 K. MD production simulations of 150 ns at 300 K and 1 bar were performed. For all MD simulations, SHAKE constraints with relative tolerance of 1×10⁻⁵ were used on all hydrogen-heavy atom bonds to permit time steps of 2 fs. Electrostatic interactions were calculated by the particle-mesh Ewald method. Lennard-Jones cutoffs were set at 1.0 nm. To determine the initial simulation time needed to reach equilibration, RMSD was calculated over the trajectory using the cpptraj program of AMBER 19.05 (Roe, D. R. & Cheatham, T. E., 3rd. PTRAJ and CPPTRAJ: Software for Processing and Analysis of Molecular Dynamics Trajectory Data. J Chem Theory Comput 9, 3084-95 (2013)) and its rate of stabilization was compared to stabilization of binding free energy. Hydrophobic interactions, hydrogen bond formations, and electrostatic interactions between Fyn and the docked molecules were analyzed using PyMol (Schrodinger, LLC. The PyMOL Molecular Graphics System, Version 1.8. (2015)) and trajectories were rendered for visualization using VMD software. Virtual docking and MD simulations were performed on the University of Alabama at Birmingham's Cheaha Supercluster using 32 conventional 2.5 GHz Intel Xeon E5 series cores in parallel with OpenMPI v1.10.2.

Multi-electrode arrays: As in a prior study (Voskobiynyk, et al. (2020) eLife 9, e57354), 30,000 neurons were plated per well in a 48-well MEA plate. At DIV 5 and DIV 9, 50% media changes were completed with BrainPhys supplemented with SM1 and L-Glutamine. Recording and treatments were completed DIV13. A 20-minute baseline recording was done using Axion AxIS Navigator software. Neurons were then treated with compound no. 69 for one hour, then 500 nM Aβ oligomers was applied and neurons were returned to the incubator for four hours before being recorded for 20 more minutes. All recordings were dual filtered at 0.01 and 5,000 Hz Butterworth filters. Action potentials were set using an adaptive threshold >6 standard deviations from the electrode's mean signal. Waveforms were exported from AxIS Navigator to Offline Sorter, where individual units were identified and sorted. Analysis of action potential frequency was completed in NeuroExplorer. Mean baseline frequency for each well was calculated from unit frequencies, then each unit in the post-treatment recording was normalized to the baseline average of its well. Data were log transformed before analysis. Researchers were blind to experimental conditions during analysis.

j. Statistics

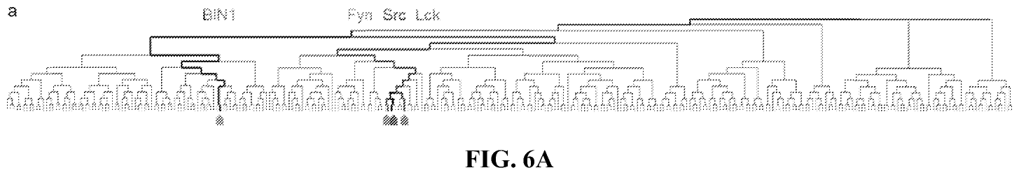

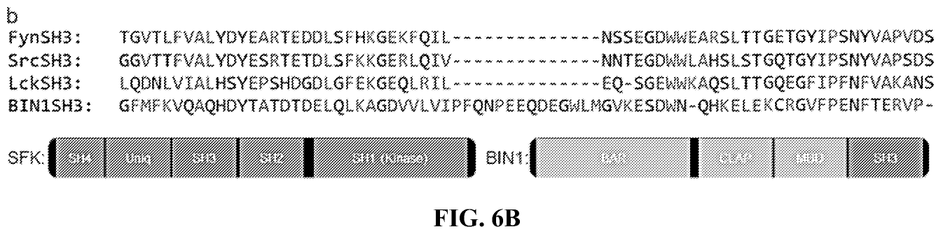

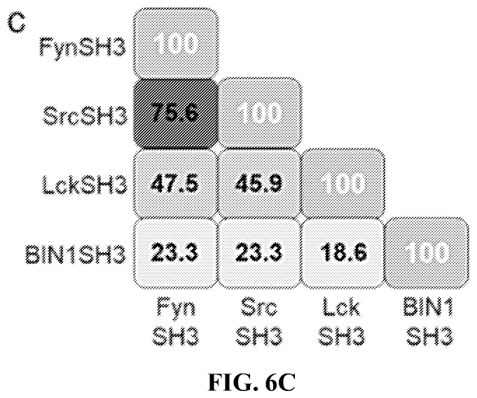

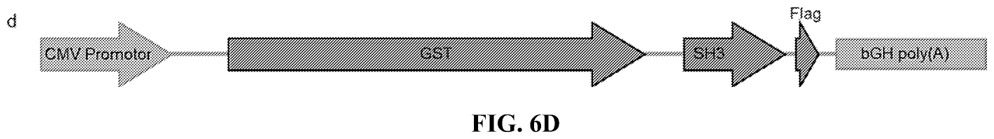

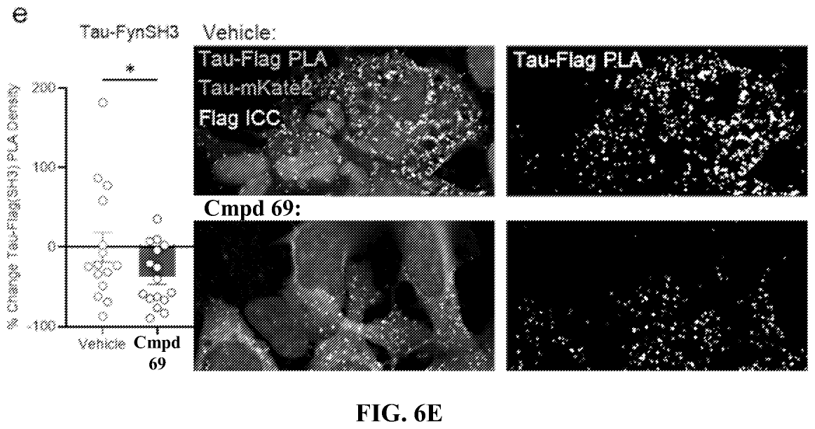

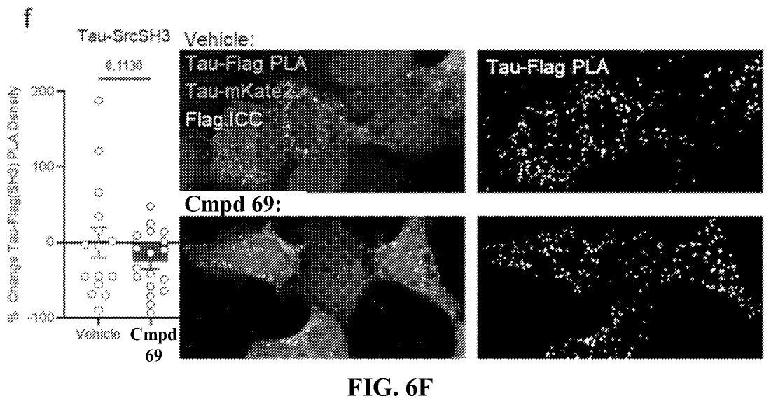

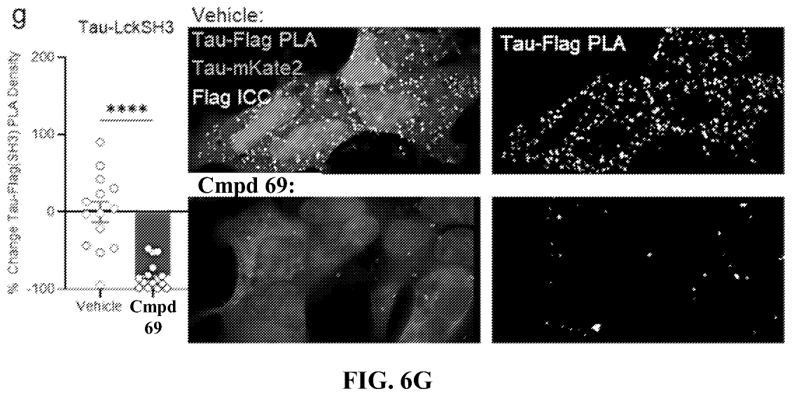

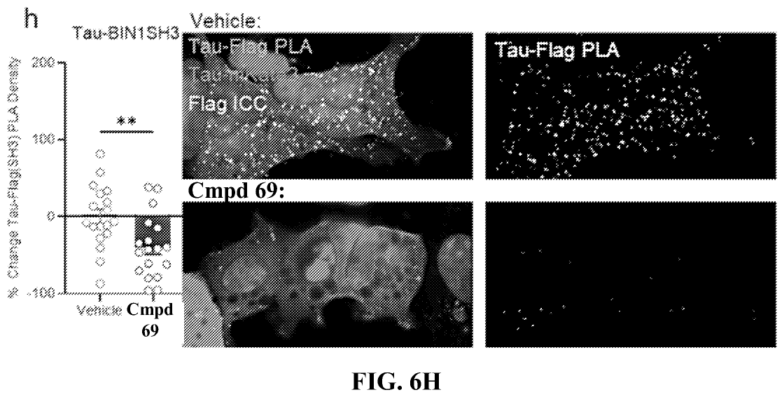

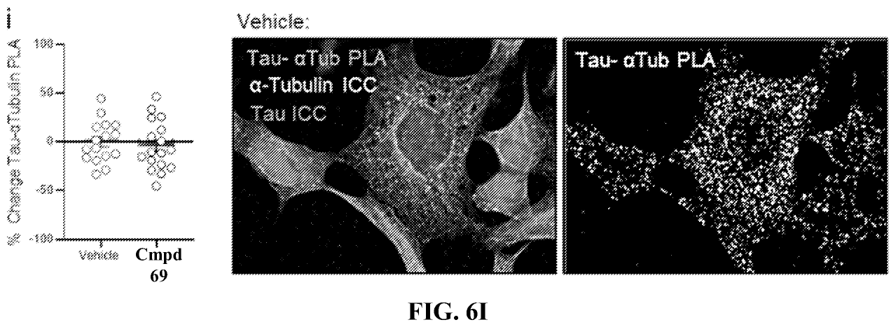

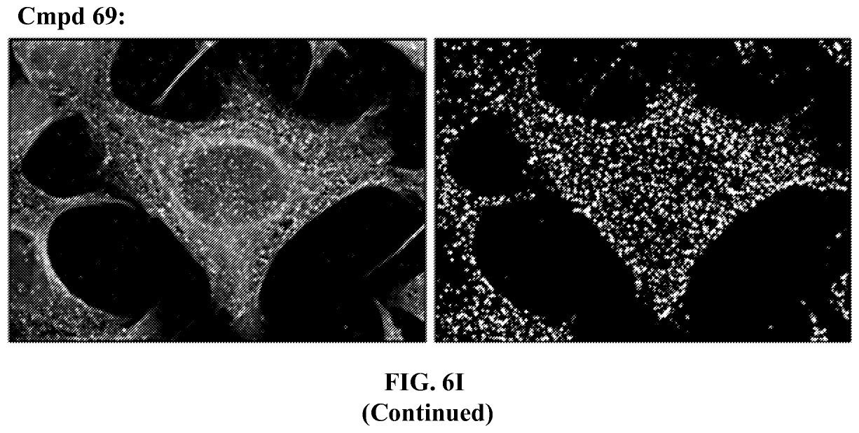

Statistics and sample sizes are described in each figure legend. Except where noted, i-test or ANOVA was used, usually with main effects of drug and Aβ for 2-way ANOVA. Data were organized in Microsoft Excel and analyses were completed in GraphPad Prism 9. One-tailed t-test was used for each Tau-SH3 interaction in FIG. 6A-I, as there was evidence that compound no. 69 would decrease rather than increase Tau-SH3 interactions. For all, α was 0.05. Data were presented as mean±SEM.

### 3. Results

a. Prioritization Of Tau-FynSH3 Inhibitor Hits From HTS

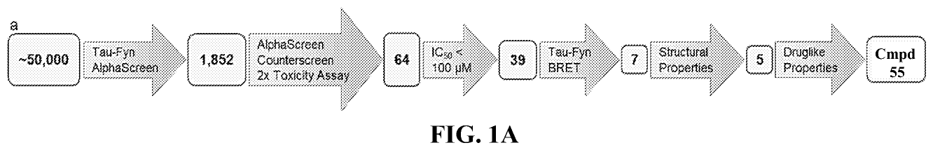

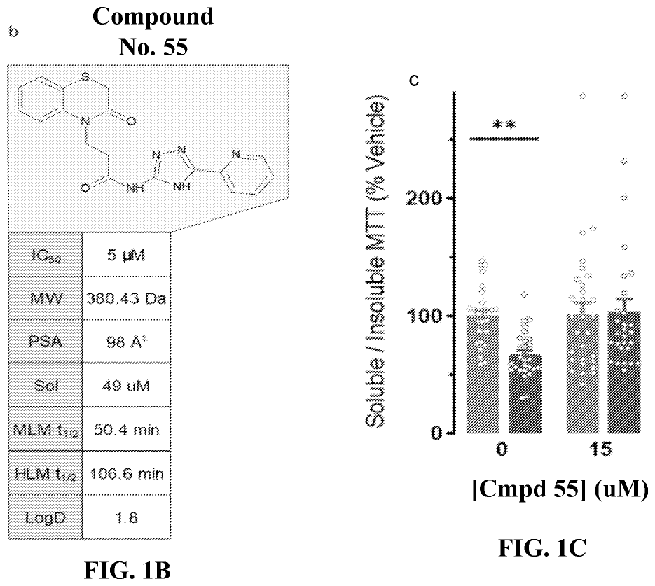

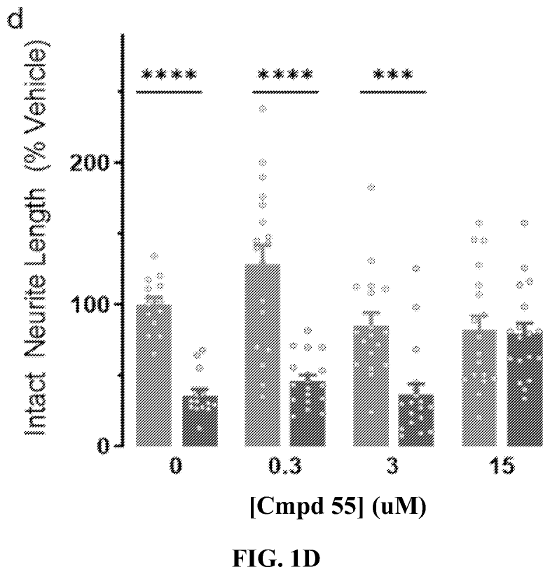

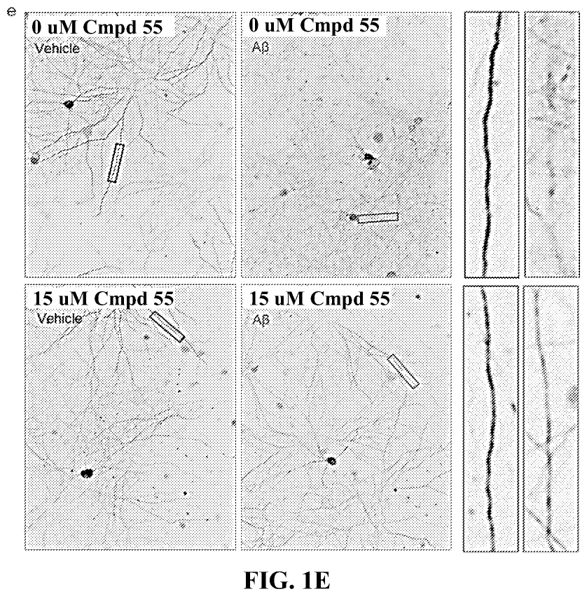

˜50,000 compounds from the Enamine and Southern Research libraries were screened to identify Tau-FynSH3 interaction inhibitors using an AlphaScreen assay (Cochran, et al. (2014) J. Biomol. Screen. 19, 1338-1349). Compounds that were >3 standard deviations above mean inhibition were selected, yielding 1,852 hits (FIG. 1A). Those hits were further screened using four assays: a repeat Tau-FynSH3 AlphaScreen with fresh compound, a counterscreen with covalently linked GST-His to identify compounds that nonspecifically inhibited AlphaScreen chemistry, and two toxicity assays in LL47 and THP-1 cells. Compounds showing >50% inhibition in the repeat Tau-FynSH3 AlphaScreen that also had IC₅₀ in the counterscreen and EC₅₀ in both toxicity assays >2-fold higher than the IC₅₀ for Tau-FynSH3 were advanced. 64 compounds met all four criteria, with many displaying no activity in the counterscreen and toxicity assays. These 64 compounds were then re-run through the Tau-FynSH3 AlphaScreen in triplicate, and 39 compounds with IC₅₀<100 M were advanced. These 39 compounds were tested in a live-cell BRET assay to measure Tau-Fyn interaction in cells. Seven compounds were active in the BRET, five of which had promising modifiable structures (Cochran, et al. (2014) J Biomol. Screen. 19, 1338-1349).

A computational analysis of the top five hits was then completed to assess the physical attributes of the compounds emerging from the HTS. The target profile for drug-like properties included evaluation of molecular weight (MW), total polar surface area (PSA, the surface sum of all polar atoms on the molecule), solubility, metabolic stability, and partition coefficient (LogD). In general, MW<500 kDa and PSA <70 Å² were targeted, which generally allows for blood-brain barrier (BBB) permeability. Compounds with PSA >100 A² are generally poor at permeating cell membranes. Solubility was targeted at >10 μM at pH 7.4 and 40-60% of compound remaining after 60 minutes of incubation in mouse or human liver microsomes (an indicator of compound stability after first-pass metabolism). LogD is the log of the ratio of concentrations of a compound in two immiscible solvents at equilibrium. Values ranging from 2-4 were targeted, typical for CNS drugs. Based on these criteria, compound no. 55 was selected as the top hit for optimization. Compound no. 55 had an IC₅₀ of 5.0 μM in the Tau-FynSH3 AlphaScreen and demonstrated acceptable drug-like properties, with a MW of 380.43 kDa, PSA of 96 Å², solubility of 49 μM, metabolic stability in liver microsomes (Mouse t_{1/2}=50.4 min; Human t_{1/2}=106.6 min), and LogD of 1.8 (FIG. 1B).

Next, it was tested whether compound no. 55 was biologically active in assays with relevance to AD. Two assays of Aβ toxicity in primary neurons were used, with which protective effects of a Tau-SH3 inhibitor peptide had been previously identified (Rush, et al. (2020) Neurobiol Dis 134, 104668). Compound no. 55 prevented Aβ-induced membrane trafficking dysfunction using a modified MTT assay (FIG. 1C) and prevented Aβ-induced neurite degeneration using a MAP2 ICC-based neurite measuring algorithm (FIG. 1D and FIG. 1E). Altogether, these results support compound no. 55 as a promising compound with micromolar potency as a Tau-Fyn interaction inhibitor, reasonable drug-like properties, and biological activity in reducing Aβ toxicity.

Referring to FIG. 1A-E, top hit compound no. 55 has attractive druglike properties and prevents Aβ toxicity. FIG. 1A shows the workflow for identification of compound no. 55 as a top hit from a HTS of ˜50,000 compounds. FIG. 1B shows the structure and druglike properties of compound no. 55. FIG. 1C shows that 15 M compound no. 55 ameliorates Aβ toxicity in a modified MTT assay (n=28-30 wells per group from N=3 dissections. 2-way ANOVA main effect of Aβ p=0.043, F(1,111)=4.20, ** p<0.01 by Sidak posthoc). FIG. 1D shows that 15 M compound no. 55 ameliorates Aβ-induced neurite degeneration (n=14-18 wells per group from N=2 dissections. 2-way ANOVA main effect of Aβ p<0.0001, F(1,128)=66.65, *** p<0.001, **** p<0.0001 by Sidak posthoc). FIG. 1E shows representative images of MAP2-stained neurites for neurite degeneration assay. Data displayed as Mean±SEM.

b. Medicinal Chemistry Optimization of Compound No. 55

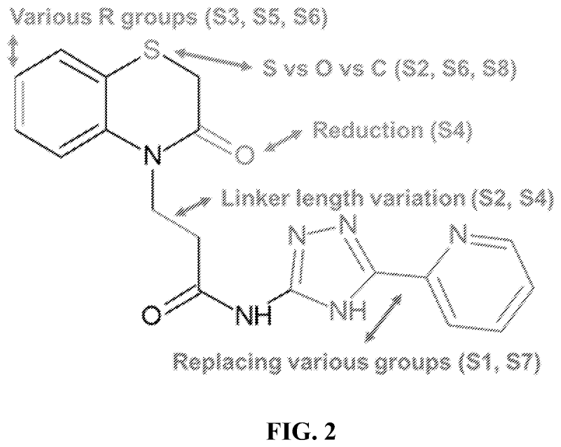

After identifying compound no. 55 as the most promising HTS hit and verifying its biological activity, a medicinal chemistry optimization program was initiated. To generate structure-activity relationships and to improve overall drug-like properties, various modifications were performed on compound no. 55 (FIG. 2). Altogether, 92 analogs with diverse modifications of compound no. 55 were designed, synthesized, and tested for Tau-FynSH3 inhibitory activity in the AlphaScreen, of which 29 are shown in Table 2 below. Sveral moieties to modify in compound no. 55 were identified (FIG. 2): the 5-(pyridin-2-yl)-4H-1,2,4-triazol-3-amine moiety was replaced with various groups, the ethyl linker was replaced with a methylene linker, the ring sulfur was replaced with oxygen and carbon, the cyclic ketone was removed, and various aromatic R groups were introduced in the left-hand phenyl ring.

TABLE 2

| | IC₅₀ | PSA | Solubility | MLM t_{1/2} |
|---|---|---|---|---|
| No. | (μM) | (A) | (μM) | (min) |
| 55 | 5 | 98 | 49 | 50.4 |
| 37 | >100 | 77 | N.D. | N.D. |
| 56 | 0.84 | 104 | 62 | 210 |
| 43 | >100 | 108 | N.D. | N.D. |
| 1 | 1.97 | 108 | 80 | 256 |
| 3 | >100 | 98 | N.D. | N.D. |
| 2 | 2.72 | 98 | N.D. | N.D. |
| 14 | 0.9 | 98 | 68 | 52 |
| 23 | 0.79 | 98 | 53 | 37 |
| 17 | 1.03 | 108 | 46 | 53 |
| 21 | 5.42 | 98 | N.D. | N.D. |
| 13 | 0.93 | 98 | 17 | 61 |
| 12 | 0.99 | 98 | 41 | 146 |
| 20 | 0.55 | 81 | 14 | 9.2 |
| 57 | 3.26 | 64 | 72.2 | 1.6 |
| 58 | 0.45 | 81 | <1 | 13.9 |
| 59 | 0.36 | 81 | 2.1 | 11.6 |
| 60 | 0.3 | 81 | <1 | 9.7 |
| 61 | 0.35 | 105 | 3.2 | 45.1 |
| 62 | 0.44 | 108 | N.D. | N.D. |
| 63 | 0.23 | 133 | N.D. | N.D. |
| 64 | <0.2 | 119 | 69.2 | >300 |
| 65 | 4.9 | 81 | 48.3 | 67.4 |
| 66 | 3.8 | 91 | 42.4 | 148.8 |
| 67 | 0.38 | 69 | 5.2 | 3 |
| 47 | 0.32 | 78 | 80.2 | 2 |
| 68 | >100 | 66 | N.D. | N.D. |
| 45 | 1.25 | 81 | 4 | 19 |
| 69 | 0.91 | 91 | 54 | 37 |

First, the 5-(pyridine-2-yl)-4H-1,2,4-triazole moiety of compound no. 55 was replaced with substituted aryl, heteroaryl, and biphenyl rings. All modifications resulted in no observed AlphaScreen activity, as seen in compound no. 37, demonstrating the importance of that moiety.

Next, whether the ethyl linker could be shortened was evaluated, and also whether replacing the ring sulfur atom with carbon or oxygen to improve drug-like properties. Five new compounds (1-3, 43, and 56), with all possible combinations of these modifications. Modification of the ethyl linker of compound no. 55 to a methylene linker gave compound no. 56, which showed a five-fold improvement in activity, along with improved solubility and metabolic stability in liver microsomes. However, there was no activity with an ethyl linker with either oxygen or carbon in the ring. With a methylene linker, there was activity with both oxygen and carbon, though progressively less activity than sulfur. Solubility and metabolic stability, however, were improved by the ring oxygen.

Thus, a methylene linker improves activity and a ring sulfur has the highest AlphaScreen activity (0.84 μM). From these modifications, compound no. 56 was identified, which, in addition to a five-fold improvement in activity in the Tau-FynSH3 AlphaScreen, also offers improved solubility and metabolic stability compared to compound no. 55.

Then, various substituents such as Cl, F, CF₃, and CH₃ were introduced at the 6- and 7-positions of the left-hand phenyl ring to create analogs (12-14, 17, 21, and 23). While none of the substitutions dramatically increased activity, several had a decreased PSA calculation, an indicator of potential brain permeability. The most interesting compound was compound no. 12, with a 7-fluoro substituent, which had the lowest PSA, submicromolar IC₅₀, and the highest metabolic stability of the daughters (MLM t_{1/2}=146 minutes). It was then empirically determined whether the PSA was sufficiently low to allow BBB permeability by orally administering compound no. 12 to mice (5 mg/kg). While compound no. 12 was detected in plasma, it did not cross the BBB and was not present in brain (data not shown).

To reduce the PSA, the cyclic ketone of compound no. 56 was removed, resulting in compound no. 20, which showed inhibition in the AlphaScreen (IC₅₀=0.55 M) and low PSA, but had low solubility and metabolic stability. Since compound no. 20 was promising, a negative control was also prepared, compound no. 57, with an ethyl linker that, as predicted, had six-fold lower activity. When orally administered, compound no. 20 was detected in both plasma and brain, though relatively little crossed into the brain, with a maximum brain/plasma ratio of 0.06.

Compound no. 20 was then modified to optimize its other drug-like properties for further biological testing. For example, various groups were introduced into the aromatic ring, though all resulting compounds were either insufficiently soluble (58-61) or had high PSA (61-64). The left-hand phenyl ring was also removed, which maintained low PSA and improved solubility and metabolic stability, but activity was reduced 8-10-fold (65, 66). Additional modifications of the 5-(pyridin-2-yl)-4H-1,2,4-triazol-3-amine moiety yielded some analogs with improved activity, but also had reduced metabolic stability (67, 68), or lost activity (68).

Finally, the ring sulfur of compound no. 20 was replaced with oxygen and carbon (45, 69). Without wishing to be bound by theory, it was expected that this would result in slightly less activity, but improved solubility and metabolic stability, as in the second series. As expected, analogs with oxygen and carbon in the ring were less active. The analogue with the oxygen substitution, compound no. 69, had improved solubility and metabolic stability with submicromolar activity. Oral administration of compound no. 69 did result in its presence in both plasma and brain with a brain/plasma ratio of 0.067, similar to compound no. 20 (see Table 2). In summary, 92 analogs of compound no. 55 were synthesized, leading to the identification of compound no. 69 as a promising candidate for further testing.

c. Compound No. 69 Blocks Aβ-Induced Neuronal Dysfunction

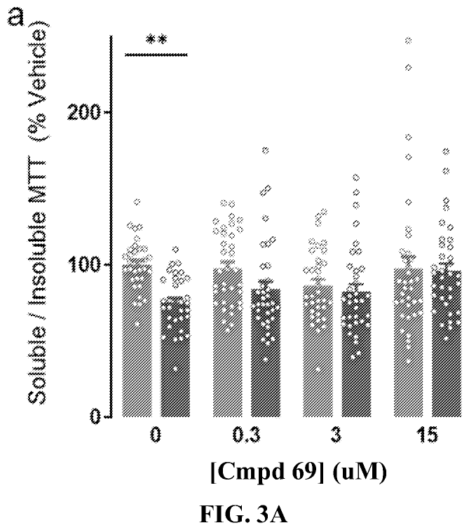

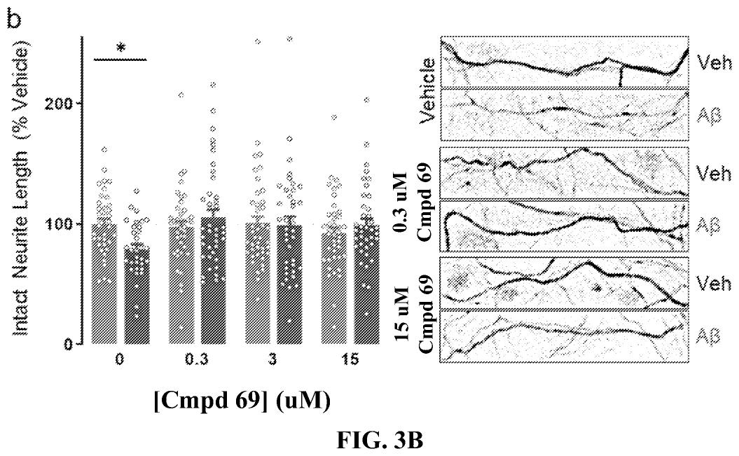

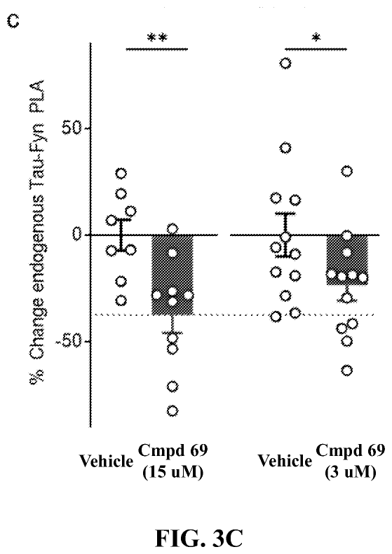

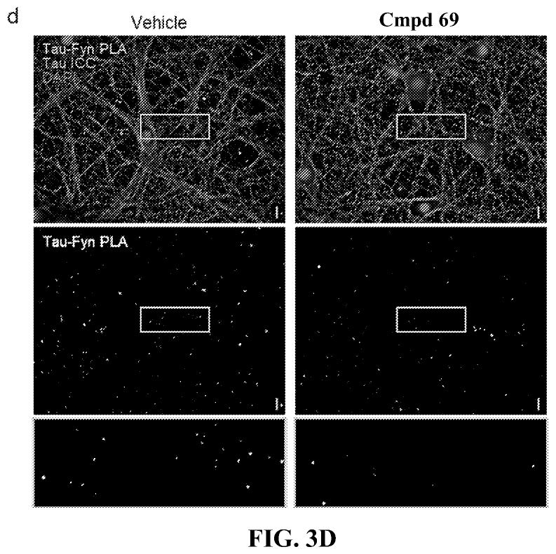

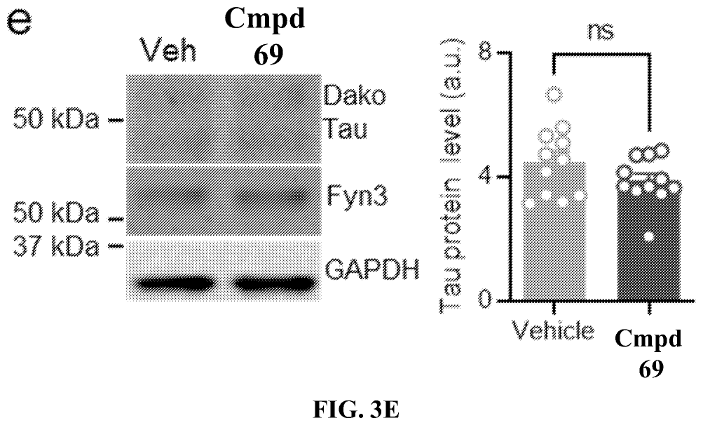

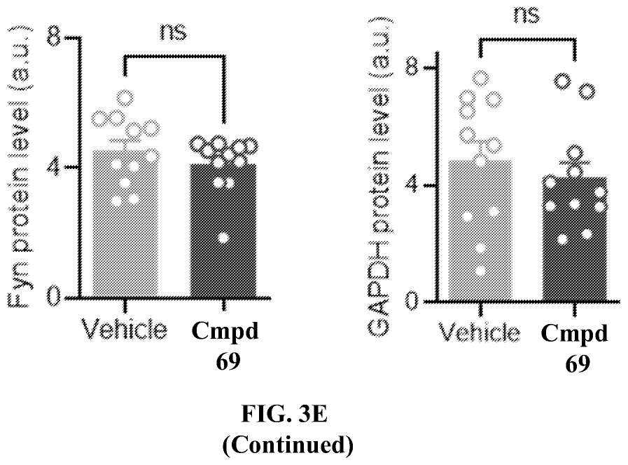

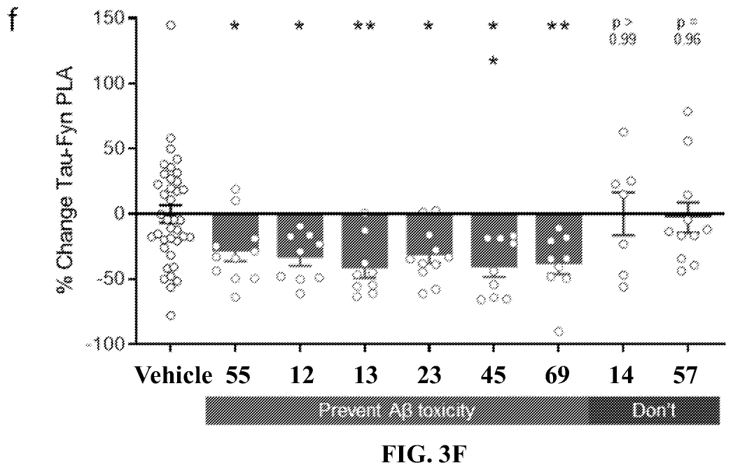

To determine whether compound no. 69 prevented Aβ toxicity, two primary neuron assays were used. Compound no. 69 prevented Aβ-induced membrane trafficking dysfunction in the MTT assay (FIG. 3A) and Aβ-induced neurite degeneration in the MAP2 assay (FIG. 3B and FIG. 3C), like compound no. 55, but with higher potency.

Next, target engagement of the Tau-Fyn interaction was evaluated using a proximity ligation assay (PLA) in primary neurons. Compound no. 69 inhibited endogenous Tau-Fyn interaction at both 3 μM and 15 μM (FIG. 3C and FIG. 3D). Importantly, compound no. 69 did not lower Tau or Fyn levels in neurons (FIG. 3E), supporting a mechanism based on true inhibition of the protein-protein interaction, not simply reduction of the interaction by lowering expression of Tau or Fyn.

Referring to FIG. 3A-F, compounds that inhibit Tau-Fyn interaction in cells also ameliorate Aβ toxicity. FIG. 3A shows that compound no. 69 ameliorates Aβ toxicity (n=32-36 wells per group from N=3 dissections. 2-way ANOVA main effect of AD p=0.0017, F(1, 268)=10.06, ** p<0.01 by Sidak posthoc), while compound no. 57 does not (n=27-30 wells per group from N=3 dissections. 2-way ANOVA main effect of Aβ p<0.0001, F(1,225)=46.33, * p<0.05, ** p<0.01, *** p<0.001 by Sidak posthoc). FIG. 3B shows that compound no. 69 ameliorates Aβ-induced neurite degeneration. (n=33-42 wells per group from N=4 dissections. 2-way ANOVA interaction p=0.04, F(1,304)=2.76, * p<0.05 by Sidak posthoc), with representative images of MAP2-stained neurites. FIG. 3C shows that both 15 μM (n=8-10 per group from N=2 dissections. t-test p=0.0046) and 3 μM (n=12 per group from N=2 dissections) reduce interactions between endogenous Tau and Fyn (One-tailed t-test p=0.034). FIG. 3D shows that compound no. 69 reduces endogenous Tau-Fyn PLA in primary neurons. Representative images of endogenous Tau-Fyn PLA in primary neurons treated with 15 μM compound no. 69 are shown. Yellow box is inset on right. Scale bar=10 μm. FIG. 3E shows that 15 μM compound no. 69 does not reduce Tau, Fyn, or GAPDH levels in primary neurons (n=11 per group from N=2 dissections. Student t-test p >0.05). FIG. 3F shows that compounds that prevent Aβ toxicity significantly reduce Tau-Fyn PLA while compounds that do not prevent Aβ toxicity do not reduce Tau-Fyn PLA in HEK-293 cells (n=7-11 per group from N=2-3 passages. ANOVA p=0.0002, F(8,105)=4.22, * p<0.05, ** p<0.01 by Holm-Sidak posthoc). Data displayed as Mean±SEM.

### d. Effects on Aβ Toxicity Associate With Tau-SH3 Inhibition

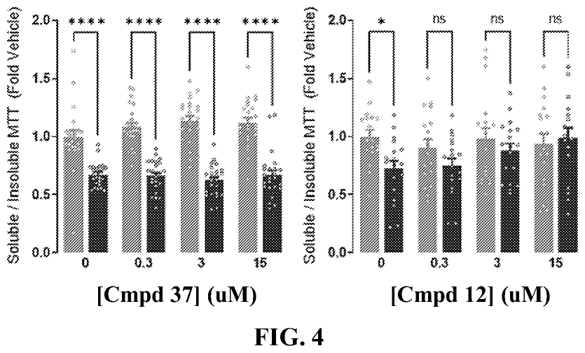

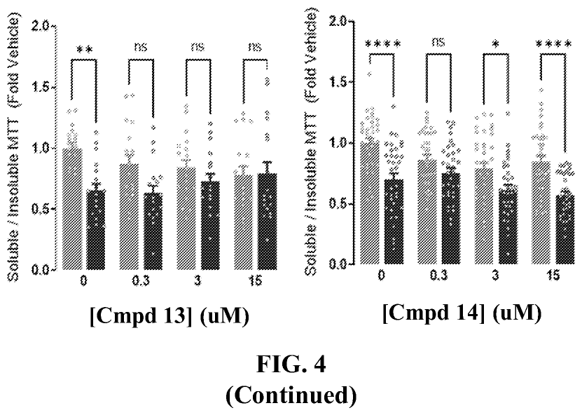

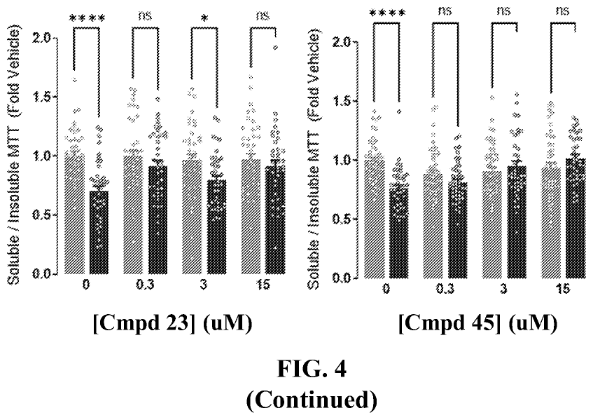

To further explore the relationship between preventing Aβ toxicity and inhibiting Tau-SH3 interactions in cells, eight compounds with varying druglike properties were selected to test in cells. Each compound was tested in two assays: the MTT assay in primary neurons to assess Aβ toxicity and Tau-Fyn PLA in HEK-293 cells to assess interaction inhibition. Six of these compounds inhibited Aβ toxicity (55, 12, 13, 23, 45, 69) while two did not (14, 57) (FIG. 4). Each of the compounds that ameliorated Aβ toxicity also reduced Tau-Fyn interaction in cells. On the other hand, the two compounds that did not ameliorate Aβ toxicity also did not reduce Tau-Fyn interaction in cells. These results are consistent with an association between the ability of compounds to inhibit Tau-Fyn interaction and to ameliorate Aβ toxicity.

Referring to FIG. 4, compound no. 37 (p<0.0001, F(1,184)=260.4, n=24 per group) and compound no. 14 (p<0.0001 F(1,268)=51.03, n=33-35 per group) did not ameliorate Aβ toxicity. The remaining compounds did ameliorate Aβ toxicity: compound no. 12 (p=0.0229, F(1,135)=5.3, n=17-18 per group), compound no. 13 (p=0.0005, F(1,135)=12.91. n=17-18 per group), compound no. 23 (p<0.0001, F(1,309)=22.42, n=37-40 per group), and compound no. 45 (p=0.0499 F(1,358)=3.87, n=42-48 per group). All: 2-way ANOVA main effect of Aβ p and F listed, *:p<0.05, **:p<0.01, ***:p<0.001, ****:p<0.0001 Sidak posthoc.

e. Compound No. 69 Binds Tau Not FynSH3

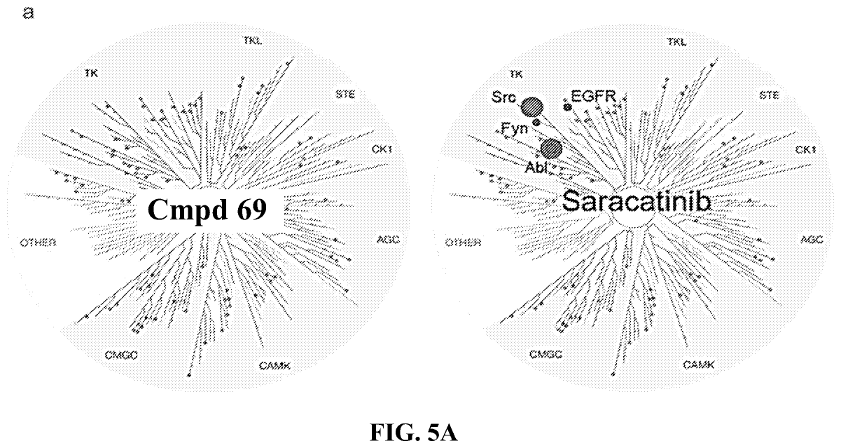

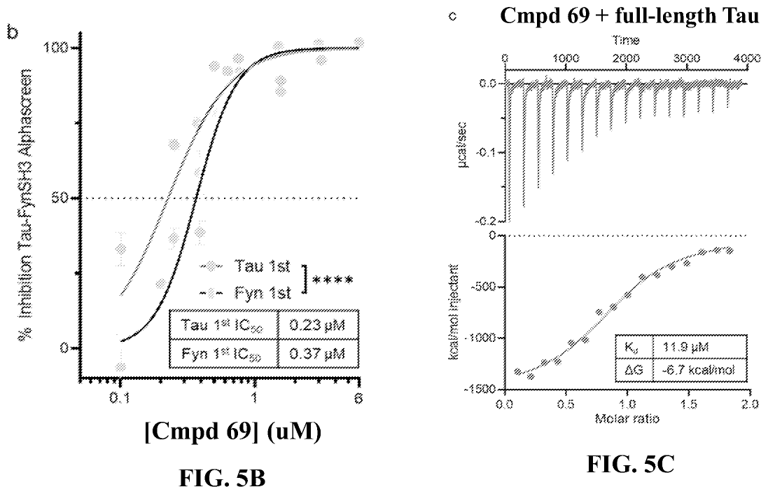

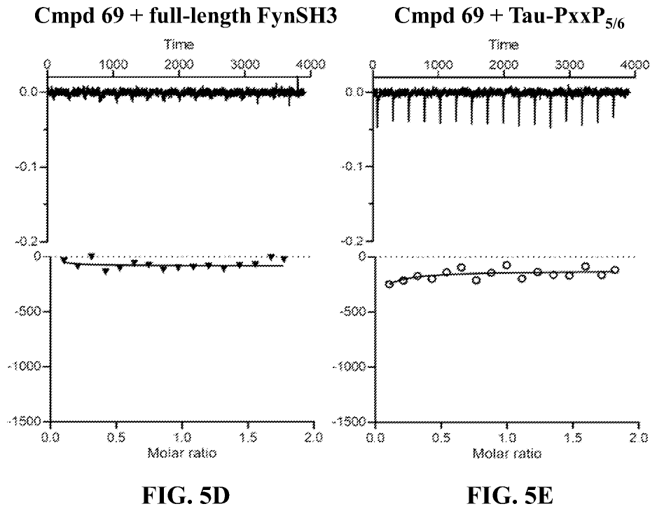

Having identified compound no. 69 as a novel Tau-FynSH3 interaction inhibitor that ameliorates Aβ toxicity in neurons, several aspects of its mechanism of action were explored. First, whether compound no. 69 acts as a kinase inhibitor was investigated, as Fyn kinase inhibition is a potential therapeutic approach under investigation for AD (Nygaard, H.B. (2018) Biological Psychiatry 83, 369-376). At 3 μM, which is sufficient to ameliorate Aβ toxicity and inhibit Tau-SH3 interaction, compound no. 69 did not inhibit Fyn or any of the 97 kinases in the DiscoverX KINOMEscan™, while a positive control, saracatinib, inhibited several Src family kinases (SFKs) as expected (FIG. 5A, full data not shown). Thus, compound no. 69's amelioration of Aβ toxicity is not due to inhibition of Fyn or other kinases and is likely due to inhibition of Tau-SH3 interactions.

Next, the structural basis for compound no. 69 effects was examined; specifically, whether it inhibits Tau-Fyn interaction by binding to Tau or to Fyn. Since Tau is natively unstructured and FynSH3 has a well-structured binding pocket, it was initially suspected that compound no. 69 would be more likely to bind FynSH3. This prediction was initially tested with molecular docking and dynamics, which showed no strong binding of compound no. 69 to FynSH3 (data not shown). Next, an AlphaScreen preincubation assay was used, which distinguishes between compounds that bind Tau and FynSH3 because compounds show greater potency when preincubated with their binding partner than when preincubated with the other protein (Cochran, et al. (2014) J. Biomol. Screen. 19, 1338-1349). Compound no. 69 was more potent when pre-incubated with Tau (IC₅₀=0.23 μM) than with FynSH3 (IC₅₀=0.37 μM). further suggesting that compound no. 69 more likely binds Tau (FIG. 5B).

Finally, isothermal titration calorimetry (ITC) was used to definitively quantify compound no. 69 binding to Tau and to Fyn. Compound no. 69 bound full-length Tau with K_{d}=11.9 μM and ΔG=−6.7 kcal/mol (FIG. 5C), while it did not bind FynSH3 (FIG. 5D). Altogether, these data demonstrate that compound no. 69 inhibits Tau-Fyn interaction by binding to Tau rather than FynSH3.

To begin addressing the location of the compound no. 69 binding site on Tau, whether the compound could bind the peptide incorporating Tau's 5^{th}/6^{th} PxxP (amino acids 209-225) was tested, which is the primary site for Fyn interaction (Reynolds, et al. (2008) J Biol. Chem. 283, 18177-86; Cochran, et al. (2014) J Biomol. Screen. 19, 1338-1349). No interaction was observed between compound no. 69 and the Tau-PxxP5/6 peptide (FIG. 5E), suggesting that the compound either requires additional tertiary structural elements or allosterically inhibits the interaction by binding another region of Tau.

Referring to FIG. 5A-E, compound no. 69 binds Tau not FynSH3. FIG. 5A shows that compound no. 69 is not a kinase inhibitor while positive control SFK inhibitor saracatinib inhibits Src, Fyn, Abl, and EGFR kinase activity. FIG. 5B shows that preincubating compound no. 69 with Tau in the Tau-FynSH3 AlphaScreen resulted in more potent inhibition than preincubating compound no. 69 with FynSH3 (n=3 wells per group in N=2 runs. 2-way ANOVA main effect of preincubation p<0.0001, F(1,75)=80.22). Data displayed as Mean±SEM. FIG. 5C-E show the ITC reaction heat and fitting curves of compound no. 69 with full-length Tau (FIG. 5C), FynSH3 (FIG. 5D), and Tau-PxxP_{5/6} (FIG. 5E). For compound no. 69 with full-length Tau, n=0.9, K_{d}=11.9 μM, ΔG=−6.7 kcal/mol, and ΔH=−1.5 kcal/mol. Results were confirmed with three independent experiments.

### f. Compound no. 69 is a Selective Tau-SH3 Interaction Inhibitor

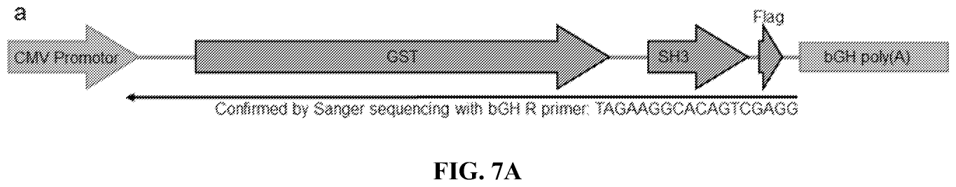

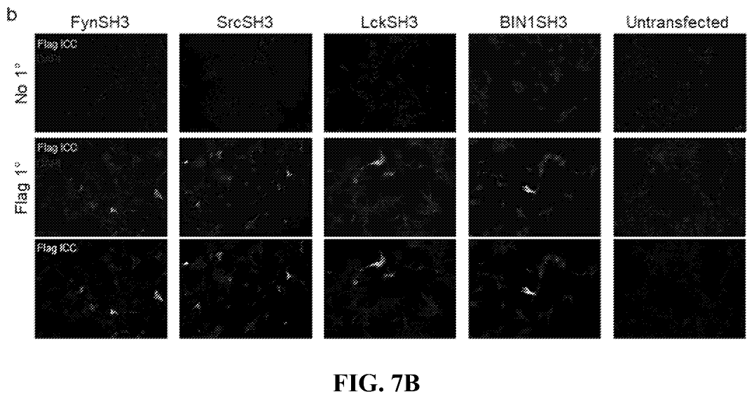

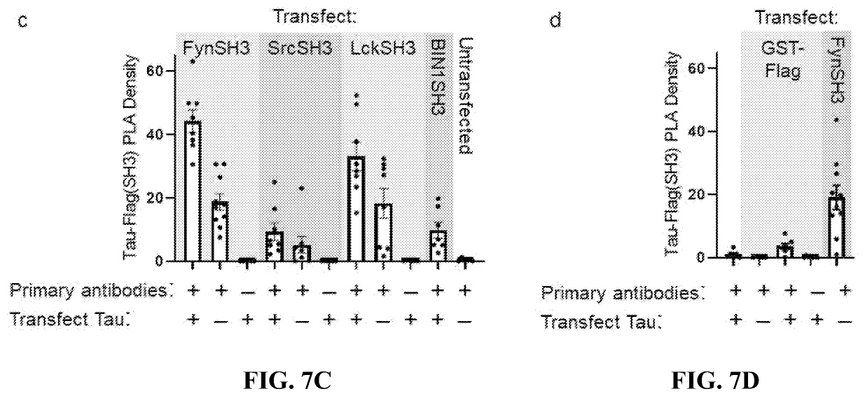

Since compound no. 69 binds Tau, it was next determined whether it inhibits other Tau-SH3 interactions besides Tau-Fyn. Three other SH3 domains of interest were identified: Src-family kinases with either type A SH3 (Src) or type B SH3 (Lck) domains, and BIN1, a non-kinase SH3 domain-containing protein that is a genetic risk factor for AD and has less homology to the Src family kinases (FIG. 6A-C). Plasmids were designed for each of these SH3 domains fused to a GST and Flag tags (FIG. 6D) and PLA with antibodies for Tau and Flag was use to measure each Tau-SH3 interaction (FIG. 7A-D). Compound no. 69 again inhibited Tau-FynSH3 (FIG. 6E), slightly but insignificantly inhibited Tau-SrcSH3 (FIG. 6F), strongly inhibited Tau-LckSH3 (FIG. 6G), and moderately inhibited Tau-BIN1 (FIG. 6H). Interestingly, a recent modeling study (Wang, et al. (2019) Journal of Molecular Graphics and Modelling 90, 265-272) predicted that Fyn, Lck, and BIN1 bind most strongly at Tau's 5^{th} PxxP motif, while Src binds strongly at the 7^{th} PxxP, indicating that compound no. 69 may primarily inhibit interactions that occur at the 5^{th}/6^{th} motif.

Given the finding that compound no. 69 inhibits multiple Tau-SH3 interactions, whether it inhibits other Tau protein-protein interactions was evaluated. Tau's interaction with tubulin via the more carboxy-terminal microtubule-binding domain mediates its canonical function as a microtubule-stabilizing protein. Endogenous Tau-tubulin interactions were measured in cultured cells using PLA. Compound no. 69 did not inhibit Tau-tubulin binding (FIG. 6I). Thus, compound no. 69 does not broadly inhibit all Tau protein-protein interactions, but rather selectively inhibits its interactions with certain SH3 domain-containing proteins.

Referring to FIG. 6A-I, compound no. 69 is a selective Tau-SH3 interaction inhibitor. FIG. 6A shows a phylogenetic tree of the 306 SH3 domains in 225 genes in the human genome identified in the UniProt database, produced by ClustalOmega. SFKs Fyn (light green), Src (dark green), and Lck (blue) are closely related, while BIN1 (brown) is distantly related. FIG. 6B shows a multiple sequence alignment of the SH3 domains examined. FIG. 6C shows a percent identity matrix of the SH3 domains. FIG. 6D shows a diagram of plasmids expressing FynSH3, SrcSH3, LckSH3, and BIN1SH3. FIG. 6E shows that compound no. 69 inhibits Tau-FynSH3 interaction 37% (n=16 per group, * p=0.04). FIG. 6F shows that compound no. 69 does not inhibit Tau-SrcSH3 interaction (n=15-18 per group, p=0.11). FIG. 6G shows that compound no. 69 inhibits Tau-LckSH3 interaction (n=14-15 per group, **** p<0.0001). FIG. 611 shows that compound no. 69 inhibits Tau-BIN1SH3 interaction (n=18 per group, p=0.003). FIG. 61 shows that compound no. 69 does not inhibit Tau-tubulin interaction (n=16-17 per group, p=0.30). Referring to FIG. 6E-I, N=3 unique passages of HEK-293 cells, one-tailed t-test. Data displayed as Mean±SEM.

Referring to FIG. 7A-D, controls for SH3 PLAs are shown. Specifically, FIG. 7A shows the plasmid design for all SH3 constructs. All plasmids were confirmed with Sanger sequencing with a common primer beginning in the bGH polyA tail and reading reverse through the coding region. Plasmid expression in HEK-293 cells was confirmed by immunocytochemistry (FIG. 7B). Tau-Flag PLA was completed to measure Tau-SH3 interaction (FIG. 7C). With just the SH3 domain overexpressed, there was moderate Tau-Flag PLA (SH3 interacting with endogenous Tau), and overexpressing Tau increased Tau-Flag PLA. Untransfected cells and transfected cells without primary antibody had little to no PLA (n=5-10 images per group). Referring to FIG. 7D, a negative control GST-Flag plasmid lacking an SH3 domain showed little to no PLA compared to Tau-Flag PLA in cells transfected with GST-Fyn-SH3 (n=5-10 images per group).

### g. Tau-SH3 Inhibition Prevents Aβ-Induced Hyperexcitability

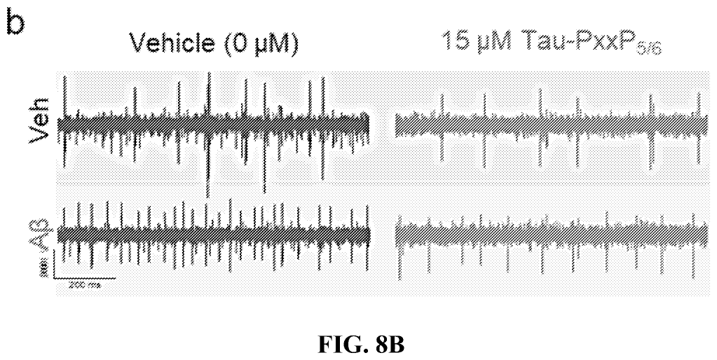

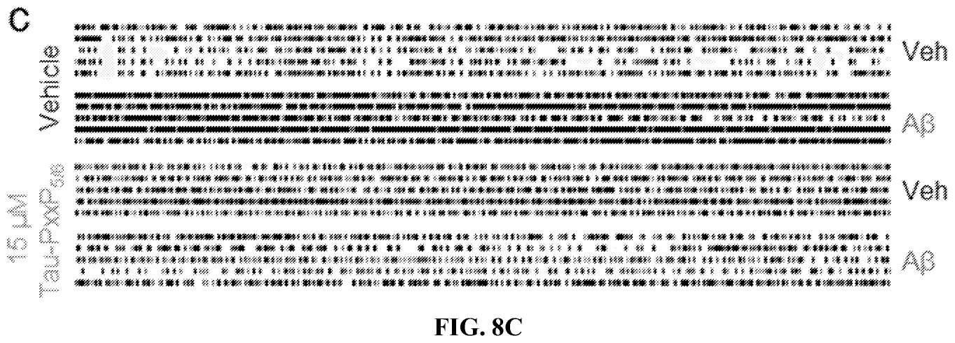

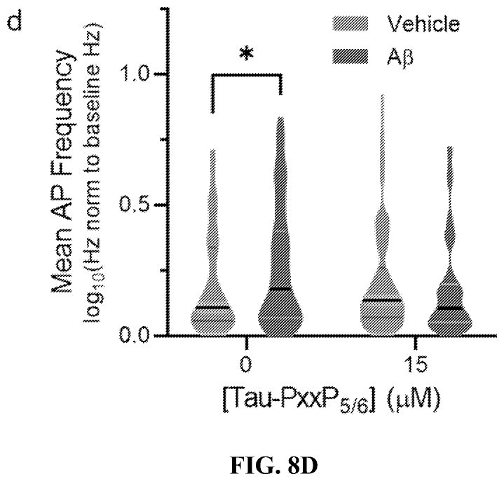

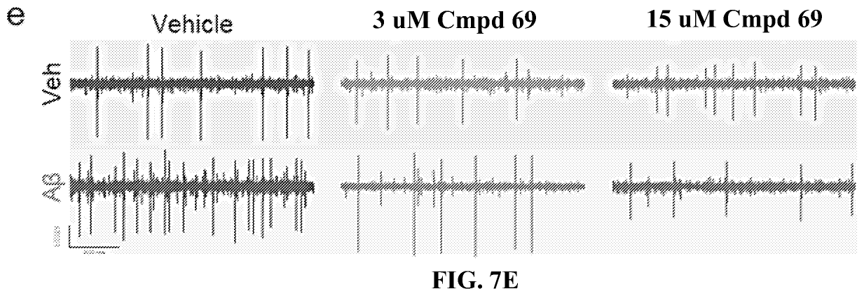

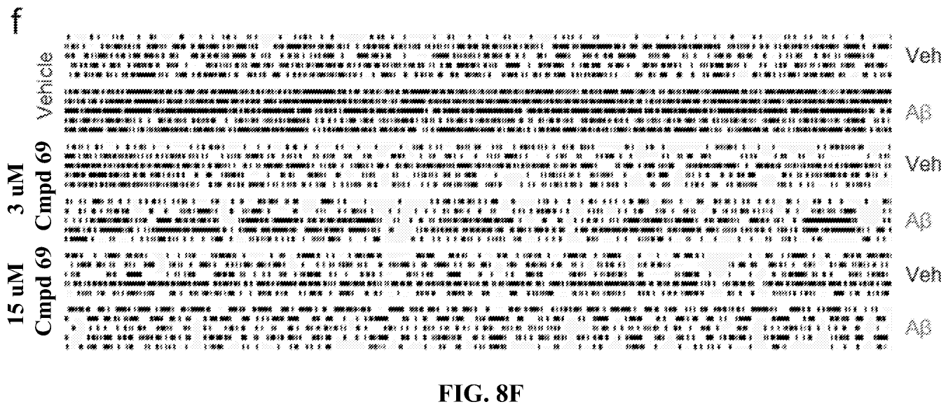

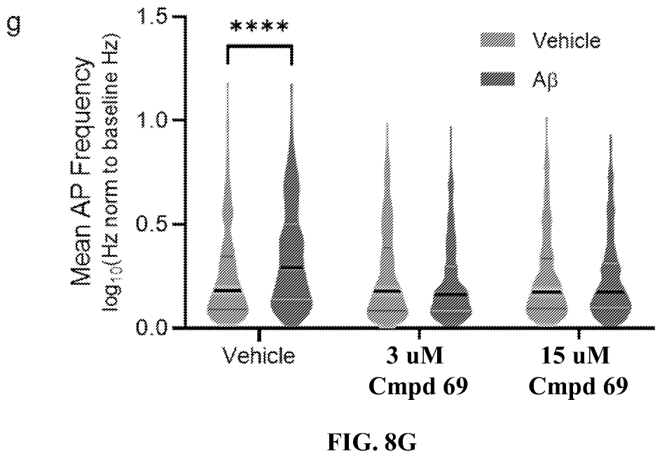

Network hyperexcitability is a common feature in AD that can be prevented by Tau reduction, but the effects of Tau-SH3 inhibitors on Aβ-induced hyperexcitability have not been examined. Here, a 48-well multi-electrode array (FIG. 8A) was utilized to measure changes in neuronal activity after application of 500 nM Aβ oligomers. In vehicle-treated neurons, Aβ induced an increase in neuronal activity (FIG. 8B). The effects of the Tau-SH3 inhibitor peptide were initially examined based on the 5^{th}/6^{th} PXXP motif that was previously described (Rush, et al. (2020) Neurobiol Dis 134, 104668). Preincubation with Tau-PxxP_{5/6} for one hour prevented Aβ-induced hyperactivity (FIG. 8B-D). Next, whether compound no. 69 had a similar effect was tested. Compound no. 69 also prevented Aβ-induced network8hyperexcitability at both 3 and 15 μM (FIG. 8E-G). Without wishing to be bound by theory, the results demonstrate that Tau-SH3 inhibition prevents multiple hallmarks of AD pathogenesis induced by Aβ in neurons, namely metabolic dysfunction (MTT), neurite degeneration, and network hyperexcitability.

Referring to FIG. 8A-G, compound no. 69 ameliorates Aβ-induced neurite degeneration and network hyperexcitability. FIG. 8A shows a timeline of MEA experiments completed on DIV13. FIG. 8B shows representative traces of one second of neurons firing post-treatment with Tau-PxxP_{5/6}. FIG. 8C shows representative raster plots of five neurons per group firing over the 20-minute post-treatment recording. FIG. 8D shows quantification of neuronal firing. Tau-PxxP_{5/6} ameliorates Aβ-induced network hyperexcitability (n=63-115 neurons per group. 2-way ANOVA interaction p=0.02, * p<0.05 by Sidak posthoc). FIG. 8E shows representative traces of one second of neurons firing post-treatment with compound no. 69. FIG. 8F shows representative raster plots of five neurons per group firing over the 20-minute post-treatment recording. FIG. 8G shows quantification of neuronal firing. Compound no. 69 ameliorates Aβ-induced network hyperexcitability (n=257-326 neurons per group from N=2 dissections. 2-way ANOVA interaction p<0.0001, F(2,1709)=13.47, **** p<0.0001 by Sidak posthoc).

h. Discussion

The study described herein addresses the potential of inhibiting Tau-SH3 interactions with small-molecule compounds as a therapeutic approach for Aβ-induced dysfunction. Several novel compounds that inhibit Tau-SH3 interactions were identified, and those with inhibitory activity in cell-based assays also ameliorated AD toxicity in neurons. The optimized compound, (69), bound to Tau and inhibited endogenous Tau-SH3 interaction in neurons without inhibiting Tau-tubulin interactions. Both compound no. 69 and a peptide inhibitor of Tau-SH3 interactions prevented Aβ-induced increases in neuronal firing, providing evidence for a role of Tau-SH3 interactions in AD-related network hyperexcitability.

Small molecules make attractive therapeutics as they are generally less expensive and more easily administered than biologics. Traditionally, protein-protein interactions have been considered challenging targets for small molecule drug discovery, particularly when the interaction involves large surface areas. However, many protein-protein interactions depend on a small “hot spot” on the larger binding surface (Arkin, et al. (2014) Chemistry & biology 21, 1102-1114, and a variety have been successfully targeted by small molecule compounds with several even progressing to clinical trials (Dong, G. & Sheng, C. Overview of Protein-Protein Interactions and Small-Molecule Inhibitors Under Clinical Development. in Targeting Protein-Protein Interactions by Small Molecules (eds. Sheng, C. & Georg, G. I.) 3-28 (Springer Singapore, Singapore, 2018)). Another interaction between proline-rich and SH3 domain-containing proteins, Sam68(PxxP)-FynSH3, is inhibited by UCS15A, a unique Src family kinase-targeting small molecule that does not inhibit SFK kinase activity or stability (Oneyama, et al. (2002) Oncogene 21, 2037-50) and is structurally distinct from compound no. 69 (Sharma, S. V. et al. (2001) Oncogene 20, 2068-2079). UCS15A inhibits Sam68(PxxP)-FynSH3 interaction by binding the proline-rich domain of Sam68 rather than FynSH3 (Oneyama, et al. (2002) Oncogene 21, 2037-50). Together with the finding herein that compound no. 69 binds Tau, this demonstrates that proline-rich domains can serve as targets for small-molecule inhibitors of SH3 interactions.

Interestingly, compound no. 69 does not impact all Tau interactions and seems to be selective for certain Tau-SH3 interactions. Compound no. 69 did not affect Tau-tubulin interaction, which is mediated by the more carboxy-terminal microtubule-binding domain. Compound no. 69 inhibited Tau interactions with FynSH3, LckSH3, and BIN1SH3, without inhibiting interactions with SrcSH3. Notably, both FynSH3 and BINISH3 primarily bind Tau's 5^{th}/6^{th} PxxP motif in cells (Cochran, et al. (2014). J. Biomol. Screen. 19, 1338-1349; Lasorsa, et al. (2018) Front Mol Neurosci 11, 421 (2018); Usardi, et al. (2011) FEBS J. 278, 2927-2937), and FynSH3, BIN1SH3, and LckSH3 all are predicted to have strongest binding at the 5^{th} pxxP in silico (Wang, et al. (2019) Journal of Molecular Graphics and Modelling 90, 265-272). In contrast, SrcSH3 binds most strongly at the 7^{th} PxxP (Wang, et al. (2019) Journal of Molecular Graphics and Modelling 90, 265-272). Thus, compound no. 69 may selectively inhibit interactions with the 5^{th}/6^{th} PxxP. Compound no. 69 did not bind a peptide containing this sequence (FIG. 5E), so it may act allosterically or require other elements of Tau sequence (perhaps through interaction events such as the “paperclip” conformation of Tau, which is compacted by proline-targeting kinases (Jeganathan, et al. (2008) J. Biol. Chem. 283, 32066-32076).

There are potential liabilities to targeting Tau-SH3 interactions. For example, Tau-Fyn interaction in oligodendrocytes promotes their maturation (Klein, et al. (2002) J Neurosci 22, 698-707), so it will be important to determine if compound no. 69 causes oligodendrocyte dysfunction. Alternatively, other SH3-PxxP interactions could be impacted by compound no. 69. For example, Lck's interaction with the proline-rich domain of T cell-specific adapter (TSAd) promotes immune activation (Andersen, et al. (2019) J Biol Chem 294, 15480-15494), so it will be important to determine if compound no. 69 disrupts TSAd-Lck interactions or immune function. Though this series of compounds still need to be tested in vivo, compound no. 69 binding to Tau reduces these concerns as manipulations targeting Tau do not usually cause oligodendrocyte or immune dysfunction. Even the minor abnormalities caused by Tau ablation are not present when Tau is heterozygously expressed (Li, et al. (2014) Neurobiol. Aging 35, 2617-2624; Tai, C. et al. Tau Reduction Prevents Key Features of Autism in Mouse Models. Neuron (2020); Ikegami, et al. (2000) Neurosci. Lett. 279, 129-132; Lei, et al. (2014) Mol Neurodegener 9, 29), so partially inhibiting Tau's interaction with a subset of proteins is not likely to raise major safety concerns. To this point, Tau reduction with antisense oligonucleotides is safe in preclinical models (DeVos, et al. (2013) J. Neurosci. 33, 12887-12897; DeVos, et al. (2017) Sci Tansl Med 9; Rush, et al. (2020) Neurobiol Dis 134, 104668), and a Tau antisense oligonucleotide, BIIB080, has been in clinical trials since 2017 with no adverse effects reported.

Targeting Tau-SH3 interactions may have fewer downsides than targeting SH3-targeting therapeutic strategies. Fyn kinase inhibition with saracatinib is currently in clinical trials for AD and prevents dysfunction in preclinical AD models (Nygaard, H. B. (2018) Biological Psychiatry 83, 369-376). However, there is likely a relatively narrow therapeutic window for Fyn inhibition, and a Phase IIa clinical trial with saracatinib showed no clinical benefit in AD at a tolerable dose (van Dyck, et al. (2019) JAMA Neurol 76, 1219-1229). Interestingly, saracatinib was recently reported to reduce Tau-Fyn interaction in cells (Tang, et al. (2020) Acta Neuropathologica Communications 8, 96), so it is possible that its beneficial effects in preclinical models result (at least in part) from inhibiting Tau-Fyn interaction in addition to or rather than by inhibiting Fyn kinase.

One important finding here is that Tau-SH3 inhibition prevents Aβ-induced network hyperexcitability. This is the first evidence directly relating Tau-SH3 interactions to network hyperexcitability and adds to the evidence that Tau is critical for pathogenic network hyperexcitability in neurodegeneration. Network hyperexcitability occurs early in AD pathogenesis, can be driven by Aβ, and contributes to neuronal excitotoxicity (Palop and Mucke (2016) Nat Rev Neurosci 17, 777-792). Tau reduction prevents network hyperexcitability (Ittner, et al. (2010) Cell 142, 387-397 (2010); Roberson, et al. (2011) J Neurosci 31, 700-11; DeVos, et al. (2013) J Neurosci. 33, 12887-12897; Li, et al. (2014) Neurobiol. Aging 35, 2617-2624; Gheyara, et al. (2014) Ann. Neurol. 76, 443-456; Holth, et al. (2013) J. Neurosci. 33, 1651-1659), and the effects of compound no. 69 support the idea that prevention of Tau-SH3 interactions may underlie the beneficial effects of Tau reduction in the context of neurodegeneration and network hyperexcitability.

Without wishing to be bound by theory, this study provides preclinical evidence supporting the idea of inhibiting Tau-SH3 interactions as a potential novel therapeutic strategy for AD. It provides a broad outline of a pipeline for identifying and optimizing small molecule protein-protein interaction inhibitors for use in cellular and animal models of Alzheimer's disease that ultimately could be used in the clinic. Finally, this study provides mechanistic insights into Aj-induced dysfunction, indicating that Tau's interactions with SH3 domain-containing proteins are critical for Aβ toxicity and network hyperexcitability, which could underlie the beneficial effects of Tau reduction.

### 4. Additional Data

A summary of the additional data generated as to the exemplary compounds is shown in Table 3 below.

TABLE 3

| | Alpha | | | | ADME |
|---|---|---|---|---|---|
| | screen | | | | t_{1/2} (min) |
| | IC₅₀ | | | | Sol (μM) |
| No. | (μM) | MTT | PLA | MW | LogD |
| 1 | 1.97 | — | — | 350.34 | MLMt_{1/2} = 256 |
| | | | | | HLMt_{1/2} => 300 |
| | | | | | Sol = 80 |
| | | | | | LogD = 1.8 |
| 2 | 2.72 | — | — | 348.37 | |
| 3 | NA | — | — | 362.39 | |
| 4 | NA | — | — | 365.41 | |
| 5 | 3.24 | — | — | 379.44 | |
| 6 | NA | — | — | 379.44 | |
| 7 | 1.92 | — | — | 398.42 | MLMt_{1/2} = 22 |
| | | | | | HLMt_{1/2} = 78 |
| | | | | | Sol = 69 |
| | | | | | LogD = 2.0 |
| 8 | 2.72 | Active | ND | 414.87 | MLMt_{1/2} = 18 |
| | | | | | HLMt_{1/2} = 62 |
| | | | | | Sol = 24 |
| | | | | | LogD = 2.3 |
| 9 | 2.97 | — | — | 416.41 | MLMt_{1/2} = 20 |
| | | | | | HLMt_{1/2} = 53 |
| | | | | | Sol = 81 |
| | | | | | LogD = 1.8 |
| 10 | 6.78 | — | — | 414.87 | |
| 11 | 3.45 | — | — | 448.42 | |
| 12 | 0.99 | Active | Active | 384.39 | MLMt_{1/2} = 146 |
| | | | | | HLMt_{1/2} => 300 |
| | | | | | Sol = 41 |
| | | | | | LogD = 2.4 |
| 13 | 0.93 | Active | Active | 400.84 | MLMt_{1/2} = 61 |
| | | | | | HLMt_{1/2} => 120 |
| | | | | | Sol = 17 |
| | | | | | LogD = 3.1 |
| 14 | 0.90 | Active | Inactive | 402.38 | MLMt_{1/2} = 52 |
| | | | | | HLMt_{1/2} = 137 |
| | | | | | Sol = 68 |
| | | | | | LogD = 1.9 |
| 15 | 1.08 | — | — | 384.39 | MLMt_{1/2} = 53 |
| | | | | | HLMt_{1/2} = 238 |
| | | | | | Sol = 65 |
| | | | | | LogD = 1.8 |
| 16 | 2.58 | — | — | 400.84 | MLMt_{1/2} = 26 |
| | | | | | HLMt_{1/2} = 74 |
| | | | | | Sol = 9 |
| | | | | | LogD = 2.3 |
| 17 | 5.42 | — | — | 434.40 | |
| 18 | 3.53 | — | — | 380.43 | MLMt_{1/2} = 40 |
| | | | | | HLMt_{1/2} = 152 |
| | | | | | Sol = 59 |
| | | | | | LogD = 1.3 |
| 19 | 1.50 | — | — | 394.45 | MLMt_{1/2} = 8 |
| | | | | | HLMt_{1/2} = 28 |
| | | | | | Sol = 20 |
| | | | | | LogD = 1.3 |
| 20 | 0.552 | Insoluble | ND | 352.42 | MLMt_{1/2} = 9 |
| | | | | | HLMt_{1/2} = 85 |
| | | | | | Sol = 14 |
| | | | | | LogD = 2.3 |
| 21 | 1.03 | — | — | 396.43 | MLMt_{1/2} = 53 |
| | | | | | HLMt_{1/2} = 175 |
| | | | | | Sol = 46 |
| | | | | | LogD = 1.7 |
| 22 | 0.9.4 | Active | Active | 396.43 | MLMt_{1/2} = 56 |
| | | | | | HLMt_{1/2} = 150 |
| | | | | | Sol = 80 |
| | | | | | LogD = 1.8 |
| 23 | 0.79 | Active | Active | 380.43 | MLMt_{1/2} = 37 |
| | | | | | HLMt_{1/2} = 133 |
| | | | | | Sol = 57 |
| | | | | | LogD = 2.2 |
| 24 | Inactive | — | — | 366.40 | |
| 25 | Inactive | — | — | 366.40 | |
| 26 | 1.01 | — | — | 352.37 | MLMt_{1/2} = 82 |
| | | | | | HLMt_{1/2} => 300 |
| | | | | | Sol = 22 |
| | | | | | LogD = 1.5 |
| 27 | ND | — | — | 323.37 | |
| 28 | Inactive | — | — | 307.31 | |
| 29 | Inactive | — | — | 305.34 | |
| 30 | 10.6 | — | — | 309.35 | |
| 31 | ND | — | — | 293.29 | |
| 32 | ND | — | — | 357.82 | |
| 33 | ND | — | — | 341.36 | |
| 34 | ND | — | — | 337.40 | |
| 35 | ND | — | — | 353.40 | |
| 36 | Inactive | — | — | 380.39 | |
| 37 | Inactive | — | — | 379.44 | |
| 38 | Inactive | — | — | 389.47 | |
| 39 | Inactive | — | — | 388.49 | |
| 40 | Inactive | — | — | 304.33 | |
| 41 | Inactive | — | — | 378.45 | |
| 42 | Inactive | — | — | 390.46 | |
| 43 | Inactive | — | — | 364.37 | |
| 44 | Inactive | — | — | 411.48 | |
| 45 | 1.25 | Active | Active | 334.38 | MLMt_{1/2} = 19 |
| | | | | | HLMt_{1/2} = 66 |
| | | | | | Sol = 4 |
| | | | | | LogD = 2.3 |
| 46 | 2.96 | | | 368.33 | |
| 47 | 0.91 | Active | Active | 336.36 | MLMt_{1/2} = 37 |
| | | | | | HLMt_{1/2} = 93 |
| | | | | | Sol = 54 |
| | | | | | LogD = 1.8 |
| 48 | 1.2 | | | 382.81 | |
| 49 | 1.6 | | | 380.36 | |
| 50 | 1.6 | | | 378.39 | |
| 51 | 1.6 | | | 366.36 | |
| 52 | 1.6 | | | 384.78 | |
| 53 | 1.6 | | | 364.37 | |
| 54 | 1.4 | | | 362.39 | |

### G. References

Rapoport, M., Dawson, H. N., Binder, L. I., Vitek, M. P. & Ferreira, A. Tau is essential to β-amyloid-induced neurotoxicity. Proc. Natl. Acad. Sci. USA 99, 6364-6369 (2002).
Roberson, E. D. et al. Reducing endogenous tau ameliorates amyloid β-induced deficits in an Alzheimer's disease mouse model. Science 316, 750-754 (2007).
Ittner, L. M. et al. Dendritic function of tau mediates amyloid-β toxicity in Alzheimer's disease mouse models. Cell 142, 387-397 (2010).
Roberson, E. D. et al. Amyloid-beta/Fyn-induced synaptic, network, and cognitive impairments depend on tau levels in multiple mouse models of Alzheimer's disease. J Neurosci 31, 700-11 (2011).
Leroy, K. et al. Lack of tau proteins rescues neuronal cell death and decreases amyloidogenic processing of APP in APP/PS1 mice. Am. J Pathol. 181, 1928-1940 (2012).
Meilandt, W. J. et al. Enkephalin Elevations Contribute to Neuronal and Behavioral Impairments in a Transgenic Mouse Model of Alzheimer's Disease. The Journal of Neuroscience 28, 5007 (2008).
Nussbaum, J. M. et al. Prion-like behaviour and tau-dependent cytotoxicity of pyroglutamylated amyloid-β. Nature 485, 651-655 (2012).
Palop, J. J. et al. Aberrant excitatory neuronal activity and compensatory remodeling of inhibitory hippocampal circuits in mouse models of Alzheimer's disease. Neuron 55, 697-711 (2007).
DeVos, S. L. et al. Antisense reduction of tau in adult mice protects against seizures. J. Neurosci. 33, 12887-12897 (2013).
DeVos, S. L. et al. Tau reduction prevents neuronal loss and reverses pathological tau deposition and seeding in mice with tauopathy. Sci Transl Med 9(2017).
Li, Z., Hall, A. M., Kelinske, M. & Roberson, E. D. Seizure resistance without parkinsonism in aged mice after tau reduction. Neurobiol. Aging 35, 2617-2624 (2014).
Gheyara, A. L. et al. Tau reduction prevents disease in a mouse model of Dravet syndrome. Ann. Neurol. 76, 443-456 (2014).
Holth, J. K. et al. Tau loss attenuates neuronal network hyperexcitability in mouse and Drosophila genetic models of epilepsy. J. Neurosci. 33, 1651-1659 (2013).
Singh, B. et al. Tau is required for progressive synaptic and memory deficits in a transgenic mouse model of alpha-synucleinopathy. Acta Neuropathol (2019).
Tai, C. et al. Tau Reduction Prevents Key Features of Autism in Mouse Models. Neuron (2020).
Lee, G., Newman, S T., Gard, D. L., Band, H. & Panchamoorthy, G. Tau interacts with src-family non-receptor tyrosine kinases. J Cell Sci. 111, 3167-3177 (1998).
Ittner, L. M. et al. Dendritic function of tau mediates amyloid-beta toxicity in Alzheimer's disease mouse models. Cell 142, 387-397 (2010).
Reynolds, C. H. et al. Phosphorylation regulates tau interactions with Src homology 3 domains of phosphatidylinositol 3-kinase, phospholipase Cgamma1, Grb2, and Src family kinases. J. Biol. Chem. 283, 18177-86 (2008).
Shirazi, S. K. & Wood, J. G. The protein tyrosine kinase, fyn, in Alzheimer's disease pathology. Neuroreport 4, 435-437 (1993).
Ho, G. J. et al. Altered p59Fyn kinase expression accompanies disease progression in Alzheimer's disease: Implications for its functional role. Neurobiol. Aging 26, 625-635 (2005).
Rush, T. et al. A peptide inhibitor of Tau-SH3 interactions ameliorates amyloid-beta toxicity. Neurobiol Dis 134, 104668 (2020).
Lambert, M. P. et al. Diffusible, nonfibrillar ligands derived from Aβ₁₋₄₂ are potent central nervous system neurotoxins. Proc. Natl. Acad. Sci. USA 95, 6448-6453 (1998).
Chin, J. et al. Fyn kinase modulates synaptotoxicity, but not aberrant sprouting, in human amyloid precursor protein transgenic mice. J Neurosci. 24, 4692-4697 (2004).
Voskobiynyk, Y. et al. Alzheimer's disease risk gene BIN1 induces Tau-dependent network hyperexcitability. eLife 9, e57354 (2020).
Cochran, J. N. et al. AlphaScreen HTS and live-cell bioluminescence resonance energy transfer (BRET) assays for identification of Tau-Fyn SH3 interaction inhibitors for Alzheimer disease. J. Biomol. Screen. 19, 1338-1349 (2014).
Nygaard, H. B. Targeting Fyn Kinase in Alzheimer's Disease. Biological Psychiatry 83, 369-376 (2018).
Wang, X., Chang, C., Wang, D. & Hong, S. Systematic profiling of SH3-mediated Tau-Partner interaction network in Alzheimer's disease by integrating in silico analysis and in vitro assay. Journal of Molecular Graphics and Modelling 90, 265-272 (2019).
Arkin, M. R., Tang, Y. & Wells, J. A. Small-molecule inhibitors of protein-protein interactions: progressing toward the reality. Chemistry & biology 21, 1102-1114 (2014).
Dong, G. & Sheng, C. Overview of Protein-Protein Interactions and Small-Molecule Inhibitors Under Clinical Development. in Targeting Protein-Protein Interactions by Small Molecules (eds. Sheng, C. & Georg, G. I.) 3-28 (Springer Singapore, Singapore, 2018).
Oneyama, C., Nakano, H. & Sharma, S. V. UCS15A, a novel small molecule, SH3 domain-mediated protein-protein interaction blocking drug. Oncogene 21, 2037-50 (2002).
Sharma, S. V. et al. UCS15A, a non-kinase inhibitor of Src signal transduction. Oncogene 20, 2068-2079 (2001).
Lasorsa, A. et al. Structural Basis of Tau Interaction With BIN1 and Regulation by Tau Phosphorylation. Front Mol Neurosci 11, 421 (2018).
Usardi, A. et al. Tyrosine phosphorylation of tau regulates its interactions with Fyn SH2 domains, but not SH3 domains, altering the cellular localization of tau. FEBS J. 278, 2927-2937 (2011).
Jeganathan, S. et al. Proline-directed pseudo-phosphorylation at AT8 and PHF1 epitopes induces a compaction of the paperclip folding of Tau and generates a pathological (MC-1) conformation. J Biol. Chem. 283, 32066-32076 (2008).
Klein, C. et al. Process outgrowth of oligodendrocytes is promoted by interaction of fyn kinase with the cytoskeletal protein tau. J Neurosci 22, 698-707 (2002).
Andersen, T. C. B. et al. The SH3 domains of the protein kinases ITK and LCK compete for adjacent sites on T cell-specific adapter protein. J Biol Chem 294, 15480-15494 (2019).
Ikegami, S., Harada, A. & Hirokawa, N. Muscle weakness, hyperactivity, and impairment in fear conditioning in tau-deficient mice. Neurosci. Lett. 279, 129-132 (2000).
Lei, P. et al. Motor and cognitive deficits in aged tau knockout mice in two background strains. Mol Neurodegener 9, 29 (2014).
van Dyck, C. H. et al. Effect of AZD0530 on Cerebral Metabolic Decline in Alzheimer Disease: A Randomized Clinical Trial. JAMA Neurol 76, 1219-1229 (2019).
Tang, S. J. et al. Fyn kinase inhibition reduces protein aggregation, increases synapse density and improves memory in transgenic and traumatic Tauopathy. Acta Neuropathologica Communications 8, 96 (2020).
Palop, J. J. & Mucke, L. Network abnormalities and intemeuron dysfunction in Alzheimer disease. Nat Rev Neurosci 17, 777-792 (2016).
Studier, F. W. Protein production by auto-induction in high density shaking cultures. Protein Expr Purif 41, 207-34 (2005).
Arold, S. T. et al. The role of the Src homology 3-Src homology 2 interface in the regulation of Src kinases. J Biol Chem 276, 17199-205 (2001).
Case, D. A. et al. AMBER 2015. (University of California, San Francisco. 2015).
Maier, J. A. et al. ffl4SB: Improving the Accuracy of Protein Side Chain and Backbone Parameters from ff99SB. J Chem Theory Comput 11, 3696-3713 (2015).
Wang, J., Wolf, R. M., Caldwell, J. W., Kollman, P. A. & Case, D. A. Development and testing of a general amber force field. . Comput Chem 25, 1157-74 (2004).
Roe, D. R. & Cheatham, T. E., 3rd. PTRAJ and CPPTRAJ: Software for Processing and Analysis of Molecular Dynamics Trajectory Data. J Chem Theory Comput 9, 3084-95 (2013).
Schrodinger, LLC. The PyMOL Molecular Graphics System, Version 1.8. (2015).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound having a structure represented by a formula: 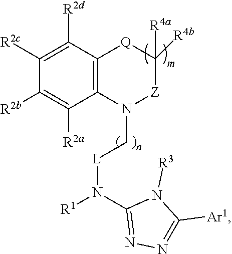
wherein each of n and m is independently 0, 1, or 2;
wherein Q is selected from the group consisting of —O—, —S—, and —CH₂—;
wherein Z is selected from the group consisting of —C(O)—, —CH(OH)—, and —CH₂—;
wherein L is selected from the group consisting of —C(O)— and —CH₂—;
wherein each of R¹ and R³ is independently selected from the group consisting of hydrogen and C1-C4 alkyl;
wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³;
wherein each occurrence of R²⁰, R²¹, R²², and R²³, when present, is independently selected from the group consisting of hydrogen and C1-C4 allyl; and
wherein each occurrence of R^{4a} and R^{4b}, when present, is independently selected from hydrogen, C1-C4 allyl, and C1-C4 haloalkyl;
wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and
wherein each occurrence of R²⁴, R²⁵, R²⁶, and R²⁷, when present, is independently selected from the group consisting of hydrogen and C1-C4 allyl,
provided that when Q is —O— then Z is —CH(OH)— or —CH₂—, and
provided that when Q is —O— Z is —CH₂—, then each of R^{2a}, R^{2b}, R^{2c}, and R^{2d} is hydrogen, and Ar¹ is 2-pyridinyl, and
provided that when Q is —CH₂ then Z is —C(O)—, and
provided that when n is 1 or 2, m is 1, L is —C(O)—, Q is —S—, and Ar¹ is 2-pyridinyl, then Z is —CH₂—,
or a pharmaceutically acceptable salt thereof.

2. The compound of claim 1, wherein each of n and m is 1.

3. (canceled)

4. The compound of claim 1, wherein Q is —S—.

5. (canceled)

6. The compound of claim 1, wherein Z is —CH₂—.

7. The compound of claim 1, wherein L is —C(O)—.

8. The compound of claim 1, wherein each of R¹ and R³ is hydrogen.

9. The compound of claim 1, wherein each of R^{2a}, R^{2b}, R^{2c}, and R^{2d} is selected from the group consisting of hydrogen and halogen.

10. (canceled)

11. The compound of claim 1, wherein each of R^{2a} and R^{2d} is hydrogen and each of R^{2b} and R^{2c} is independently selected from the group consisting of hydrogen, halogen, —CN, —NH₂, —OH, —NO₂, C1-C4 alkyl, C2-C4 alkenyl, C1-C4 haloalkyl, C1-C4 cyanoallyl, C1-C4 hydroxyalkyl, C1-C4 haloalloxy, C1-C4 alloxy, C1-C4 alkylamino, (C1-C4)(C1-C4) dialkylamino, C1-C4 aminoalkyl, —C(O)R²⁰, —CO₂R²¹, —C(O)NR²², and —SO₂R²³.

12. The compound of claim 1, wherein each occurrence of R^{4a} and R^{4b}, when present, is hydrogen.

13. The compound of claim 1, wherein Ar¹ is heteroaryl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷.

14. (canceled)

15. The compound of claim 1, wherein Ar¹ is 2-pyridinyl substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷.

16. The compound of claim 1, wherein the compound has a structure represented by a formula: 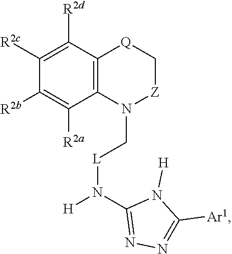
or a pharmaceutically acceptable salt thereof.

17. The compound of claim 1, wherein the compound has a structure represented by a formula: 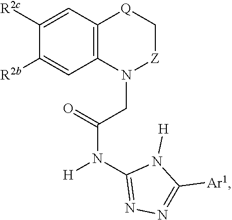
or a pharmaceutically acceptable salt thereof.

18. The compound of claim 1, wherein the compound has a structure represented by a formula: 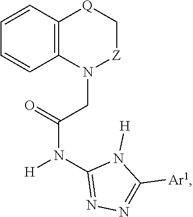
or a pharmaceutically acceptable salt thereof.

19. The compound of claim 1, wherein the compound has a structure represented by a formula: 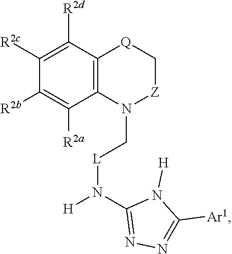
or a pharmaceutically acceptable salt thereof.

20. The compound of claim 1, wherein the compound has a structure selected from the group consisting of: 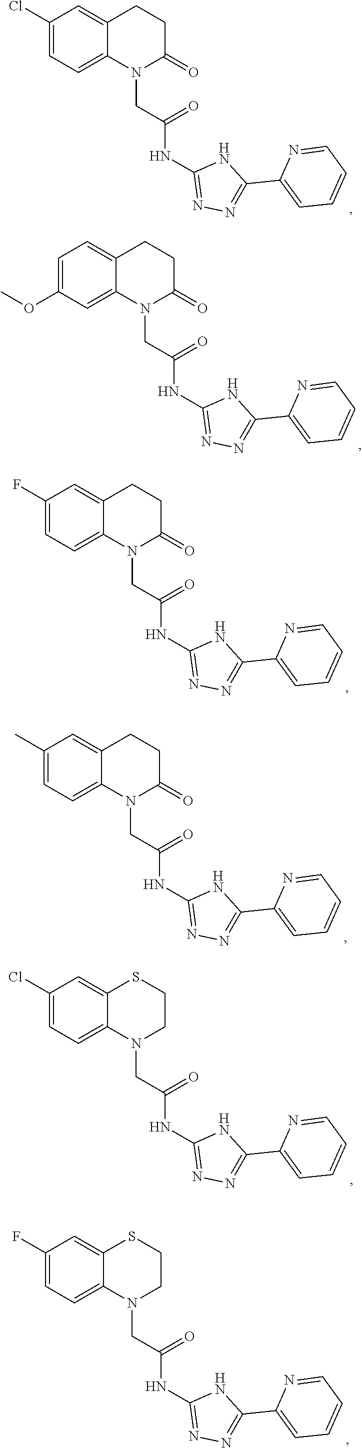 [SMILES: O=C(CN1C(=O)CCc2cc(Cl)ccc21)Nc1nnc(-c2ccccn2)[nH]1 ; COc1ccc2c(c1)N(CC(=O)Nc1nnc(-c3ccccn3)[nH]1)C(=O)CC2 ; O=C(CN1C(=O)CCc2cc(F)ccc21)Nc1nnc(-c2ccccn2)[nH]1 ; Cc1ccc2c(c1)CCC(=O)N2CC(=O)Nc1nnc(-c2ccccn2)[nH]1 ; O=C(CN1CCSc2cc(Cl)ccc21)Nc1nnc(-c2ccccn2)[nH]1 ; O=C(CN1CCSc2cc(F)ccc21)Nc1nnc(-c2ccccn2)[nH]1] 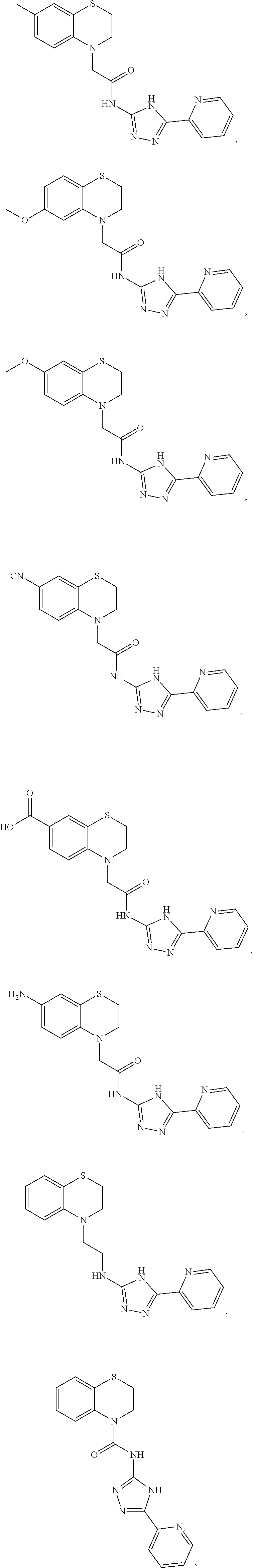 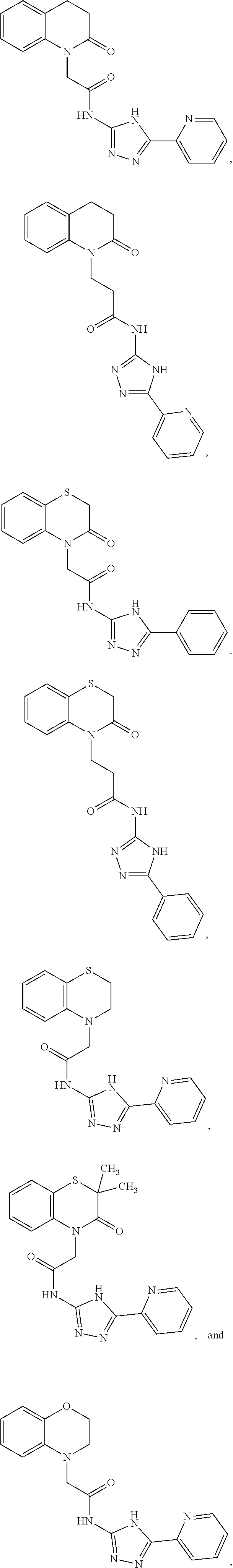
or a pharmaceutically acceptable salt thereof.

21. The compound of claim 1, wherein the compound is: 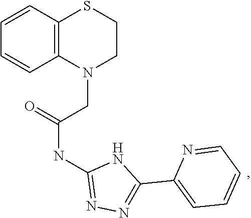 [SMILES: O=C(CN1CCSc2ccccc21)Nc1nnc(-c2ccccn2)[nH]1]
or a pharmaceutically acceptable salt thereof.

22. A pharmaceutical composition comprising an effective amount of the compound of claim 1 or a pharmaceutically acceptable salt thereof, and a pharmaceutically acceptable carrier.

23-68. (canceled)

69. A method for the treatment of a neurological disorder in a subject, the method comprising the step of administering to the subject an effective amount of a compound having a structure represented by a formula: 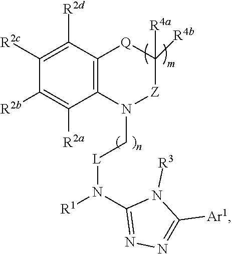
wherein each of n and m is independently 0, 1, or 2;
wherein Q is selected from the group consisting of —O—, —S—, and —CH₂—;
wherein Z is selected from the group consisting of —C(O)—, —CH(OH)—, and —CH₂—;
wherein L is selected from the group consisting of —C(O)— and —CH₂—;
wherein each of R¹ and R³ is independently selected from the group consisting of hydrogen and C1-C4 alkyl;
wherein each of R^{2a}, R^{2b}, R^{2c}, and R^{2d} is independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁰, —CO₂R²¹, —C(O)NR²², and —SO₂R²³;
wherein each occurrence of R²⁰, R²¹, R²², and R²³, when present, is independently selected from the group consisting of hydrogen and C1-C4 allyl; and
wherein each occurrence of R^{4a} and R^{4b}, when present, is independently selected from hydrogen, C1-C4 allyl, and C1-C4 haloalkyl;
wherein Ar¹ is selected from the group consisting of aryl and heteroaryl, and is substituted with 0, 1, 2, or 3 groups independently selected from the group consisting of hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, C1-C4 aminoalkyl, —C(O)R²⁴, —CO₂R²⁵, —C(O)NR²⁶, and —SO₂R²⁷; and
wherein each occurrence of R²⁴, R²⁵, R²⁶, and R²⁷, when present, is independently selected from the group consisting of hydrogen and C1-C4 allyl,
or a pharmaceutically acceptable salt thereof.

70. The method of claim 69, wherein the compound is selected from: 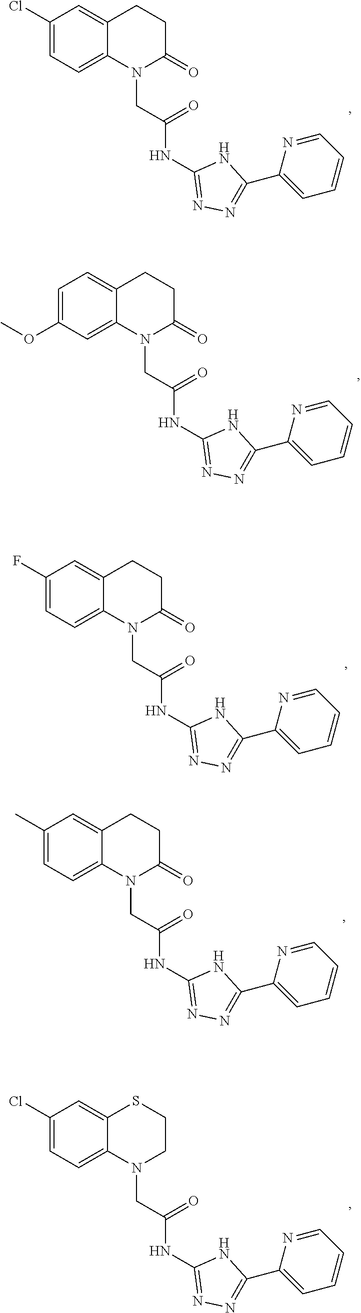 [SMILES: O=C(CN1C(=O)CCc2cc(Cl)ccc21)Nc1nnc(-c2ccccn2)[nH]1 ; COc1ccc2c(c1)N(CC(=O)Nc1nnc(-c3ccccn3)[nH]1)C(=O)CC2 ; O=C(CN1C(=O)CCc2cc(F)ccc21)Nc1nnc(-c2ccccn2)[nH]1 ; Cc1ccc2c(c1)CCC(=O)N2CC(=O)Nc1nnc(-c2ccccn2)[nH]1 ; O=C(CN1CCSc2cc(Cl)ccc21)Nc1nnc(-c2ccccn2)[nH]1] 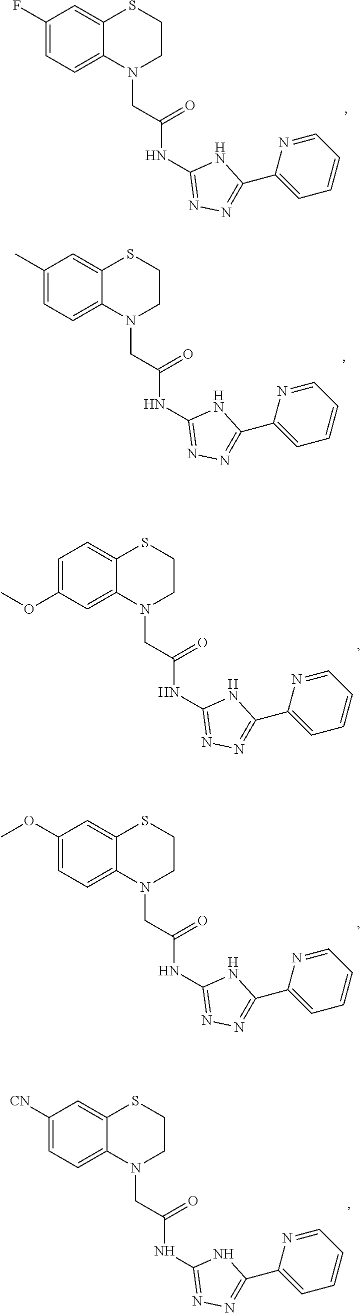 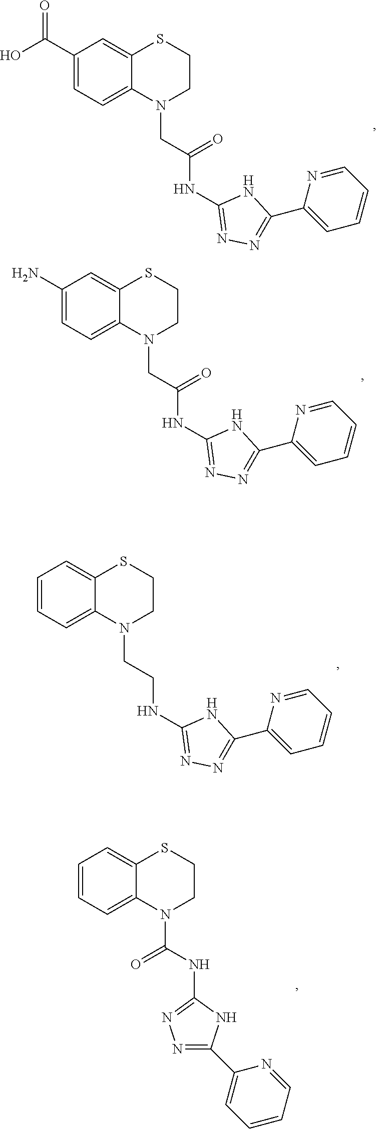 [SMILES: O=C(CN1CCSc2cc(C(=O)O)ccc21)Nc1nnc(-c2ccccn2)[nH]1 ; Nc1ccc2c(c1)SCCN2CC(=O)Nc1nnc(-c2ccccn2)[nH]1 ; c1ccc(-c2nnc(NCCN3CCSc4ccccc43)[nH]2)nc1 ; O=C(Nc1nnc(-c2ccccn2)[nH]1)N1CCSc2ccccc21] 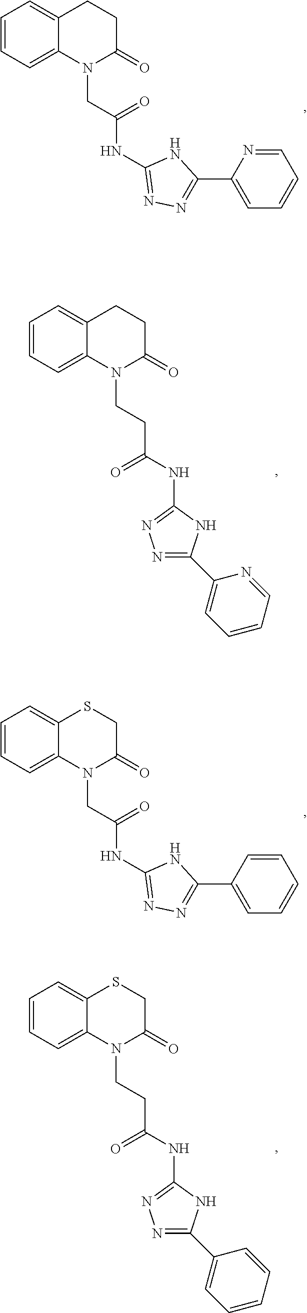 [SMILES: O=C(CN1C(=O)CCc2ccccc21)Nc1nnc(-c2ccccn2)[nH]1 ; O=C(CCN1C(=O)CCc2ccccc21)Nc1nnc(-c2ccccn2)[nH]1 ; O=C(CN1C(=O)CSc2ccccc21)Nc1nnc(-c2ccccc2)[nH]1 ; O=C(CCN1C(=O)CSc2ccccc21)Nc1nnc(-c2ccccc2)[nH]1] 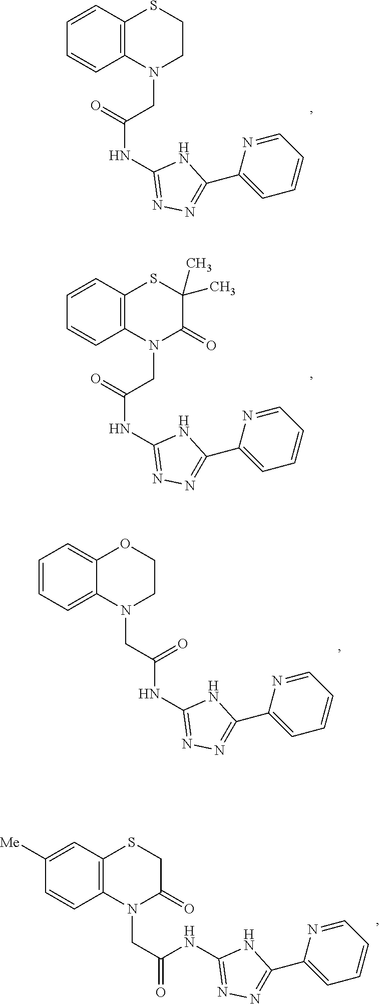 [SMILES: O=C(CN1CCSc2ccccc21)Nc1nnc(-c2ccccn2)[nH]1 ; CC1(C)Sc2ccccc2N(CC(=O)Nc2nnc(-c3ccccn3)[nH]2)C1=O ; O=C(CN1CCOc2ccccc21)Nc1nnc(-c2ccccn2)[nH]1 ; Cc1ccc2c(c1)SCC(=O)N2CC(=O)Nc1nnc(-c2ccccn2)[nH]1] 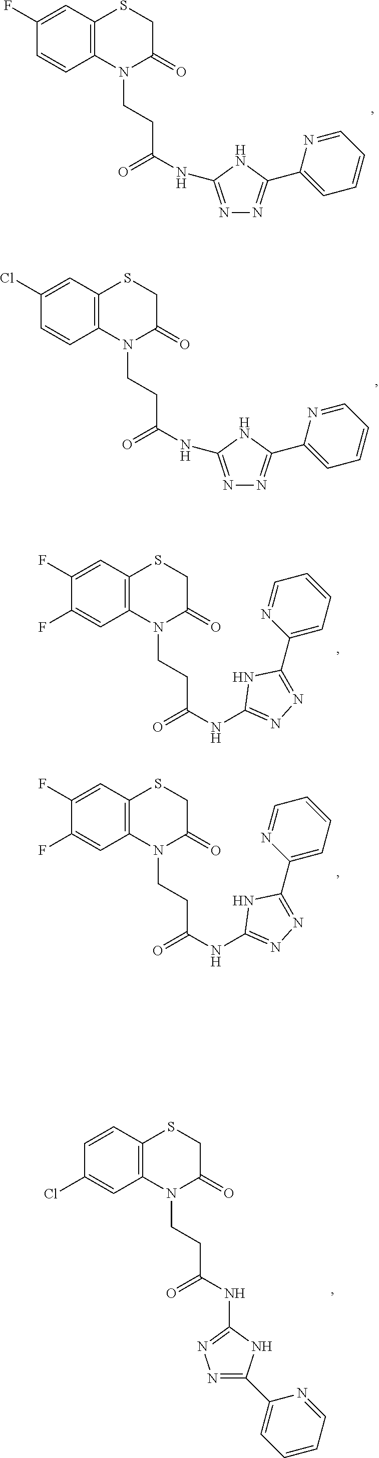 [SMILES: O=C(CCN1C(=O)CSc2cc(F)ccc21)Nc1nnc(-c2ccccn2)[nH]1 ; O=C(CCN1C(=O)CSc2cc(Cl)ccc21)Nc1nnc(-c2ccccn2)[nH]1 ; O=C(CCN1C(=O)CSc2cc(F)c(F)cc21)Nc1nnc(-c2ccccn2)[nH]1 ; O=C(CCN1C(=O)CSc2cc(F)c(F)cc21)Nc1nnc(-c2ccccn2)[nH]1 ; O=C(CCN1C(=O)CSc2ccc(Cl)cc21)Nc1nnc(-c2ccccn2)[nH]1] 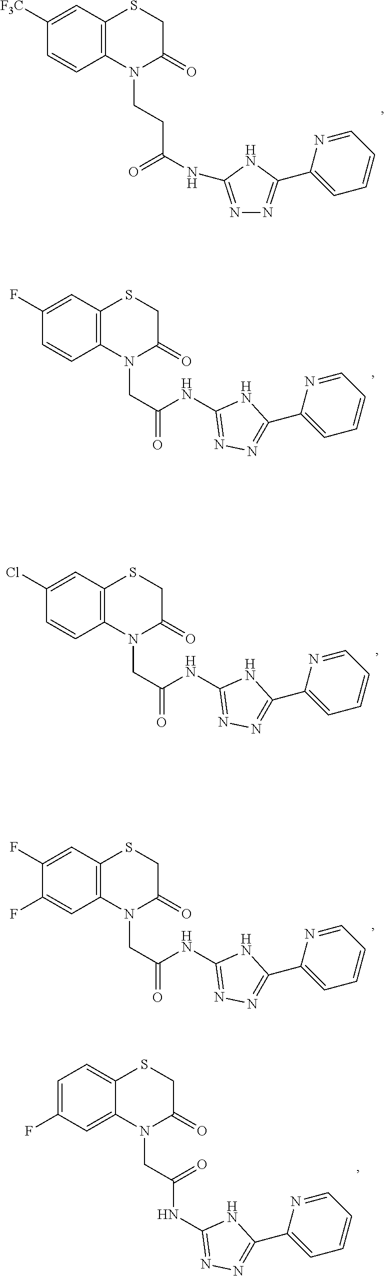 [SMILES: O=C(CCN1C(=O)CSc2cc(C(F)(F)F)ccc21)Nc1nnc(-c2ccccn2)[nH]1 ; O=C(CN1C(=O)CSc2cc(F)ccc21)Nc1nnc(-c2ccccn2)[nH]1 ; O=C(CN1C(=O)CSc2cc(Cl)ccc21)Nc1nnc(-c2ccccn2)[nH]1 ; O=C(CN1C(=O)CSc2cc(F)c(F)cc21)Nc1nnc(-c2ccccn2)[nH]1 ; O=C(CN1C(=O)CSc2ccc(F)cc21)Nc1nnc(-c2ccccn2)[nH]1] 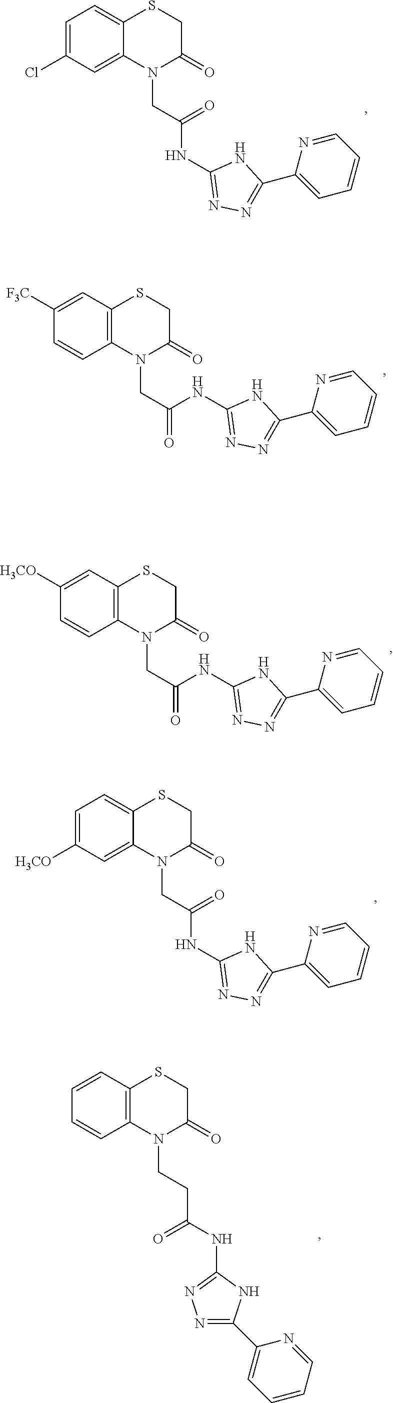 [SMILES: O=C(CN1C(=O)CSc2ccc(Cl)cc21)Nc1nnc(-c2ccccn2)[nH]1 ; O=C(CN1C(=O)CSc2cc(C(F)(F)F)ccc21)Nc1nnc(-c2ccccn2)[nH]1 ; COc1ccc2c(c1)SCC(=O)N2CC(=O)Nc1nnc(-c2ccccn2)[nH]1 ; COc1ccc2c(c1)N(CC(=O)Nc1nnc(-c3ccccn3)[nH]1)C(=O)CS2 ; O=C(CCN1C(=O)CSc2ccccc21)Nc1nnc(-c2ccccn2)[nH]1] 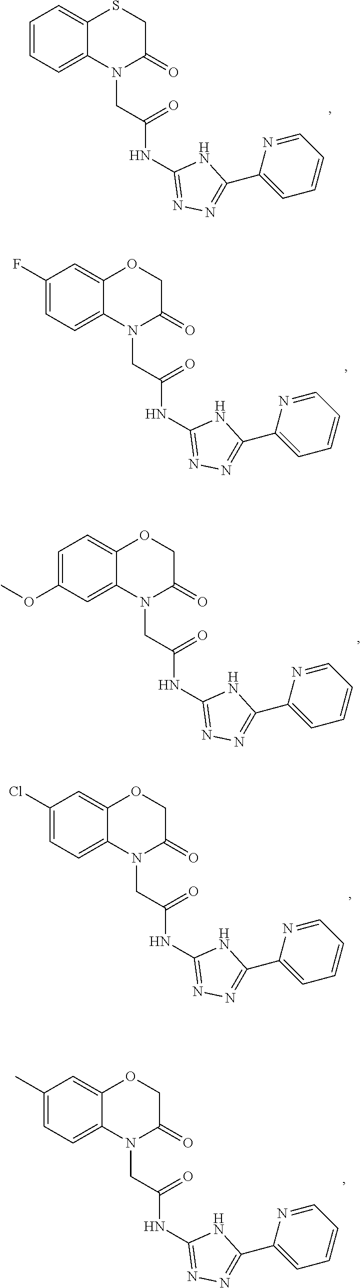 [SMILES: O=C(CN1C(=O)CSc2ccccc21)Nc1nnc(-c2ccccn2)[nH]1 ; O=C(CN1C(=O)COc2cc(F)ccc21)Nc1nnc(-c2ccccn2)[nH]1 ; COc1ccc2c(c1)N(CC(=O)Nc1nnc(-c3ccccn3)[nH]1)C(=O)CO2 ; O=C(CN1C(=O)COc2cc(Cl)ccc21)Nc1nnc(-c2ccccn2)[nH]1 ; Cc1ccc2c(c1)OCC(=O)N2CC(=O)Nc1nnc(-c2ccccn2)[nH]1] 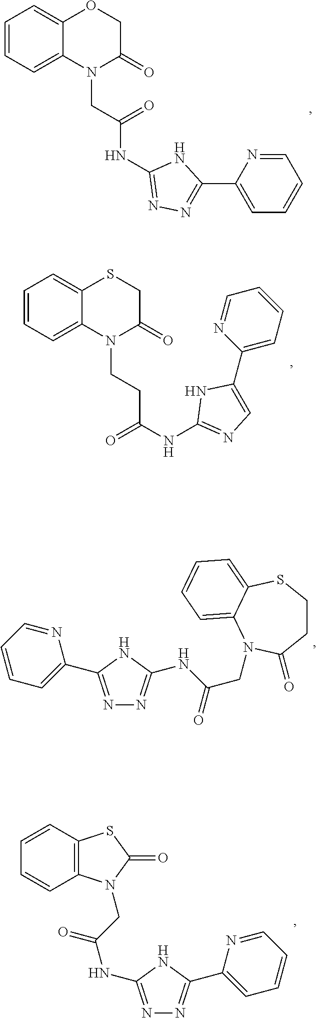 [SMILES: O=C(CN1C(=O)COc2ccccc21)Nc1nnc(-c2ccccn2)[nH]1 ; O=C(CCN1C(=O)CSc2ccccc21)Nc1ncc(-c2ccccn2)[nH]1 ; O=C(CN1C(=O)CCSc2ccccc21)Nc1nnc(-c2ccccn2)[nH]1 ; O=C(Cn1c(=O)sc2ccccc21)Nc1nnc(-c2ccccn2)[nH]1] 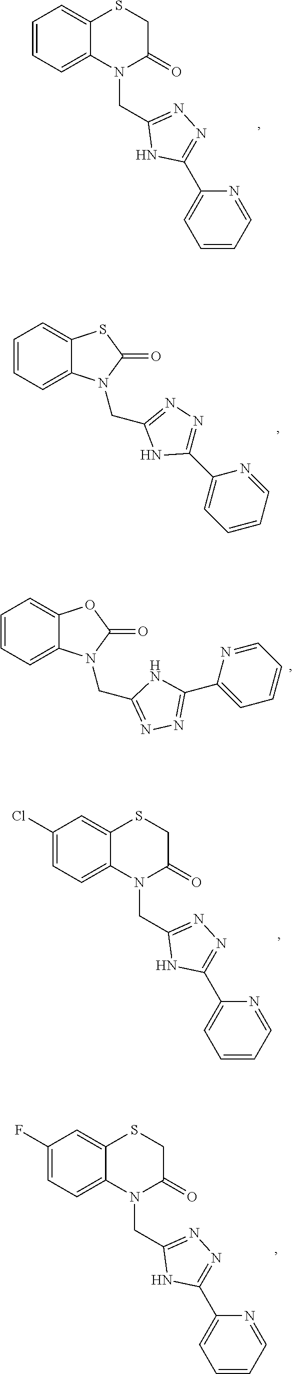 [SMILES: O=C1CSc2ccccc2N1Cc1nnc(-c2ccccn2)[nH]1 ; O=c1sc2ccccc2n1Cc1nnc(-c2ccccn2)[nH]1 ; O=c1oc2ccccc2n1Cc1nnc(-c2ccccn2)[nH]1 ; O=C1CSc2cc(Cl)ccc2N1Cc1nnc(-c2ccccn2)[nH]1 ; O=C1CSc2cc(F)ccc2N1Cc1nnc(-c2ccccn2)[nH]1] 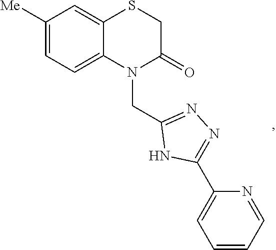 [SMILES: Cc1ccc2c(c1)SCC(=O)N2Cc1nnc(-c2ccccn2)[nH]1] 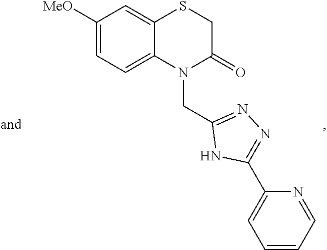 [SMILES: COc1ccc2c(c1)SCC(=O)N2Cc1nnc(-c2ccccn2)[nH]1]
or a pharmaceutically acceptable salt thereof.

71. (canceled)
